SEQUENCE LISTING (659 sequences)

SEQ ID NO: 1 - DNA, 18001 bp, Homo sapien
gaagtcatcagcaatgcaactgttcacatggaggatactccctgcttgaggggtcagacaggcctgctgggcaacccaggaggcttggatgaccgtctaccccagtgtttttgggatggaaagttccacattctgagaaccctcagtccctgggcaacctggggtggttagtcaccacagcttgtggcttgggcccatgacagcaggtagaaatgacgtggactgccgccagccgggcacagtggctcacgcctgtaatcccagcactttgggaggctgaggcatgtggatcacttgaggtcaggagttcgaaaccagcctggtcaacacggtgaaaccccatctctgctaaaaaaaaaaaatatatatatataaattagccaggcatggtgacgtgcacctgtggtcccagctactcaggaggctgaggcacaagaatcacttgaacccgggaggtggaggttgcagtgagattgcaccagtgcactctccagcctggcaacagagcaagactctgtctcaaacaaacaaaacaaaacaaacaaaaagacgtaagatgtggaccgctggagaatgggggtgctgcctgcagtcaaaacggagtgggggtgcccagctcagggccagaatgatcctattcccggcacttctcagtgaggctctgtggctcacctaagaaaccagcctcccttgcaggcaacggcctagctggcctggtctggaggctctcttcaaatatttacatccacacccaagatacagtcttgagatttgactcgcatgattgctatgggacaagttttcatctgcagtttaaatctgtttcccaacttacattaggggtttggaattctagatcgtatttgaagtgttggtgccacacacaccttaacacctgcacgctggcaacaaaaccgtccgctctgcagcacagctggggtcacctgacctttctcctgtcccccccacttgagctcagtggctgggcagcaggggatgcatggccactggccggccaggtgcagctctcagctggggtgttcagaggacgcctgtgtcctcccctcccccatccctctgtcacccttggaggcagagaactttgcccgtcagtcccatggggaatgtcaacaggcaggggcagcactgcagagatttcatcatggtctcccaggccctcaggctcctctgccttctgcttgggcttcagggctgcctggctgcaggtgcgtccggggaggttttctccataaacttggtggaagggcagtgggcaaatccaggagccagcccgggcttcccaaaccccgcccttgctccggacacccccatccaccaggagggttttctggcggctcctgttcaatttctttccttctagaaaccagcatccaggcacaggaggggaggcccttcttggtggcccaggctttggcgggattatttttcaaagaactttaggagtgggtggtgctttcctggcccccatgggcccctgcctgtgaggtcggacaagcgcagggagtctggggcctctcagagtgcaggaagtgcgcacagggtgctcccaggctggggagcacaggtaggggacggtgcgtgggggatggcgcctggggcatgggggatggggtgtgggaaacggcatgtggggcgtaggggatggggtgtggaggatcgggggtggggatggcgtgtggggtgtgggggatgggccgtgggggggtggggcctgggaaacagcatgtggggcatggggtgtgggggtgaggtgtgggaaagtgtgtggggtgtgggggatggggcatggaaagggcgtgtggggtgcaggggatggggcatggaggtgtgggggatggggtgtgtggggtgtcggggatggggcatgtggggtgtgggggatggggcatggaaagggcgtgtggggtgcagaggatggggcatgggggggtggggatggcgagtggggctggggcctgggaatggtgagtggggcatggggatggcgagtagggggtgtggcgtgaggatggctagtggggcgtggggatggcgtgtggggatggcgagtggggggtgggctgtgagggacagtgcctgggatgtggggctgcagccctagctcacagcatggccttatgaccccggccaccttcctgccccaggcggggtcgctaaggcctcaggaggagaaacacgggacatgccgtggaagccggggcctcacagaggtgagcagggactgccactggttttgtcctggggcccagtgggggccaacatcacctccttcccctcccatggcaaagagccagcccgcggggtggctactgcagtgccccccaaggagggtgttccctgctcgagaggaagtgaccgctccagcttggccttccctgggactggggtgcaggcgattttatcttctttgctccattctgttccttccagataatcgtgtgttcttcatcaggttttcctcagttcttgagagcttttctgatgcaaatctgctttcaccccagggcggtcaccggctctgctcacaccagcctccaagggtgtgggtgtcccgggagtgtgggtgtcccgggggcgtgggtgtcccaggagtgtgggtgtcccgggggcgtgggtgtcccgggagtgtgggtgtcccgggggcgtgggtgtcccgggagtgtgggtgtcccggaggcgagggtgtcccgggagtgtgggtgtcccgggggagtgggtgtcccgggagtgtgggtgtcccggaggcgagggtgtcccgggagtgtgggtgtcccgggggcgtgggtgtcccgggagtgtgggtgtcccgggggagtgggtgtcccgggagtgtgggtgtcccggaggcgagggtgtcccgggagtgtgggtgtcccgggggagtgggtgtcccgggagtgtgggtgtcccggaggcgagggtgtcccgggagtgtgggtgtcccgggggcgtgggtgtcccgggagcgtgggtgtcccgggggcgtgggtgtcccgggagtgtgggtgtcccgggggcgtgggtgtcccgggagtgtgggtgtcccgggggcgtgggtgtcccgggagtgtgggtgtcccgggagtgtgggtgttccggaggcgagggtgtcccgggagtgtgcgtgtcccgggggcgtgggtgtcccgggggcgtgggtgtcccgggggcgtgggtgttccggaggcgagggtatcccagaagtgtgagtgtcccaggggcgtgggtgtcccgggggtgtgggtgtcccgggggcgtgggtgtcccgggagtgtgggtgttccggaggtgagggtgtcccgggagtgtgggtgttccggaggcgagggtgtcccgggagtgtgggtgtcccgggggcgtgggtgtcccgggagtgtgggtgttccggaggtgagggtgtcccgggagtgtgggtgttccggaggcgagggtgtcccgggagtgtgggtgtcccaggggcgtgggtgtcccgggagtgtgggtgttccggaggcgagggtgtcccgggagtgtgggtgttccggaggcgagggtgtcccgggagtgtgggtgtcccgggggcgtgggtgtcccgggggttgtgggtgtcccgggagtgtgggtgttccggaggcgagggtgtcccgggagtgtgggtgttccggaggcgagggtgtcccgggagtgtgggtgtcccgggggtgtgggtgtcccgggggtgtgggtgtcccgggagtgtgggtgtcccgggggagtgggtgtcccgggagtgtgggtgttccggaggcgagggtgtcccaggagcgtgggtgtcccggaggcgagggtgtcccgggagcgtgggtgtcccgggggcgtgggtgtcccgggagtgtgggtgtcccgggggagtgggtgtcccgggagtgtgggtgtcccggaggcgagggtgtcccaggagtgtgggtgtcccgggggcgtgggtgtcccgggagtgtgggtgttccagaggcgagggtatcccagaagtgtgagtgtcccgggggtgtgggtgtcccgggggtcgtgggtgtcccgggagtgtgggtgttccagaggcgagggtgtcccgggagtgtgggtgtcccaggggtgtgggtgtcccgggggcgtgggtgtcccgggagtgtgggtgtcccgggggagtgggtgtcccgggagtgtgggtgttccggaggcgagggtgtcccgggagtgtgggtgttccggaggcgagggtgtcccgggagcgtgggtgtcccgggggcgtgggtgtcccgggagcgtgggtgtcccaggggtgtgggtgtcccgggggcgtgggtgtcccgggagtgtgggtgtcccgggggagtggatgtcccgggagtgtgggtgttccggaggcgagggtgtcccgggagtgtgggtgttccggaggcgagggtgtcccgggagtgtgggtgtcccgggggcgtgggtgtcccgggagtgtgggtgtcccgggggcgtgggtatcccagaagtgtgagtgtcccaggggcgtgggtgtccggggggcgtgggtgtcccgggggtgtgggtgtcccgggggtcgtgggtgtcccgggagcgtgggtgtcggggactgcagggacatgggcctcccctcccactcctgccgcccagggcacctcctgtgaggactcggagtccgtgagttcccacctccttgagcccgattctttggtgtccccgcctgcatcctcagcctccttccaaaccagaccagttctctaggggcgtcgacgtgtgaaactgattttaaagaaaacaggcagtggcctttctctcggccccacgtggcccagtagcgctcaccttccgtcccttcttccgcgctcagtaaccaatttaggccgctcctgcagaactcgggctcctgcccaccggcccacagcgtccacctgaggcctcgtcctcccagcaaaggtcgtccctccggaacgcgcctcctgcggcctctccagagcccctcccgcgcgtcctctcagccccgctcgcctcctcccggggcctccctctcccgcctgcccccaggcccgtctcccctcgcgggctgaggcaggttcgggcagcacggccgccccggggcgggggtcactctccaccaccgcgtggtgcccacagctcacggcgctcccgggtgacggtcccctcggctgtagggcgtcctgaagagcggcctgctcggagctgagcgcacggggttgcctcgccctgggcgtctctggccctcaccagccccgtcttcccatgggcaaaacggcggtcctgtttgtccacaagtaaccgtcggggttacggaggggccaggagctgcggcggggggctgtgctctcaggaccggccccaggaggatccgcgcgaggtctggagctctcaggggtcgcgggggacagaggggccccaagcggaggcggggaaggcggcagaagcccaggaccgccaagagctggcgaggaagcccggggctcgctgtcgggggagccgggcaggggccgcgcctcggaccaggacggaggcctggggaaggcggatctggccgccggagacgcggtgcgggtggagacgagggatttggatttccgcgggcggctgtacggatttccacgcgcggttcacgtgggccccagggggttgcccggcacccggggccgcgccgccttctcctcgccggcatcgacccgcagcctcacgtttacgcggcggcgcccgcagcccccttcggcccggcttccgcgcgtgcccccgagcgcgccctcgggatcagcccccggaagcagagaggccaggccgggaaggatgggcgacgggggtggctgacccgggagcacggcagggaggacacccagccaggcccgcgagcagcgccgctcccctcctccaggacgggcgggaacctgcgatgcccccgccgcgtgggccgtggggcggtctccgaggcactgggcggggcacgcggtgggcgcttcacggaactcgcatttcccagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacggtgagcccagcctcggggcgccccgcgccgcggacactgcaggcggcggtgaaccaggccgcgtggggccgcctgcgtctctttggctgcggctgtgggcggcgaacacgcagcggcgcccgcgcggcgctttctgcgggggtcgctttccgcccggggtgactccgctttcctgggcgatgccccccacccccaggcacgcgctctccccgtgcggccgcaccgcgcatgccggttttcacatcagaaaatacgatttgcaaagcacacttagggtgtcccccttaacttcccaagggagtccccccagtccccgaagggtccagggcagcctgcgcatcgcagacgcgcgcggctcgcagaagggacgtggtgagaagctggcccacagcatgccaccagcggcacctcctcagggcacgtgtcggggagaaacaacacttagggacctgggactttctccagctcacgctcacgggtcacctcacactccaagatcacctcaaagaggacacctcacacagggcacacttcacactcacaggtcacctcacactcacaggacacctcacactcacagggcacacttcacactcacgggtcacctcacactccaagatcacctcaaagaggacacctcacacagggcacacttcacactcacaggtcacctcacactcacaggacacctcacactcacagggcacacttcacactcacgggtcacctcacactccaagatcacctcaaagaggacacctcacacagggcacacttcacactcacgggtcacctcacactcacaggacacctcacacaagacacctcacacggggcacacttcacactcacaggtcacctcacacccacaggacacctcacacagggcacacttcacactcacgggtcacctcacactcacaggacacctcacacaagacacctcacacggggcacacttcacactcacaggtcacctcacacccacaggacacctcacacagggcacacttcacactcacgggtcacctcacactcacaggacacctcacactcagggcgcacttcacactcacgggtcacctcacacccacaggacacctcacagaggtcacctcacacaggacacctcacactcagggtgcacttcaaacccacaggtcatttcacctcacactcacaggacacctcacacaagataccacacggggcacacttcacactcacaggtcacctcacactcacaggacacctcacagaggtcacctcacacggggcacacttcacactcacaggtcacctcacacccacaggacacctcacagaggtcacctcacacccacaggacacctcacacaggacacctcacagaggtcacctcacacccacaggacacctcacactcataggtcacctcagtcttacaggacaactcacactcacaggtcaccttactctcacaggacacctcacactcacaggtcaccttactctcacaggacacctcactctcacaggacacctcacacagggcacacttcactcccacaggtcaccatacctcacacagatcacctcatactcacagatcacttcattctcacaggatacctcacactcagggcacacttcacactcacaggtcacacctcacacagatcatctcattctcacaggacacctccctctcacaggtcacctcacactcacaggacacctcacagaggtcacctcacacccacaggacacctcacagaggtcacctcacacggggcacacttcacactcaggtcacctcacacccacaggacacctcacagaggtcacctcacacccacaggacaactcacagaggtcacctcacacaggacacctcacaaaggtcacctcacacccacaggacacctcacactcataggtcacctcagtcttacaggacaactcacactcacaggtcaccttactctcacaggacacctcacactcacaggtcaccttactctcacaggacacctcacacagggcacacttcactcccacaggtcaccatacctcacacagatcacctcatactcacagatcacttcattctcacaggatacctcacactcagggcacacttcacactcacaggtcacacctcacacagatcatctcattctcacaggacacctccctctcacaggtcaccttacactcatctcacactcacaggtcgccacacctcacactcacaggatgcctcacactcacagaaccacatctcatatgcacaagacacctcacactcaggacacctcatgctcaaagaagcctcacactcacaggaggtccagctgtctgaggcaaaggctaacatgaccctttccagacaaattgaggatggtcatgcctagcatttttatacacctagttttgaaagcatttctcatctgttgtattctcacagcaccccgtgagtttaagttcaggtggccaacagtttcttcagcaatcacttttttctgtggagtgcttttgctgtttgtggaatattttgcatctgctactgcaccctctccccgtatgtgtggccaccctgtcagaggtggagctgtggctcagagcctgtgtacctcgtcccaggtccacagctcagcgacagaagagtcagggttgaacctcgggtgttctgacttgggagcaggaaatgtgtggtcacccatagttccagatgtcctggggaggggccaagattagaagaaacctacctcagctccagaggaaagtctggcttcctgagcccaccccgccagacccaggtccaagtcccccaaccccagttcatggtgtgtccagtgcttaccgttgggtgctctggtgaaggtgcatctcacgaggcttgctctcttgttccttcagaagctgttctggatttcttacagtggtgagtggatgatcaccaccagtcctgcctgcaacccttctcagcttactgacaccagcccactccacagatggggaccagtgtgcctcaagtccatgccagaatgggggctcctgcaaggaccagctccagtcctatatctgcttctgcctccctgccttcgagggccggaactgtgagacgcgtaaggccccactttgggtcccatatttgcagagggccctggggagctggtggaggtggcctggccaaccgggctgcagggtgcacaacctggtggggtgtgtaggccgggcattcagggctcagccccagttggaaattggtctaggtgaccttgaaatcccttccagtctgaggtctttgacagggacccaaggttctgattatcagactcagtggccccttgggctcccggccctgggcaattctcagccctcgagatggcccagctgagagtccctgtgtccctgtcccacttccacatcccaccacgcaggaccgcttggtaaacttccccttctctactttccattacaaaggtttgaggggtttgttttttttttaaccatctgaatattaaattatcacaaagtttgaggcccccaacctcccttgggttcagtaattcactagaaggactcatagaatccactgaagtggatacactcacaggtaccgtttattacagcaaaggatgcaggcttaagtctgcagagggaccaggcacaagcttccccttgtcctctccctgtggggtcatgtggacagtccttaattctcccagaatgacgtgtgacgagacgtgggaagtactgccaacttgggaagctctacgagccccggtgtccagaggttttatcagggctcaatcacatagacccagctgaccacccgcatggctgacctcagtctcagcccctccagaggctacgccgatagtgcggcccaaggccccaccatacatcacattgtcagctagaccatccagcatggctcaaggcccaggtaaacaccaacattccctcaggcaagaccttccaagggcttagcggtcatttcccaggagccaaggcaaaggctaccctttctctggcacagcagttcatccttgaccacccaagaccacattcttacactgaatgagctctcctgtgcagcagccattttcttctctaagcagaagagagcccagcaagctggaggaggctgaagagagaggcttcctgctggtcatctgggtccagaatgcctggagatctctgctcagccctggtgcccagcagccctggtgtgcatcctgcagggcaggccttcccgccggagtcctggacttgctcagggccactccccttgcccatgtcaaccaaagtcaggctgccggttctgcttcttctgtctgagcccatgaccagtgctgggactaactgtccccaggcgggctcacggtggtacgaggccagcttggagaactgtctcagctctctggtcctctcgtcagttgggtctctgattggaaagtcccttggacacttttaccatccccattggactttcactttcccccaggctcccatcagctgctcggaagagtggtcaccctggaggccactgcccaccagccaggcaccccccaaatgcaaccgcagccagcactgccagccactggcaaggctgttcagacatgtggctcctctgatccacgccttgtcctttggatcagtccacggagcaggtggtgccaagctcaggctctgtcacccacagctcagtgccaccttccaggcagaacaccactgctgacccagggcatggccaccccgggggctggctctcgctgacccccagaagcccctctcagggtgtccccttcctgtccccagacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacaggtgaccaggcttcatgtcccagtcccagatgacaccagtccctgtcccactacggattatcttactggacaaaagacgggtgggagtggcttcacatctactgagcactaactatgcactgaccaattgtgaggtgggatctgggcaccaagggtggcacaggccagagcgacagtgactaggatgggcaccctgggggcaatccctgaatggcctcaggccccctgccaattctaggcagaccaggggagccaagcaaggcactatctcacgtccaactcccactcgcaggacctccgccagggttcatgaatctacttcggcacagccaatgtctgtactgactgctgcccactctgcattccaaaactcgtaaaggctcctgggaaaatgggatgtttctccaaaccagcctggaacgaatgggctgcacttccaaaagcagggacaccccacacccactgtgtctcaaagaggcggacgtgcccaccctggccacacagcctgggactcagcctgccacctcctcgggcttcctttctggcccaagaccttgattgaagcagatcaaaactaagcatgggatcaaaacaacacagtttgattcatctttaggtagaatttcattcaccttctactaaagtcaaacaacacatcttctccctgaaaagtgagcagagggcggttttaagacgtaagccctctgtttcctccaaaaccagccctgaccattgtctcctcagccagccacttcttcaagggcctctcatggccgggccccaccagtcaggcccagccgaggccctgccttccaccacccctgggccctgggagctcctgctcctgggggcctcccatagcctcggcctcaaggcctctcagaggatgggtgtttctgaatctttcctagtggcacgttcatccctcacaaatctctgcatctttctgacttttgttttacacagttgaatatccatgtggaaaaatacctattctagaaaaaagaaatgccagcaaaccccaaggccgaattgtggggggcaaggtgtgccccaaaggggagtgtccatggcaggtaaggcttcccctggcttcaggattccaagccctgagggtcttgaagccttttgaatgtgaacaacagctctggaagggaaaatgggcaggtcagccccaagcccaccaggctccaagtcagcacacctagcacctccagctcgcggcacccccatgcttttagtggggcaaggaaggagaaaagaaaacgacactcactgagggtctaccctgtgcagagaaccctgcgagatgccccatccgagttgtcacgtcgtcctcacggttactctttgaggtgggatctttgcctgatctttgcaaaatcaggagcattggatcaaagctatgtgaagatcctgtgaggtgaacagtgaaatctcacagcgacatttgtattcttgggccgtgcccaagagcacgtctcggctagagaggggcacagcctcccagagccaggtctgagcagctttgcctgggagggatctgcaaagaccccaggatttcagaaagaaattgtgcaatgccagaggttccttggcatgcccgggagggcgagtcatcagagaaacaatgacagcaatgtgacttccacacctcctgtccccccgcccaggtcctgttgttggtgaatggagctcagttgtgtggggggaccctgatcaacaccatctgggtggtctccgcggcccactgtttcgacaaaatcaagaactggaggaacctgatcgcggtgctgggtgggtaccactctcccctgtccgaccgcggtgctgggtgggtgccactcttccctgtccgaccgcggtgctgggtgggtgccactctcccctgtccgaccgcggtgctgggtgggtgccactctcccctgtccgaccgcggtgctgggtgggtgccactctccgctgtccgaccgcggtgctgggtgggtaccactctcccctgtctgaccgcagctctcaagtgtctcaggggctgtggctctgggcttcgtgctgtcacttccacagacagacagacatccccaaaaggggagcaaccatgctgggcacgactgctgtggccaccgtgctctcagccactttcccatgcccaaataaaacgataaaagactgggggcttctgcccatcctgcctcacttgaccaagagcccagaagaggatgcgacacccagggcctcatgggaccaccggctggcaggggttctgctcactgggtttatgggtgagacgagcactcccaggagggccactgggccgggaagaactgtggagaatcggggcacgccctgtcctcccagctgccagggcacagcatcccttccccacctcaacacccagaccccagattcaccccagttcacttgtccccacacgagccacaggctgccacctggggcaggctggccccaccttggggttagatgcaggtccccttgccccagaaggagactgcagcccctgcagacctagaaatggccacagcccatccccatgcaccagggggtgaggtggcaggtggtggaaagggcctgaggggggcttcttccttccaggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctagcccagcagccctggcctgtggagagaaagccaaggctgcgtcgaactgtcctggcaccaaatcccatatattcttctgcagttaatggggtagaggagggcatgggagggagggagaggtggggagggagacagagacagaaacagagagagacagagacagagagagactgagggagagactctgaggacatggagagagactcaaagagactccaagattcaaagagactaatagagacacagagatggaatagaaaagatgagaggcagaggcagacaggcgctggacagaggggcaggggagtgccaaggttgtcctggaggcagacagcccagctgagcctccttacctcccttcagccaagcccacctgcacgtgatctgctggcctcaggctgctgctctgccttcattgctggagacagtagaggcatgaacacacatggatgcacacacacacacgccaatgcacacacacagagatatgcacacacacggatgcacacacagatggtcacacagagatacgcaaacacaccgatgcacacgcacatagagatatgcacacacagatgcacacacagatatacacatggatgcacgcacatgccaatgcacgcacacatcagtgcacacggatgcacagagatatgcacacaccgatgtgcgcacacacagatatgcacacacatggatgagcacacacacaccaatgcgcacacacaccgatgtacacacacagatgcacacacagatgcacacacaccgatgctgactccatgtgtgctgtcctctgaaggcggttgtttagctctcacttttctggttcttatccattatcatcttcacttcagacaattcagaagcatcaccatgcatggtggcgaatgcccccaaactctcccccaaatgtatttctcccttcgctgggtgccgggctgcacagactattccccacctgcttcccagcttcacaataaacggctgcgtctcctccgcacacctgtggtgcctgccacccactgggttgcccatgattcatttttggagcccccggtgctcatcctctgagatgctcttttctttcacaattttcaacatcactgaaatgaaccctcacatggaagctattttttaaaaacaaaagctgtttgatagatgtttgaggctgtagctcccaggatcctgtggaattggatgttctctccctgccacagcccttgtcaatgatatttcacagagaccctgggagcacctgctcaagagtcagggacacacgcatcactaaatgcaagttcccaggccctggctgcagtgggaggacctggcaagctgcactcttgctgagtccccagggtggtggaagaagaatgagaaacacatgaacagagaaatggggaggtgacaaacagtgcccccactcagactccggcaagcacggctcagagagtggactcgatgccatccctgcagggccgtcctgggcaccactggcactcacagcagcaaggtgggcaccattggcactcacagcagcaaggcaggcaccagcaacccacctcgggggcactcaggcatcatctacttcagagcagacagggtctatgaactacagccgtgggctgcttccaaggcaccctgctcttgtaaataaagttttatgggaacacacccatattagtgtccatggagtggccgtggcagagacgtccagccggacagaccagctgacccgccaagcccagcatggttagtgtcaggacctctgctgaagatgcttgctgaccctggccagaccccggttcctaatgccccctaaacgggacgggagccagtggcgggccctgatccaggtcagagctggctctgctttctcttttgtccgagtgaccatgcctcagtttcctcatgtgtaaaacaggagcccaccgtgatgcttatggtgggatgagatcagcatggatggaacaaggccctggaagggcccatgccatggtcatcgacagcaaagccactctgcagacagatgcttcagtgaattggtagaaaattctgcaaccagaatgcccggggctcctgagggcctaagcccagcccagggttctggaagccactctgacttcttgggagtggaagttggcaggactcttcctgggaagaagcggagggtggggatgagaggacagttcaggagcccacccagacccacaggaggaaactaggggagtcatgcggggtcctggtggagcgccagcctcccttcctgccaatgggaaatgcaggcgcccacctcatggtgctgccggaggagggggcccgggactccccagaggcttcgctgaagggcctgggcgcccccaaaggctacatgtttcatatgggacgtgccacctgccacggctcagctccagctttctgtgagtggcgagatagaatacggggaggccactggccatgggcctgggacagggtgggatgaggcggcaggcttgggccaccaaagccagcatcgccacccagcattgatgacaaagactgcgtgtctgccatgagcatcctgctgttggtgcacacaccgcattggtctctccatacaaacatgcctagaggcgatgtcagagggtggagaccaggagaggcaggagtcagacatctggtgccaccaggaaggcccttctcagaggaccaggctgtgcgtggtgcccgccgtgggaggccagcctggcgttggcatccagcatcatcagtttgtgcagtcgggtggggctcagtgagtgcctcctgtgtgccaggcacaatgacgcacaatgtgtgcacaccaggctcatgtgcaggtggctgcgagacagggcgacccatcaaggcagatgcaccatgaggcagtggccagtgctgtgggtgttaggggcattgctccccggccactacggcatagcaggcagtgatcgccacactggccaagctttagaccatttattccagagaccccagaggcaaaaagcccggctgcacctcccagtgactcccacagccattgagcagagacactcaggaccttgtgatgggaggtttctgcactggagaacgagcccagaagccctctcagcctcggaacagtgtggccagtggtgggcaggtcaggaggggcttcagacacagcctgtccctccagatggtcacgggaaggtcactccccacagaagtacgttttggggccatgcgggcacagaaggtttgggggtgggtggggcaggtgccagcctggcctgtgggaggccatggtgcagatgccaagccccccccgtgacatgagaccacctgataccacccagagagtggctgtgagcggaagggcccgcccagaaacaagcagggccttggggcagaagtcctgggctcagatcccacgctcactgccagcggcctcggctcaggcttctgcgctctctaaacttagttttctcttctggaaaaatgatggggaaaatgatatttgtatgtgaggactgagagttaaatgtaaacatctggaaactacaaaatgagcacgaaatgatgtttttattcttagaacagaaagtccccacacccgcggccctggtgactgatgaggatgaggttctgcggggcctctctggccgcccagctctgcctggggaaggtggggcca

SEQ ID NO: 2 - DNA, 3075 bp, Homo sapien
agtcccatggggaatgtcaacaggcaggggcagcactgcagagatttcatcatggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctagcccagcagccctggcctgtggagagaaagccaaggctgcgtcgaactgtcctggcaccaaatcccatatattcttctgcagttaatggggtagaggagggcatgggagggagggagaggtggggagggagacagagacagaaacagagagagacagagacagagagagactgagggagagactctgaggacatggagagagactcaaagagactccaagattcaaagagactaatagagacacagagatggaatagaaaagatgagaggcagaggcagacaggcgctggacagaggggcaggggagtgccaaggttgtcctggaggcagacagcccagctgagcctccttacctcccttcagccaagcccacctgcacgtgatctgctggcctcaggctgctgctctgccttcattgctggagacagtagaggcatgaacacacatggatgcacacacacacacgccaatgcacacacacagagatatgcacacacacggatgcacacacagatggtcacacagagatacgcaaacacaccgatgcacacgcacatagagatatgcacacacagatgcacacacagatatacacatggatgcacgcacatgccaatgcacgcacacatcagtgcacacggatgcacagagatatgcacacaccgatgtgcgcacacacagatatgcacacacatggatgagcacacacacaccaatgcgcacacacaccgatgtacacacacagatgcacacacagatgcacacacaccgatgctgactccatgtgtgctgtcctctgaaggcggttgtttagctctcacttttctggttcttatccattatcatcttcacttcagacaattcagaagcatcaccatgcatggtggcgaatgcccccaaactctcccccaaatgtatttctcccttcgctgggtgccgggctgcacagactattccccacctgcttcccagcttcacaataaacggctgcgtctcctccgcacacctgtggtgcctgccacccactgggttgcccatgattcatttttggagcccccggtgctcatcctctgagatgctcttttctttcacaattttcaacatcactgaaatgaaccctcacatggaagctattttttaaaaacaaaagctgtttgatagatgtttgaggctgtagctcccaggatcctgtggaattggatgttctctccctgccacagcccttgtcaatgatatttcacagagaccctgggagcacctgctcaagagtcagggacacacgcatcactaaatgcaagttcccaggccctggctgcagtgggaggacctggcaagctgcactcttgctgagtccccagggtggtggaagaagaatgagaaacacatgaacagagaaatggggaggtgacaaacagtgcccccactcagactccggcaagcacggctcagagagtggactcgatgccatccctgcagggccgtcctgggcaccactggcactcacagcagcaaggtgggcaccattggcactcacagcagcaaggcaggcaccagcaacccacctcgggggcactcaggcatcatctacttcagagcagacagggtctatgaactacagccgtgggctgcttccaaggcaccctgctcttgtaaataaagttttatgggaacacaaaaaaaaaaaaaaaaa

SEQ ID NO: 3 - DNA, 564 bp, Homo sapien
atcatggtctcccaggccctcaggctcctctgccttctgcttgggcttcagggctgcctggctgcaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgtggtgagtggatgatcaccaccagtcctgcctgcaacccttctcagcttactgacaccagcccactccacagatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaata

SEQ ID NO: 4 - DNA, 15001 bp, Macaca mulatta
  misc_feature (3049)..(4102): n is a, c, g, or t
tcaaaacagagtggggctgcccagctcagggccagaatgatcctattcccagcacttctcagtcaggctctgtggctcaactaagaaaccggcctcccttgcagacaatggcctagctggcctggtctcctggaggctctcttcaaatatttacatccacacccaagatatgctctccagaattgactcgcattattgctatgggccaagttttcatctgcagtttaaatctgtttcccaacctacgttcctatgtcctaggggtttggaattctagatcgtatttgaagtgttggtgtcacacacacaccttaacacctgcacgctggcaacaaaaccatccgctttgcagcacaactggggccgcctgacctttctcctgtcctccctgcttgagctcagcagctgggcagcaggggatgcatggccactggccggccaggtgcagctctcagctggggtgttcagaggacgcctctgtccccccctcccccatccctctgtcgcccttggaggcagagaactttgcccgccagtcccatgcggaatgtcaacaggcagaggcagcgctgcagagatttcatcatggtctctcgagccctcgggctcctctgccttctgcttgggcttcagggctgtctggctgcaggtgcgtccggggagattttccccataaacttggtggaagggcagtgggcaaatccaggagccgacccgggcttcccaaaccgtccttgctctggacacccccattcaccaggagggttttctggtggctcctgttcaattgttttccttccagaaaccagcatccaggcacaggaggggaggcccttcttagtagcccaggctttggtgggattatttttcaaagaactttaggagtgggtggtgctttcttggcccccatgggcccctgcctgttaggttggacaagcacagggagtcgggggcctctcagagtatgggaggtgctcacaggctgctcccaggctggggaggacaagtgtgtgggggatggtgcctggggcatgggggatggggtgtggaggatgggggttggggatggcatgtggggtgtggaggatgggccatgagggggtgggtcctgggaaacggtatgtggggtatgagggatggggcgtggggtgcgggaggggggtgtgggaaagtgtgtggggtgtgggggatgggatgtgggaagtggcatgtggagtgcaaggaatggggcatggaggtgttgagcatggggtgtgtcgggtgtgtggggtgtgggggagggggaatggaaagggtgtgtggtgtgtgggggatggggtgaggggatggcgtgggaggtggggcatggggatggcaggtgtggcgtggggatggcgagtagggggtggggcgtggggatggtgactgtggggtggggatggcgagtggggctggggcctgggaatggtgagtggggtggggatggcgagtacagggtgtggcatggggatggcgaatggggcatgaggatggcgtgtggggatggcgagcaggggggtgggctgtgagggacagtgcctgagatgtggggctgcagccccagctcacacatggccttatgaccccagccaccttcctgccccaggcggggtcgctgaggcctcaggaggagaaaacacaggacctgctgtggaagccagggcctcacagaggtgagcagggactgccactggtttagtcccggggcccagtgggggccaacatcacctccttggcctcccatggcaaggagccagcccgcggggtggctactgcactgccccccaaggagggtgttccctgctcaagaggaagtgaccgctccagttcagccttccctgggactggggtgcaggtgaccttatcttctttgttaaatcctgttccttccagacaatcctgtgttattcatcaggtttgcctcagttcttgagagcttttctgatgcaaatctgctttcatcccagggcggtaggggctcagctcacgccagcctccaggggtgtgggtgtcctagaagtgtgggtgtcccgggggcgtgggtgtccctggagtgtgggtgtcctgggggcatgggtgtcccagagcgtgggtgtccctggagtgtgggtgtcccaggggcgtgggtgtcccggaggcatgggtgtcccggggcgtgggtgtcccggggcgtgggtgtcccaggggcgtgggtgtcccggaagtgtgggtgtcccggggcgtgggtggcttgggggcatgggtgtcccgggggcgtgggtggcttgggggcgtgggtgtcccgggggtgtgggtgtcccgggagcgggtgtcccgggagtgtgagtgtcctgggggtgtgggtgtcccgggagtgtgagtgtcccaggggcctggatgtcgggggactgcagggacacccttcccactcctgctgcccggggcacctcccctgaggactccgcctccaagagctcccacctcctggattctttggtgacccccgcctgcatcctcagcctccttccaaaccagaccggttctctagggacgtggacgtgtgaaactgattttaaaggaaacagacggtggcgtttctctgggccccacgtggcccagtagcgcccaccttccgtcccttcttccgcgctcagtaaccgatttaggccgctcctgcagaactcgggctcctgcccacctaccacctgcgtccacctgaggcctcgtcctcccagcaaaggtcgtccctcccgaacgcgcctcctgcggcctctccagagcccctcccgcgcgtcctctcggcctcctcccgggcctccctctcccgcctgccccacggcccggccagtctcccctcgcgggctgaggcgggttcaggcagcgcggccgccccgggggtcactcctcgtccaccaccgcgtggtgcccacagctcacagctcccgggagacggtcccctcagctgcagggcgtcctgaagaacggcctgctcagagctgagcgcacgggcttgcctcgccctgggcgcccttggccctcgccgaccccgttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccctaccaggcacacgctctccctacgcggccactccgcgcatgccggttttcacatcagaaaatacgatttgaaaagcacacttagggtgtcccccttaacttcctaagggaggccccccaatcccataaggatccggggcagtctgcgcatcacggatgcgcggctcacagaagggacgtggtgagaagctggcctgggggagctgctcgcggcccacaccatgccaccagcggcacctccgcagggcacatgtcggggagaaaaaacatgtggggacctggggctttctccacctcacactcacgggtcacctcacacaggacacctcacactcagggtgcacttcaaactcacaggtcattacacctcacacaggacgcctcacacaagacacctcacatggtgcacttcacactcacaggtcacctcacattcgacacctcacactgagcacacttcacactcgggacacctcacactcagggtgcacttcaaactcacaggtcattacacctcacacaggacgcctcacacaagacacctcacacagggcacacttcacactcacgggtcacctcacattcgacacctaacacagatcacctcactcagaggacacctcacactgggcacacttcacactcaggacacctcacactcagggtgcacttcagactcacaggtcatgacacctcacacagatcacctcactcttataggacacttcacactcacagatcacctcactctcacaggacacttcggacaggacacacttcacacaggccacctcaggttatgtcaaactcacaggtcccctcacacagtcacctcacacagtgtacacttcacactcacaggtcccctcacacaggtcacctcacacagtcacctcacacagtgtacacttcacactcacaggtcccctcacacaggacacctcacacagtcacctcacacagtgcacacttcacacaggtcccctcacacagtcacctcacacacagggcacacttcacactcacaggtcccctcacacaggtcacctcacacaagatgcacttcacactcatgggtcccatcacacacaggacacctcacactcgtcacctcacacatgttacatcaaactctcaggtcccctcacacagtcacctcacacacagggcacacttcacattcgcaggtcacctcacaccaggcccacgactctcacaggtcaccacacctcacacacatcagctcacatagatcatctcaccctcacaggacatccatcacactcacaggtcacgtcacactcatacctcacccacaggtcacctcacacaaatcaccttgctcacacagatcacctcacacgcagggcacacttcacactcacaggttccctcacacaggacacctcacactcacagatcacctcaacaccgatcacctcacacaggggacacttcattctcacaggtcacctcacataggacatctcacacacaggtcacctcactcacagatcacttcacacagggcacacttcactcacagatcacacctcacctcccgctcacagatcacctcactctcacagggcacctcactctcacaggacacctcatacagggcacacttcactcccacaggtcaccatacctcacacagatcagatcacttcattctcacaggatacctcacactcagggcacacttcacactcacaggtcacctcacacaaggcacccttcacacaggtcaccacacctcacacagatcatctcactctcacaggacccctcacactcagattatctcacactcaggtcaccacacctcacactcttaggatgtctcacgcaggatgcctcacagtcacagagaaccacatctcatatgcacaagacacttcacattcacaggacacctcatgctcacaggaagcctcacactcacaggaagtccagctgtctgagacaaaggctaacatgaccctttccgggcaaattgaggatggtcatgcctagcatttttatccacctagttttcaaagcatttctcatctgttgtattctcacagcaccctgtgagtttaagtttaggtggccaacagtttcttcagcaatcacttttttctgtggagtgcttttgctgtttgtggaagattttgcatctgctactgcaccctctcccggtgtcagccggtgtgtgtggccaccctgtcagagatggagctgtggctcaaagcctgtgtacctcatcccaggtccacagctcagcgacagaagagtcagggctgaacctcgggtgttctgacctgggagcaggaaatgtgtggtcacccatagtttcagaagtcctggggaggggccaagattggaagaaatctacctcagctctgcaggaaagtctggcttcctgagcccaccccgccaggcccaggtccaagttccccaaccccagctcgtggtttgtccagtgctcaccgttgggtgcactggtgaaggtgctcacgaggctttctcttttgttccctcagaagctgttctggatttcttacagtggtgagtagatgatcgccaccaatcctgcctgcaacccttctcctcagcgtactgacgccagcccattccacagatggggaccagtgtgcctcaaatccgtgccagaatgggggctcctgcaaggaccagctccagtcctatatctgcttctgcctcccttccttcgagggccggaactgtgagaagagtgaggccccactttgggtcccatatttgcagagggcctggccaaccgggttgcagggtgcacaacctggtggggtgtgtggaccgggcattctgagctcagccccagttggaatttggtctaggtgaccttgaagtcccttctagtctgaggtctttgacagggacccaaggttctaattctcagactcagtggccccttgggctcccggccctgggcaattctcagccctcgagatggcccagctgagagtccctgtgtccctgtcccacttccacgtcccaccaggcagcaccgcttggtaaacttccccttctctactttccattacaaaggtttgaggtgttttttgttttgtttgtttgtttttggttttgttttgttttgtttaccatctgaatattaaattattgcaaagtttgaggcccccaacttcccttaggttcagtaattcactagaaggactcatagaacccactgaagtggatacactcacagttaccatttattacagcaaaggaagctgacttaagtctgcagaggaaccgggcacaaacttcccattgtcccctccctgtggggtcatgtggacacttctcccagaaagacgtgtgatgagacgtgggaagtactgccaacttgggaagctctatgagccccggtgtccagaggttttatcagggctcaatcacacagacccagctgaccacccacacggctgacctcagtctcagcccctccagaggccaagccaatagtgtggcccgaggccctgccatcatcacattgtcagctagaccatccagcatggcccaaggtccgggtaaacaccaacattccctcagggcttagcgatcacttcccaggaaatgtgtggtcacccttccaagggcttagcgatcacttcccaggaaatgtgtggtcacccttccaagggcttagcgatcacttcccaggaaatgtgtggtcacccttccaagggcttagtgatcacttcccaggagccaaggcaaaggctaccctttccctgggaacagcagctcatccttgaccacccaaggtggttcattctcacactgaacgagctctccggcacagcagccactttcttctctaagtagaagagagcccagcaaggtggggcaggctgaagagagaggcttcctgctggtcatctgggtccagaatgcctggggatctctgctcagccctggtgcccagcagccctggtgtgcatcctgcagggcaggccttcccgccggagtcctggacttactcagggccactgcccttgcccacatcaatcaaagtcgggctgccggttctgctgcttctgtctgagcccatggccagtgctgggactgactgtccctaggcgggctcgcggtggcatgaggccagcttggagaactgtctcagcgctctggtcctctcgtcagttgagtctctgattggaagtcccttggatacttttaccatccctacgggactttcactttcccccaggctcccctcagcttcccatcagctgctcggaagagtggtcaccctggaggccactgcccaccagccaggcacccccccaaatgcaactgcagccagcgctgcccccgactggcaaggctgttcagacgtgactcctctgatccaggccttgtcctttggatcagtccacggagcaggcggtgccaagctcaggctctgtcgcccacagctcagtgccccttccaggcagaacgccgctgctgacttagggcatggcatcccccggggctggctctcactgacccaaagaggcccctctcagggtatccccttcctgtccgcagacaaggatgaccagctgatctgcgtgaacgagaacggcggctgtgagcagtactgcagtgaccacgcgggtgccaagcgctcctgttggtgccacgaggggtactcgctgctggcagacggggtgtcctgcatgcccacaggtgaccaggcttcatgtcccagtcccagatgacaccagtccctgtcccactacggattctcttactggacaaaagacgggtgggggtggcttcacatctgagcaccaaccatgcgctgaccaaccgtgaggcaggatctgggcaccaagggtggcacaggccagagcgacagtgactaggatgggcaccctgggggcagtccctgaatggcctcaggccccctacccatgctaggcagaccaggggagccaagcaaggctctatctcacgtccaactcccactcgcaggacctccgctggggttcgtgaatctaccttggcacaggcagtgtctgtactgactgctgcccgctctgaattccaaaacttgtaaaggctcctgggaaaatgggatgtttctccaaaccagcctggaacaaatgggctgcacttccaaaggcagggacaccccacgcccactgtgtctcgaagaggtggacgtgcccaccctggccacacagcctgggactcagcccaccacctcctcaggttttctttctggcccacgaccttgattggagcagatcaaaactaagcgtgggatcaaaacaacagagttgtttgtgacgttgattcatctttaggtagaatttcattcaccttttactaaagtcaagcaacacattttccccctgaaaagtgagcagagggcaatattaagacgtaagccctccatctcctccaaaaccagccctgaccattgtctcctcagccagccacttccgcaagggcctctcatggcccagccccaccagtcaggcccagccccaccagtcaggcccagccgaggccctgctttccaccatccctgggccctggcagctcctgctcctgggggcctcccatagcctcggcctcaaggcctctcagaggatgggtgtttctgaatctttcctagtggctcgttcatccttcacaaatttctgcatctttctgacttttgttttacacagttgaatatccatgtggaaaaatacctattctggaaaaaagaaatgccagcaaaccccaaggccgaattgtcgggggcagggtgtgccccaaaggggagtgtccatggcaggtaaggcttcccttggcttcaggattctaagccctgagggtcttggagccttttgaatgtgagctgaacaacagttctggaagggaaaatgggcaggtcagccccaaggccaccaggctccaagtcagcccacctagaacctctagctcgctgcacccccatgctttcagtggggcaaggaaggagaaaagaaggcgacactcgctgagggtctaccctgtgcagagaaccctgcgagatgcccctcccgagttgtcacgtcgtcctcactgttactctttgaggtgggatctttgcctgatctttgcaaaatcaggagcattggatcaaagctatgtgaagatcccgtgaggtgaacagtgaaatctcacagcgacgtttgtattgttgggctgtgcccaagagcacgtctcggctagagaggggcgcagcctcccagagccaggtctgagcagctttgcctgggagggatctgcaaagaccccaggatttcagaaacaaattgtgcaatgccagaggtcccttggcgtgcccgggagggcgagtcatcagagaaacaatgacagtaatgtgacttccatgcctcctgtccccccgcccaggtcctgttgttggtgaatggagctcagctgtgtggagggaccctgataaacaccatctgggtggtctctgcggcccactgtttcgacaaaatcaagagctggaggaacttgaccgcggtgctgggtaggtgccgctctcccctgtgtgaccgcggtgctgggtaggtgccgctctcccctgtgtgaccgcggtgctgggtaggtaccactctcctctgaccgcgttgctgggtgggtactgctctcccatctgactgctgtgctggttacgcgccgttctcccgtctgacgatggtgctgagtaggcgccactctcccctgtctgaccacggctctcaagtgtctcaggggccgcagctctgggcttcgtgctgtcacttccacagacagacagacatctccaaaaggggagcaactgtgctaggcatgactgctgtggccaccgtcctctcagccactttcccatgcccaaataaaatggtaaaagacaggggttctgcccatcctgcctcacctggccaagagcccataggaggatgcaacttccagggcttcatgggaccactgggtggcagggactgtgctcactgggtttacaggtgagatgaacattcccaggagggcacttggctgggaagaactgtggagaatcagggcacaccctgcccccccagctgccaggtcgcagcaccccttccccacctcaacgcccaggccccagattcaccccagttcacacgtccccatgtgagccacaggctgccacctgcggcaggctggccaggtcaccttggggttggatgcaggcccccctcaccccaaaaggagactgcagcccctgcagacctagaaatggccacagcccgtccccatgcaccagggggccaggcagcaggtagtgggatgggcctgagcaaggctccctccttccaggcgagcacgacctcagcgagcacgaaggggatgagcagagccggcgggtggcgcaggtcatcatccccagcacgtatgtcctgggcgccaccaaccacgacatcgcgctgctccgcctgcagcagcccgtggtcctcactgaccatgtggtgcccctctgcctgcccgaacggacgttctccgagaggacgctggccttcgtgcgcttctcgttggtcagcggctggggtcagctgctggaccgtggtgccacagccctggagctcatggccctcaacgtgccccggctgatgacccaggactgcctgcagcagtcacagaaggcagaagcctccccgaatatcacggagtacatgttctgtgccggctactcggacggcagcagggactcctgcaagggggacagtggaggcccacacgccacccgctaccggggcacgtggtacctgacaggcatcgtcagctggggccagggctgcgcggccgtgggccacttcggggtgtacaccagggtctcccagtacatcgagtggctgcaaaagctcatgcactcagagccacgcccaggcgtcctcctgcgagccccatttccctagcctagcagccctgccccctggagagaaagccaaggctgtgtagaactgttctggcacaaaatcccatcgattcttctgcagttcatggggtagaggagggcatgggagggagggagaggtggggagggagacagagacagaaacagagagacaaagagacagggagagactgagggagaggttctgaggacatggagagactcaaagagactccaagattcaaagagcctaatagagacacagagaaggaatcgaaaagatgagatgcagaggcagacaggcgctggacagaggggcaggggaatgctgcggttgtcctggaggcagacagcccagctgagcctccttatctctcttcagccaagcccacctgcccgtgatctgctggcctcaggctgctgttctgccttcattgctggagacactagaggcatgtacacacgtggatgcatacacacacaccaatgcacacacacagagatatgcacacacacggatgcacacacagagggtcacacagagatatgcaaacacactgacacacacatacagagatatgcacatacacagatgcatatacacagatatgcgcacacacggatgcgtgcacaccacaccaatgcacacacacactaatgcacccacacggatgcagagagatatgcacacaccgatgtgcacatacacagatatgcacacacatggatgagtgcacacacaccaatgtacacacacagatatgcacacacggatgcacacacaccgatgctgactccatgtgtgctgtcctccaaaggcggttgtttagctctcacttttctcgttcttatccattatcatcttcatttcagacaattcagaagcatcaccatgcatgttggcaaatgccccaaactctcccccaaatgtgccgggctgcacaggccgttccccaccggcttcccaacttcacaataaatggctgcatctcctccgcacgcctgtggggcctgccacccaccgtgtagcctgtgattcattttcagagcctccagtgctcatcctctgagatgcttttttctttcacagttttcagcatcactgaaatgaaccctcacatggcagctgttctttttaaaaacaaaagctctttgatagatgtttgaggctgtagctcccaggaccctgtggaattggttgttctctccctgccacagcccttgtcaatgatatttcgcagagaccctgggagcacctgcttgagaatcagggacataccactaaatgcaggttcccaggccctggctgcagtgggaggacctggcaagctgcactcttgctgagtccccagggtggtgggggaagaatgagaaacacatgagcagagaaatggggaggtgacagacactgcccgcactcagactccagcaagcatggctcagagagcggactcaacgccatccctgtagggccgtcctgggcaccagtggcgctcacagcagcaaggcaggcaccagcaacccacctcgggggcactcaggcaacatctactttagagcagacagggtccgtgaactacagctgagggctgcttctaagccacccggctcttgtaaataaagttttatgggaacacacccacgttagtgtccatggagtggccgtgacagagatgtctagccagacagaccagctgacctgccaagcccagcatgattagtgtcaggacctctgccgaagatgctggctgaccctggccagaccccagttcctaatgcccccacacagggacgggggccagtggcgggccctgatcaggtcagagctggctctgctttctcttttgtccgagtgactggggagtcatgcggggtcctggtggggtgccagcctcccttcttgccaatgggaaatgcaggcacccacctcacggtgctgctgaaggagggggcccgggactctccagaaactttgctgaagggcctgggcaccctcgaaggctacatttcttatgggacgtgccacctgccatggctcagctccagctttctgtgagtggcgagatagaatacagggaggccactggccatgggcctgcgacagggtggggcgaggcagcaggctcgggcctccaaagccagcatcaccacccagcgttgatgaaaaagactgcatgtctgccatgagcatcctgctgctggtgcacacaccacattggtctctccatacaaacgtgcctagaggcgatgtcggagtgtggagaccacgagaggcaggagtcagacatctggtgccaccaggaaggcccctctcagaggactgggctgtgcgtggtgcccaccgtgggaggctaccctggcgttggcacccagtgccatcagtttgtgtagtcgggtggggcccagtgagcacctcctgtgtgccaggcacaatgacgcacaatgtgtgcacaccaggcccaggtgcaggtggctgcgagacgggcaacacatcaaggcagacacaccgtgaggcagtggccagcactgtgggttttaggggcgttgctccggccactacggcatagcaggtagtgattgccacactggccaagttttagaccatttattccagggaccccagaagcaaaaatcctggctgcacctcccggtgactcccacagccattgagtggagacgctcagggacctggtgacaggaggtttctgtgctggacaatgagcccagaagccctctcagccttggaacagtgtggccagtggtgggcaggtcaggaggggtttcagacagagcctgtccctccagatggtcaggggagggctactccccacagaagtacatgttgggaccatgtgggcacagaaggtttgggggtgggtggggcaggtaccagcctggcctgtgggagaccgtggtgcagatgccaagcccccccgtgacatcagaccacctgacaccacccagagaatggc

SEQ ID NO: 5 - DNA, 20 bp, Artificial sequence
  Primer
gggaccctgatcaacaccat

SEQ ID NO: 6 - DNA, 24 bp, Artificial sequence
  Primer
ccagttcttgattttgtcgaaaca

SEQ ID NO: 7 - DNA, 18 bp, Artificial sequence
  Probe
tgggtggtctccgcggcc

SEQ ID NO: 8-20 - skipped (13 entries)

SEQ ID NO: 21 - DNA, 16 bp, Artificial sequence
  Synthetic oligonucleotide
gatgaaatctctgcag

SEQ ID NO: 22 - DNA, 16 bp, Artificial sequence
  Synthetic oligonucleotide
agaccatgatgaaatc

SEQ ID NO: 23 - DNA, 16 bp, Artificial sequence
  Synthetic oligonucleotide
gcctggatgctggttt

SEQ ID NO: 24 - DNA, 14 bp, Artificial sequence
  Synthetic oligonucleotide
cctggatgctggtt

SEQ ID NO: 25 - DNA, 14 bp, Artificial sequence
  Synthetic oligonucleotide
gcctggatgctggt

SEQ ID NO: 26 - DNA, 16 bp, Artificial sequence
  Synthetic oligonucleotide
tggagcggtcacttcc

SEQ ID NO: 27 - DNA, 16 bp, Artificial sequence
  Synthetic oligonucleotide
aggaggctgaggatgc

SEQ ID NO: 28 - DNA, 16 bp, Artificial sequence
  Synthetic oligonucleotide
ctgcaggagcggccta

SEQ ID NO: 29 - DNA, 16 bp, Artificial sequence
  Synthetic oligonucleotide
cgtattttctgatgtg

SEQ ID NO: 30 - DNA, 16 bp, Artificial sequence
  Synthetic oligonucleotide
gaggtgacccgtgagc

SEQ ID NO: 31 - DNA, 14 bp, Artificial sequence
  Synthetic oligonucleotide
aggtgacccgtgag

SEQ ID NO: 32 - DNA, 14 bp, Artificial sequence
  Synthetic oligonucleotide
ctgctcacagccgc

SEQ ID NO: 33 - DNA, 14 bp, Artificial sequence
  Synthetic oligonucleotide
gcagtactgctcac

SEQ ID NO: 34 - DNA, 16 bp, Artificial sequence
  Synthetic oligonucleotide
aatggtcagggctggt

SEQ ID NO: 35 - DNA, 14 bp, Artificial sequence
  Synthetic oligonucleotide
atggtcagggctgg

SEQ ID NO: 36 - DNA, 14 bp, Artificial sequence
  Synthetic oligonucleotide
ggtttgctggcatt

SEQ ID NO: 37 - DNA, 16 bp, Artificial sequence
  Synthetic oligonucleotide
acaattcggccttggg

SEQ ID NO: 38 - DNA, 16 bp, Artificial sequence
  Synthetic oligonucleotide
gctcagacctggctct

SEQ ID NO: 39 - DNA, 16 bp, Artificial sequence
  Synthetic oligonucleotide
agctgctcagacctgg

SEQ ID NO: 40 - DNA, 14 bp, Artificial sequence
  Synthetic oligonucleotide
gctgctcagacctg

SEQ ID NO: 41 - DNA, 16 bp, Artificial sequence
  Synthetic oligonucleotide
ccacccagatggtgtt

SEQ ID NO: 42 - DNA, 16 bp, Artificial sequence
  Synthetic oligonucleotide
cgaaacagtgggccgc

SEQ ID NO: 43 - DNA, 14 bp, Artificial sequence
  Synthetic oligonucleotide
gaaacagtgggccg

SEQ ID NO: 44 - DNA, 16 bp, Artificial sequence
  Synthetic oligonucleotide
gtgctcgctgaggtcg

SEQ ID NO: 45 - DNA, 16 bp, Artificial sequence
  Synthetic oligonucleotide
ccatgagctccagggc

SEQ ID NO: 46 - DNA, 14 bp, Artificial sequence
  Synthetic oligonucleotide
ctgggtcatcagcc

SEQ ID NO: 47 - DNA, 14 bp, Artificial sequence
  Synthetic oligonucleotide
gtcctgggtcatca

SEQ ID NO: 48 - DNA, 16 bp, Artificial sequence
  Synthetic oligonucleotide
cagaacatgtactccg

SEQ ID NO: 49 - DNA, 16 bp, Artificial sequence
  Synthetic oligonucleotide
cgagtagccggcacag

SEQ ID NO: 50 - DNA, 16 bp, Artificial sequence
  Synthetic oligonucleotide
tccactgtcccccttg

SEQ ID NO: 51 - DNA, 14 bp, Artificial sequence
  Synthetic oligonucleotide
cctggtgtacaccc

SEQ ID NO: 52 - DNA, 14 bp, Artificial sequence
  Synthetic oligonucleotide
gtactgggagaccc

SEQ ID NO: 53 - DNA, 16 bp, Artificial sequence
  Synthetic oligonucleotide
cccctctgtccagcgc

SEQ ID NO: 54 - DNA, 14 bp, Artificial sequence
  Synthetic oligonucleotide
ccctctgtccagcg

SEQ ID NO: 55 - DNA, 14 bp, Artificial sequence
  Synthetic oligonucleotide
aggccagcagatca

SEQ ID NO: 56 - DNA, 14 bp, Artificial sequence
  Synthetic oligonucleotide
gcctgaggccagca

SEQ ID NO: 57 - DNA, 16 bp, Artificial sequence
  Synthetic oligonucleotide
atggagtcagcatcgg

SEQ ID NO: 58 - DNA, 14 bp, Artificial sequence
  Synthetic oligonucleotide
tggagtcagcatcg

SEQ ID NO: 59 - DNA, 16 bp, Artificial sequence
  Synthetic oligonucleotide
gctaaacaaccgcctt

SEQ ID NO: 60 - DNA, 14 bp, Artificial sequence
  Synthetic oligonucleotide
ctaaacaaccgcct

SEQ ID NO: 61 - DNA, 16 bp, Artificial sequence
  Synthetic oligonucleotide
tgaagatgataatgga

SEQ ID NO: 62 - DNA, 14 bp, Artificial sequence
  Synthetic oligonucleotide
gaagatgataatgg

SEQ ID NO: 63 - DNA, 16 bp, Artificial sequence
  Synthetic oligonucleotide
ttctgaattgtctgaa

SEQ ID NO: 64 - DNA, 16 bp, Artificial sequence
  Synthetic oligonucleotide
gtgatgcttctgaatt

SEQ ID NO: 65 - DNA, 16 bp, Artificial sequence
  Synthetic oligonucleotide
tggtgatgcttctgaa

SEQ ID NO: 66 - DNA, 14 bp, Artificial sequence
  Synthetic oligonucleotide
ggtgatgcttctga

SEQ ID NO: 67 - DNA, 16 bp, Artificial sequence
  Synthetic oligonucleotide
atggtgatgcttctga

SEQ ID NO: 68 - DNA, 14 bp, Artificial sequence
  Synthetic oligonucleotide
tggtgatgcttctg

SEQ ID NO: 69 - DNA, 16 bp, Artificial sequence
  Synthetic oligonucleotide
tgcatggtgatgcttc

SEQ ID NO: 70 - DNA, 14 bp, Artificial sequence
  Synthetic oligonucleotide
gcatggtgatgctt

SEQ ID NO: 71 - DNA, 16 bp, Artificial sequence
  Synthetic oligonucleotide
atgcatggtgatgctt

SEQ ID NO: 72 - DNA, 20 bp, Artificial sequence
  Synthetic oligonucleotide
tgcagcccggcacccagcga

SEQ ID NO: 73 - DNA, 16 bp, Artificial sequence
  Synthetic oligonucleotide
ctgtgcagcccggcac

SEQ ID NO: 74 - DNA, 14 bp, Artificial sequence
  Synthetic oligonucleotide
tgtgcagcccggca

SEQ ID NO: 75 - DNA, 14 bp, Artificial sequence
  Synthetic oligonucleotide
agctgctcagacct

SEQ ID NO: 76 - DNA, 14 bp, Artificial sequence
  Synthetic oligonucleotide
aagctgctcagacc

SEQ ID NO: 77 - DNA, 16 bp, Artificial sequence
  Synthetic oligonucleotide
actgctcacagccgcc

SEQ ID NO: 78 - DNA, 16 bp, Artificial sequence
  Synthetic oligonucleotide
acccagcaccgcggtc

SEQ ID NO: 79 - DNA, 14 bp, Artificial sequence
  Synthetic oligonucleotide
gagtagccggcaca

SEQ ID NO: 80 - DNA, 14 bp, Artificial sequence
  Synthetic oligonucleotide
tgcatggtgatgct

SEQ ID NO: 81 - DNA, 16 bp, Artificial sequence
  Synthetic oligonucleotide
ggctctgctcatcccc

SEQ ID NO: 82 - DNA, 20 bp, Artificial sequence
  Synthetic oligonucleotide
ggcacactggtccccatcac

SEQ ID NO: 83 - DNA, 20 bp, Artificial sequence
  Synthetic oligonucleotide
cctgcagccaggcagccctg

SEQ ID NO: 84 - DNA, 20 bp, Artificial sequence
  Synthetic oligonucleotide
ctggtccttgcaggagcccc

SEQ ID NO: 85 - DNA, 20 bp, Artificial sequence
  Synthetic oligonucleotide
caggagcccccattctggca

SEQ ID NO: 86 - DNA, 16 bp, Artificial sequence
  Synthetic oligonucleotide
gaggccagcagatcac

SEQ ID NO: 87 - DNA, 16 bp, Artificial sequence
  Synthetic oligonucleotide
agcctgaggccagcag

SEQ ID NO: 88 - DNA, 16 bp, Artificial sequence
  Synthetic oligonucleotide
aggcacactggtcccc

SEQ ID NO: 89 - DNA, 16 bp, Artificial sequence
  Synthetic oligonucleotide
ctgctcagacctggct

SEQ ID NO: 90 - DNA, 20 bp, Artificial sequence
  Synthetic oligonucleotide
cctgtgcctggatgctggtt

SEQ ID NO: 91 - DNA, 20 bp, Artificial sequence
  Synthetic oligonucleotide
ctcctgtgcctggatgctgg

SEQ ID NO: 92 - DNA, 20 bp, Artificial sequence
  Synthetic oligonucleotide
cctcctgtgcctggatgctg

SEQ ID NO: 93 - DNA, 20 bp, Artificial sequence
  Synthetic oligonucleotide
ccctcctgtgcctggatgct

SEQ ID NO: 94 - DNA, 20 bp, Artificial sequence
  Synthetic oligonucleotide
ggcagtccctgctcacctct

SEQ ID NO: 95 - DNA, 20 bp, Artificial sequence
  Synthetic oligonucleotide
gcatcagaaaagctctcaag

SEQ ID NO: 96 - DNA, 20 bp, Artificial sequence
  Synthetic oligonucleotide
gtctggtttggaaggaggct

SEQ ID NO: 97 - DNA, 20 bp, Artificial sequence
  Synthetic oligonucleotide
ggttactgagcgcggaagaa

SEQ ID NO: 98 - DNA, 20 bp, Artificial sequence
  Synthetic oligonucleotide
gttctgcaggagcggcctaa

SEQ ID NO: 99 - DNA, 20 bp, Artificial sequence
  Synthetic oligonucleotide
gagttctgcaggagcggcct

SEQ ID NO: 100 - DNA, 20 bp, Artificial sequence
  Synthetic oligonucleotide
cgagttctgcaggagcggcc

SEQ ID NO: 101 - DNA, 20 bp, Artificial sequence
  Synthetic oligonucleotide
ggagggacgacctttgctgg

SEQ ID NO: 102 - DNA, 20 bp, Artificial sequence
  Synthetic oligonucleotide
gaccactcttccgagcagct

SEQ ID NO: 103 - DNA, 20 bp, Artificial sequence
  Synthetic oligonucleotide
ggtcactgcagtactgctca

SEQ ID NO: 104 - DNA, 20 bp, Artificial sequence
  Synthetic oligonucleotide
taggtatttttccacatgga

SEQ ID NO: 105 - DNA, 20 bp, Artificial sequence
  Synthetic oligonucleotide
ccccacaattcggccttggg

SEQ ID NO: 106 - DNA, 20 bp, Artificial sequence
  Synthetic oligonucleotide
ctgagctccattcaccaaca

SEQ ID NO: 107 - DNA, 20 bp, Artificial sequence
  Synthetic oligonucleotide
cgccggctctgctcatcccc

SEQ ID NO: 108 - DNA, 20 bp, Artificial sequence
  Synthetic oligonucleotide
agtccttgctgccatccgag

SEQ ID NO: 109 - DNA, 20 bp, Artificial sequence
  Synthetic oligonucleotide
ggagaccctggtgtacaccc

SEQ ID NO: 110 - DNA, 20 bp, Artificial sequence
  Synthetic oligonucleotide
atgtactgggagaccctggt

SEQ ID NO: 111 - DNA, 20 bp, Artificial sequence
  Synthetic oligonucleotide
cagccttggctttctctcca

SEQ ID NO: 112 - DNA, 20 bp, Artificial sequence
  Synthetic oligonucleotide
cctgaggccagcagatcacg

SEQ ID NO: 113 - DNA, 20 bp, Artificial sequence
  Synthetic oligonucleotide
cagcctgaggccagcagatc

SEQ ID NO: 114 - DNA, 20 bp, Artificial sequence
  Synthetic oligonucleotide
cacacatggagtcagcatcg

SEQ ID NO: 115 - DNA, 20 bp, Artificial sequence
  Synthetic oligonucleotide
tgagagctaaacaaccgcct

SEQ ID NO: 116 - DNA, 20 bp, Artificial sequence
  Synthetic oligonucleotide
gtgagagctaaacaaccgcc

SEQ ID NO: 117 - DNA, 20 bp, Artificial sequence
  Synthetic oligonucleotide
tgatgcttctgaattgtctg

SEQ ID NO: 118 - DNA, 20 bp, Artificial sequence
  Synthetic oligonucleotide
gtgatgcttctgaattgtct

SEQ ID NO: 119 - DNA, 20 bp, Artificial sequence
  Synthetic oligonucleotide
tgcatggtgatgcttctgaa

SEQ ID NO: 120 - DNA, 20 bp, Artificial sequence
  Synthetic oligonucleotide
atgcatggtgatgcttctga

SEQ ID NO: 121 - DNA, 20 bp, Artificial sequence
  Synthetic oligonucleotide
catgcatggtgatgcttctg

SEQ ID NO: 122 - DNA, 20 bp, Artificial sequence
  Synthetic oligonucleotide
ggcattcgccaccatgcatg

SEQ ID NO: 123 - DNA, 20 bp, Artificial sequence
  Synthetic oligonucleotide
tcctgcagccaggcagccct

SEQ ID NO: 124 - DNA, 14 bp, Artificial sequence
  Synthetic oligonucleotide
gtgcctggatgctg

SEQ ID NO: 125 - DNA, 14 bp, Artificial sequence
  Synthetic oligonucleotide
ctgtgcctggatgc

SEQ ID NO: 126 - DNA, 14 bp, Artificial sequence
  Synthetic oligonucleotide
agtggcagtccctg

SEQ ID NO: 127 - DNA, 14 bp, Artificial sequence
  Synthetic oligonucleotide
ccagtggcagtccc

SEQ ID NO: 128 - DNA, 14 bp, Artificial sequence
  Synthetic oligonucleotide
ggtgatgttggccc

SEQ ID NO: 129 - DNA, 14 bp, Artificial sequence
  Synthetic oligonucleotide
gctctcaagaactg

SEQ ID NO: 130 - DNA, 14 bp, Artificial sequence
  Synthetic oligonucleotide
gcatcagaaaagct

SEQ ID NO: 131 - DNA, 14 bp, Artificial sequence
  Synthetic oligonucleotide
agatttgcatcaga

SEQ ID NO: 132 - DNA, 14 bp, Artificial sequence
  Synthetic oligonucleotide
gaggatgcaggcgg

SEQ ID NO: 133 - DNA, 14 bp, Artificial sequence
  Synthetic oligonucleotide
tctggtttggaagg

SEQ ID NO: 134 - DNA, 14 bp, Artificial sequence
  Synthetic oligonucleotide
gttactgagcgcgg

SEQ ID NO: 135 - DNA, 14 bp, Artificial sequence
  Synthetic oligonucleotide
tctgcaggagcggc

SEQ ID NO: 136 - DNA, 14 bp, Artificial sequence
  Synthetic oligonucleotide
ttctgcaggagcgg

SEQ ID NO: 137 - DNA, 14 bp, Artificial sequence
  Synthetic oligonucleotide
gttctgcaggagcg

SEQ ID NO: 138 - DNA, 14 bp, Artificial sequence
  Synthetic oligonucleotide
agttctgcaggagc

SEQ ID NO: 139 - DNA, 14 bp, Artificial sequence
  Synthetic oligonucleotide
gagttctgcaggag

SEQ ID NO: 140 - DNA, 14 bp, Artificial sequence
  Synthetic oligonucleotide
ggacgaggcctcag

SEQ ID NO: 141 - DNA, 14 bp, Artificial sequence
  Synthetic oligonucleotide
gctgtgggcaccac

SEQ ID NO: 142 - DNA, 14 bp, Artificial sequence
  Synthetic oligonucleotide
agctgtgggcacca

SEQ ID NO: 143 - DNA, 14 bp, Artificial sequence
  Synthetic oligonucleotide
gagctgtgggcacc

SEQ ID NO: 144 - DNA, 14 bp, Artificial sequence
  Synthetic oligonucleotide
gctccgagcaggcc

SEQ ID NO: 145 - DNA, 14 bp, Artificial sequence
  Synthetic oligonucleotide
cggccgcagctcct

SEQ ID NO: 146 - DNA, 14 bp, Artificial sequence
  Synthetic oligonucleotide
ccggccgcagctcc

SEQ ID NO: 147 - DNA, 14 bp, Artificial sequence
  Synthetic oligonucleotide
agatcagctggtca

SEQ ID NO: 148 - DNA, 14 bp, Artificial sequence
  Synthetic oligonucleotide
gctcacagccgccg

SEQ ID NO: 149 - DNA, 14 bp, Artificial sequence
  Synthetic oligonucleotide
tgctcacagccgcc

SEQ ID NO: 150 - DNA, 14 bp, Artificial sequence
  Synthetic oligonucleotide
actgctcacagccg

SEQ ID NO: 151 - DNA, 14 bp, Artificial sequence
  Synthetic oligonucleotide
ggtcactgcagtac

SEQ ID NO: 152 - DNA, 14 bp, Artificial sequence
  Synthetic oligonucleotide
ggatattcaactgt

SEQ ID NO: 153 - DNA, 14 bp, Artificial sequence
  Synthetic oligonucleotide
aggtatttttccac

SEQ ID NO: 154 - DNA, 14 bp, Artificial sequence
  Synthetic oligonucleotide
tcaccaacaacagg

SEQ ID NO: 155 - DNA, 14 bp, Artificial sequence
  Synthetic oligonucleotide
acctgcgccacccg

SEQ ID NO: 156 - DNA, 14 bp, Artificial sequence
  Synthetic oligonucleotide
gacctgcgccaccc

SEQ ID NO: 157 - DNA, 14 bp, Artificial sequence
  Synthetic oligonucleotide
ccgttcgggcaggc

SEQ ID NO: 158 - DNA, 14 bp, Artificial sequence
  Synthetic oligonucleotide
tgggtcatcagccg

SEQ ID NO: 159 - DNA, 14 bp, Artificial sequence
  Synthetic oligonucleotide
acatgtactccgtg

SEQ ID NO: 160 - DNA, 14 bp, Artificial sequence
  Synthetic oligonucleotide
gaacatgtactccg

SEQ ID NO: 161 - DNA, 14 bp, Artificial sequence
  Synthetic oligonucleotide
cgagtagccggcac

SEQ ID NO: 162 - DNA, 14 bp, Artificial sequence
  Synthetic oligonucleotide
ccgagtagccggca

SEQ ID NO: 163 - DNA, 14 bp, Artificial sequence
  Synthetic oligonucleotide
gagcttttgcagcc

SEQ ID NO: 164 - DNA, 14 bp, Artificial sequence
  Synthetic oligonucleotide
gtctccagcaatga

SEQ ID NO: 165 - DNA, 14 bp, Artificial sequence
  Synthetic oligonucleotide
acatggagtcagca

SEQ ID NO: 166 - DNA, 14 bp, Artificial sequence
  Synthetic oligonucleotide
gcacacatggagtc

SEQ ID NO: 167 - DNA, 14 bp, Artificial sequence
  Synthetic oligonucleotide
gctaaacaaccgcc

SEQ ID NO: 168 - DNA, 14 bp, Artificial sequence
  Synthetic oligonucleotide
agctaaacaaccgc

SEQ ID NO: 169 - DNA, 14 bp, Artificial sequence
  Synthetic oligonucleotide
gagctaaacaaccg

SEQ ID NO: 170 - DNA, 14 bp, Artificial sequence
  Synthetic oligonucleotide
agagctaaacaacc

SEQ ID NO: 171 - DNA, 14 bp, Artificial sequence
  Synthetic oligonucleotide
cttctgaattgtct

SEQ ID NO: 172 - DNA, 14 bp, Artificial sequence
  Synthetic oligonucleotide
gcttctgaattgtc

SEQ ID NO: 173 - DNA, 14 bp, Artificial sequence
  Synthetic oligonucleotide
atgcttctgaattg

SEQ ID NO: 174 - DNA, 14 bp, Artificial sequence
  Synthetic oligonucleotide
gtgatgcttctgaa

SEQ ID NO: 175 - DNA, 14 bp, Artificial sequence
  Synthetic oligonucleotide
atggtgatgcttct

SEQ ID NO: 176 - DNA, 14 bp, Artificial sequence
  Synthetic oligonucleotide
catggtgatgcttc

SEQ ID NO: 177 - DNA, 14 bp, Artificial sequence
  Synthetic oligonucleotide
atgcatggtgatgc

SEQ ID NO: 178 - DNA, 14 bp, Artificial sequence
  Synthetic oligonucleotide
ggtgcccaggacgg

SEQ ID NO: 179 - DNA, 14 bp, Artificial sequence
  Synthetic oligonucleotide
cgaggcgcggcccc

SEQ ID NO: 180 - DNA, 14 bp, Artificial sequence
  Synthetic oligonucleotide
ccgaggcgcggccc

SEQ ID NO: 181 - DNA, 14 bp, Artificial sequence
  Synthetic oligonucleotide
gtctccggcggcca

SEQ ID NO: 182 - DNA, 14 bp, Artificial sequence
  Synthetic oligonucleotide
gctgtgagaataca

SEQ ID NO: 183 - DNA, 14 bp, Artificial sequence
  Synthetic oligonucleotide
gaaactgttggcca

SEQ ID NO: 184 - DNA, 14 bp, Artificial sequence
  Synthetic oligonucleotide
agaaactgttggcc

SEQ ID NO: 185 - DNA, 14 bp, Artificial sequence
  Synthetic oligonucleotide
tgggtgaccacaca

SEQ ID NO: 186 - DNA, 14 bp, Artificial sequence
  Synthetic oligonucleotide
ggttgtgcaccctg

SEQ ID NO: 187 - DNA, 14 bp, Artificial sequence
  Synthetic oligonucleotide
caggttgtgcaccc

SEQ ID NO: 188 - DNA, 14 bp, Artificial sequence
  Synthetic oligonucleotide
agtttaccaagcgg

SEQ ID NO: 189 - DNA, 14 bp, Artificial sequence
  Synthetic oligonucleotide
aagtttaccaagcg

SEQ ID NO: 190 - DNA, 14 bp, Artificial sequence
  Synthetic oligonucleotide
cctctggacaccgg

SEQ ID NO: 191 - DNA, 14 bp, Artificial sequence
  Synthetic oligonucleotide
acctctggacaccg

SEQ ID NO: 192 - DNA, 14 bp, Artificial sequence
  Synthetic oligonucleotide
gtgattgagccctg

SEQ ID NO: 193 - DNA, 14 bp, Artificial sequence
  Synthetic oligonucleotide
ggtctagctgacaa

SEQ ID NO: 194 - DNA, 14 bp, Artificial sequence
  Synthetic oligonucleotide
ggatgcacaccagg

SEQ ID NO: 195 - DNA, 14 bp, Artificial sequence
  Synthetic oligonucleotide
aggatgcacaccag

SEQ ID NO: 196 - DNA, 14 bp, Artificial sequence
  Synthetic oligonucleotide
ggtgtcatctggga

SEQ ID NO: 197 - DNA, 14 bp, Artificial sequence
  Synthetic oligonucleotide
ctgtcgctctggcc

SEQ ID NO: 198 - DNA, 14 bp, Artificial sequence
  Synthetic oligonucleotide
ggaagtgcagccca

SEQ ID NO: 199 - DNA, 14 bp, Artificial sequence
  Synthetic oligonucleotide
tggaagtgcagccc

SEQ ID NO: 200 - DNA, 14 bp, Artificial sequence
  Synthetic oligonucleotide
gttgttttgatccc

SEQ ID NO: 201 - DNA, 14 bp, Artificial sequence
  Synthetic oligonucleotide
ggtcagggctggtt

SEQ ID NO: 202 - DNA, 14 bp, Artificial sequence
  Synthetic oligonucleotide
ggagacaatggtca

SEQ ID NO: 203 - DNA, 14 bp, Artificial sequence
  Synthetic oligonucleotide
aggagacaatggtc

SEQ ID NO: 204 - DNA, 14 bp, Artificial sequence
  Synthetic oligonucleotide
tctctgcacagggt

SEQ ID NO: 205 - DNA, 14 bp, Artificial sequence
  Synthetic oligonucleotide
gatccaatgctcct

SEQ ID NO: 206 - DNA, 14 bp, Artificial sequence
  Synthetic oligonucleotide
tgatccaatgctcc

SEQ ID NO: 207 - DNA, 14 bp, Artificial sequence
  Synthetic oligonucleotide
gctttgatccaatg

SEQ ID NO: 208 - DNA, 14 bp, Artificial sequence
  Synthetic oligonucleotide
tagctttgatccaa

SEQ ID NO: 209 - DNA, 14 bp, Artificial sequence
  Synthetic oligonucleotide
atagctttgatcca

SEQ ID NO: 210 - DNA, 14 bp, Artificial sequence
  Synthetic oligonucleotide
tcttcacatagctt

SEQ ID NO: 211 - DNA, 14 bp, Artificial sequence
  Synthetic oligonucleotide
tcgctgtgagattt

SEQ ID NO: 212 - DNA, 14 bp, Artificial sequence
  Synthetic oligonucleotide
ggcattgcacaatt

SEQ ID NO: 213 - DNA, 17 bp, Artificial sequence
  Synthetic oligonucleotide
gtgcctggatgctggtt

SEQ ID NO: 214 - DNA, 17 bp, Artificial sequence
  Synthetic oligonucleotide
tgtgcctggatgctggt

SEQ ID NO: 215 - DNA, 17 bp, Artificial sequence
  Synthetic oligonucleotide
gcatcagaaaagctctc

SEQ ID NO: 216 - DNA, 17 bp, Artificial sequence
  Synthetic oligonucleotide
ggttactgagcgcggaa

SEQ ID NO: 217 - DNA, 17 bp, Artificial sequence
  Synthetic oligonucleotide
gttctgcaggagcggcc

SEQ ID NO: 218 - DNA, 17 bp, Artificial sequence
  Synthetic oligonucleotide
agttctgcaggagcggc

SEQ ID NO: 219 - DNA, 20 bp, Artificial sequence
  Synthetic oligonucleotide
agacaatggtcagggctggt

SEQ ID NO: 220 - DNA, 20 bp, Artificial sequence
  Synthetic oligonucleotide
gctttgatccaatgctcctg

SEQ ID NO: 221 - DNA, 17 bp, Artificial sequence
  Synthetic oligonucleotide
gctgctcagacctggct

SEQ ID NO: 222 - DNA, 17 bp, Artificial sequence
  Synthetic oligonucleotide
gcccctctgtccagcgc

SEQ ID NO: 223 - DNA, 17 bp, Artificial sequence
  Synthetic oligonucleotide
tgcccctctgtccagcg

SEQ ID NO: 224 - DNA, 17 bp, Artificial sequence
  Synthetic oligonucleotide
ctgcccctctgtccagc

SEQ ID NO: 225 - DNA, 20 bp, Artificial sequence
  Synthetic oligonucleotide
gcctgaggccagcagatcac

SEQ ID NO: 226 - DNA, 17 bp, Artificial sequence
  Synthetic oligonucleotide
gcttctgaattgtctga

SEQ ID NO: 227 - DNA, 17 bp, Artificial sequence
  Synthetic oligonucleotide
tgcttctgaattgtctg

SEQ ID NO: 228 - DNA, 17 bp, Artificial sequence
  Synthetic oligonucleotide
catggtgatgcttctga

SEQ ID NO: 229 - DNA, 17 bp, Artificial sequence
  Synthetic oligonucleotide
gcatggtgatgcttctg

SEQ ID NO: 230 - DNA, 17 bp, Artificial sequence
  Synthetic oligonucleotide
tgcatggtgatgcttct

SEQ ID NO: 231 - DNA, 17 bp, Artificial sequence
  Synthetic oligonucleotide
atgcatggtgatgcttc

SEQ ID NO: 232 - DNA, 16 bp, Artificial sequence
  Synthetic oligonucleotide
tgcctggatgctggtt

SEQ ID NO: 233 - DNA, 16 bp, Artificial sequence
  Synthetic oligonucleotide
gcagatttgcatcaga

SEQ ID NO: 234 - DNA, 16 bp, Artificial sequence
  Synthetic oligonucleotide
ggttactgagcgcgga

SEQ ID NO: 235 - DNA, 16 bp, Artificial sequence
  Synthetic oligonucleotide
tctgcaggagcggcct

SEQ ID NO: 236 - DNA, 16 bp, Artificial sequence
  Synthetic oligonucleotide
ttctgcaggagcggcc

SEQ ID NO: 237 - DNA, 16 bp, Artificial sequence
  Synthetic oligonucleotide
gacctcgcgcggatcc

SEQ ID NO: 238 - DNA, 16 bp, Artificial sequence
  Synthetic oligonucleotide
ccgaggcgcggcccct

SEQ ID NO: 239 - DNA, 16 bp, Artificial sequence
  Synthetic oligonucleotide
tccgaggcgcggcccc

SEQ ID NO: 240 - DNA, 16 bp, Artificial sequence
  Synthetic oligonucleotide
agaaactgttggccac

SEQ ID NO: 241 - DNA, 16 bp, Artificial sequence
  Synthetic oligonucleotide
aagaaactgttggcca

SEQ ID NO: 242 - DNA, 16 bp, Artificial sequence
  Synthetic oligonucleotide
agtgattgctgaagaa

SEQ ID NO: 243 - DNA, 16 bp, Artificial sequence
  Synthetic oligonucleotide
ggcacactggtcccca

SEQ ID NO: 244 - DNA, 16 bp, Artificial sequence
  Synthetic oligonucleotide
cagatataggactgga

SEQ ID NO: 245 - DNA, 16 bp, Artificial sequence
  Synthetic oligonucleotide
ccaggttgtgcaccct

SEQ ID NO: 246 - DNA, 16 bp, Artificial sequence
  Synthetic oligonucleotide
cctgtcaaagacctca

SEQ ID NO: 247 - DNA, 16 bp, Artificial sequence
  Synthetic oligonucleotide
ggatgcacaccagggc

SEQ ID NO: 248 - DNA, 16 bp, Artificial sequence
  Synthetic oligonucleotide
gggtttgctggcattt

SEQ ID NO: 249 - DNA, 16 bp, Artificial sequence
  Synthetic oligonucleotide
cgtgctcgctgaggtc

SEQ ID NO: 250 - DNA, 16 bp, Artificial sequence
  Synthetic oligonucleotide
gaacatgtactccgtg

SEQ ID NO: 251 - DNA, 16 bp, Artificial sequence
  Synthetic oligonucleotide
tccgagtagccggcac

SEQ ID NO: 252 - DNA, 16 bp, Artificial sequence
  Synthetic oligonucleotide
tgtactgggagaccct

SEQ ID NO: 253 - DNA, 16 bp, Artificial sequence
  Synthetic oligonucleotide
gagctaaacaaccgcc

SEQ ID NO: 254 - DNA, 16 bp, Artificial sequence
  Synthetic oligonucleotide
agagctaaacaaccgc

SEQ ID NO: 255 - DNA, 16 bp, Artificial sequence
  Synthetic oligonucleotide
gagagctaaacaaccg

SEQ ID NO: 256 - DNA, 16 bp, Artificial sequence
  Synthetic oligonucleotide
gcttctgaattgtctg

SEQ ID NO: 257 - DNA, 16 bp, Artificial sequence
  Synthetic oligonucleotide
atgcttctgaattgtc

SEQ ID NO: 258 - DNA, 16 bp, Artificial sequence
  Synthetic oligonucleotide
gatgcttctgaattgt

SEQ ID NO: 259 - DNA, 16 bp, Artificial sequence
  Synthetic oligonucleotide
catggtgatgcttctg

SEQ ID NO: 260 - DNA, 16 bp, Artificial sequence
  Synthetic oligonucleotide
gcatggtgatgcttct

SEQ ID NO: 261 - DNA, 16 bp, Artificial sequence
  Synthetic oligonucleotide
catgcatggtgatgct

SEQ ID NO: 262 - DNA, 16 bp, Artificial sequence
  Synthetic oligonucleotide
aagctgctcagacctg

SEQ ID NO: 263 - DNA, 16 bp, Artificial sequence
  Synthetic oligonucleotide
aaagctgctcagacct

SEQ ID NO: 264 - DNA, 16 bp, Artificial sequence
  Synthetic oligonucleotide
ccctggtgtacacccc

SEQ ID NO: 265 - DNA, 16 bp, Artificial sequence
  Synthetic oligonucleotide
accctggtgtacaccc

SEQ ID NO: 266 - DNA, 20 bp, Artificial sequence
  Synthetic oligonucleotide
ccttccctgaaggttcctcc

SEQ ID NO: 267 - DNA, 17 bp, Artificial sequence
  Synthetic oligonucleotide
tggatattcaactgtgg

SEQ ID NO: 268 - DNA, 17 bp, Artificial sequence
  Synthetic oligonucleotide
gcctggatgctggtttc

SEQ ID NO: 269 - DNA, 17 bp, Artificial sequence
  Synthetic oligonucleotide
tgcctggatgctggttt

SEQ ID NO: 270 - DNA, 16 bp, Artificial sequence
  Synthetic oligonucleotide
gtgcctggatgctggt

SEQ ID NO: 271 - DNA, 17 bp, Artificial sequence
  Synthetic oligonucleotide
cctgtgcctggatgctg

SEQ ID NO: 272 - DNA, 17 bp, Artificial sequence
  Synthetic oligonucleotide
tcctgtgcctggatgct

SEQ ID NO: 273 - DNA, 17 bp, Artificial sequence
  Synthetic oligonucleotide
ctcctgtgcctggatgc

SEQ ID NO: 274 - DNA, 16 bp, Artificial sequence
  Synthetic oligonucleotide
ctcctgtgcctggatg

SEQ ID NO: 275 - DNA, 17 bp, Artificial sequence
  Synthetic oligonucleotide
cagtccctgctcacctc

SEQ ID NO: 276 - DNA, 16 bp, Artificial sequence
  Synthetic oligonucleotide
cagtccctgctcacct

SEQ ID NO: 277 - DNA, 17 bp, Artificial sequence
  Synthetic oligonucleotide
cagtggcagtccctgct

SEQ ID NO: 278 - DNA, 17 bp, Artificial sequence
  Synthetic oligonucleotide
accagtggcagtccctg

SEQ ID NO: 279 - DNA, 17 bp, Artificial sequence
  Synthetic oligonucleotide
cccaggacaaaaccagt

SEQ ID NO: 280 - DNA, 17 bp, Artificial sequence
  Synthetic oligonucleotide
aggtgatgttggccccc

SEQ ID NO: 281 - DNA, 17 bp, Artificial sequence
  Synthetic oligonucleotide
agcagggaacaccctcc

SEQ ID NO: 282 - DNA, 16 bp, Artificial sequence
  Synthetic oligonucleotide
gagcggtcacttcctc

SEQ ID NO: 283 - DNA, 16 bp, Artificial sequence
  Synthetic oligonucleotide
ggagcggtcacttcct

SEQ ID NO: 284 - DNA, 17 bp, Artificial sequence
  Synthetic oligonucleotide
tggagcggtcacttcct

SEQ ID NO: 285 - DNA, 17 bp, Artificial sequence
  Synthetic oligonucleotide
agctctcaagaactgag

SEQ ID NO: 286 - DNA, 16 bp, Artificial sequence
  Synthetic oligonucleotide
atcagaaaagctctca

SEQ ID NO: 287 - DNA, 16 bp, Artificial sequence
  Synthetic oligonucleotide
gcatcagaaaagctct

SEQ ID NO: 288 - DNA, 17 bp, Artificial sequence
  Synthetic oligonucleotide
tgcatcagaaaagctct

SEQ ID NO: 289 - DNA, 17 bp, Artificial sequence
  Synthetic oligonucleotide
cagatttgcatcagaaa

SEQ ID NO: 290 - DNA, 17 bp, Artificial sequence
  Synthetic oligonucleotide
gcagatttgcatcagaa

SEQ ID NO: 291 - DNA, 16 bp, Artificial sequence
  Synthetic oligonucleotide
gaggctgaggatgcag

SEQ ID NO: 292 - DNA, 16 bp, Artificial sequence
  Synthetic oligonucleotide
ggaggctgaggatgca

SEQ ID NO: 293 - DNA, 17 bp, Artificial sequence
  Synthetic oligonucleotide
aggaggctgaggatgca

SEQ ID NO: 294 - DNA, 17 bp, Artificial sequence
  Synthetic oligonucleotide
ctggtttggaaggaggc

SEQ ID NO: 295 - DNA, 16 bp, Artificial sequence
  Synthetic oligonucleotide
ctggtttggaaggagg

SEQ ID NO: 296 - DNA, 17 bp, Artificial sequence
  Synthetic oligonucleotide
gtctggtttggaaggag

SEQ ID NO: 297 - DNA, 17 bp, Artificial sequence
  Synthetic oligonucleotide
gcgctactgggccacgt

SEQ ID NO: 298 - DNA, 17 bp, Artificial sequence
  Synthetic oligonucleotide
ttactgagcgcggaaga

SEQ ID NO: 299 - DNA, 16 bp, Artificial sequence
  Synthetic oligonucleotide
ttactgagcgcggaag

SEQ ID NO: 300 - DNA, 16 bp, Artificial sequence
  Synthetic oligonucleotide
gcaggagcggcctaaa

SEQ ID NO: 301 - DNA, 16 bp, Artificial sequence
  Synthetic oligonucleotide
tgcaggagcggcctaa

SEQ ID NO: 302 - DNA, 17 bp, Artificial sequence
  Synthetic oligonucleotide
ctgcaggagcggcctaa

SEQ ID NO: 303 - DNA, 17 bp, Artificial sequence
  Synthetic oligonucleotide
tctgcaggagcggccta

SEQ ID NO: 304 - DNA, 17 bp, Artificial sequence
  Synthetic oligonucleotide
ttctgcaggagcggcct

SEQ ID NO: 305 - DNA, 17 bp, Artificial sequence
  Synthetic oligonucleotide
gagttctgcaggagcgg

SEQ ID NO: 306 - DNA, 17 bp, Artificial sequence
  Synthetic oligonucleotide
cgagttctgcaggagcg

SEQ ID NO: 307 - DNA, 17 bp, Artificial sequence
  Synthetic oligonucleotide
ccgagttctgcaggagc

SEQ ID NO: 308 - DNA, 17 bp, Artificial sequence
  Synthetic oligonucleotide
aggacgaggcctcaggt

SEQ ID NO: 309 - DNA, 17 bp, Artificial sequence
  Synthetic oligonucleotide
agggacgacctttgctg

SEQ ID NO: 310 - DNA, 16 bp, Artificial sequence
  Synthetic oligonucleotide
agggacgacctttgct

SEQ ID NO: 311 - DNA, 17 bp, Artificial sequence
  Synthetic oligonucleotide
gtgggcaccacgcggtg

SEQ ID NO: 312 - DNA, 17 bp, Artificial sequence
  Synthetic oligonucleotide
tgtgggcaccacgcggt

SEQ ID NO: 313 - DNA, 17 bp, Artificial sequence
  Synthetic oligonucleotide
agctgtgggcaccacgc

SEQ ID NO: 314 - DNA, 17 bp, Artificial sequence
  Synthetic oligonucleotide
gagctgtgggcaccacg

SEQ ID NO: 315 - DNA, 17 bp, Artificial sequence
  Synthetic oligonucleotide
tgagctgtgggcaccac

SEQ ID NO: 316 - DNA, 17 bp, Artificial sequence
  Synthetic oligonucleotide
gacctcgcgcggatcct

SEQ ID NO: 317 - DNA, 17 bp, Artificial sequence
  Synthetic oligonucleotide
ccgaggcgcggcccctg

SEQ ID NO: 318 - DNA, 17 bp, Artificial sequence
  Synthetic oligonucleotide
tccgaggcgcggcccct

SEQ ID NO: 319 - DNA, 17 bp, Artificial sequence
  Synthetic oligonucleotide
cgtctccggcggccaga

SEQ ID NO: 320 - DNA, 17 bp, Artificial sequence
  Synthetic oligonucleotide
acagccgcccgcggaaa

SEQ ID NO: 321 - DNA, 17 bp, Artificial sequence
  Synthetic oligonucleotide
ccggccgcagctcctcc

SEQ ID NO: 322 - DNA, 17 bp, Artificial sequence
  Synthetic oligonucleotide
cccggccgcagctcctc

SEQ ID NO: 323 - DNA, 17 bp, Artificial sequence
  Synthetic oligonucleotide
tccttgcactccctctc

SEQ ID NO: 324 - DNA, 17 bp, Artificial sequence
  Synthetic oligonucleotide
tctcccgggcctcctcg

SEQ ID NO: 325 - DNA, 17 bp, Artificial sequence
  Synthetic oligonucleotide
tgatgtgaaaaccggca

SEQ ID NO: 326 - DNA, 16 bp, Artificial sequence
  Synthetic oligonucleotide
tattttctgatgtgaa

SEQ ID NO: 327 - DNA, 16 bp, Artificial sequence
  Synthetic oligonucleotide
gtattttctgatgtga

SEQ ID NO: 328 - DNA, 17 bp, Artificial sequence
  Synthetic oligonucleotide
cgtattttctgatgtga

SEQ ID NO: 329 - DNA, 16 bp, Artificial sequence
  Synthetic oligonucleotide
ggtgacccgtgagcgt

SEQ ID NO: 330 - DNA, 16 bp, Artificial sequence
  Synthetic oligonucleotide
aggtgacccgtgagcg

SEQ ID NO: 331 - DNA, 17 bp, Artificial sequence
  Synthetic oligonucleotide
gaggtgacccgtgagcg

SEQ ID NO: 332 - DNA, 17 bp, Artificial sequence
  Synthetic oligonucleotide
ggcatgaccatcctcaa

SEQ ID NO: 333 - DNA, 17 bp, Artificial sequence
  Synthetic oligonucleotide
tgctaggcatgaccatc

SEQ ID NO: 334 - DNA, 17 bp, Artificial sequence
  Synthetic oligonucleotide
tgctgtgagaatacaac

SEQ ID NO: 335 - DNA, 17 bp, Artificial sequence
  Synthetic oligonucleotide
ggtgctgtgagaataca

SEQ ID NO: 336 - DNA, 17 bp, Artificial sequence
  Synthetic oligonucleotide
agaaactgttggccacc

SEQ ID NO: 337 - DNA, 17 bp, Artificial sequence
  Synthetic oligonucleotide
aagaaactgttggccac

SEQ ID NO: 338 - DNA, 17 bp, Artificial sequence
  Synthetic oligonucleotide
agtgattgctgaagaaa

SEQ ID NO: 339 - DNA, 17 bp, Artificial sequence
  Synthetic oligonucleotide
gcagtagcagatgcaaa

SEQ ID NO: 340 - DNA, 17 bp, Artificial sequence
  Synthetic oligonucleotide
atgggtgaccacacatt

SEQ ID NO: 341 - DNA, 17 bp, Artificial sequence
  Synthetic oligonucleotide
ggcacactggtccccat

SEQ ID NO: 342 - DNA, 17 bp, Artificial sequence
  Synthetic oligonucleotide
aggcacactggtcccca

SEQ ID NO: 343 - DNA, 17 bp, Artificial sequence
  Synthetic oligonucleotide
tgaggcacactggtccc

SEQ ID NO: 344 - DNA, 17 bp, Artificial sequence
  Synthetic oligonucleotide
tgcaggagcccccattc

SEQ ID NO: 345 - DNA, 17 bp, Artificial sequence
  Synthetic oligonucleotide
tggagctggtccttgca

SEQ ID NO: 346 - DNA, 17 bp, Artificial sequence
  Synthetic oligonucleotide
aggactggagctggtcc

SEQ ID NO: 347 - DNA, 17 bp, Artificial sequence
  Synthetic oligonucleotide
cagatataggactggag

SEQ ID NO: 348 - DNA, 17 bp, Artificial sequence
  Synthetic oligonucleotide
agcagatataggactgg

SEQ ID NO: 349 - DNA, 17 bp, Artificial sequence
  Synthetic oligonucleotide
acagttccggccctcga

SEQ ID NO: 350 - DNA, 17 bp, Artificial sequence
  Synthetic oligonucleotide
ctcacagttccggccct

SEQ ID NO: 351 - DNA, 17 bp, Artificial sequence
  Synthetic oligonucleotide
aggttgtgcaccctgca

SEQ ID NO: 352 - DNA, 17 bp, Artificial sequence
  Synthetic oligonucleotide
ccaggttgtgcaccctg

SEQ ID NO: 353 - DNA, 17 bp, Artificial sequence
  Synthetic oligonucleotide
cctgtcaaagacctcag

SEQ ID NO: 354 - DNA, 17 bp, Artificial sequence
  Synthetic oligonucleotide
gaagtttaccaagcggt

SEQ ID NO: 355 - DNA, 17 bp, Artificial sequence
  Synthetic oligonucleotide
aacctctggacaccggg

SEQ ID NO: 356 - DNA, 17 bp, Artificial sequence
  Synthetic oligonucleotide
tgtgattgagccctgat

SEQ ID NO: 357 - DNA, 17 bp, Artificial sequence
  Synthetic oligonucleotide
tggtctagctgacaatg

SEQ ID NO: 358 - DNA, 17 bp, Artificial sequence
  Synthetic oligonucleotide
tgctggatggtctagct

SEQ ID NO: 359 - DNA, 17 bp, Artificial sequence
  Synthetic oligonucleotide
ggcattctggacccaga

SEQ ID NO: 360 - DNA, 17 bp, Artificial sequence
  Synthetic oligonucleotide
aggatgcacaccagggc

SEQ ID NO: 361 - DNA, 17 bp, Artificial sequence
  Synthetic oligonucleotide
caggatgcacaccaggg

SEQ ID NO: 362 - DNA, 17 bp, Artificial sequence
  Synthetic oligonucleotide
aagtccaggactccggc

SEQ ID NO: 363 - DNA, 17 bp, Artificial sequence
  Synthetic oligonucleotide
ccgagcagctgatggga

SEQ ID NO: 364 - DNA, 17 bp, Artificial sequence
  Synthetic oligonucleotide
ccactcttccgagcagc

SEQ ID NO: 365 - DNA, 16 bp, Artificial sequence
  Synthetic oligonucleotide
ccactcttccgagcag

SEQ ID NO: 366 - DNA, 17 bp, Artificial sequence
  Synthetic oligonucleotide
atcagctggtcatcctt

SEQ ID NO: 367 - DNA, 17 bp, Artificial sequence
  Synthetic oligonucleotide
cagatcagctggtcatc

SEQ ID NO: 368 - DNA, 17 bp, Artificial sequence
  Synthetic oligonucleotide
tgctcacagccgccgtt

SEQ ID NO: 369 - DNA, 17 bp, Artificial sequence
  Synthetic oligonucleotide
ctgctcacagccgccgt

SEQ ID NO: 370 - DNA, 17 bp, Artificial sequence
  Synthetic oligonucleotide
actgctcacagccgccg

SEQ ID NO: 371 - DNA, 17 bp, Artificial sequence
  Synthetic oligonucleotide
tactgctcacagccgcc

SEQ ID NO: 372 - DNA, 17 bp, Artificial sequence
  Synthetic oligonucleotide
agtactgctcacagccg

SEQ ID NO: 373 - DNA, 17 bp, Artificial sequence
  Synthetic oligonucleotide
tgcagtactgctcacag

SEQ ID NO: 374 - DNA, 17 bp, Artificial sequence
  Synthetic oligonucleotide
tcactgcagtactgctc

SEQ ID NO: 375 - DNA, 17 bp, Artificial sequence
  Synthetic oligonucleotide
tggtcactgcagtactg

SEQ ID NO: 376 - DNA, 17 bp, Artificial sequence
  Synthetic oligonucleotide
caccccgtctgccagca

SEQ ID NO: 377 - DNA, 17 bp, Artificial sequence
  Synthetic oligonucleotide
tggtgtcatctgggact

SEQ ID NO: 378 - DNA, 17 bp, Artificial sequence
  Synthetic oligonucleotide
atccgtagtgggacagg

SEQ ID NO: 379 - DNA, 17 bp, Artificial sequence
  Synthetic oligonucleotide
tgtcgctctggcctgtg

SEQ ID NO: 380 - DNA, 17 bp, Artificial sequence
  Synthetic oligonucleotide
actgtcgctctggcctg

SEQ ID NO: 381 - DNA, 17 bp, Artificial sequence
  Synthetic oligonucleotide
gtcactgtcgctctggc

SEQ ID NO: 382 - DNA, 17 bp, Artificial sequence
  Synthetic oligonucleotide
aggtcctgcgagtggga

SEQ ID NO: 383 - DNA, 17 bp, Artificial sequence
  Synthetic oligonucleotide
ggaggtcctgcgagtgg

SEQ ID NO: 384 - DNA, 17 bp, Artificial sequence
  Synthetic oligonucleotide
agcagtcagtacagaca

SEQ ID NO: 385 - DNA, 17 bp, Artificial sequence
  Synthetic oligonucleotide
tggaagtgcagcccatt

SEQ ID NO: 386 - DNA, 17 bp, Artificial sequence
  Synthetic oligonucleotide
ttggaagtgcagcccat

SEQ ID NO: 387 - DNA, 17 bp, Artificial sequence
  Synthetic oligonucleotide
tggtcagggctggtttt

SEQ ID NO: 388 - DNA, 16 bp, Artificial sequence
  Synthetic oligonucleotide
tggtcagggctggttt

SEQ ID NO: 389 - DNA, 17 bp, Artificial sequence
  Synthetic oligonucleotide
aatggtcagggctggtt

SEQ ID NO: 390 - DNA, 16 bp, Artificial sequence
  Synthetic oligonucleotide
atggtcagggctggtt

SEQ ID NO: 391 - DNA, 17 bp, Artificial sequence
  Synthetic oligonucleotide
caatggtcagggctggt

SEQ ID NO: 392 - DNA, 16 bp, Artificial sequence
  Synthetic oligonucleotide
caatggtcagggctgg

SEQ ID NO: 393 - DNA, 17 bp, Artificial sequence
  Synthetic oligonucleotide
acaatggtcagggctgg

SEQ ID NO: 394 - DNA, 16 bp, Artificial sequence
  Synthetic oligonucleotide
acaatggtcagggctg

SEQ ID NO: 395 - DNA, 17 bp, Artificial sequence
  Synthetic oligonucleotide
aggagacaatggtcagg

SEQ ID NO: 396 - DNA, 17 bp, Artificial sequence
  Synthetic oligonucleotide
gaggagacaatggtcag

SEQ ID NO: 397 - DNA, 17 bp, Artificial sequence
  Synthetic oligonucleotide
taggtatttttccacat

SEQ ID NO: 398 - DNA, 16 bp, Artificial sequence
  Synthetic oligonucleotide
gtttgctggcatttct

SEQ ID NO: 399 - DNA, 17 bp, Artificial sequence
  Synthetic oligonucleotide
gggtttgctggcatttc

SEQ ID NO: 400 - DNA, 16 bp, Artificial sequence
  Synthetic oligonucleotide
ggtttgctggcatttc

SEQ ID NO: 401 - DNA, 17 bp, Artificial sequence
  Synthetic oligonucleotide
tgacttggagcctggtg

SEQ ID NO: 402 - DNA, 17 bp, Artificial sequence
  Synthetic oligonucleotide
ttctctgcacagggtag

SEQ ID NO: 403 - DNA, 17 bp, Artificial sequence
  Synthetic oligonucleotide
tgatccaatgctcctga

SEQ ID NO: 404 - DNA, 17 bp, Artificial sequence
  Synthetic oligonucleotide
ttgatccaatgctcctg

SEQ ID NO: 405 - DNA, 17 bp, Artificial sequence
  Synthetic oligonucleotide
tttgatccaatgctcct

SEQ ID NO: 406 - DNA, 16 bp, Artificial sequence
  Synthetic oligonucleotide
tttgatccaatgctcc

SEQ ID NO: 407 - DNA, 17 bp, Artificial sequence
  Synthetic oligonucleotide
agctttgatccaatgct

SEQ ID NO: 408 - DNA, 17 bp, Artificial sequence
  Synthetic oligonucleotide
atagctttgatccaatg

SEQ ID NO: 409 - DNA, 17 bp, Artificial sequence
  Synthetic oligonucleotide
catagctttgatccaat

SEQ ID NO: 410 - DNA, 17 bp, Artificial sequence
  Synthetic oligonucleotide
atcttcacatagctttg

SEQ ID NO: 411 - DNA, 17 bp, Artificial sequence
  Synthetic oligonucleotide
gtcgctgtgagatttca

SEQ ID NO: 412 - DNA, 16 bp, Artificial sequence
  Synthetic oligonucleotide
tcagacctggctctgg

SEQ ID NO: 413 - DNA, 16 bp, Artificial sequence
  Synthetic oligonucleotide
ctcagacctggctctg

SEQ ID NO: 414 - DNA, 17 bp, Artificial sequence
  Synthetic oligonucleotide
gctcagacctggctctg

SEQ ID NO: 415 - DNA, 16 bp, Artificial sequence
  Synthetic oligonucleotide
gctgctcagacctggc

SEQ ID NO: 416 - DNA, 17 bp, Artificial sequence
  Synthetic oligonucleotide
agctgctcagacctggc

SEQ ID NO: 417 - DNA, 17 bp, Artificial sequence
  Synthetic oligonucleotide
aagctgctcagacctgg

SEQ ID NO: 418 - DNA, 17 bp, Artificial sequence
  Synthetic oligonucleotide
aaagctgctcagacctg

SEQ ID NO: 419 - DNA, 17 bp, Artificial sequence
  Synthetic oligonucleotide
ctctggcattgcacaat

SEQ ID NO: 420 - DNA, 17 bp, Artificial sequence
  Synthetic oligonucleotide
ttcaccaacaacaggac

SEQ ID NO: 421 - DNA, 17 bp, Artificial sequence
  Synthetic oligonucleotide
gagctccattcaccaac

SEQ ID NO: 422 - DNA, 16 bp, Artificial sequence
  Synthetic oligonucleotide
gagctccattcaccaa

SEQ ID NO: 423 - DNA, 16 bp, Artificial sequence
  Synthetic oligonucleotide
gctcgctgaggtcgtg

SEQ ID NO: 424 - DNA, 17 bp, Artificial sequence
  Synthetic oligonucleotide
gtgctcgctgaggtcgt

SEQ ID NO: 425 - DNA, 16 bp, Artificial sequence
  Synthetic oligonucleotide
tgctcgctgaggtcgt

SEQ ID NO: 426 - DNA, 17 bp, Artificial sequence
  Synthetic oligonucleotide
cgtgctcgctgaggtcg

SEQ ID NO: 427 - DNA, 17 bp, Artificial sequence
  Synthetic oligonucleotide
gccggctctgctcatcc

SEQ ID NO: 428 - DNA, 17 bp, Artificial sequence
  Synthetic oligonucleotide
tgcgccacccgccggct

SEQ ID NO: 429 - DNA, 17 bp, Artificial sequence
  Synthetic oligonucleotide
gacctgcgccacccgcc

SEQ ID NO: 430 - DNA, 17 bp, Artificial sequence
  Synthetic oligonucleotide
tgacctgcgccacccgc

SEQ ID NO: 431 - DNA, 17 bp, Artificial sequence
  Synthetic oligonucleotide
caggcggagcagcgcga

SEQ ID NO: 432 - DNA, 17 bp, Artificial sequence
  Synthetic oligonucleotide
tccgttcgggcaggcag

SEQ ID NO: 433 - DNA, 17 bp, Artificial sequence
  Synthetic oligonucleotide
cctgggtcatcagccgg

SEQ ID NO: 434 - DNA, 17 bp, Artificial sequence
  Synthetic oligonucleotide
agtcctgggtcatcagc

SEQ ID NO: 435 - DNA, 17 bp, Artificial sequence
  Synthetic oligonucleotide
ggcagtcctgggtcatc

SEQ ID NO: 436 - DNA, 17 bp, Artificial sequence
  Synthetic oligonucleotide
acatgtactccgtgata

SEQ ID NO: 437 - DNA, 17 bp, Artificial sequence
  Synthetic oligonucleotide
aacatgtactccgtgat

SEQ ID NO: 438 - DNA, 17 bp, Artificial sequence
  Synthetic oligonucleotide
agaacatgtactccgtg

SEQ ID NO: 439 - DNA, 16 bp, Artificial sequence
  Synthetic oligonucleotide
agaacatgtactccgt

SEQ ID NO: 440 - DNA, 17 bp, Artificial sequence
  Synthetic oligonucleotide
cagaacatgtactccgt

SEQ ID NO: 441 - DNA, 16 bp, Artificial sequence
  Synthetic oligonucleotide
agtagccggcacagaa

SEQ ID NO: 442 - DNA, 16 bp, Artificial sequence
  Synthetic oligonucleotide
gagtagccggcacaga

SEQ ID NO: 443 - DNA, 17 bp, Artificial sequence
  Synthetic oligonucleotide
cgagtagccggcacaga

SEQ ID NO: 444 - DNA, 17 bp, Artificial sequence
  Synthetic oligonucleotide
ccgagtagccggcacag

SEQ ID NO: 445 - DNA, 17 bp, Artificial sequence
  Synthetic oligonucleotide
tccgagtagccggcaca

SEQ ID NO: 446 - DNA, 17 bp, Artificial sequence
  Synthetic oligonucleotide
cccccttgcaggagtcc

SEQ ID NO: 447 - DNA, 17 bp, Artificial sequence
  Synthetic oligonucleotide
tcccccttgcaggagtc

SEQ ID NO: 448 - DNA, 17 bp, Artificial sequence
  Synthetic oligonucleotide
tccactgtcccccttgc

SEQ ID NO: 449 - DNA, 17 bp, Artificial sequence
  Synthetic oligonucleotide
accctggtgtacacccc

SEQ ID NO: 450 - DNA, 17 bp, Artificial sequence
  Synthetic oligonucleotide
agaccctggtgtacacc

SEQ ID NO: 451 - DNA, 17 bp, Artificial sequence
  Synthetic oligonucleotide
tactgggagaccctggt

SEQ ID NO: 452 - DNA, 16 bp, Artificial sequence
  Synthetic oligonucleotide
tactgggagaccctgg

SEQ ID NO: 453 - DNA, 17 bp, Artificial sequence
  Synthetic oligonucleotide
tgtactgggagaccctg

SEQ ID NO: 454 - DNA, 16 bp, Artificial sequence
  Synthetic oligonucleotide
gtactgggagaccctg

SEQ ID NO: 455 - DNA, 17 bp, Artificial sequence
  Synthetic oligonucleotide
tcgatgtactgggagac

SEQ ID NO: 456 - DNA, 17 bp, Artificial sequence
  Synthetic oligonucleotide
ctcgatgtactgggaga

SEQ ID NO: 457 - DNA, 17 bp, Artificial sequence
  Synthetic oligonucleotide
gagcttttgcagccact

SEQ ID NO: 458 - DNA, 17 bp, Artificial sequence
  Synthetic oligonucleotide
tgagcttttgcagccac

SEQ ID NO: 459 - DNA, 17 bp, Artificial sequence
  Synthetic oligonucleotide
gccttggctttctctcc

SEQ ID NO: 460 - DNA, 16 bp, Artificial sequence
  Synthetic oligonucleotide
gccttggctttctctc

SEQ ID NO: 461 - DNA, 17 bp, Artificial sequence
  Synthetic oligonucleotide
cccctctgtccagcgcc

SEQ ID NO: 462 - DNA, 16 bp, Artificial sequence
  Synthetic oligonucleotide
gcccctctgtccagcg

SEQ ID NO: 463 - DNA, 16 bp, Artificial sequence
  Synthetic oligonucleotide
tgcccctctgtccagc

SEQ ID NO: 464 - DNA, 16 bp, Artificial sequence
  Synthetic oligonucleotide
ctgcccctctgtccag

SEQ ID NO: 465 - DNA, 17 bp, Artificial sequence
  Synthetic oligonucleotide
gaggccagcagatcacg

SEQ ID NO: 466 - DNA, 17 bp, Artificial sequence
  Synthetic oligonucleotide
agcctgaggccagcaga

SEQ ID NO: 467 - DNA, 17 bp, Artificial sequence
  Synthetic oligonucleotide
tccagcaatgaaggcag

SEQ ID NO: 468 - DNA, 17 bp, Artificial sequence
  Synthetic oligonucleotide
tgtctccagcaatgaag

SEQ ID NO: 469 - DNA, 17 bp, Artificial sequence
  Synthetic oligonucleotide
cacatggagtcagcatc

SEQ ID NO: 470 - DNA, 17 bp, Artificial sequence
  Synthetic oligonucleotide
acacatggagtcagcat

SEQ ID NO: 471 - DNA, 17 bp, Artificial sequence
  Synthetic oligonucleotide
gaggacagcacacatgg

SEQ ID NO: 472 - DNA, 17 bp, Artificial sequence
  Synthetic oligonucleotide
agctaaacaaccgcctt

SEQ ID NO: 473 - DNA, 17 bp, Artificial sequence
  Synthetic oligonucleotide
gagctaaacaaccgcct

SEQ ID NO: 474 - DNA, 17 bp, Artificial sequence
  Synthetic oligonucleotide
agagctaaacaaccgcc

SEQ ID NO: 475 - DNA, 17 bp, Artificial sequence
  Synthetic oligonucleotide
gagagctaaacaaccgc

SEQ ID NO: 476 - DNA, 16 bp, Artificial sequence
  Synthetic oligonucleotide
aagatgataatggata

SEQ ID NO: 477 - DNA, 17 bp, Artificial sequence
  Synthetic oligonucleotide
tgaagatgataatggat

SEQ ID NO: 478 - DNA, 16 bp, Artificial sequence
  Synthetic oligonucleotide
gaagatgataatggat

SEQ ID NO: 479 - DNA, 17 bp, Artificial sequence
  Synthetic oligonucleotide
atgcttctgaattgtct

SEQ ID NO: 480 - DNA, 17 bp, Artificial sequence
  Synthetic oligonucleotide
gatgcttctgaattgtc

SEQ ID NO: 481 - DNA, 17 bp, Artificial sequence
  Synthetic oligonucleotide
ggtgatgcttctgaatt

SEQ ID NO: 482 - DNA, 17 bp, Artificial sequence
  Synthetic oligonucleotide
tggtgatgcttctgaat

SEQ ID NO: 483 - DNA, 17 bp, Artificial sequence
  Synthetic oligonucleotide
atggtgatgcttctgaa

SEQ ID NO: 484 - DNA, 17 bp, Artificial sequence
  Synthetic oligonucleotide
catgcatggtgatgctt

SEQ ID NO: 485 - DNA, 17 bp, Artificial sequence
  Synthetic oligonucleotide
cattcgccaccatgcat

SEQ ID NO: 486 - DNA, 16 bp, Artificial sequence
  Synthetic oligonucleotide
cattcgccaccatgca

SEQ ID NO: 487 - DNA, 17 bp, Artificial sequence
  Synthetic oligonucleotide
tggtgcccaggacggcc

SEQ ID NO: 488 - DNA, 16 bp, Artificial sequence
  Synthetic oligonucleotide
accatgatgaaatctc

SEQ ID NO: 489 - DNA, 16 bp, Artificial sequence
  Synthetic oligonucleotide
gaccatgatgaaatct

SEQ ID NO: 490 - DNA, 17 bp, Artificial sequence
  Synthetic oligonucleotide
agaccatgatgaaatct

SEQ ID NO: 491 - DNA, 16 bp, Artificial sequence
  Synthetic oligonucleotide
accagtggcagtccct

SEQ ID NO: 492 - DNA, 16 bp, Artificial sequence
  Synthetic oligonucleotide
tgcatcagaaaagctc

SEQ ID NO: 493 - DNA, 16 bp, Artificial sequence
  Synthetic oligonucleotide
cagatttgcatcagaa

SEQ ID NO: 494 - DNA, 16 bp, Artificial sequence
  Synthetic oligonucleotide
gtctggtttggaagga

SEQ ID NO: 495 - DNA, 16 bp, Artificial sequence
  Synthetic oligonucleotide
cccggccgcagctcct

SEQ ID NO: 496 - DNA, 16 bp, Artificial sequence
  Synthetic oligonucleotide
atgggtgaccacacat

SEQ ID NO: 497 - DNA, 16 bp, Artificial sequence
  Synthetic oligonucleotide
aagtttaccaagcggt

SEQ ID NO: 498 - DNA, 16 bp, Artificial sequence
  Synthetic oligonucleotide
gaagtttaccaagcgg

SEQ ID NO: 499 - DNA, 16 bp, Artificial sequence
  Synthetic oligonucleotide
acctctggacaccggg

SEQ ID NO: 500 - DNA, 16 bp, Artificial sequence
  Synthetic oligonucleotide
gaggagacaatggtca

SEQ ID NO: 501 - DNA, 16 bp, Artificial sequence
  Synthetic oligonucleotide
tggatattcaactgtg

SEQ ID NO: 502 - DNA, 16 bp, Artificial sequence
  Synthetic oligonucleotide
taggtatttttccaca

SEQ ID NO: 503 - DNA, 16 bp, Artificial sequence
  Synthetic oligonucleotide
atagctttgatccaat

SEQ ID NO: 504 - DNA, 16 bp, Artificial sequence
  Synthetic oligonucleotide
catagctttgatccaa

SEQ ID NO: 505 - DNA, 16 bp, Artificial sequence
  Synthetic oligonucleotide
aacatgtactccgtga

SEQ ID NO: 506 - DNA, 16 bp, Artificial sequence
  Synthetic oligonucleotide
cacatggagtcagcat

SEQ ID NO: 507 - DNA, 16 bp, Artificial sequence
  Synthetic oligonucleotide
agctaaacaaccgcct

SEQ ID NO: 508 - DNA, 14 bp, Artificial sequence
  Synthetic oligonucleotide
tgtgcagcccggca

SEQ ID NO: 509 - DNA, 16 bp, Artificial sequence
  Synthetic oligonucleotide
gtgtgaggtgacctgt

SEQ ID NO: 510 - DNA, 16 bp, Artificial sequence
  Synthetic oligonucleotide
agtgtgaggtgacctg

SEQ ID NO: 511 - DNA, 16 bp, Artificial sequence
  Synthetic oligonucleotide
gagtgtgaggtgacct

SEQ ID NO: 512 - DNA, 16 bp, Artificial sequence
  Synthetic oligonucleotide
gtgagtgtgaggtgac

SEQ ID NO: 513 - DNA, 16 bp, Artificial sequence
  Synthetic oligonucleotide
tgtgagtgtgaggtga

SEQ ID NO: 514 - DNA, 16 bp, Artificial sequence
  Synthetic oligonucleotide
ctgtgagtgtgaggtg

SEQ ID NO: 515 - DNA, 16 bp, Artificial sequence
  Synthetic oligonucleotide
cctgtgagtgtgaggt

SEQ ID NO: 516 - DNA, 16 bp, Artificial sequence
  Synthetic oligonucleotide
tcctgtgagtgtgagg

SEQ ID NO: 517 - DNA, 16 bp, Artificial sequence
  Synthetic oligonucleotide
gtcctgtgagtgtgag

SEQ ID NO: 518 - DNA, 16 bp, Artificial sequence
  Synthetic oligonucleotide
tgtcctgtgagtgtga

SEQ ID NO: 519 - DNA, 16 bp, Artificial sequence
  Synthetic oligonucleotide
gtgtcctgtgagtgtg

SEQ ID NO: 520 - DNA, 16 bp, Artificial sequence
  Synthetic oligonucleotide
ggtgtcctgtgagtgt

SEQ ID NO: 521 - DNA, 16 bp, Artificial sequence
  Synthetic oligonucleotide
gaggtgtcctgtgagt

SEQ ID NO: 522 - DNA, 16 bp, Artificial sequence
  Synthetic oligonucleotide
tgaggtgtcctgtgag

SEQ ID NO: 523 - DNA, 16 bp, Artificial sequence
  Synthetic oligonucleotide
gtgaggtgtcctgtga

SEQ ID NO: 524 - DNA, 16 bp, Artificial sequence
  Synthetic oligonucleotide
tgtgaggtgtcctgtg

SEQ ID NO: 525 - DNA, 16 bp, Artificial sequence
  Synthetic oligonucleotide
gtgtgaggtgtcctgt

SEQ ID NO: 526 - DNA, 16 bp, Artificial sequence
  Synthetic oligonucleotide
agtgtgaggtgtcctg

SEQ ID NO: 527 - DNA, 16 bp, Artificial sequence
  Synthetic oligonucleotide
gagtgtgaggtgtcct

SEQ ID NO: 528 - DNA, 16 bp, Artificial sequence
  Synthetic oligonucleotide
tgagtgtgaggtgtcc

SEQ ID NO: 529 - DNA, 16 bp, Artificial sequence
  Synthetic oligonucleotide
tgtgagtgtgaggtgt

SEQ ID NO: 530 - DNA, 16 bp, Artificial sequence
  Synthetic oligonucleotide
gccctgtgagtgtgag

SEQ ID NO: 531 - DNA, 16 bp, Artificial sequence
  Synthetic oligonucleotide
gtgccctgtgagtgtg

SEQ ID NO: 532 - DNA, 16 bp, Artificial sequence
  Synthetic oligonucleotide
cgtgagtgtgaagtgt

SEQ ID NO: 533 - DNA, 16 bp, Artificial sequence
  Synthetic oligonucleotide
ccgtgagtgtgaagtg

SEQ ID NO: 534 - DNA, 16 bp, Artificial sequence
  Synthetic oligonucleotide
cccgtgagtgtgaagt

SEQ ID NO: 535 - DNA, 16 bp, Artificial sequence
  Synthetic oligonucleotide
acccgtgagtgtgaag

SEQ ID NO: 536 - DNA, 16 bp, Artificial sequence
  Synthetic oligonucleotide
gacccgtgagtgtgaa

SEQ ID NO: 537 - DNA, 16 bp, Artificial sequence
  Synthetic oligonucleotide
tgacccgtgagtgtga

SEQ ID NO: 538 - DNA, 16 bp, Artificial sequence
  Synthetic oligonucleotide
gtgacccgtgagtgtg

SEQ ID NO: 539 - DNA, 16 bp, Artificial sequence
  Synthetic oligonucleotide
ggtgacccgtgagtgt

SEQ ID NO: 540 - DNA, 16 bp, Artificial sequence
  Synthetic oligonucleotide
aggtgacccgtgagtg

SEQ ID NO: 541 - DNA, 16 bp, Artificial sequence
  Synthetic oligonucleotide
gaggtgacccgtgagt

SEQ ID NO: 542 - DNA, 16 bp, Artificial sequence
  Synthetic oligonucleotide
tgaggtgacccgtgag

SEQ ID NO: 543 - DNA, 16 bp, Artificial sequence
  Synthetic oligonucleotide
gtgaggtgacccgtga

SEQ ID NO: 544 - DNA, 16 bp, Artificial sequence
  Synthetic oligonucleotide
tgtgaggtgacccgtg

SEQ ID NO: 545 - DNA, 16 bp, Artificial sequence
  Synthetic oligonucleotide
gtgtgaggtgacccgt

SEQ ID NO: 546 - DNA, 16 bp, Artificial sequence
  Synthetic oligonucleotide
agtgtgaggtgacccg

SEQ ID NO: 547 - DNA, 16 bp, Artificial sequence
  Synthetic oligonucleotide
gagtgtgaggtgaccc

SEQ ID NO: 548 - DNA, 16 bp, Artificial sequence
  Synthetic oligonucleotide
tgagtgtgaagtgtgc

SEQ ID NO: 549 - DNA, 16 bp, Artificial sequence
  Synthetic oligonucleotide
gtgagtgtgaagtgtg

SEQ ID NO: 550 - DNA, 16 bp, Artificial sequence
  Synthetic oligonucleotide
tgtgagtgtgaagtgt

SEQ ID NO: 551 - DNA, 16 bp, Artificial sequence
  Synthetic oligonucleotide
ctgtgagtgtgaagtg

SEQ ID NO: 552 - DNA, 16 bp, Artificial sequence
  Synthetic oligonucleotide
cctgtgagtgtgaagt

SEQ ID NO: 553 - DNA, 16 bp, Artificial sequence
  Synthetic oligonucleotide
acctgtgagtgtgaag

SEQ ID NO: 554 - DNA, 16 bp, Artificial sequence
  Synthetic oligonucleotide
gacctgtgagtgtgaa

SEQ ID NO: 555 - DNA, 16 bp, Artificial sequence
  Synthetic oligonucleotide
tgacctgtgagtgtga

SEQ ID NO: 556 - DNA, 16 bp, Artificial sequence
  Synthetic oligonucleotide
gtgacctgtgagtgtg

SEQ ID NO: 557 - DNA, 16 bp, Artificial sequence
  Synthetic oligonucleotide
ggtgacctgtgagtgt

SEQ ID NO: 558 - DNA, 16 bp, Artificial sequence
  Synthetic oligonucleotide
aggtgacctgtgagtg

SEQ ID NO: 559 - DNA, 16 bp, Artificial sequence
  Synthetic oligonucleotide
gaggtgacctgtgagt

SEQ ID NO: 560 - DNA, 16 bp, Artificial sequence
  Synthetic oligonucleotide
tgaggtgacctgtgag

SEQ ID NO: 561 - DNA, 16 bp, Artificial sequence
  Synthetic oligonucleotide
gtgaggtgacctgtga

SEQ ID NO: 562 - DNA, 16 bp, Artificial sequence
  Synthetic oligonucleotide
tgtgaggtgacctgtg

SEQ ID NO: 563 - DNA, 16 bp, Artificial sequence
  Synthetic oligonucleotide
tgaagtgtgccctgtg

SEQ ID NO: 564 - DNA, 16 bp, Artificial sequence
  Synthetic oligonucleotide
gtgaagtgtgccctgt

SEQ ID NO: 565 - DNA, 16 bp, Artificial sequence
  Synthetic oligonucleotide
tgtgaagtgtgccctg

SEQ ID NO: 566 - DNA, 16 bp, Artificial sequence
  Synthetic oligonucleotide
gtgtgaagtgtgccct

SEQ ID NO: 567 - DNA, 16 bp, Artificial sequence
  Synthetic oligonucleotide
agtgtgaagtgtgccc

SEQ ID NO: 568 - DNA, 16 bp, Artificial sequence
  Synthetic oligonucleotide
gagtgtgaagtgtgcc

SEQ ID NO: 569 - DNA, 16 bp, Artificial sequence
  Synthetic oligonucleotide
gtgtgaggtgtcctct

SEQ ID NO: 570 - DNA, 16 bp, Artificial sequence
  Synthetic oligonucleotide
tgtgtgaggtgtcctc

SEQ ID NO: 571 - DNA, 16 bp, Artificial sequence
  Synthetic oligonucleotide
ctgtgtgaggtgtcct

SEQ ID NO: 572 - DNA, 16 bp, Artificial sequence
  Synthetic oligonucleotide
cctgtgtgaggtgtcc

SEQ ID NO: 573 - DNA, 16 bp, Artificial sequence
  Synthetic oligonucleotide
ccctgtgtgaggtgtc

SEQ ID NO: 574 - DNA, 16 bp, Artificial sequence
  Synthetic oligonucleotide
gccctgtgtgaggtgt

SEQ ID NO: 575 - DNA, 16 bp, Artificial sequence
  Synthetic oligonucleotide
tgccctgtgtgaggtg

SEQ ID NO: 576 - DNA, 16 bp, Artificial sequence
  Synthetic oligonucleotide
gtgccctgtgtgaggt

SEQ ID NO: 577 - DNA, 16 bp, Artificial sequence
  Synthetic oligonucleotide
tgtgccctgtgtgagg

SEQ ID NO: 578 - DNA, 16 bp, Artificial sequence
  Synthetic oligonucleotide
gtgtgccctgtgtgag

SEQ ID NO: 579 - DNA, 16 bp, Artificial sequence
  Synthetic oligonucleotide
agtgtgccctgtgtga

SEQ ID NO: 580 - DNA, 16 bp, Artificial sequence
  Synthetic oligonucleotide
aagtgtgccctgtgtg

SEQ ID NO: 581 - DNA, 16 bp, Artificial sequence
  Synthetic oligonucleotide
gaagtgtgccctgtgt

SEQ ID NO: 582 - DNA, 16 bp, Artificial sequence
  Synthetic oligonucleotide
tgtgtgaggtgtcctg

SEQ ID NO: 583 - DNA, 16 bp, Artificial sequence
  Synthetic oligonucleotide
tgtgaggtgtcttgtg

SEQ ID NO: 584 - DNA, 16 bp, Artificial sequence
  Synthetic oligonucleotide
gtgtgaggtgtcttgt

SEQ ID NO: 585 - DNA, 16 bp, Artificial sequence
  Synthetic oligonucleotide
gaagtgtgccccgtgt

SEQ ID NO: 586 - DNA, 16 bp, Artificial sequence
  Synthetic oligonucleotide
gtgaagtgtgccccgt

SEQ ID NO: 587 - DNA, 16 bp, Artificial sequence
  Synthetic oligonucleotide
gtgtgaagtgtgcccc

SEQ ID NO: 588 - DNA, 16 bp, Artificial sequence
  Synthetic oligonucleotide
gggtgtgaggtgacct

SEQ ID NO: 589 - DNA, 16 bp, Artificial sequence
  Synthetic oligonucleotide
tgggtgtgaggtgacc

SEQ ID NO: 590 - DNA, 16 bp, Artificial sequence
  Synthetic oligonucleotide
gtgggtgtgaggtgac

SEQ ID NO: 591 - DNA, 16 bp, Artificial sequence
  Synthetic oligonucleotide
tgtgggtgtgaggtga

SEQ ID NO: 592 - DNA, 16 bp, Artificial sequence
  Synthetic oligonucleotide
ctgtgggtgtgaggtg

SEQ ID NO: 593 - DNA, 16 bp, Artificial sequence
  Synthetic oligonucleotide
cctgtgggtgtgaggt

SEQ ID NO: 594 - DNA, 16 bp, Artificial sequence
  Synthetic oligonucleotide
tcctgtgggtgtgagg

SEQ ID NO: 595 - DNA, 16 bp, Artificial sequence
  Synthetic oligonucleotide
gtcctgtgggtgtgag

SEQ ID NO: 596 - DNA, 16 bp, Artificial sequence
  Synthetic oligonucleotide
tgtcctgtgggtgtga

SEQ ID NO: 597 - DNA, 16 bp, Artificial sequence
  Synthetic oligonucleotide
gtgtcctgtgggtgtg

SEQ ID NO: 598 - DNA, 16 bp, Artificial sequence
  Synthetic oligonucleotide
ggtgtcctgtgggtgt

SEQ ID NO: 599 - DNA, 16 bp, Artificial sequence
  Synthetic oligonucleotide
aggtgtcctgtgggtg

SEQ ID NO: 600 - DNA, 16 bp, Artificial sequence
  Synthetic oligonucleotide
gaggtgtcctgtgggt

SEQ ID NO: 601 - DNA, 16 bp, Artificial sequence
  Synthetic oligonucleotide
tgaggtgtcctgtggg

SEQ ID NO: 602 - DNA, 16 bp, Artificial sequence
  Synthetic oligonucleotide
gtgaggtgtcctgtgg

SEQ ID NO: 603 - DNA, 16 bp, Artificial sequence
  Synthetic oligonucleotide
ctgtgaggtgtcctgt

SEQ ID NO: 604 - DNA, 16 bp, Artificial sequence
  Synthetic oligonucleotide
tctgtgaggtgtcctg

SEQ ID NO: 605 - DNA, 16 bp, Artificial sequence
  Synthetic oligonucleotide
ctctgtgaggtgtcct

SEQ ID NO: 606 - DNA, 16 bp, Artificial sequence
  Synthetic oligonucleotide
cctctgtgaggtgtcc

SEQ ID NO: 607 - DNA, 16 bp, Artificial sequence
  Synthetic oligonucleotide
gacctctgtgaggtgt

SEQ ID NO: 608 - DNA, 16 bp, Artificial sequence
  Synthetic oligonucleotide
gtgacctctgtgaggt

SEQ ID NO: 609 - DNA, 16 bp, Artificial sequence
  Synthetic oligonucleotide
aggtgacctctgtgag

SEQ ID NO: 610 - DNA, 16 bp, Artificial sequence
  Synthetic oligonucleotide
tgaggtgacctctgtg

SEQ ID NO: 611 - DNA, 16 bp, Artificial sequence
  Synthetic oligonucleotide
tgtgaggtgacctctg

SEQ ID NO: 612 - DNA, 16 bp, Artificial sequence
  Synthetic oligonucleotide
gtgtgaggtgacctct

SEQ ID NO: 613 - DNA, 16 bp, Artificial sequence
  Synthetic oligonucleotide
tgtgtgaggtgacctc

SEQ ID NO: 614 - DNA, 16 bp, Artificial sequence
  Synthetic oligonucleotide
cctgtgtgaggtgacc

SEQ ID NO: 615 - DNA, 16 bp, Artificial sequence
  Synthetic oligonucleotide
tgaggtgtcctgtgtg

SEQ ID NO: 616 - DNA, 16 bp, Artificial sequence
  Synthetic oligonucleotide
gtgaggtgtcctgtgt

SEQ ID NO: 617 - DNA, 16 bp, Artificial sequence
  Synthetic oligonucleotide
gacctgtgagtgtgag

SEQ ID NO: 618 - DNA, 16 bp, Artificial sequence
  Synthetic oligonucleotide
ggtgtcctgtgagagt

SEQ ID NO: 619 - DNA, 16 bp, Artificial sequence
  Synthetic oligonucleotide
gaggtgtcctgtgaga

SEQ ID NO: 620 - DNA, 16 bp, Artificial sequence
  Synthetic oligonucleotide
acctgtgagtgtgagg

SEQ ID NO: 621 - DNA, 16 bp, Artificial sequence
  Synthetic oligonucleotide
cgggacacccacaccc

SEQ ID NO: 622 - DNA, 16 bp, Artificial sequence
  Synthetic oligonucleotide
cccgggacacccacac

SEQ ID NO: 623 - DNA, 16 bp, Artificial sequence
  Synthetic oligonucleotide
ctcccgggacacccac

SEQ ID NO: 624 - DNA, 16 bp, Artificial sequence
  Synthetic oligonucleotide
actcccgggacaccca

SEQ ID NO: 625 - DNA, 16 bp, Artificial sequence
  Synthetic oligonucleotide
cactcccgggacaccc

SEQ ID NO: 626 - DNA, 16 bp, Artificial sequence
  Synthetic oligonucleotide
acactcccgggacacc

SEQ ID NO: 627 - DNA, 16 bp, Artificial sequence
  Synthetic oligonucleotide
cacactcccgggacac

SEQ ID NO: 628 - DNA, 16 bp, Artificial sequence
  Synthetic oligonucleotide
acccacactcccggga

SEQ ID NO: 629 - DNA, 16 bp, Artificial sequence
  Synthetic oligonucleotide
acacccacactcccgg

SEQ ID NO: 630 - DNA, 16 bp, Artificial sequence
  Synthetic oligonucleotide
gggacacccacactcc

SEQ ID NO: 631 - DNA, 16 bp, Artificial sequence
  Synthetic oligonucleotide
ccgggacacccacact

SEQ ID NO: 632 - DNA, 16 bp, Artificial sequence
  Synthetic oligonucleotide
ccccgggacacccaca

SEQ ID NO: 633 - DNA, 16 bp, Artificial sequence
  Synthetic oligonucleotide
cccccgggacacccac

SEQ ID NO: 634 - DNA, 16 bp, Artificial sequence
  Synthetic oligonucleotide
cgcccccgggacaccc

SEQ ID NO: 635 - DNA, 16 bp, Artificial sequence
  Synthetic oligonucleotide
ccacgcccccgggaca

SEQ ID NO: 636 - DNA, 16 bp, Artificial sequence
  Synthetic oligonucleotide
cacccacgcccccggg

SEQ ID NO: 637 - DNA, 16 bp, Artificial sequence
  Synthetic oligonucleotide
ggacacccacgccccc

SEQ ID NO: 638 - DNA, 16 bp, Artificial sequence
  Synthetic oligonucleotide
gggacacccacgcccc

SEQ ID NO: 639 - DNA, 16 bp, Artificial sequence
  Synthetic oligonucleotide
cgggacacccacgccc

SEQ ID NO: 640 - DNA, 16 bp, Artificial sequence
  Synthetic oligonucleotide
ccgggacacccacgcc

SEQ ID NO: 641 - DNA, 16 bp, Artificial sequence
  Synthetic oligonucleotide
cccgggacacccacgc

SEQ ID NO: 642 - DNA, 16 bp, Artificial sequence
  Synthetic oligonucleotide
cctccgggacacccac

SEQ ID NO: 643 - DNA, 16 bp, Artificial sequence
  Synthetic oligonucleotide
gcctccgggacaccca

SEQ ID NO: 644 - DNA, 16 bp, Artificial sequence
  Synthetic oligonucleotide
acaccctcgcctccgg

SEQ ID NO: 645 - DNA, 16 bp, Artificial sequence
  Synthetic oligonucleotide
gggacaccctcgcctc

SEQ ID NO: 646 - DNA, 16 bp, Artificial sequence
  Synthetic oligonucleotide
ccgggacaccctcgcc

SEQ ID NO: 647 - DNA, 16 bp, Artificial sequence
  Synthetic oligonucleotide
tcccgggacaccctcg

SEQ ID NO: 648 - DNA, 16 bp, Artificial sequence
  Synthetic oligonucleotide
actcccgggacaccct

SEQ ID NO: 649 - DNA, 16 bp, Artificial sequence
  Synthetic oligonucleotide
cgctcccgggacaccc

SEQ ID NO: 650 - DNA, 16 bp, Artificial sequence
  Synthetic oligonucleotide
ccccgggacacccacg

SEQ ID NO: 651 - DNA, 16 bp, Artificial sequence
  Synthetic oligonucleotide
ggaacacccacactcc

SEQ ID NO: 652 - DNA, 16 bp, Artificial sequence
  Synthetic oligonucleotide
tccggaacacccacac

SEQ ID NO: 653 - DNA, 16 bp, Artificial sequence
  Synthetic oligonucleotide
gcctccggaacaccca

SEQ ID NO: 654 - DNA, 16 bp, Artificial sequence
  Synthetic oligonucleotide
ctcgcctccggaacac

SEQ ID NO: 655 - DNA, 16 bp, Artificial sequence
  Synthetic oligonucleotide
accctcgcctccggaa

SEQ ID NO: 656 - DNA, 16 bp, Artificial sequence
  Synthetic oligonucleotide
acccccgggacaccca

SEQ ID NO: 657 - DNA, 16 bp, Artificial sequence
  Synthetic oligonucleotide
ccacacccccgggaca

SEQ ID NO: 658 - DNA, 16 bp, Artificial sequence
  Synthetic oligonucleotide
cacccacacccccggg

SEQ ID NO: 659 - DNA, 16 bp, Artificial sequence
  Synthetic oligonucleotide
gtgtgtgcatatctct

(19) United States
(12) Patent Application Publication
(10) Pub. No.: US 2015/0031747 A1
(43) Pub. Date: Jan. 29, 2015

(54) METHODS AND COMPOSITIONS FOR MODULATING FACTOR VII EXPRESSION

(71) Applicant: Isis Pharmaceuticals, Inc., Carlsbad, CA (US)

(72) Inventors: Eric E. Swayze, Encinitas, CA (US); Susan M. Freier, San Diego, CA (US)

(73) Assignee: Isis Pharmaceuticals, Inc., Carlsbad, CA (US)

(21) Appl. No.: 14/377,614

(22) PCT Filed: Feb. 8, 2013
(86) PCT No.: PCT/US13/25381

§ 371 Date: Aug. 8, 2014

Related U.S. Application Data
(60) Provisional application No. 61/596,688, filed on Feb. 8, 2012.

(51) Int. Cl. C12N 15/113

(52) U.S. Cl. CPC C12N 15/1137; C12N 2310/11; C12N 2310/315; C12N 2310/3341; C12N 2310/321; C12N 2310/346
USPC 514/44 A; 536/24.5

(57) ABSTRACT

Disclosed herein are antisense compounds and methods for decreasing Factor VII and treating, preventing, or slowing progression of thromboembolic complications, hyperproliferative disorders, or inflammatory conditions in an individual in need thereof.

### SEQUENCE LISTING

The present application is being filed along with a Sequence Listing in electronic format. The Sequence Listing is provided as a file entitled BIOL0169WOSEQ.txt created Feb. 6, 2013, which is 164 Kb in size. The information in the electronic format of the sequence listing is incorporated herein by reference in its entirety.

### FIELD

Embodiments described herein provide methods, compounds, and compositions for reducing expression of Factor VII mRNA and protein in an animal. Such methods, compounds, and compositions are useful to treat, prevent, or ameliorate thromboembolic complications, hyperproliferative disorders, and inflammatory conditions.

### BACKGROUND

The circulatory system requires mechanisms that prevent blood loss, as well as those that counteract inappropriate intravascular obstructions. Generally, coagulation comprises a cascade of reactions culminating in the conversion of soluble fibrinogen to an insoluble fibrin gel. The steps of the cascade involve the conversion of an inactive zymogen to an activated enzyme. The active enzyme then catalyzes the next step in the cascade.

### Coagulation Cascade

The coagulation cascade may be initiated through two branches, the tissue factor pathway (also “extrinsic pathway”), which is the primary pathway, and the contact activation pathway (also “intrinsic pathway”).

The tissue factor pathway is initiated by the cell surface receptor tissue factor (TF, also referred to as factor III), which is expressed constitutively by extravascular cells (pericytes, cardiomyocytes, smooth muscle cells, and keratinocytes) and expressed by vascular monocytes and endothelial cells upon induction by inflammatory cytokines or endotoxin. (Drake et al., Am J Pathol 1989, 134:1087-1097). TF is the high affinity cellular receptor for coagulation factor VIIa, a serine protease. In the absence of TF, VIIa has very low catalytic activity, and binding to TF is necessary to render VIIa functional through an allosteric mechanism. (Drake et al., Am J Pathol 1989, 134:1087-1097). The TF-VIIa complex activates factor X to Xa. Xa in turn associates with its co-factor factor Va into a prothrombinase complex which in turn activates prothrombin, (also known as factor II or factor 2) to thrombin (also known as factor Ha, or factor 2a). Thrombin activates platelets, converts fibrinogen to fibrin and promotes fibrin cross-linking by activating factor XIII, thus forming a stable plug at sites where TF is exposed on extravascular cells. In addition, thrombin reinforces the coagulation cascade response by activating factors V and VIII.

The contact activation pathway is triggered by activation of factor XII to XIIa. Factor XIIa converts XI to XIa, and XIa converts IX to IXa. IXa associates with its cofactor VIIIa to convert X to Xa. The two pathways converge at this point as factor Xa associates factor Va to activate prothrombin (factor II) to thrombin (factor IIa).

### Inhibition of Coagulation

At least three mechanisms keep the coagulation cascade in check, namely the action of activated protein C, antithrombin, and tissue factor pathway inhibitor. Activated protein C is a serine protease that degrades cofactors Va and VIIIa. Protein C is activated by thrombin with thrombomodulin, and requires coenzyme Protein S to function. Antithrombin is a serine protease inhibitor (serpin) that inhibits serine proteases: thrombin, Xa, XIIa, XIa and IXa. Tissue factor pathway inhibitor inhibits the action of Xa and the TF-VIIa complex. (Schwartz A L et al., Trends Cardiovasc Med. 1997; 7:234-239.)

### Disease

Thrombosis is the pathological development of blood clots, and an embolism occurs when a blood clot migrates to another part of the body and interferes with organ function. Thromboembolism may cause conditions such as deep vein thrombosis, pulmonary embolism, myocardial infarction, and stroke. Significantly, thromboembolism is a major cause of morbidity affecting over 2 million Americans every year. (Adcock et al. American Journal of Clinical Pathology. 1997; 108:434-49). While most cases of thrombosis are due to acquired extrinsic problems, for example, surgery, cancer, immobility, some cases are due to a genetic predisposition, for example, antiphospholipid syndrome and the autosomal dominant condition, Factor V Leiden. (Bertina R M et al. Nature 1994; 369:64-67.)

### Treatment

The most commonly used anticoagulants, warfarin, heparin, and low molecular weight heparin (LMWH) all possess significant drawbacks.

Warfarin is typically used to treat patients suffering from atrial fibrillation. The drug interacts with vitamin K-dependent coagulation factors which include factors II, VII, IX and X. Anticoagulant proteins C and S are also inhibited by warfarin. Drug therapy using warfarin is further complicated by the fact that warfarin interacts with other medications, including drugs used to treat atrial fibrillation, such as amiodarone. Because therapy with warfarin is difficult to predict, patients must be carefully monitored in order to detect any signs of anomalous bleeding.

Heparin functions by activating antithrombin which inhibits both thrombin and factor X. (Bjork I, Lindahl U. Mol Cell Biochem. 1982 48: 161-182.) Treatment with heparin may cause an immunological reaction that makes platelets aggregate within blood vessels that can lead to thrombosis. This side effect is known as heparin-induced thrombocytopenia (HIT) and requires patient monitoring. Prolonged treatment with heparin may also lead to osteoporosis. LMWH can also inhibit Factor 2, but to a lesser degree than unfractioned heparin (UFH). LMWH has been implicated in the development of HIT.

Thus, current anticoagulant agents lack predictability and specificity and, therefore, require careful patient monitoring to prevent adverse side effects, such as bleeding complications. There are currently no anticoagulants which target only the intrinsic or extrinsic pathway.

### SUMMARY

Provided herein are methods for modulating expression of Factor VII mRNA and protein. In certain embodiments, Factor VII specific inhibitors modulate expression of Factor VII mRNA and protein. In certain embodiments, Factor VII specific inhibitors are nucleic acids, proteins, or small molecules.

In certain embodiments, modulation occurs in a cell or tissue. In certain embodiments, the cell or tissue is in an animal. In certain embodiments, the animal is a human. In certain embodiments, Factor VII mRNA levels are reduced. In certain embodiments, Factor VII protein levels are reduced. In certain embodiments, both Factor VII mRNA and protein levels are reduced. Such reduction may occur in a time-dependent or in a dose-dependent manner.

Also provided are methods for preventing, treating, and ameliorating diseases, disorders, and conditions. In certain embodiments, such diseases, disorders, and conditions are thromboembolic complications, hyperproliferative disorders, and inflammatory conditions. Certain such thromboembolic complications include thrombosis, embolism, and thromboembolism, such as, deep vein thrombosis, pulmonary embolism, myocardial infarction, stroke, cancer, rheumatoid arthritis, and fibrosis. Certain such hyperproliferative disorders include cancer, psoriasis, hyperplasia and the like. Certain such inflammatory conditions include rheumatoid arthritis, liver fibrosis, sepsis, myocardial ischemia/reperfusion injury, adult respiratory distress syndrome, nephritis, graft rejection, inflammatory bowel disease, multiple sclerosis, arteriosclerosis, and vasculitis.

Such diseases, disorders, and conditions can have one or more risk factors, causes, or outcomes in common. Certain risk factors and causes for development of a thromboembolic complication include immobility, surgery (particularly orthopedic surgery), malignancy, pregnancy, older age, use of oral contraceptives, atrial fibrillation, previous thromboembolic complication, chronic inflammatory disease, and inherited or acquired prothrombotic clotting disorders. Certain outcomes associated with development of a thromboembolic complication include decreased blood flow through an affected vessel, death of tissue, and death of the individual. Certain risk factors and causes for development of a hyperproliferative disorder include genetic factors, such as gene mutations and chromosomal aberrations, which may or may not be inherited; and environmental factors, which include, but are not limited to, exposure to known mutagens, such as high energy radiation from radioactive elements, X-rays, gamma rays, microwaves, and ultraviolet light; certain industrial chemicals; pollutants such as cigarette smoke; certain pesticides; drugs, and viruses. Certain outcomes associated with development of a hyperproliferative disorder include non-malignant tumors, pre-malignant tumors, and malignant tissues in an individual. Certain risk factors and causes for development of an inflammatory condition include any noxious stimulus that causes a cellular response to an underlying pathophysiologic condition, which includes but is not limited to bacterial and viral infections, and allergens. Certain outcomes associated with development of an inflammatory condition include redness, pain, swelling at the affected area, loss of function, morbidity and mortality of the individual.

In certain embodiments, methods of treatment include administering a Factor VII specific inhibitor to an individual in need thereof. In certain embodiments, the Factor VII specific inhibitor is a nucleic acid. In certain embodiments, the nucleic acid is an antisense compound. In certain embodiments, the antisense compound is a modified oligonucleotide.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or”, unless stated otherwise. Additionally, as used herein, the use of “and” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this disclosure, including, but not limited to, patents, patent applications, published patent applications, articles, books, treatises, and GENBANK Accession Numbers and associated sequence information obtainable through databases such as National Center for Biotechnology Information (NCBI) and other data referred to throughout in the disclosure are hereby expressly incorporated by reference for the portions of the document discussed herein, as well as in their entirety.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis.

Unless otherwise indicated, the following terms have the following meanings:

“2′-O-methoxyethyl” (also 2′-MOE and MOE and 2′-O(CH₂)₂—OCH₃) refers to an O-methoxy-ethyl modification of the 2′ position of a furanosyl ring. A 2′-O-methoxyethyl modified sugar is a modified sugar.

“2′-deoxyribonucleoside” means a nucleoside comprising 2′-H furanosyl sugar moiety, as found in naturally occurring deoxyribonucleosides (DNA).

“2′-MOE nucleoside” (also 2′-O-methoxyethyl nucleoside) means a nucleoside comprising a 2′-MOE modified sugar moiety.

“3′-fluoro-HNA” (also “F—HNA” or “3′-F—HNA”) means the sugar moiety of a nucleoside having the following structure:

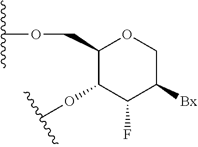

wherein Bx is a nucleobase.

“5-methylcytosine” means a cytosine modified with a methyl group attached to the 5′ position. A 5-methylcytosine is a modified nucleobase.

“About” means within ±7% of a value. For example, if it is stated, “the compounds affected at least about 70% inhibition of Factor VII,” it is implied that the Factor VII levels are inhibited within a range of 63% and 77%.

“Active pharmaceutical agent” means the substance or substances in a pharmaceutical composition that provide a therapeutic benefit when administered to an individual. For example, in certain embodiments an antisense oligonucleotide targeted to Factor VII is an active pharmaceutical agent.

“Active target region” or “target region” means a region to which one or more active antisense compounds is targeted. “Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Amelioration” or “ameliorate” or “ameliorating” refers to a lessening of at least one indicator, sign, or symptom of an associated disease, disorder, or condition. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antidote compound” refers to a compound capable of decreasing the intensity or duration of any antisense activity.

“Antidote oligonucleotide” means an antidote compound comprising an oligonucleotide that is complementary to and capable of hybridizing with an antisense compound.

“Antidote protein” means an antidote compound comprising a peptide.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, snoRNAs, miRNAs, and satellite repeats.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furanosyl ring modified by the bridging of two atoms. A bicyclic sugar is a modified sugar.

“Bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“Cap structure” or “terminal cap moiety” means chemical modifications, which have been incorporated at either terminus of an antisense compound.

“cEt” or “constrained ethyl” means a bicyclic nucleoside sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O-2′ bridge.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses parallel or sequential administration.

“Coagulation factor” means any of factors I, II, III, IV, V, VII, VIII, IX, X, XI, XII, XIII, or TAFI in the blood coagulation cascade. “Coagulation factor nucleic acid” means any nucleic acid encoding a coagulation factor. For example, in certain embodiments, a coagulation factor nucleic acid includes, without limitation, a DNA sequence encoding a coagulation factor (including genomic DNA comprising introns and exons), an RNA sequence transcribed from DNA encoding a coagulation factor, and an mRNA sequence encoding a coagulation factor. “Coagulation factor mRNA” means an mRNA encoding a coagulation factor protein.

“Complementarity” means the capacity for pairing between nucleobases of a first nucleic acid and a second nucleic acid.

“Contiguous nucleobases” means nucleobases immediately adjacent to each other.

“Diluent” means an ingredient in a composition that lacks pharmacological activity, but is pharmaceutically necessary or desirable. For example, the diluent in an injected composition may be a liquid, e.g. saline solution.

“Dose” means a specified quantity of a pharmaceutical agent provided in a single administration, or

in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Factor VII nucleic acid” or “Factor 7 nucleic acid” or “F VII nucleic acid” or “F 7 nucleic acid” means any nucleic acid encoding Factor VII. For example, in certain embodiments, a Factor VII nucleic acid includes, a DNA sequence encoding Factor VII, an RNA sequence transcribed from DNA encoding Factor VII (including genomic DNA comprising introns and exons), and an mRNA sequence encoding Factor VII. “Factor VII mRNA” means an mRNA encoding a Factor VII protein.

“Factor VII specific inhibitor” refers to any agent capable of specifically inhibiting the expression of Factor VII mRNA and/or Factor VII protein at the molecular level. For example, Factor VII specific inhibitors include nucleic acids (including antisense compounds), peptides, antibodies, small molecules, and other agents capable of inhibiting the expression of Factor VII mRNA and/or Factor VII protein. In certain embodiments, by specifically modulating Factor VII mRNA expression and/or Factor VII protein expression, Factor VII specific inhibitors may affect other components of the coagulation cascade including downstream components. Similarly, in certain embodiments, Factor VII specific inhibitors may affect other molecular processes in an animal

“Factor VII specific inhibitor antidote” means a compound capable of decreasing the effect of a Factor VII specific inhibitor. In certain embodiments, a Factor VII specific inhibitor antidote is selected from a Factor VII peptide; a Factor VII antidote oligonucleotide; including a Factor VII antidote compound complementary to a Factor VII antisense compound; and any compound or protein that affects the intrinsic or extrinsic coagulation pathway.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Furanosyl” means a structure comprising a 5-membered ring comprising four carbon atoms and one oxygen atom.

“Gapmer” means a chimeric antisense compound in which an internal region having a plurality of nucleosides that support RNaseH cleavage is positioned between external regions having one or more nucleosides, wherein the nucleosides comprising the internal region are chemically distinct from the nucleoside or nucleosides comprising external regions. The internal region may be referred to as a “gap” and the external regions may be referred to as the “wings.”

“Gap-widened” means a chimeric antisense compound having a gap segment of 12 or more contiguous 2′-deoxyribonucleosides positioned between and immediately adjacent to 5′ and 3′ wing segments having from one to six nucleosides.

“Hybridization” means the annealing of complementary nucleic acid molecules. In certain embodiments, complementary nucleic acid molecules include an antisense compound and a target nucleic acid.

“Hyperproliferative disorder” refers to disorders characterized by an abnormal or pathological proliferation of cells, for example, cancer, psoriasis, hyperplasia and the like.

“Identifying an animal at risk for developing a hyperproliferative disorder” means identifying an animal having been diagnosed with a hyperproliferative disorder, or identifying an animal predisposed to develop a hyperproliferative disorder. Individuals predisposed to develop a hyperproliferative disorder include those having one or more risk factors for hyperproliferative disorders including genetic factors, such as gene mutations and chromosomal aberrations, which may or may not be inherited; and environmental factors, which include, but are not limited to, exposure to known mutagens, such as high energy radiation from radioactive elements, X-rays, gamma rays, microwaves, and ultraviolet light; certain industrial chemicals; pollutants such as cigarette smoke; certain pesticides; drugs, and viruses. Such identification may be accomplished by any method including evaluating an individual's medical history and standard clinical tests or assessments.

“Identifying an animal at risk for developing an inflammatory condition” means identifying an animal having been diagnosed with an inflammatory condition, or identifying an animal predisposed to develop an inflammatory condition. Individuals predisposed to develop an inflammatory condition include those having one or more risk factors for inflammatory disorders including contact with any noxious stimulus that causes a cellular response to an underlying pathophysiologic condition, which includes but is not limited to bacterial and viral infections, and allergens. Such identification may be accomplished by any method including evaluating an individual's medical history and standard clinical tests or assessments.

“Identifying an animal at risk for developing a thromboembolic complication” means identifying an animal having been diagnosed with a thromboembolic complication, or identifying an animal predisposed to develop a thromboembolic complication. Individuals predisposed to develop a thromboembolic complication include those having one or more risk factors for thromboembolic complications including immobility, surgery (particularly orthopedic surgery), malignancy, pregnancy, older age, use of oral contraceptives, and inherited or acquired prothrombotic clotting disorders. Such identification may be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Individual in need thereof” refers to a human or non-human animal selected for treatment or therapy that is in need of such treatment or therapy.

“Inflammatory condition” refers to a disease, disease state, syndrome, or other condition resulting in inflammation. For example, rheumatoid arthritis and liver fibrosis are inflammatory conditions. Other examples of inflammatory conditions include sepsis, myocardial ischemia/reperfusion injury, adult respiratory distress syndrome, nephritis, graft rejection, inflammatory bowel disease, multiple sclerosis, arteriosclerosis, and vasculitis.

“Internucleoside linkage” refers to the chemical bond between nucleosides.

“ISIS 473589” means a Factor VII reducing agent that is a modified antisense oligonucleotide having the nucleobase sequence (from 5′ to 3′) “GCTAAACAACCGCCTT”, incorporated herein as SEQ ID NO: 59, consisting of a combination of sixteen 2′-deoxyribonucleosides, MOE nucleosides, and cEt nucleosides, wherein each internucleoside linkage is a phosphorothioate internucleoside linkage and each cytosine is a 5-methylcytosine. From the 5′ end to the 3′ end, each nucleoside of ISIS 473589 has the following sugar moiety: cEt, 2′-deoxyribose, cEt, 2′-deoxyribose, cEt, 2′-deoxyribose, 2′-deoxyribose, 2′-deoxyribose, 2′-deoxyribose, 2′-deoxyribose, 2′-deoxyribose, 2′-deoxyribose, 2′-deoxyribose, 2′-deoxyribose, MOE, MOE. The chemical modifications can also be represented by the formula: Gks mCds Tks Ads Aks Ads mCds Ads Ads mCds mCds Gds mCds mCds Tes Te, wherein ‘k’ indicates a cEt sugar moiety; ‘d’ indicates a deoxyribose moiety; ‘e’ indicates a MOE sugar moiety; ‘mC’ indicates a 5-methylcytosine; and ‘s’ indicates a phosphorothioate linkage (P═S).

“ISIS 490279” means a Factor VII reducing agent that is a modified antisense oligonucleotide having the nucleobase sequence (from 5′ to 3′) “CCCTCCTGTGCCTGGATGCT”, incorporated herein as SEQ ID NO: 93, a 5-10-5 MOE gapmer, wherein each internucleoside linkage is a phosphorothioate internucleoside linkage and each cytosine is a 5-methylcytosine, and each of nucleosides 1-5 and 16-20 comprise a 2′-O-methoxyethyl moiety. The chemical modifications can also be represented by the formula: mCes mCes mCes Tes mCes mCds Tds Gds Tds Gds mCds mCds Tds Gds Gds Aes Tes Ges mCes Te, wherein ‘d’ indicates a deoxyribose moiety; ‘e’ indicates a MOE sugar moiety; ‘mC’ indicates a 5-methylcytosine; and ‘s’ indicates a phosphorothioate linkage (P═S).

“ISIS 540175” means a Factor VII reducing agent that is a modified antisense oligonucleotide having the nucleobase sequence (from 5′ to 3′) “GGACACCCACGCCCCC”, incorporated herein as SEQ ID NO:637, consisting of a combination of sixteen deoxynucleosides, MOE nucleosides, and cEt nucleosides, wherein each internucleoside linkage is a phosphorothioate internucleoside linkage and each cytosine is a 5-methylcytosine. From the 5′ end to the 3′ end, each nucleoside of ISIS 540175 has the following sugar moiety: MOE, MOE, cEt, 2′-deoxyribose, 2′-deoxyribose, 2′-deoxyribose, 2′-deoxyribose, 2′-deoxyribose, 2′-deoxyribose, 2′-deoxyribose, 2′-deoxyribose, 2′-deoxyribose, 2′-deoxyribose, cEt, cEt, MOE. The chemical modifications can also be represented by the formula: Ges Ges Aks mCds Ads mCds mCds mCds Ads mCds Gds mCds mCds mCks mCks mCe, wherein ‘k’ indicates a cEt sugar moiety; ‘d’ indicates a deoxyribose; ‘e’ indicates a MOE sugar moiety; ‘mC’ indicates a 5-methylcytosine; and ‘s’ indicates a phosphorothioate linkage (P═S).

“Linked nucleosides” means adjacent nucleosides which are bonded together.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising a modified internucleoside linkage, a modified sugar, or a modified nucleobase.

“Modified sugar” refers to a substitution or change from a natural sugar.

“MOE nucleoside” means a nucleoside comprising a 2′-substituted sugar moiety comprising MOE at the 2′-position.

“Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non furanose sugar units. Nucleotide mimetic includes those structures used to replace the nucleoside and the linkageat one or more positions of an oligomeric compound such as for example peptide nucleic acids or morpholinos (morpholinos linked by —N(H)—C(═O)—O— or other non phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system.

“Nucleotide” means a nucleoside having a phosphate group covalently linked to the sugar portion of the nucleoside.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Peptide” means a molecule formed by linking at least two amino acids by amide bonds. Peptide refers to polypeptides and proteins.

“Pharmaceutical composition” means a mixture of substances suitable for administering to an individual. For example, a pharmaceutical composition may comprise one or more active pharmaceutical agents and a sterile aqueous solution.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage (P═S) is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” or “preventing” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Sugar moiety” means a naturally occurring sugar moiety or a modified sugar moiety of a nucleoside.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treat” or “treating” refers to administering a pharmaceutical composition to effect an alteration or improvement of a disease, disorder, or condition.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### Certain Embodiments

Certain embodiments provide methods for decreasing Factor VII mRNA and protein expression.

Certain embodiments provide methods for the treatment, prevention, or amelioration of diseases, disorders, and conditions associated with Factor VII in an individual in need thereof. Also contemplated are methods for the preparation of a medicament for the treatment, prevention, or amelioration of a disease, disorder, or conditions associated with Factor VII. Factor VII associated diseases, disorders, and conditions include thromboembolic complications, hyperproliferative disorders, and inflammatory conditions. Certain such thromboembolic complications include thrombosis, embolism, and thromboembolism, such as, deep vein thrombosis, pulmonary embolism, myocardial infarction, stroke, cancer, rheumatoid arthritis, and fibrosis. Certain such hyperproliferative disorders include cancer, psoriasis, hyperplasia and the like. Certain such inflammatory conditions include rheumatoid arthritis, liver fibrosis, sepsis, myocardial ischemia/reperfusion injury, adult respiratory distress syndrome, nephritis, graft rejection, inflammatory bowel disease, multiple sclerosis, arteriosclerosis, and vasculitis.

Such diseases, disorders, and conditions can have one or more risk factors, causes, or outcomes in common. Certain risk factors and causes for development of a thromboembolic complication include immobility, surgery (particularly orthopedic surgery), malignancy, pregnancy, older age, use of oral contraceptives, atrial fibrillation, previous thromboembolic complication, chronic inflammatory disease, and inherited or acquired prothrombotic clotting disorders. Certain outcomes associated with development of a thromboembolic complication include decreased blood flow through an affected vessel, death of tissue, and death of the individual. Certain risk factors and causes for development of a hyperproliferative disorder include genetic factors, such as gene mutations and chromosomal aberrations, which may or may not be inherited; and environmental factors, which include, but are not limited to, exposure to known mutagens, such as high energy radiation from radioactive elements, X-rays, gamma rays, microwaves, and ultraviolet light; certain industrial chemicals; pollutants such as cigarette smoke; certain pesticides; drugs, and viruses. Certain outcomes associated with development of a hyperproliferative disorder include non-malignant tumors, pre-malignant tumors and malignant tissues in an individual. Certain risk factors and causes for development of an inflammatory condition include any noxious stimulus that causes a cellular response to an underlying pathophysiologic condition, which includes but is not limited to bacterial and viral infections, and allergens. Inflammation is mediated by cytokines, which are secreted by the host macrophages, T-lymphocytes, endothelial cells. Certain outcomes associated with development of an inflammatory condition include redness, pain, swelling at the affected area, loss of function, morbidity and mortality of the individual.

Certain embodiments provide for the use of a Factor VII specific inhibitor for treating, preventing, or ameliorating a Factor VII associated disease. In certain embodiments, Factor VII specific inhibitors are nucleic acids (including antisense compounds), peptides, antibodies, small molecules, and other agents capable of inhibiting the expression of Factor VII mRNA and/or Factor VII protein.

In certain embodiments, methods of treatment include administering a Factor VII specific inhibitor to an individual in need thereof.

In certain embodiments, provided herein are methods and compounds for the preparation of a medicament for the treatment, prevention, or amelioration of a disease, disorder, or condition associated with Factor VII. Factor VII associated diseases, disorders, and conditions include thromboembolic complications, hyperproliferative disorders, and inflammatory conditions. Thromboembolic complications include thrombosis, embolism, thromboembolism, deep vein thrombosis, pulmonary embolism, myocardial infarction, and stroke. Hyperproliferative disorders include cancer. Inflammatory conditions include rheumatoid arthritis and fibrosis.

Embodiments described herein provide a Factor VII specific inhibitor for use in treating, preventing, or ameliorating a Factor VII associated disease. In certain embodiments, Factor VII specific inhibitors are nucleic acids (including antisense compounds), peptides, antibodies, small molecules, and other agents capable of inhibiting the expression of Factor VII mRNA and/or Factor VII protein.

Embodiments described herein provide a Factor VII specific inhibitor, as described herein, for use in treating, preventing, or ameliorating thromboembolic complications such as thrombosis, embolism, thromboembolism, deep vein thrombosis, pulmonary embolism, myocardial infarction, and stroke.

Embodiments described herein provide a Factor VII specific inhibitor, as described herein, for use in treating, preventing, or ameliorating a thromboembolic complication, as described herein, by combination therapy with an additional agent or therapy, as described herein. Agents or therapies can be co-administered or administered concomitantly.

Embodiments described herein provide the use of a Factor VII specific inhibitor, as described herein, in the manufacture of a medicament for treating, preventing, or ameliorating a thromboembolic complication, as described herein, by combination therapy with an additional agent or therapy, as described herein. Agents or therapies can be co-administered or administered concomitantly.

Embodiments described herein provide the use of a Factor VII specific inhibitor, as described herein, in the manufacture of a medicament for treating, preventing, or ameliorating a thromboembolic complication, as described herein, in a patient who is subsequently administered an additional agent or therapy, as described herein.

Embodiments described herein provide a Factor VII specific inhibitor, as described herein, for use in treating, preventing, or ameliorating hyperproliferative disorder such as cancer, psoriasis, and hyperplasia.

Embodiments described herein provide a Factor VII specific inhibitor, as described herein, for use in treating, preventing, or ameliorating a hyperproliferative disorder, as described herein, by combination therapy with an additional agent or therapy, as described herein. Agents or therapies can be co-administered or administered concomitantly.

Embodiments described herein provide the use of a Factor VII specific inhibitor, as described herein, in the manufacture of a medicament for treating, preventing, or ameliorating a hyperproliferative disorder, as described herein, by combination therapy with an additional agent or therapy, as described herein. Agents or therapies can be co-administered or administered concomitantly.

Embodiments described herein provide the use of a Factor VII specific inhibitor, as described herein, in the manufacture of a medicament for treating, preventing, or ameliorating a hyperproliferative disorder, as described herein, in a patient who is subsequently administered an additional agent or therapy, as described herein.

Embodiments described herein provide a Factor VII specific inhibitor, as described herein, for use in treating, preventing, or ameliorating inflammatory conditions such as rheumatoid arthritis, liver fibrosis, sepsis, myocardial ischemia/reperfusion injury, adult respiratory distress syndrome, nephritis, graft rejection, inflammatory bowel disease, multiple sclerosis, arteriosclerosis, and vasculitis.

Embodiments described herein provide a Factor VII specific inhibitor, as described herein, for use in treating, preventing, or ameliorating an inflammatory condition, as described herein, by combination therapy with an additional agent or therapy, as described herein. Agents or therapies can be co-administered or administered concomitantly.

Embodiments described herein provide the use of a Factor VII specific inhibitor, as described herein, in the manufacture of a medicament for treating, preventing, or ameliorating an inflammatory condition, as described herein, by combination therapy with an additional agent or therapy, as described herein. Agents or therapies can be co-administered or administered concomitantly.

Embodiments described herein provide the use of a Factor VII specific inhibitor, as described herein, in the manufacture of a medicament for treating, preventing, or ameliorating an inflammatory condition, as described herein, in a patient who is subsequently administered an additional agent or therapy, as described herein.

In certain embodiments, Factor VII specific inhibitors are peptides or proteins, such as, but not limited to, GP 1-49 (Martin, D. M. et al., Biochemistry. 1993. 32: 13949-13955); peptide-(285-305), peptide-(44-50), peptide-(194-214), peptide-(208-229), and peptide-(376-390) (Kumar, A. et al., J. Biol. Chem. 1991. 266: 915-921); modified Factor VII (U.S. Pat. No. 5,824,639); and modified Factor VII (USPPN 2004/0197370).

In certain embodiments, Factor VII specific inhibitors are antibodies, such as, but not limited to, GP 1-49 (Martin, D. M. et al., Biochemistry. 1993. 32: 13949-13955); peptide-(285-305), peptide-(44-50), peptide-(194-214), peptide-(208-229), and peptide-(376-390) (Kumar, A. et al., J. Biol. Chem. 1991. 266: 915-921); modified Factor VII (U.S. Pat. No. 5,824,639); and modified Factor VII (USPPN 2004/0197370).

In certain embodiments, Factor VII specific inhibitors are small molecules, such as, but not limited to, curcumin (Koizume, S. et al., Mol. Cancer. Res. 2009. 7: 1928-1936); thrombin (Hultin, M. B. and Jesty, J. Blood 1981. 57: 476-482); phospholipase C Hubbard A. R. and Parr, L. J. Br. J. Haematol. 1989. 73: 360-364); ruthenium red (Chu, A. J. et al; Br. J. Pharmacol. 2001. 133: 659-664); and 1-hydroxy-7-hydroxycarbamoylquinoxaline-2,3(1H,4H)-dione compounds (U.S. Pat. No. 5,859,010).

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 16, at least 18, at least 19, or at least 20 contiguous nucleobases complementary to an equal length portion of nucleobases 1381 to 1406 of SEQ ID NO: 1. In certain embodiments, the nucleobase sequence of the modified oligonucleotide is at least 90% complementary to SEQ ID NO: 1.

In certain embodiments, the modified oligonucleotide consists of 15 to 30, 18 to 24, 19 to 22, or 20 linked nucleosides.

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 15128 to 15150 of SEQ ID NO: 1. In certain embodiments, the nucleobase sequence of the modified oligonucleotide is at least 90% complementary to SEQ ID NO: 1.

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2592 to 2607, 2626 to 2641, 2660 to 2675, 2796 to 2811, 2966 to 2981, 3000 to 3015, 3034 to 3049, 3068 to 3083, 3153 to 3168, 3170 to 3185, 3272 to 3287, 3374 to 3389, 3578 to 3593, 3851 to 3866, 3953 to 3968, 4124 to 4139, 4260 to 4275, 4311 to 4326, 4447 to 4462, and 4532 to 4547 of SEQ ID NO: 1. In certain embodiments, the nucleobase sequence of the modified oligonucleotide is at least 90% complementary to SEQ ID NO: 1.

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2592 to 2607, 2626 to 2641, 2660 to 2675, 2796 to 2811, 2966 to 2981, 3000 to 3015, 3034 to 3049, 3068 to 3083, 3153 to 3168, 3170 to 3185, 3272 to 3287, 3374 to 3389, 3578 to 3593, 3851 to 3866, 3953 to 3968, 4124 to 4139, 4260 to 4275, 4311 to 4326, 4447 to 4462, or 4532 to 4547 of SEQ ID NO: 1. In certain embodiments, the nucleobase sequence of the modified oligonucleotide is at least 90% complementary to SEQ ID NO: 1.

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 1387 to 1406, 15128 to 15143, 15192 to 15207, and 15131 to 15146 of SEQ ID NO: 1. In certain embodiments, the nucleobase sequence of the modified oligonucleotide is at least 90% complementary to SEQ ID NO: 1.

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2692 to 2707, 2760 to 2775, 2862 to 2877, 2930 to 2945, 3117 to 3132, 3338 to 3353, 3440 to 3455, 3508 to 3523, 3542 to 3557, 3628 to 3643, 3662 to 3677, 3781 to 3796, 3815 to 3830, 3917 to 3932, 4190 to 4205, 4224 to 4239, 4377 to 4392, and/or 4411 to 4426 of SEQ ID NO: 1. In certain embodiments, the nucleobase sequence of the modified oligonucleotide is at least 90% complementary to SEQ ID NO: 1.

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 3109 to 3124, 3194 to 3209, 3330 to 3345, 3432 to 3447, 3500 to 3515, 3534 to 3549, 3620 to 3635, 3654 to 3669, 3773 to 3788, 4182 to 4197, 4216 to 4231, 4369 to 4384, and/or 4403 to 4418 of SEQ ID NO: 1. In certain embodiments, the nucleobase sequence of the modified oligonucleotide is at least 90% complementary to SEQ ID NO: 1.

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2565 to 2580, 2633 to 2648, 2667 to 2682, 2735 to 2750, 2803 to 2818, 2837 to 2852, 2905 to 2920, 3007 to 3022, 3041 to 3056, 3075 to 3090, 3092 to 3107, 3279 to 3294, 3381 to 3396, 3483 to 3498, 3603 to 3618, 3722 to 3737, 3756 to 3771, 3858 to 3873, 3892 to 3907, 3960 to 3975, 4046 to 4061, 4131 to 4146, 4165 to 4180, 4318 to 4333, and/or 4454 to 4469 of SEQ ID NO: 1. In certain embodiments, the nucleobase sequence of the modified oligonucleotide is at least 90% complementary to SEQ ID NO: 1.

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2558 to 4600 of SEQ ID NO: 1. In certain embodiments, the nucleobase sequence of the modified oligonucleotide is at least 90% complementary to SEQ ID NO: 1.

In certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 15128 to 15150, 15181 to 15224, 15128 to 15150, 2560 to 2609, 2684 to 2717, or 3103 to 3131 of SEQ ID NO: 1. In certain embodiments, the nucleobase sequence of the modified oligonucleotide is at least 90% complementary to SEQ ID NO: 1.

In certain embodiments, the modified oligonucleotide consists of 13 to 25, 14 to 25, 15 to 25, or 16 linked nucleosides.

In certain embodiments, the nucleobase sequence of the modified oligonucleotide is at least 91%, at least 92%, at least 93%, at least 94%, at least 95%, at least 96%, at least 97%, at least 98%, at least 99%, or 100% complementary to SEQ ID NO: 1.

In certain embodiments, provided herein are compounds comprising a modified oligonucleotide consisting of 12 to 30 linked nucleosides and having a nucleobase sequence comprising at least 12, at least 14, at least 15, or at least 16 contiguous nucleobases of the nucleobase sequence of SEQ ID NO: 59.

In certain embodiments, provided herein are compounds comprising a modified oligonucleotide consisting of 12 to 30 linked nucleosides and having a nucleobase sequence comprising at least 12, at least 14, at least 16, at least 18, at least 19, or at least 20 contiguous nucleobases of the nucleobase sequence of SEQ ID NO: 93.

In certain embodiments, provided herein are compounds comprising a modified oligonucleotide consisting of 12 to 30 linked nucleosides and having a nucleobase sequence comprising at least 12, at least 14, at least 15, or at least 16 contiguous nucleobases of the nucleobase sequence of SEQ ID NO: 637.

In certain embodiments, provided herein are compounds comprising a modified oligonucleotide consisting of 12 to 30 linked nucleosides and having a nucleobase sequence comprising at least 12, at least 14, at least 15, or at least 16 contiguous nucleobases of any of the nucleobase sequences of SEQ ID NO: 59, 93, 259, 254, 624, 637, 644, or 653.

In certain embodiments, provided herein are compounds comprising a modified oligonucleotide consisting of 12 to 30 linked nucleosides and having a nucleobase sequence comprising at least 12, at least 14, at least 15, or at least 16 contiguous nucleobases of any of the nucleobase sequences of SEQ ID NO: 21-559.

In certain embodiments, the compound consists of a single-stranded modified oligonucleotide.

In certain embodiments, at least one internucleoside linkage is a modified internucleoside linkage. In certain embodiments, each internucleoside linkage is a phosphorothioate internucleoside linkage.

In certain embodiments, at least one nucleoside comprises a modified nucleobase. In certain embodiments, the modified nucleobase is a 5-methylcytosine.

In certain embodiments, the modified oligonucleotide comprises at least one modified sugar. In certain embodiments, the modified sugar is any of a 2′-O-methoxyethyl, a constrained ethyl, or a 3′-fluoro-HNA.

In certain embodiments, the compound comprises at least one 2′-O-methoxyethyl nucleoside, a constrained ethyl nucleoside, or a 3′-fluoro-HNA nucleoside. In certain embodiments, provided herein are compounds comprising a modified oligonucleotide according to the following formula:

Gks mCds Tks Ads Aks Ads mCds Ads Ads mCds mCds Gds mCds mCds Tes Te;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

In certain embodiments, provided herein are compounds consisting of a modified oligonucleotide according to the following formula:

Gks mCds Tks Ads Aks Ads mCds Ads Ads mCds mCds Gds mCds mCds Tes Te;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

In certain embodiments, provided herein are compounds comprising of a modified oligonucleotide according to the following formula:

mCes mCes mCes Tes mCes mCds Tds Gds Tds Gds mCds mCds Tds Gds Gds Aes Tes Ges mCes Te;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

In certain embodiments, provided herein are compounds consisting of a modified oligonucleotide according to the following formula:

mCes mCes mCes Tes mCes mCds Tds Gds Tds Gds mCds mCds Tds Gds Gds Aes Tes Ges mCes Te;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

In certain embodiments, provided herein are compounds comprising of a modified oligonucleotide according to the following formula:

Ges Ges Aks mCds Ads mCds mCds mCds Ads mCds Gds mCds mCds mCks mCks mCe;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

In certain embodiments, provided herein are compounds consisting of a modified oligonucleotide according to the following formula:

Ges Ges Aks mCds Ads mCds mCds mCds Ads mCds Gds mCds mCds mCks mCks mCe;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

In certain embodiments, provided herein are compositions comprising a compound as described herein or a salt thereof and a pharmaceutically acceptable carrier or diluent.

In certain embodiments, provided herein are compounds and compositions as described herein for use in therapy.

In certain embodiments, provided herein are compounds and compositions as described herein for use in treating, preventing, or slowing progression of a thromboembolic complication.

In certain embodiments, provided herein are compounds and compositions as described herein for use in treating, preventing, or slowing progression of a hyperproliferative disorder.

In certain embodiments, provided herein are compounds and compositions as described herein for use in treating, preventing, or slowing progression of an inflammatory condition.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that it is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a Factor VII nucleic acid is 12 to 30 subunits in length. In other words, such antisense compounds are from 12 to 30 linked subunits. In other embodiments, the antisense compound is 8 to 80, 12 to 50, 15 to 30, 18 to 24, 19 to 22, or 20 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleosides.

In certain embodiments, antisense oligonucleotides targeted to a Factor VII nucleic acid may be shortened or truncated. For example, a single subunit may be deleted from the 5′ end (5′ truncation), or alternatively from the 3′ end (3′ truncation). A shortened or truncated antisense compound targeted to a Factor VII nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other; for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al. (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a Factor VII nucleic acid have chemically modified subunits arranged in patterns, or motifs, to confer to the antisense compounds properties, such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer, an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as a substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may, in some embodiments, include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH2)n-O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30, or more nucleotides. Thus, gapmers described herein include, but are not limited to, for example, 5-10-5, 4-8-4, 4-12-3, 4-12-4, 3-14-3, 2-13-5, 2-16-2, 1-18-1, 3-10-3, 2-10-2, 1-10-1 or 2-8-2.

In certain embodiments, the antisense compound has a “wingmer” motif, having a wing-gap or gap-wing configuration, i.e. an X-Y or Y-Z configuration, as described above, for the gapmer configuration. Thus, wingmer configurations described herein include, but are not limited to, for example, 5-10, 8-4, 4-12, 12-4, 3-14, 16-2, 18-1, 10-3, 2-10, 1-10, 8-2, 2-13, or 5-13.

In certain embodiments, antisense compounds targeted to a Factor VII nucleic acid possess a 5-10-5 gapmer motif.

In certain embodiments, antisense compounds targeted to a Factor VII nucleic acid possess a 3-14-3 gapmer motif.

In certain embodiments, antisense compounds targeted to a Factor VII nucleic acid possess a 2-13-5 gapmer motif.

In certain embodiments, antisense compounds targeted to a Factor VII nucleic acid possess a 2-12-2 gapmer motif.

In certain embodiments, an antisense compound targeted to a Factor VII nucleic acid has a gap-widened motif.

In certain embodiments, a gap-widened antisense oligonucleotide targeted to a Factor VII nucleic acid has a gap segment of fourteen 2′-deoxyribonucleotides positioned immediately adjacent to and between wing segments of three chemically modified nucleosides. In certain embodiments, the chemical modification comprises a 2′-sugar modification. In another embodiment, the chemical modification comprises a 2′-MOE sugar modification.

In certain embodiments, a gap-widened antisense oligonucleotide targeted to a Factor VII nucleic acid has a gap segment of thirteen 2′-deoxyribonucleotides positioned immediately adjacent to and between a 5′ wing segment of two chemically modified nucleosides and a 3′ wing segment of five chemically modified nucleosides. In certain embodiments, the chemical modification comprises a 2′-sugar modification. In another embodiment, the chemical modification comprises a 2′-MOE sugar modification.

In certain embodiments, the compounds or compositions comprise modified oligonucleotides consisting of 10 to 30 linked nucleosides and having a nucleobase sequence comprising a portion at least 8, 9, 10, 11, 12, 13, 14, 15, 16, 17, 18, 19, 20, 21, 22, 23, 24, 25, 26, 27, 28, 29 or 30 contiguous nucleobases complementary to an equal length portion of any one of the nucleobase ranges: 1381 to 1406, 15128 to 15150, 1387 to 1406, 15128 to 15143, 15192 to 15207, 15131 to 15146, 2592 to 2607, 2626 to 2641, 2660 to 2675, 2796 to 2811, 2966 to 2981, 3000 to 3015, 3034 to 3049, 3068 to 3083, 3153 to 3168, 3170 to 3185, 3272 to 3287, 3374 to 3389, 3578 to 3593, 3851 to 3866, 3953 to 3968, 4124 to 4139, 4260 to 4275, 4311 to 4326, 4447 to 4462, 4532 to 4547, 2692 to 2707, 2760 to 2775, 2862 to 2877, 2930 to 2945, 3117 to 3132, 3338 to 3353, 3440 to 3455, 3508 to 3523, 3542 to 3557, 3628 to 3643, 3662 to 3677, 3781 to 3796, 3815 to 3830, 3917 to 3932, 4190 to 4205, 4224 to 4239, 4377 to 4392, 4411 to 4426, 3109 to 3124, 3194 to 3209, 3330 to 3345, 3432 to 3447, 3500 to 3515, 3534 to 3549, 3620 to 3635, 3654 to 3669, 3773 to 3788, 4182 to 4197, 4216 to 4231, 4369 to 4384, 4403 to 4418, 2565 to 2580, 2633 to 2648, 2667 to 2682, 2735 to 2750, 2803 to 2818, 2837 to 2852, 2905 to 2920, 3007 to 3022, 3041 to 3056, 3075 to 3090, 3092 to 3107, 3279 to 3294, 3381 to 3396, 3483 to 3498, 3603 to 3618, 3722 to 3737, 3756 to 3771, 3858 to 3873, 3892 to 3907, 3960 to 3975, 4046 to 4061, 4131 to 4146, 4165 to 4180, 4318 to 4333, 4454 to 4469, 2558 to 4600, 15128 to 15150, 15181 to 15224, 15128 to 15150, 2560 to 2609, 2684 to 2717, and/or 3103 to 3131 wherein the nucleobase sequence is complementary to SEQ ID NO: 1. In certain embodiments, such oligonucleotides have a gap segment of 9, 10, or more linked deoxynucleosides. In certain embodiments, such gap segment is between two wing segments that independently have 1, 2, 3, 4, or 5 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside. In certain embodiments, the modified nucleoside is a 2′-substituted nucleoside. In certain embodiments, 2′ substituted nucleosides include nucleosides with bicyclic sugar modifications. In certain embodiments, the modified nucleoside is a 2′-MOE nucleoside. In certain embodiments, the modified nucleoside is a constrained ethyl (cEt) nucleoside. In certain embodiments, the modified nucleoside is a F—HNA nucleoside. In certain embodiments, each modified nucleoside in each wing segment is independently a 2′-MOE nucleoside or a nucleoside with a bicyclic sugar modification such as a constrained ethyl (cEt) nucleoside or LNA nucleoside. In certain embodiments, each modified nucleoside in each wing segment is independently a 2′-MOE nucleoside, a nucleoside with a bicyclic sugar modification such as a constrained ethyl (cEt) nucleoside or LNA nucleoside, or a 2′-deoxyribonucleoside.

In certain embodiments, the compounds or compositions comprise a modified oligonucleotide consisting of 10 to 30 linked nucleosides and having a nucleobase sequence comprising at least 8, 9, 10, 11, 12, 13, 14, 15, 16, 17, 18, 19, or 20 contiguous nucleobases of any of SEQ ID NOs: 21-559. In certain embodiments, such oligonucleotides have a gap segment of 8, 9, 10, or more linked deoxynucleosides. In certain embodiments, such gap segment is between two wing segments that independently have 1, 2, 3, 4, 5, 6, 7, or 8 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside. In certain embodiments, the modified nucleoside is a 2′-substituted nucleoside. In certain embodiments, 2′ substituted nucleosides include nucleosides with bicyclic sugar modifications. In certain embodiments, the modified nucleoside is a 2′-MOE nucleoside. In certain embodiments, the modified nucleoside is a constrained ethyl (cEt) nucleoside. In certain embodiments, the modified nucleoside is a F—HNA nucleoside. In certain embodiments, each modified nucleoside in each wing segment is independently a 2′-MOE nucleoside, a nucleoside with a bicyclic sugar modification such as a constrained ethyl (cEt) nucleoside or LNA nucleoside, or a 2′-deoxyribonucleoside.

In certain embodiments, the modified oligonucleotide is 16 nucleosides in length and has a gap segment of 9 linked nucleosides. In certain embodiments, the modified oligonucleotide is 16 nucleosides in length and has a gap segment of 10 linked nucleosides. In certain embodiments, the modified oligonucleotide is 20 nucleosides in length and has a gap segment of 10 linked nucleosides. In certain embodiments, the modified oligonucleotide has a wing segment on the 5′ end and 3′ end of the gap each independently having 1, 2, 3, 4, or 5 sugar modified nucleosides. In certain embodiments, each sugar modified nucleoside is independently a 2′-MOE nucleoside, a nucleoside with a bicyclic sugar moiety such as a constrained ethyl (cEt) nucleoside or LNA nucleoside, or a F—HNA nucleoside. In certain embodiments, each modified nucleoside in each wing segment is independently a 2′-MOE nucleoside, a nucleoside with a bicyclic sugar modification such as a constrained ethyl (cEt) nucleoside or LNA nucleoside, a 2′-deoxyribonucleoside, or a F—HNA nucleoside.

In certain embodiments, the compounds or compositions comprise a salt of the modified oligonucleotide.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar.

In certain embodiments, the modified oligonucleotide consists of 16 linked nucleosides, the gap segment consisting of 10 linked deoxynucleosides, the 5′ wing segment consisting of three linked nucleosides, the 3′ wing segment consisting of three linked nucleosides, each nucleoside of each wing segment comprises a 2′-O-methoxyethyl sugar and/or a constrained ethyl (cEt) sugar, each internucleoside linkage is a phosphorothioate linkage and each cytosine is a 5-methylcytosine. In some aspects, each of the three linked nucleosides of the 5′ wing segment is a 2′-O-methoxyethyl nucleoside and each of the three linked nucleosides of the 3′ wing segment is a constrained ethyl (cEt) nucleoside. In other aspects, the three linked nucleosides of the 5′ wing segment are a 2′-O-methoxyethyl nucleoside, a constrained ethyl (cEt) nucleoside, and a constrained ethyl (cEt) nucleoside in the 5′ to 3′ direction, and the three linked nucleosides of the 3′ wing segment are a constrained ethyl (cEt) nucleoside, a constrained ethyl (cEt) nucleoside, and a 2′-O-methoxyethyl nucleoside in the 5′ to 3′ direction. In other aspects, the three linked nucleosides of the 5′ wing segment are a 2′-O-methoxyethyl nucleoside, 2′-O-methoxyethyl nucleoside, and a constrained ethyl (cEt) nucleoside in the 5′ to 3′ direction, and the three linked nucleosides of the 3′ wing segment are a constrained ethyl (cEt) nucleoside, a constrained ethyl (cEt) nucleoside, and a 2′-O-methoxyethyl nucleoside in the 5′ to 3′ direction.

In certain embodiments, the modified oligonucleotide consists of 16 linked nucleosides, the gap segment consisting of 10 linked deoxynucleosides, the 5′ wing segment consisting of one nucleoside, the 3′ wing segment consisting of five linked nucleosides, each nucleoside of each wing segment comprises a 2′-O-methoxyethyl sugar and/or a constrained ethyl (cEt) sugar, each internucleoside linkage is a phosphorothioate linkage and each cytosine is a 5-methylcytosine. In some aspects, the nucleoside of the 5′ wing segment is a constrained ethyl (cEt) nucleoside and the five linked nucleosides of the 3′ wing segment are a constrained ethyl (cEt) nucleoside, 2′-O-methoxyethyl nucleoside, a constrained ethyl (cEt) nucleoside, a 2′-O-methoxyethyl nucleoside, and a 2′-O-methoxyethyl nucleoside in the 5′ to 3′ direction.

In certain embodiments, the modified oligonucleotide consists of 16 linked nucleosides, the gap segment consisting of 9 linked deoxynucleosides, the 5′ wing segment consisting of five linked nucleosides, the 3′ wing segment consisting of two linked nucleosides, each nucleoside of each wing segment comprises a 2′-O-methoxyethyl sugar, a 2′-deoxyribose, and/or a constrained ethyl (cEt) sugar, each internucleoside linkage is a phosphorothioate linkage and each cytosine is a 5-methylcytosine. In some aspects, the five linked nucleosides of the 5′ wing segment are a constrained ethyl (cEt) nucleoside, a 2′-deoxynucleoside, a constrained ethyl (cEt) nucleoside, a 2′-deoxynucleoside, and a constrained ethyl (cEt) sugar and the two linked nucleosides of the 3′ wing segment are a 2′-O-methoxyethyl nucleoside and a 2′-O-methoxyethyl sugar in the 5′ to 3′ direction.

In certain embodiments, the compounds or compositions comprise a modified oligonucleotide consisting of 16 linked nucleosides having a nucleobase sequence comprising a portion of at least 8 contiguous nucleobases complementary to an equal length portion of any one of the nucleobase ranges: 1381 to 1406, 15128 to 15150, 1387 to 1406, 15128 to 15143, 15192 to 15207, 15131 to 15146, 2592 to 2607, 2626 to 2641, 2660 to 2675, 2796 to 2811, 2966 to 2981, 3000 to 3015, 3034 to 3049, 3068 to 3083, 3153 to 3168, 3170 to 3185, 3272 to 3287, 3374 to 3389, 3578 to 3593, 3851 to 3866, 3953 to 3968, 4124 to 4139, 4260 to 4275, 4311 to 4326, 4447 to 4462, 4532 to 4547, 2692 to 2707, 2760 to 2775, 2862 to 2877, 2930 to 2945, 3117 to 3132, 3338 to 3353, 3440 to 3455, 3508 to 3523, 3542 to 3557, 3628 to 3643, 3662 to 3677, 3781 to 3796, 3815 to 3830, 3917 to 3932, 4190 to 4205, 4224 to 4239, 4377 to 4392, 4411 to 4426, 3109 to 3124, 3194 to 3209, 3330 to 3345, 3432 to 3447, 3500 to 3515, 3534 to 3549, 3620 to 3635, 3654 to 3669, 3773 to 3788, 4182 to 4197, 4216 to 4231, 4369 to 4384, 4403 to 4418, 2565 to 2580, 2633 to 2648, 2667 to 2682, 2735 to 2750, 2803 to 2818, 2837 to 2852, 2905 to 2920, 3007 to 3022, 3041 to 3056, 3075 to 3090, 3092 to 3107, 3279 to 3294, 3381 to 3396, 3483 to 3498, 3603 to 3618, 3722 to 3737, 3756 to 3771, 3858 to 3873, 3892 to 3907, 3960 to 3975, 4046 to 4061, 4131 to 4146, 4165 to 4180, 4318 to 4333, 4454 to 4469, 2558 to 4600, 15128 to 15150, 15181 to 15224, 15128 to 15150, 2560 to 2609, 2684 to 2717, and/or 3103 to 3131 wherein the nucleobase sequence is complementary to SEQ ID NO: 1 and wherein the modified oligonucleotide comprises: a) a gap segment consisting of ten linked deoxynucleosides; b) a 5′ wing segment consisting of three linked nucleosides; and c) a 3′ wing segment consisting of three linked nucleosides. In some aspects, the gap segment is positioned between the 5′ wing segment and the 3′ wing segment; each of the three linked nucleosides of the 5′ wing segment is a 2′-O-methoxyethyl sugar and each of the three linked nucleosides of the 3′ wing segment is a constrained ethyl (cEt) sugar; each internucleoside linkage is a phosphorothioate linkage; and each cytosine residue is a 5-methylcytosine. In other aspects, the gap segment is positioned between the 5′ wing segment and the 3′ wing segment; the three linked nucleosides of the 5′ wing segment are a 2′-O-methoxyethyl sugar, a constrained ethyl (cEt) sugar, and a constrained ethyl (cEt) sugar in the 5′ to 3′ direction; the three linked nucleosides of the 3′ wing segment are a constrained ethyl (cEt) sugar, a constrained ethyl (cEt) sugar, and a 2′-O-methoxyethyl sugar in the 5′ to 3′ direction; each internucleoside linkage is a phosphorothioate linkage; and each cytosine residue is a 5-methylcytosine.

In certain embodiments, the compounds or compositions comprise a modified oligonucleotide consisting of 16 linked nucleosides having a nucleobase sequence comprising a portion of at least 8 contiguous nucleobases complementary to an equal length portion of any one of the nucleobase ranges: 1381 to 1406, 15128 to 15150, 1387 to 1406, 15128 to 15143, 15192 to 15207, 15131 to 15146, 2592 to 2607, 2626 to 2641, 2660 to 2675, 2796 to 2811, 2966 to 2981, 3000 to 3015, 3034 to 3049, 3068 to 3083, 3153 to 3168, 3170 to 3185, 3272 to 3287, 3374 to 3389, 3578 to 3593, 3851 to 3866, 3953 to 3968, 4124 to 4139, 4260 to 4275, 4311 to 4326, 4447 to 4462, 4532 to 4547, 2692 to 2707, 2760 to 2775, 2862 to 2877, 2930 to 2945, 3117 to 3132, 3338 to 3353, 3440 to 3455, 3508 to 3523, 3542 to 3557, 3628 to 3643, 3662 to 3677, 3781 to 3796, 3815 to 3830, 3917 to 3932, 4190 to 4205, 4224 to 4239, 4377 to 4392, 4411 to 4426, 3109 to 3124, 3194 to 3209, 3330 to 3345, 3432 to 3447, 3500 to 3515, 3534 to 3549, 3620 to 3635, 3654 to 3669, 3773 to 3788, 4182 to 4197, 4216 to 4231, 4369 to 4384, 4403 to 4418, 2565 to 2580, 2633 to 2648, 2667 to 2682, 2735 to 2750, 2803 to 2818, 2837 to 2852, 2905 to 2920, 3007 to 3022, 3041 to 3056, 3075 to 3090, 3092 to 3107, 3279 to 3294, 3381 to 3396, 3483 to 3498, 3603 to 3618, 3722 to 3737, 3756 to 3771, 3858 to 3873, 3892 to 3907, 3960 to 3975, 4046 to 4061, 4131 to 4146, 4165 to 4180, 4318 to 4333, 4454 to 4469, 2558 to 4600, 15128 to 15150, 15181 to 15224, 15128 to 15150, 2560 to 2609, 2684 to 2717, and/or 3103 to 3131 wherein the nucleobase sequence is complementary to SEQ ID NO: 1 and wherein the modified oligonucleotide comprises a) a gap segment consisting of ten linked deoxynucleosides; b) a 5′ wing segment consisting of two linked nucleosides; and c) a 3′ wing segment consisting of four linked nucleosides. In some aspects, the gap segment is positioned between the 5′ wing segment and the 3′ wing segment; the two linked nucleosides of the 5′ wing segment are a 2′-O-methoxyethyl sugar and a constrained ethyl (cEt) sugar in the 5′ to 3′ direction; the four linked nucleosides of the 3′ wing segment are a constrained ethyl (cEt) sugar, 2′-O-methoxyethyl sugar, constrained ethyl (cEt) sugar, and 2′-O-methoxyethyl sugar in the 5′ to 3′ direction; each internucleoside linkage is a phosphorothioate linkage; and each cytosine residue is a 5-methylcytosine.

In certain embodiments, the antisense compounds targeted to a Factor VII nucleic acid has any of the following sugar motifs:
k-d(10)-k
e-d(10)-k
k-d(10)-e
k-k-d(10)-k-k
k-k-d(10)-e-e
e-e-d(10)-k-k
k-k-k-d(10)-k-k-k
e-e-e-d(10)-k-k-k
k-k-k-d(10)-e-e-e
k-k-k-d(10)-k-k-k
e-k-k-d(10)-k-k-e
e-e-k-d(10)-k-k-e
e-d-k-d(10)-k-k-e
e-k-d(10)-k-e-k-e
k-d(10)-k-e-k-e-e
e-e-k-d(10)-k-e-k-e
e-d-d-k-d(9)-k-k-e
e-e-e-e-d(9)-k-k-e
e-e-e-e-e-d(10)-e-e-e-e-e
k-d-k-d-k-d(9)-e-e
k-d(10)-k-e-k-e-e
   wherein, ‘k’ is a constrained ethyl nucleoside, ‘e’ is a 2′-MOE substituted nucleoside, and ‘d’ is a 2′-deoxynucleoside. Other motifs and modifications may be applied to the sequences described herein, including those motifs and modifications described in U.S. Ser. No. 61/440,828 filed on Feb. 8, 2011, U.S. Ser. No. 61/470,927 filed on Apr. 1, 2011, and CORE0094WO filed concurrently herewith, all entitled “OLIGOMERIC COMPOUNDS COMPRISING BICYCLIC NUCLEOTIDES AND USES THEREOF” and U.S. Ser. No. 61/522,659 filed on Aug. 11, 2011 and CORE0099US.L2 filed concurrently herewith, both entitled “SELECTIVE ANTISENSE COMPOUNDS AND USES THEREOF,” all of which are incorporated herein by reference.

### Target Nucleic Acids, Target Regions and Nucleotide Sequences

Nucleotide sequences that encode the Factor VII gene sequence include, without limitation, the following: GENBANK Accession No. NT_{—}027140.6 truncated from nucleotides 1255000 to 1273000, incorporated herein as SEQ ID NO: 1; GENBANK Accession No. NM_{—}019616.2, incorporated herein as SEQ ID NO: 2; DB184141.1, designated herein as SEQ ID NO: 3; and GENBANK Accession No. NW_{—}001104507.1 truncated from nucleotides 691000 to 706000, designated herein as SEQ ID NO: 4.

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No.) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, a translation termination region, or other defined nucleic acid region. The structurally defined regions for Factor VII can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region, such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in Factor VII mRNA levels are indicative of inhibition of Factor VII expression. Reductions in levels of a Factor VII protein are also indicative of inhibition of target mRNA expression. Further, phenotypic changes are indicative of inhibition of Factor VII expression. For example, a prolonged PT time can be indicative of inhibition of Factor VII expression. In another example, prolonged aPTT time in conjunction with a prolonged PT time can be indicative of inhibition of Factor VII expression. In another example, a decreased level of Platelet Factor 4 (PF-4) expression can be indicative of inhibition of Factor VII expression. In another example, reduced formation of thrombus or increased time for thrombus formation can be indicative of inhibition of Factor VII expression.

### Hybridization

In some embodiments, hybridization occurs between an antisense compound disclosed herein and a Factor VII nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a Factor VII nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a Factor VII nucleic acid).

Noncomplementary nucleobases between an antisense compound and a Factor VII nucleic acid may be tolerated provided that the antisense compound remains able to specifically hybridize to a target nucleic acid. Moreover, an antisense compound may hybridize over one or more segments of a Factor VII nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a Factor VII nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, an antisense compound may be fully complementary to a Factor VII nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or nucleobases in length comprise no more than 4, no more than 3, no more than 2, or no more than 1 non-complementary nucleobase(s) relative to a target nucleic acid, such as a Factor VII nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a Factor VII nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a Factor 12 nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(Rₗ)—(CH₂)₂—N(Rₘ)(Rₙ), where each Rₗ, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Ser. Nos. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′,4′-(CH₂)_{2-2′, 4}′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the (β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, and (F) methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃₋₂′) BNA as depicted below.

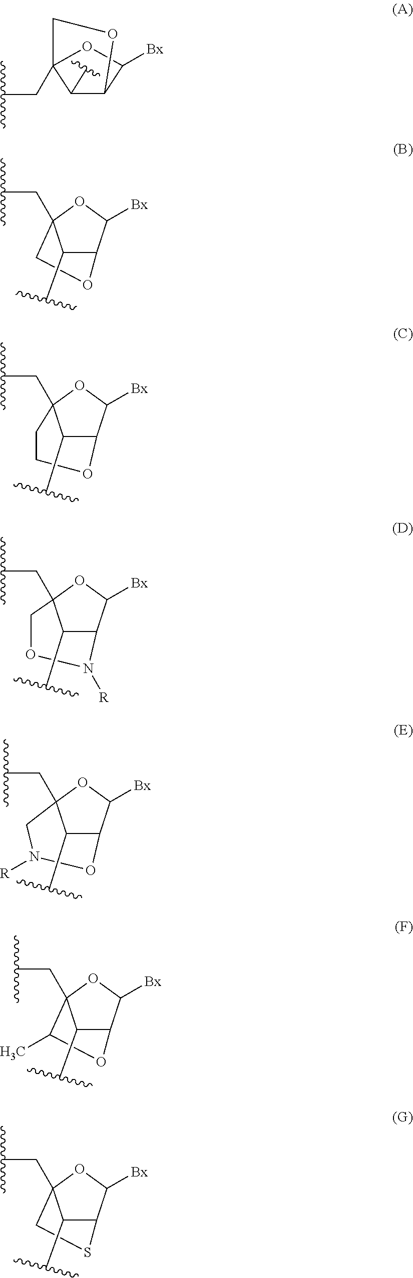

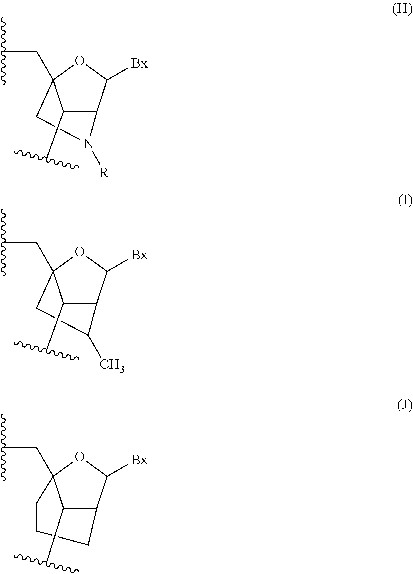

wherein Bx is the base moiety and R is independently H, a protecting group or C₁-C₁₂ alkyl.

In certain embodiments, bicyclic nucleosides are provided having Formula I:

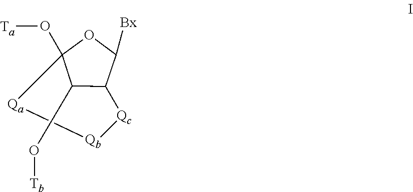

wherein:
Bx is a heterocyclic base moiety;
-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;
R_{c} is C₁-C₁₂ alkyl or an amino protecting group; and
Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

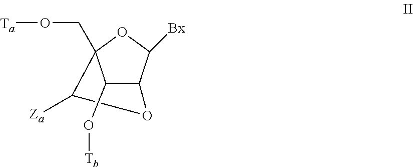

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJ_{c}C(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides are provided having Formula III:

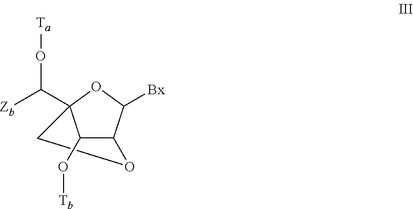

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides are provided having Formula IV:

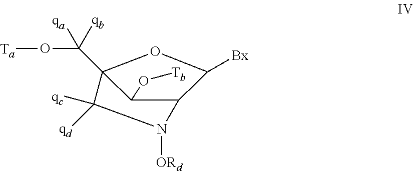

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
each q_{d}, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides are provided having Formula V:

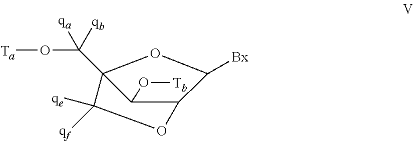

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;
or qₑ and q_{f} together are ═C(q_{g})(qₕ);

q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

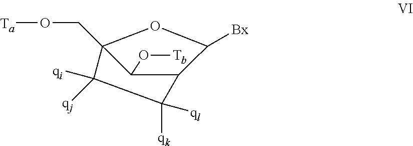

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃₋₂′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854), fluoro HNA (F-HNA) or those compounds having Formula VII:

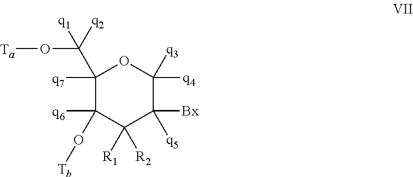

wherein independently for each of said at least one tetrahydropyran nucleoside analog of Formula VII:
Bx is a heterocyclic base moiety;
Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H; R₁ is methoxy and R₂ is H, and R₁ is H and R₂ is methoxyethoxy.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′ substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854).

Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compounds targeted to a Factor VII nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment employed in the methods described herein is a pharmaceutical composition comprising an antisense compound targeted to a Factor VII nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602, published on Jan. 16, 2003.

### Cell Culture and Antisense Compounds Treatment

The effects of antisense compounds on the level, activity or expression of Factor VII nucleic acids can be tested in vitro in a variety of cell types. Cell types used for such analyses are available from commerical vendors (e.g. American Type Culture Collection, Manassas, Va.; Zen-Bio, Inc., Research Triangle Park, N.C.; Clonetics Corporation, Walkersville, Md.) and are cultured according to the vendor's instructions using commercially available reagents (e.g. Invitrogen Life Technologies, Carlsbad, Calif.). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, Calif.). Antisense oligonucleotides are mixed with LIPOFECTIN in OPTI-MEM 1 (Invitrogen, Carlsbad, Calif.) to achieve the desired final concentration of antisense oligonucleotide and a LIPOFECTIN concentration that typically ranges 2 to 12 ug/mL per 100 nM antisense oligonucleotide.

Another reagent used to introduce antisense oligonucleotides into cultured cells includes LIPOFECTAMINE (Invitrogen, Carlsbad, Calif.). Antisense oligonucleotide is mixed with LIPOFECTAMINE in OPTI-MEM 1 reduced serum medium (Invitrogen, Carlsbad, Calif.) to achieve the desired concentration of antisense oligonucleotide and a LIPOFECTAMINE concentration that typically ranges 2 to 12 ug/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL Reagent (Invitrogen, Carlsbad, Calif.), according to the manufacturer's recommended protocols.

### Analysis of Inhibition of Target Levels or Expression

Inhibition of levels or expression of a Factor VII nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif., and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT real-time PCR reagents are obtained from Invitrogen (Carlsbad, Calif.). RT and real-time-PCR reactions are carried out by methods well known to those skilled in the art.

Gene (or RNA) target quantities obtained by real-time PCR are normalized using either the expression level of a gene whose expression is constant, such as cyclophilin A, or by quantifying total RNA using RIBOGREEN (Invitrogen, Inc. Carlsbad, Calif.). Cyclophilin A expression is quantified by real-time PCR, by being run simultaneously with the target, multiplexing, or separately. Total RNA is quantified using RIBOGREEN RNA quantification reagent (Invetrogen, Inc. Eugene, Oreg.). Methods of RNA quantification by RIBOGREEN are taught in Jones, L. J., et al., (Analytical Biochemistry, 1998, 265, 368-374). A CYTOFLUOR4000 instrument (PE Applied Biosystems) is used to measure RIBOGREEN fluorescence.

Probes and primers are designed to hybridize to a Factor VII nucleic acid. Methods for designing real-time PCR probes and primers are well known in the art, and may include the use of software such as PRIMER EXPRESS Software (Applied Biosystems, Foster City, Calif.).

### Analysis of Protein Levels

Antisense inhibition of Factor VII nucleic acids can be assessed by measuring Factor VII protein levels. Protein levels of Factor VII can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of mouse, rat, monkey, and human Factor VII are commercially available.

### In Vivo Testing of Antisense Compounds

Antisense compounds, for example, antisense oligonucleotides, are tested in animals to assess their ability to inhibit expression of Factor VII and produce phenotypic changes, such as, prolonged PT, prolonged aPTT time, decreased quantity of Platelet Factor 4 (PF-4), reduced formation of thrombus or increased time for thrombus formation, and reduction of cellular proliferation. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from liver tissue and changes in Factor VII nucleic acid expression are measured. Changes in Factor VII protein levels are also measured using a thrombin generation assay. In addition, effects on clot times, e.g. PT and aPTT, are determined using plasma from treated animals.

### Certain Indications

In certain embodiments, the invention provides methods of treating an individual comprising administering one or more pharmaceutical compositions described herein. In certain embodiments, the individual has a thromboembolic complication. In certain embodiments, the individual is at risk for a blood clotting disorder, including, but not limited to, infarction, thrombosis, embolism, thromboembolism, such as deep vein thrombosis, pulmonary embolism, myocardial infarction, and stroke. This includes individuals with an acquired problem, disease, or disorder that leads to a risk of thrombosis, for example, surgery, cancer, immobility, sepsis, atherosclerosis, atrial fibrillation, as well as genetic predisposition, for example, antiphospholipid syndrome and the autosomal dominant condition, Factor V Leiden. In certain embodiments, the individual has been identified as in need of anti-coagulation therapy. Examples of such individuals include, but are not limited to, those undergoing major orthopedic surgery (e.g., hip/knee replacement or hip fracture surgery) and patients in need of chronic treatment, such as those suffering from atrial fibrillation to prevent stroke. In certain embodiments the invention provides methods for prophylactically reducing Factor VII expression in an individual. Certain embodiments include treating an individual in need thereof by administering to an individual a therapeutically effective amount of an antisense compound targeted to a Factor VII nucleic acid.

In certain embodiments, pharmaceutical compositions comprising an antisense compound targeted to Factor VII are used for the preparation of a medicament for treating a patient suffering or susceptible to a thromboembolic complication.

In certain embodiments, the binding of Factor VII with Tissue factor to form Tissue Factor-Factor VIIa complex may lead to inflammatory conditions, such as liver fibrosis and rheumatoid arthritis and/or hyperproliferative disorders such as tumor growth and metastasis.

In certain embodiments, the individual has an inflammatory condition leading to a fibrosis complication. In certain embodiments, the individual is at risk of an excessive collagen deposition and fibrosis disorder, including, but not limited to, liver fibrosis, arterial sclerosis, chronic glomerulonephritis, cutis keloid formation, progressive systemic sclerosis (PSS), liver fibrosis, pulmonary fibrosis, cystic fibrosis, chronic graft versus host disease, scleroderma (local and systemic), Peyronie's disease, penis fibrosis, urethrostenosis after the test using a cystoscope, inner accretion after surgery, myelofibrosis, idiopathic retroperitoneal fibrosis. In certain embodiments, the individual has been identified as in need of anti-fibrotic therapy. This includes individuals with a genetic or acquired problem, disease, or disorder that leads to a risk of fibrosis, for example, α1-antitrypsin deficiency, copper storage disease (Wilson's disease), fructosemia, galactosemia, glycogen storage diseases (such as, types II, IV, VI, IX, and X), iron overload syndromes (such as, hemochromatosis), lipid abnormalities (such as, Gaucher's disease), peroxisomal disorders (such as, Zellweger syndrome), Tyrsoninemia, congenital hepatic fibrosis, bacterial infection (such as, brucellosis), parasitic infection (such as, echinococcosis), viral infections (such as, chronic hepatitis B, C), disorders affecting hepatic blood flow (such as, Budd Chiari syndrome, heart failure, hepatic veno-occlusive disease, and portal vein thrombosis), alcohol, and drugs (such as amiodarone, chlorpromazine, Isoniazid, Methotrexate, Methyldopa, Oxyphenisatin, and Tolbutamide). In certain embodiments, the individual has been identified as in need of anti-fibrotic therapy. In such embodiments, the tissue factor-Factor VIIa (TF/F7a) complex is identified to have the major procoagulant activity in fibrosis. In certain embodiments, the invention provides methods for prophylactically reducing Factor VII expression in an individual. Certain embodiments include treating an individual in need thereof by administering to an individual a therapeutically effective amount of an antisense compound targeted to a Factor VII nucleic acid.

In certain embodiments, pharmaceutical compositions comprising an antisense compound targeted to Factor VII are used for the preparation of a medicament for treating a patient suffering or susceptible to a fibrotic complication.

In certain embodiments, the individual has an inflammatory rheumatoid arthritic complication. In certain embodiments, the individual is at risk for inflammation at the joints and rheumatoid arthritis. In such embodiments, the individual suffers from pain, swelling and tenderness at the joints, fatigue, lack of appetite, low-grade fever, muscle aches and stiffness. In certain embodiments, the individual has been identified as in need of anti-inflammatory arthritic therapy. This includes individuals suffering from rheumatoid arthritis, reactive arthritis, Reiter's syndrome, psoriatic arthritis, ankylosing spondylitis, and arthritis associated with inflammatory bowel disease. In certain embodiments, the individual has been identified as in need of anti-inflammatory therapy. In such embodiments, the tissue factor-Factor VIIa (TF/F7a) complex is identified to have the major procoagulant activity in inducing arthritis. In certain embodiments the invention provides methods for prophylactically reducing Factor VII expression in an individual. Certain embodiments include treating an individual in need thereof by administering to an individual a therapeutically effective amount of an antisense compound targeted to a Factor VII nucleic acid.

In certain embodiments, pharmaceutical compositions comprising an antisense compound targeted to Factor VII are used for the preparation of a medicament for treating a patient suffering or susceptible to an inflammatory arthritic complication.

In certain embodiments, the individual has a malignant complication. In certain embodiments, the individual is at risk for tumor growth, angiogenesis and metastasis. In such embodiments, the individual suffering from hemostatic abnormalities, such as disseminated intravascular coagulation and venous thromboembolism, may suffer additional complications, such as primary and metastatic tumor growths. In such embodiments, the seeding of tumor metastases is a coagulation-dependent process. In such embodiments, the tissue factor-Factor VIIa (TF/F7a) complex is identified to have the major procoagulant activity in cancer. In certain embodiments, the individual has been identified as in need of anti-TF/F7a therapy. In certain embodiments the invention provides methods for prophylactically reducing Factor VII expression in an individual. Certain embodiments include treating an individual in need thereof by administering to an individual a therapeutically effective amount of an antisense compound targeted to a Factor VII nucleic acid.

In certain embodiments, pharmaceutical compositions comprising an antisense compound targeted to Factor VII are used for the preparation of a medicament for treating a patient suffering or susceptible to a malignant complication.

In certain embodiments, administration of a therapeutically effective amount of an antisense compound targeted to a Factor VII nucleic acid is accompanied by monitoring of Factor VII levels in the serum of an individual, to determine an individual's response to administration of the antisense compound. An individual's response to administration of the antisense compound is used by a physician to determine the amount and duration of therapeutic intervention.

In certain embodiments, administration of an antisense compound targeted to a Factor VII nucleic acid results in reduction of Factor VII expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a Factor VII nucleic acid results in a change in a measure of blood clotting, as measured by a standard test, for example, but not limited to, activated partial thromboplastin time (aPTT) test, prothrombin time (PT) test, thrombin time (TCT), bleeding time, or D-dimer. In certain embodiments, administration of a Factor VII antisense compound increases the measure by at least 15, 20, 25, 30, 35, 40, 45, 50, 55, 60, 65, 70, 75, 80, 85, 90, 95 or 99%, or a range defined by any two of these values. In some embodiments, administration of a Factor VII antisense compound decreases the measure by at least 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to Factor VII are used for the preparation of a medicament for treating a patient suffering or susceptible to a thromboembolic complication.

### Certain Combination Therapies

In certain embodiments, one or more pharmaceutical compositions described herein are co-administered with one or more other pharmaceutical agents. In certain embodiments, such one or more other pharmaceutical agents are designed to treat the same disease, disorder, or condition as the one or more pharmaceutical compositions described herein. In certain embodiments, such one or more other pharmaceutical agents are designed to treat a different disease, disorder, or condition as the one or more pharmaceutical compositions described herein. In certain embodiments, such one or more other pharmaceutical agents are designed to treat an undesired side effect of one or more pharmaceutical compositions described herein. In certain embodiments, one or more pharmaceutical compositions described herein are co-administered with another pharmaceutical agent to treat an undesired effect of that other pharmaceutical agent. In certain embodiments, one or more pharmaceutical compositions described herein are co-administered with another pharmaceutical agent to produce a combinational effect. In certain embodiments, one or more pharmaceutical compositions described herein are co-administered with another pharmaceutical agent to produce a synergistic effect.

In certain embodiments, one or more pharmaceutical compositions described herein and one or more other pharmaceutical agents are administered at the same time. In certain embodiments, one or more pharmaceutical compositions described herein and one or more other pharmaceutical agents are administered at different times. In certain embodiments, one or more pharmaceutical compositions described herein and one or more other pharmaceutical agents are prepared together in a single formulation. In certain embodiments, one or more pharmaceutical compositions described herein and one or more other pharmaceutical agents are prepared separately.

In certain embodiments, pharmaceutical agents that may be co-administered with a pharmaceutical composition described herein include anticoagulant or antiplatelet agents. In certain embodiments, pharmaceutical agents that may be co-administered with a pharmaceutical composition described herein include, but are not limited to aspirin, clopidogrel, dipyridamole, ticlopidine, warfarin (and related coumarins), heparin, direct thrombin inhibitors (such as lepirudin, bivalirudin), apixaban, lovenox, and small molecular compounds that interfere directly with the enzymatic action of particular coagulation factors (e.g. rivaroxaban, which interferes with Factor Xa). In certain embodiments, the anticoagulant or antiplatelet agent is administered prior to administration of a pharmaceutical composition described herein. In certain embodiments, the anticoagulant or antiplatelet agent is administered following administration of a pharmaceutical composition described herein. In certain embodiments the anticoagulant or antiplatelet agent is administered at the same time as a pharmaceutical composition described herein. In certain embodiments the dose of a co-administered anticoagulant or antiplatelet agent is the same as the dose that would be administered if the anticoagulant or antiplatelet agent was administered alone. In certain embodiments the dose of a co-administered anticoagulant or antiplatelet agent is lower than the dose that would be administered if the anticoagulant or antiplatelet agent was administered alone. In certain embodiments the dose of a co-administered anticoagulant or antiplatelet agent is greater than the dose that would be administered if the anticoagulant or antiplatelet agent was administered alone.

In certain embodiments, the co-administration of a second compound enhances the anticoagulant effect of a first compound, such that co-administration of the compounds results in an anticoagulant effect that is greater than the effect of administering the first compound alone. In other embodiments, the co-administration results in anticoagulant effects that are additive of the effects of the compounds when administered alone. In certain embodiments, the co-administration results in anticoagulant effects that are supra-additive of the effects of the compounds when administered alone. In certain embodiments, the first compound is an antisense compound. In certain embodiments, the second compound is an antisense compound.

In certain embodiments, pharmaceutical agents that may be co-administered with a pharmaceutical composition described herein include anti-inflammatory agents. In certain embodiments, pharmaceutical agents that may be co-administered with a pharmaceutical composition described herein include, but are not limited to serine protease inhibitor C1-INH recombinant protein, kallikrein antisense oligonucleotide, CINRYZE, BERINERT, KALBITOR, Icatibant, Ecallantide, attenuated androgens, anabolic steroids, and antifibrinolytic agents (e.g., epsilon-aminocaproic acid and tranexamic acid). In certain embodiments, the anti-inflammatory agent is administered prior to administration of a pharmaceutical composition described herein. In certain embodiments, the anti-inflammatory agent is administered following administration of a pharmaceutical composition described herein. In certain embodiments the anti-inflammatory agent is administered at the same time as a pharmaceutical composition described herein. In certain embodiments the dose of a co-administered anti-inflammatory agent is the same as the dose that would be administered if the anti-inflammatory agent was administered alone. In certain embodiments the dose of a co-administered anti-inflammatory agent is lower than the dose that would be administered if the anti-inflammatory agent was administered alone. In certain embodiments the dose of a co-administered anti-inflammatory agent is greater than the dose that would be administered if the anti-inflammatory agent was administered alone.

In certain embodiments, the co-administration of a second compound enhances the anti-inflammatory effect of a first compound, such that co-administration of the compounds results in an anti-inflammatory effect that is greater than the effect of administering the first compound alone. In other embodiments, the co-administration results in anti-inflammatory effects that are additive of the effects of the compounds when administered alone. In certain embodiments, the co-administration results in anti-inflammatory effects that are supra-additive of the effects of the compounds when administered alone. In certain embodiments, the first compound is an antisense compound. In certain embodiments, the second compound is an antisense compound.

In certain embodiments, pharmaceutical agents that may be co-administered with a pharmaceutical composition described herein include anti-hyperproliferative agents. In certain embodiments, pharmaceutical agents that may be co-administered with a pharmaceutical composition described herein include, but are not limited to all-trans retinoic acid, azacitidine, azathioprine, bleomycin, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, mechlorethamine, mercaptopurine, methotrexate, mitoxantrone, oxaliplatin, paclitaxel, pemetrexed, teniposide, tioguanine, valrubicin, vinblastine, vincristine, vindesine, or vinorelbine. In certain embodiments, the anti-hyperproliferative agent is administered prior to administration of a pharmaceutical composition described herein. In certain embodiments, the anti-hyperproliferative agent is administered following administration of a pharmaceutical composition described herein. In certain embodiments the anti-hyperproliferative agent is administered at the same time as a pharmaceutical composition described herein. In certain embodiments the dose of a co-administered anti-hyperproliferative agent is the same as the dose that would be administered if the anti-hyperproliferative agent was administered alone. In certain embodiments the dose of a co-administered anti-hyperproliferative agent is lower than the dose that would be administered if the anti-hyperproliferative agent was administered alone. In certain embodiments the dose of a co-administered anti-hyperproliferative agent is greater than the dose that would be administered if the anti-hyperproliferative agent was administered alone.

In certain embodiments, the co-administration of a second compound enhances the anti-hyperproliferative effect of a first compound, such that co-administration of the compounds results in an anti-hyperproliferative effect that is greater than the effect of administering the first compound alone. In other embodiments, the co-administration results in anti-hyperproliferative effects that are additive of the effects of the compounds when administered alone. In certain embodiments, the co-administration results in anti-hyperproliferative effects that are supra-additive of the effects of the compounds when administered alone. In certain embodiments, the first compound is an antisense compound. In certain embodiments, the second compound is an antisense compound.

In certain embodiments, an antidote is administered anytime after the administration of a Factor VII specific inhibitor. In certain embodiments, an antidote is administered anytime after the administration of an antisense oligonucleotide targeting Factor VII. In certain embodiments, the antidote is administered minutes, hours, days, weeks, or months after the administration of an antisense compound targeting Factor VII. In certain embodiments, the antidote is a complementary (e.g. a sense strand) to the antisense compound targeting Factor VII. In certain embodiments, the antidote is a Factor VII or Factor VIIa protein. In certain embodiments, the Factor VII or Factor VIIa, protein is a human Factor VII or human Factor VIIa protein.

### Certain Comparator Compositions

In certain embodiments, ISIS 407935, a 5-10-5 MOE gapmer, having a sequence of (from 5′ to 3′) ATGCATGGTGATGCTTCTGA (incorporated herein as SEQ ID NO: 120), wherein each internucleoside linkage is a phosphorothioate linkage, each cytosine is a 5-methylcytosine, and each of nucleosides 1-5 and 16-20 comprise a 2′-O-methoxyethyl moiety, which was previously described in WO 2009/061851, incorporated herein by reference, is a comparator compound.

In certain embodiments, ISIS 407936, a 5-10-5 MOE gapmer, having a sequence of (from 5′ to 3′) GGCATTCGCCACCATGCATG (incorporated herein as SEQ ID NO: 122), wherein each internucleoside linkage is a phosphorothioate linkage, each cytosine is a 5-methylcytosine, and each of nucleosides 1-5 and 16-20 comprise a 2′-O-methoxyethyl moiety, which was previously described in WO 2009/061851, incorporated herein by reference, is a comparator compound.

In certain embodiments, ISIS 407939, a 5-10-5 MOE gapmer, having a sequence of (from 5′ to 3′) TGCAGCCCGGCACCCAGCGA (incorporated herein as SEQ ID NO: 72), wherein each internucleoside linkage is a phosphorothioate linkage, each cytosine is a 5-methylcytosine, and each of nucleosides 1-5 and 16-20 comprise a 2′-O-methoxyethyl moiety, which was previously described in WO 2009/061851, incorporated herein by reference, is a comparator compound.

In certain embodiments, compounds described herein are more efficacious, potent, and/or tolerable in various in vitro and in vivo systems than ISIS 407935, ISIS 407936, and/or ISIS 407939. ISIS 407935, ISIS 407936, and ISIS 407939 were selected as a comparator compounds because they exhibited high levels of dose-dependent inhibition in various studies as described in WO 2009/061851. Thus, ISIS 407935, ISIS 407936, and ISIS 407939 were deemed highly efficacious and potent compounds. In certain embodiments, other compounds described in WO 2009/061851 are used as comparator compounds.

### Certain Compositions

In certain embodiments, ISIS 473589 is more efficacious, potent, and/or tolerable than comparator compositions, such as ISIS 407935, 407936, and/or ISIS 407939.

For example, as provided in Example 1 (hereinbelow), ISIS 473589 achieved 97% inhibition in cultured Hep3B cells when transfected using electroporation with 2,000 nM antisense oligonucleotide, whereas ISIS 407939 achieved 80% inhibition. Thus, ISIS 473589 is more efficacious than the comparator compound, ISIS 407939.

In another example, as provided in Example 13 (hereinbelow), ISIS 473589 achieved an IC₅₀ of 0.3 μM in a 5 point dose response curve (0.074 μM, 0.222 μM, 0.667 μM, 2.000 μM, and 6.000 μM) in cultured in Hep3B cells when transfected using electroporation, whereas ISIS 407939 achieved an IC₅₀ of 0.9 μM. Thus, ISIS 473589 is more potent than the comparator compound, ISIS 407939.

In another example, as provided in Example 17 (hereinbelow), ISIS 473589 achieved 96% inhibition when administered subcutaneously twice a week for 3 weeks with 10 mg/kg/week to transgenic mice harboring a Factor VII genomic DNA fragment, whereas ISIS 407935 achieved 80% inhibition. Thus, ISIS 473589 is more efficacious than the comparator compound, ISIS 407939.

In another example, as provided in Example 34 (hereinbelow), ISIS 473589 exhibited more favorable tolerability markers than ISIS 407935 when administered to CD-1 mice. ISIS 473589 was administered subcutaneously twice a week for 6 weeks at 25 mg/kg. ISIS 407935 was administered subcutaneously twice a week for 6 weeks at 50 mg/kg. After treatment, ALT, AST, and BUN levels were lower in ISIS 473589 treated mice than in ISIS 407935 treated mice. Therefore, ISIS 473589 is more tolerable than the comparator compound, ISIS 407935 in CD-1 mice.

In another example, as provided in Example 35 (hereinbelow), ISIS 473589 exhibited more favorable tolerability markers than ISIS 407935 when administered to Sprague-Dawley rats. ISIS 473589 was administered subcutaneously twice a week for 6 weeks at 25 mg/kg. ISIS 407935 was administered subcutaneously twice a week for 6 weeks at 50 mg/kg. After treatment, ALT, AST, and BUN levels were lower in ISIS 473589 treated rats than in ISIS 407935 treated rats. Therefore, ISIS 473589 is more tolerable than the comparator compound, ISIS 407935 in Sprague-Dawley rats.

In another example, as provided in Example 38 (hereinbelow), ISIS 473589 achieved 25%, 44%, 62%, and 80% mRNA inhibition and 0%, 6%, 40%, and 78% protein inhibition when administered to transgenic mice harboring a Factor VII genomic DNA fragment subcutaneously twice a week for 3 weeks at 0.625, 1.25, 2.50, and 5.00 mg/kg/week. ISIS 407935 achieved 28%, 45%, 57%, and 85% mRNA inhibition and 3%, 0%, 47%, and 65% protein inhibition when administered to transgenic mice harboring a Factor VII genomic DNA fragment subcutaneously twice a week for 3 weeks at 2.5, 5.0, 10.0, and 20.00 mg/kg/week. Therefore, ISIS 473589 is more efficacious than ISIS 407935.

In another example, as provided in Example 39 (hereinbelow), ISIS 473589 exhibited more favorable tolerability markers in cynomolgous monkeys including complement C3 measurements, kidney function, body and organ weight, and macroscopic observation upon necropsy. Treatment with ISIS 407935 resulted in reduced complement C3 levels, indicating treatment with ISIS 407935 may have resulted in repeated complement activation to a greater degree than ISIS 473589. Treatment with ISIS 407935 resulted in elevated urine protein to creatinine ratio in the monkeys, indicating treatment with ISIS 407935 perturbed kidney function, whereas treatment with 473589 did not have any effect on the kidney function outside the expected range. Treatment with ISIS 407935 resulted in a 2.2-fold increase in spleen weight, a 2.7-fold increase in liver weight, and a 1.3-fold increase in kidney weight compared to the control, indicating that ISIS 407935 had an effect on organ weights, which was not observed with ISIS 473589. ISIS 407935 was observed to result in ascites in 2 out of 4 monkeys suggesting it is less well tolerated than ISIS 473589. Therefore, ISIS 473589 is more tolerable than the comparator compound, ISIS 407935.

In certain embodiments, ISIS 490279 is more efficacious, potent, and/or tolerable than comparator compositions, such as ISIS 407935, 407936, and/or ISIS 407939.

For example, as provided in Example 29 (hereinbelow), ISIS 490279 achieved 59% inhibition when administered subcutaneously twice a week for 3 weeks with 1 mg/kg/week to transgenic mice harboring a Factor VII genomic DNA fragment, whereas ISIS 407936 achieved 28% inhibition. Thus, ISIS 490279 is more efficacious than the comparator compound, ISIS 407936.

In another example, as provided in Example 34 (hereinbelow), ISIS 490279 exhibited more favorable tolerability markers than ISIS 407935 when administered to CD-1 mice. ISIS 490279 was administered subcutaneously twice a week for 6 weeks at 50 mg/kg. ISIS 407935 was administered subcutaneously twice a week for 6 weeks at 50 mg/kg. After treatment, ALT, AST, and BUN levels were lower in ISIS 490279 treated mice than in ISIS 407935 treated mice. Therefore, ISIS 490279 is more tolerable than the comparator compound, ISIS 407935 in CD-1 mice.

In another example, as provided in Example 35 (hereinbelow), ISIS 490279 was as tolerable or more tolerable than ISIS 407935 when administered to Sprague-Dawley rats. ISIS 490279 was administered subcutaneously twice a week for 6 weeks at 50 mg/kg. ISIS 407935 was administered subcutaneously twice a week for 6 weeks at 50 mg/kg. After treatment, ALT was lower in ISIS 490279 treated rats than in ISIS 407935 treated rats. Therefore, ISIS 490279 is as tolerable or more tolerable than the comparator compound, ISIS 407935 in Sprague-Dawley rats.

In another example, as provided in Example 38 (hereinbelow), ISIS 490279 achieved 33%, 51%, 70%, and 88% mRNA inhibition and 23%, 31%, 75%, and 91% protein inhibition when administered to transgenic mice harboring a Factor VII genomic DNA fragment subcutaneously twice a week for 3 weeks at 2.5, 5.0, 10.0, and 20.00 mg/kg/week. ISIS 407935 achieved 28%, 45%, 57%, and 85% mRNA inhibition and 3%, 0%, 47%, and 65% protein inhibition when administered to transgenic mice harboring a Factor VII genomic DNA fragment subcutaneously twice a week for 3 weeks at 2.5, 5.0, 10.0, and 20.00 mg/kg/week. Therefore, ISIS 473589 is more efficacious than ISIS 407935.

In another example, as provided in Example 39 (hereinbelow), ISIS 490279 exhibited more favorable tolerability markers in cynomolgous monkeys including complement C3 measurements, kidney function, body and organ weight, and macroscopic observation upon necropsy. Treatment with ISIS 407935 resulted in reduced complement C3 levels, indicating treatment with ISIS 407935 may have resulted in repeated complement activation to a greater degree than ISIS 490279. Treatment with ISIS 407935 resulted in elevated urine protein to creatinine ratio in the monkeys, indicating treatment with ISIS 407935 perturbed kidney function, whereas treatment with 490279 did not have any effect on the kidney function outside the expected range. Treatment with ISIS 407935 resulted in a 2.2-fold increase in spleen weight, a 2.7-fold increase in liver weight, and a 1.3-fold increase in kidney weight compared to the control, indicating that ISIS 407935 had an effect on organ weights, which was not observed with ISIS 490279. ISIS 407935 was observed to result in ascites in 2 out of 4 monkeys suggesting it is less well tolerated than ISIS 490279. Therefore, ISIS 490279 is more tolerable than the comparator compound, ISIS 407935.

In certain embodiments, ISIS 540175 is more efficacious, potent, and/or tolerable than comparator compositions, such as ISIS 407935.

For example, as provided in Example 31 (hereinbelow), ISIS 540175 achieved 55% and 90% inhibition when administered subcutaneously with 0.1 mg/kg/week and 0.3 mg/kg/week to transgenic mice harboring a Factor VII genomic DNA fragment, whereas ISIS 407935 achieved 31% and 65% inhibition when administered at 0.5 mg/kg/week and 1.5 mg/kg/week. Thus, ISIS 540175 is more potent than the comparator compounds, ISIS 407935.

In another example, as provided in Example 34 (hereinbelow), ISIS 540175 exhibited more favorable tolerability markers than ISIS 407935 when administered to CD-1 mice. ISIS 540175 was administered subcutaneously twice a week for 6 weeks at 25 mg/kg. ISIS 407935 was administered subcutaneously twice a week for 6 weeks at 50 mg/kg. After treatment, ALT and AST levels were lower in ISIS 540175 treated mice than in ISIS 407935 treated mice. Therefore, ISIS 540175 is more tolerable than the comparator compound, ISIS 407935 in CD-1 mice.

In another example, as provided in Example 35 (hereinbelow), ISIS 540175 exhibited more favorable tolerability markers than ISIS 407935 when administered to Sprague-Dawley rats. ISIS 540175 was administered subcutaneously twice a week for 6 weeks at 25 mg/kg. ISIS 407935 was administered subcutaneously twice a week for 6 weeks at 50 mg/kg. After treatment, ALT, AST, and BUN levels were lower in ISIS 540175 treated rats than in ISIS 407935 treated rats. Therefore, ISIS 540175 is more tolerable than the comparator compound, ISIS 407935 in Sprague-Dawley rats.

In another example, as provided in Example 38 (hereinbelow), ISIS 540175 achieved 55%, 65%, 85%, and 95% mRNA inhibition and 24%, 49%, 83%, and 93% protein inhibition when administered to transgenic mice harboring a Factor VII genomic DNA fragment subcutaneously twice a week for 3 weeks at 0.625, 1.25, 2.50, and 5.00 mg/kg/week. ISIS 407935 achieved 28%, 45%, 57%, and 85% mRNA inhibition and 3%, 0%, 47%, and 65% protein inhibition when administered to transgenic mice harboring a Factor VII genomic DNA fragment subcutaneously twice a week for 3 weeks at 2.5, 5.0, 10.0, and 20.00 mg/kg/week. Therefore, ISIS 540175 is more efficacious than ISIS 407935.

In another example, as provided in Example 39 (hereinbelow), ISIS 540175 exhibited more favorable tolerability markers in cynomolgous monkeys including complement C3 measurements, kidney function, body and organ weight, and macroscopic observation upon necropsy. Treatment with ISIS 540175 resulted in reduced complement C3 levels, indicating treatment with ISIS 407935 may have resulted in repeated complement activation to a greater degree than ISIS 540175. Treatment with ISIS 407935 resulted in elevated urine protein to creatinine ratio in the monkeys, indicating treatment with ISIS 407935 perturbed kidney function, whereas treatment with 540175 did not have any effect on the kidney function outside the expected range. Treatment with ISIS 407935 resulted in a 2.2-fold increase in spleen weight, a 2.7-fold increase in liver weight, and a 1.3-fold increase in kidney weight compared to the control, indicating that ISIS 407935 had an effect on organ weights, which was not observed with ISIS 540175. ISIS 407935 was observed to result in ascites in 2 out of 4 monkeys suggesting it is less well tolerated than ISIS 540175. Therefore, ISIS 540175 is more tolerable than the comparator compound, ISIS 407935.

In another example, as provided in Example 40 (hereinbelow), ISIS 540175 achieved an IC₅₀ of 0.2 μM in a 5 point dose response curve (0.003 μM, 0.016 μM, 0.800 μM, 4.000 μM, and 20.000 μM) in cultured HepG2 cells when transfected using electroporation, whereas ISIS 407935 achieved an IC₅₀ of 0.4 μM. Thus, ISIS 540175 is more potent than the comparator compound, ISIS 407935.

### EXAMPLES

### Non-Limiting Disclosure and Incorporation by Reference

While certain compounds, compositions, and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Modified Antisense Oligonucleotides Comprising cEt and MOE Modifications Targeting Human Coagulation Factor VII

Antisense oligonucleotides were designed targeting a Factor VII nucleic acid and were tested for their effects on Factor VII mRNA in vitro. ISIS 407939 (described hereinabove), which was described in an earlier publication (WO 2009/061851) was also tested.

The newly designed modified antisense oligonucleotides and their motifs are described in Table 1. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO.

Each oligonucleotide listed in Table 1 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted human gene sequence. ‘n/a’ indicates that the antisense oligonucleotide is not 100% complementary with that particular gene sequence. Oligonucleotides having multiple start and stop sites target a region that is repeated within a Factor VII sequence (e.g., within SEQ ID NO: 1).

Activity of the newly designed oligonucleotides was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (forward sequence GGGACCCTGATCAACACCAT, designated herein as SEQ ID NO: 5; reverse sequence CCAGTTCTTGATTTTGTCGAAACA, designated herein as SEQ ID NO: 6; probe sequence TGGGTGGTCTCCGCGGCC, designated herein as SEQ ID NO: 7) was used to measure mRNA levels. Factor VII mRNA levels were adjusted according to total RNA content, as measured by RIBOGREEN®. Results are presented as percent inhibition of Factor VII expression, relative to untreated control cells. A total of 771 oligonucleotides were tested. Only those oligonucleotides which were selected for further studies are shown in Table 1. Each of the newly designed antisense oligonucleotides provided in Table 1 achieved greater than 80% inhibition and, therefore, are more active than ISIS 407939.

TABLE 1
Percent inhibition of human Factor VII mRNA levels by modified
antisense oligonucleotides targeted to SEQ ID NO: 1 and SEQ ID NO: 2

| SEQ | | | | | | | | |
|---|---|---|---|---|---|---|---|---|
| ID | SEQ | | | | | SEQ | SEQ | |
| NO: 1 | ID NO: | | SEQ | | | ID NO: | ID NO: | |
| Start | 1 Stop | | ID | ISIS | % | 2 Start | 2 Stop | |
| Site | Site | Sequence | NO | NO | inhibition | Site | Site | Sugar Chemistry |
| 15255 | 15274 | TGCAGCCCGGCACCCAG | 72 | 407939 | 80 | 2312 | 2331 | eeeeeddddddddddeeeee |
| | | CGA | | | | | | |
| 1147 | 1162 | GATGAAATCTCTGCAG | 21 | 473359 | 92 | 36 | 51 | kkkddddkdddddeee |
| 1154 | 1169 | AGACCATGATGAAATC | 22 | 473360 | 96 | 43 | 58 | kkkddddkdddddeee |
| 1382 | 1397 | GCCTGGATGCTGGTTT | 23 | 473168 | 94 | n/a | n/a | kkkddddddddddkkk |
| 1382 | 1397 | GCCTGGATGCTGGTTT | 23 | 473317 | 95 | n/a | n/a | kkkddddddddddeee |
| 1382 | 1397 | GCCTGGATGCTGGTTT | 23 | 473471 | 90 | n/a | n/a | kkkddddkdddddeee |
| 1382 | 1397 | GCCTGGATGCTGGTTT | 23 | 473620 | 94 | n/a | n/a | kdkdkdddddddddee |
| 1383 | 1396 | CCTGGATGCTGGTT | 24 | 473019 | 88 | n/a | n/a | kkddddddddddkk |
| 1384 | 1397 | GCCTGGATGCTGGT | 25 | 473020 | 93 | n/a | n/a | kkddddddddddkk |
| 2369 | 2384 | TGGAGCGGTCACTTCC | 26 | 473321 | 93 | n/a | n/a | kkkddddddddddeee |
| 4717 | 4732 | AGGAGGCTGAGGATGC | 27 | 473322 | 94 | n/a | n/a | kkkddddddddddeee |
| 4871 | 4886 | CTGCAGGAGCGGCCTA | 28 | 473323 | 96 | n/a | n/a | kkkddddddddddeee |
| 6411 | 6426 | CGTATTTTCTGATGTG | 29 | 473326 | 94 | n/a | n/a | kkkddddddddddeee |
| 6411 | 6426 | CGTATTTTCTGATGTG | 29 | 473480 | 92 | n/a | n/a | kkkddddkdddddeee |
| 6642 | 6657 | GAGGTGACCCGTGAGC | 30 | 473178 | 96 | n/a | n/a | kkkddddddddddkkk |
| 6642 | 6657 | GAGGTGACCCGTGAGC | 30 | 473327 | 96 | n/a | n/a | kkkddddddddddeee |
| 6642 | 6657 | GAGGTGACCCGTGAGC | 30 | 473481 | 93 | n/a | n/a | kkkddddkdddddeee |
| 6642 | 6657 | GAGGTGACCCGTGAGC | 30 | 473630 | 89 | n/a | n/a | kdkdkdddddddddee |
| 6643 | 6656 | AGGTGACCCGTGAG | 31 | 473029 | 96 | n/a | n/a | kkddddddddddkk |
| 6765 | 6778 | | | | | | | |
| 6887 | 6900 | | | | | | | |
| 6953 | 6966 | | | | | | | |
| 7071 | 7084 | | | | | | | |
| 7189 | 7202 | | | | | | | |
| 7243 | 7256 | | | | | | | |
| 11017 | 11030 | CTGCTCACAGCCGC | 32 | 472925 | 93 | 452 | 465 | kkddddddddddkk |
| 11023 | 11036 | GCAGTACTGCTCAC | 33 | 472926 | 85 | 458 | 471 | kkddddddddddkk |
| 11839 | 11854 | AATGGTCAGGGCTGGT | 34 | 473195 | 97 | n/a | n/a | kkkddddddddddkkk |
| 11840 | 11853 | ATGGTCAGGGCTGG | 35 | 473046 | 90 | n/a | n/a | kkddddddddddkk |
| 12128 | 12141 | GGTTTGCTGGCATT | 36 | 472935 | 92 | 598 | 611 | kkddddddddddkk |
| 12141 | 12156 | ACAATTCGGCCTTGGG | 37 | 473089 | 95 | 611 | 626 | kkkddddddddddkkk |
| 12629 | 12644 | GCTCAGACCTGGCTCT | 38 | 473350 | 93 | n/a | n/a | kkkddddddddddeee |
| 12633 | 12648 | AGCTGCTCAGACCTGG | 39 | 473353 | 93 | n/a | n/a | kkkddddddddddeee |
| 12634 | 12647 | GCTGCTCAGACCTG | 40 | 473055 | 91 | n/a | n/a | kkddddddddddkk |
| 12842 | 12857 | CCACCCAGATGGTGTT | 41 | 473392 | 95 | 715 | 730 | kkkddddkdddddeee |
| 12863 | 12878 | CGAAACAGTGGGCCGC | 42 | 473095 | 100 | 736 | 751 | kkkddddddddddkkk |
| 12863 | 12878 | CGAAACAGTGGGCCGC | 42 | 473244 | 99 | 736 | 751 | kkkddddddddddeee |
| 12863 | 12878 | CGAAACAGTGGGCCGC | 42 | 473393 | 99 | 736 | 751 | kkkddddkdddddeee |
| 12863 | 12878 | CGAAACAGTGGGCCGC | 42 | 473547 | 98 | 736 | 751 | kdkdkdddddddddee |
| 12864 | 12877 | GAAACAGTGGGCCG | 43 | 472942 | 87 | 737 | 750 | kkddddddddddkk |
| 13741 | 13756 | GTGCTCGCTGAGGTCG | 44 | 473098 | 97 | 798 | 813 | kkkddddddddddkkk |
| 13988 | 14003 | CCATGAGCTCCAGGGC | 45 | 473408 | 92 | 1045 | 1060 | kkkddddkdddddeee |
| 14019 | 14032 | CTGGGTCATCAGCC | 46 | 472958 | 89 | 1076 | 1089 | kkddddddddddkk |
| 14022 | 14035 | GTCCTGGGTCATCA | 47 | 472959 | 90 | 1079 | 1092 | kkddddddddddkk |
| 14079 | 14094 | CAGAACATGTACTCCG | 48 | 473566 | 94 | 1136 | 1151 | kdkdkdddddddddee |
| 14092 | 14107 | CGAGTAGCCGGCACAG | 49 | 473567 | 95 | 1149 | 1164 | kdkdkdddddddddee |
| 14128 | 14143 | TCCACTGTCCCCCTTG | 50 | 473569 | 92 | 1185 | 1200 | kdkdkdddddddddee |
| 14232 | 14245 | CCTGGTGTACACCC | 51 | 457851 | 90 | 1289 | 1302 | kkddddddddddkk |
| 14244 | 14257 | GTACTGGGAGACCC | 32 | 472970 | 91 | 1301 | 1314 | kkddddddddddkk |
| 14612 | 14627 | CCCCTCTGTCCAGCGC | 53 | 473125 | 90 | 1669 | 1684 | kkkddddddddddkkk |
| 14612 | 14627 | CCCCTCTGTCCAGCGC | 53 | 473274 | 98 | 1669 | 1684 | kkkddddddddddeee |
| 14612 | 14627 | CCCCTCTGTCCAGCGC | 53 | 473428 | 90 | 1669 | 1684 | kkkddddkdddddeee |
| 14612 | 14627 | CCCCTCTGTCCAGCGC | 53 | 473577 | 93 | 1669 | 1684 | kdkdkdddddddddee |
| 14613 | 14626 | CCCTCTGTCCAGCG | 54 | 472976 | 97 | 1670 | 1683 | kkddddddddddkk |
| 14709 | 14722 | AGGCCAGCAGATCA | 55 | 472983 | 94 | 1766 | 1779 | kkddddddddddkk |
| 14714 | 14727 | GCCTGAGGCCAGCA | 56 | 472984 | 90 | 1771 | 1784 | kkddddddddddkk |
| 15097 | 15112 | ATGGAGTCAGCATCGG | 57 | 473135 | 97 | 2154 | 2169 | kkkddddddddddkkk |
| 15098 | 15111 | TGGAGTCAGCATCG | 58 | 472986 | 95 | 2155 | 2168 | kkddddddddddkk |
| 15128 | 15143 | GCTAAACAACCGCCTT | 59 | 473137 | 95 | 2185 | 2200 | kkkddddddddddkkk |
| 15128 | 15143 | GCTAAACAACCGCCTT | 59 | 473286 | 95 | 2185 | 2200 | kkkddddddddddeee |
| 15128 | 15143 | GCTAAACAACCGCCTT | 59 | 473440 | 88 | 2185 | 2200 | kkkddddkdddddeee |
| 15128 | 15143 | GCTAAACAACCGCCTT | 59 | 473589 | 97 | 2185 | 2200 | kdkdkdddddddddee |
| 15129 | 15142 | CTAAACAACCGCCT | 60 | 472988 | 85 | 2186 | 2199 | kkddddddddddkk |
| 15164 | 15179 | TGAAGATGATAATGGA | 61 | 473140 | 96 | 2221 | 2236 | kkkddddddddddkkk |
| 15165 | 15178 | GAAGATGATAATGG | 62 | 472991 | 90 | 2222 | 2235 | kkddddddddddkk |
| 15181 | 15196 | TTCTGAATTGTCTGAA | 63 | 473444 | 94 | 2238 | 2253 | kkkddddkdddddeee |
| 15188 | 15203 | GTGATGCTTCTGAATT | 64 | 473142 | 96 | 2245 | 2260 | kkkddddddddddkkk |
| 15188 | 15203 | GTGATGCTTCTGAATT | 64 | 473291 | 95 | 2245 | 2260 | kkkddddddddddeee |
| 15188 | 15203 | GTGATGCTTCTGAATT | 64 | 473594 | 95 | 2245 | 2260 | kdkdkdddddddddee |
| 15190 | 15205 | TGGTGATGCTTCTGAA | 65 | 473143 | 97 | 2247 | 2262 | kkkddddddddddkkk |
| 15190 | 15205 | TGGTGATGCTTCTGAA | 65 | 473292 | 96 | 2247 | 2262 | kkkddddddddddeee |
| 15190 | 15205 | TGGTGATGCTTCTGAA | 65 | 473446 | 96 | 2247 | 2262 | kkkddddkdddddeee |
| 15190 | 15205 | TGGTGATGCTTCTGAA | 65 | 473595 | 84 | 2247 | 2262 | kdkdkdddddddddee |
| 15191 | 15204 | GGTGATGCTTCTGA | 66 | 472994 | 96 | 2248 | 2261 | kkddddddddddkk |
| 15191 | 15206 | ATGGTGATGCTTCTGA | 67 | 473144 | 98 | 2248 | 2263 | kkkddddddddddkkk |
| 15191 | 15206 | ATGGTGATGCTTCTGA | 67 | 473293 | 96 | 2248 | 2263 | kkkddddddddddeee |
| 15192 | 15205 | TGGTGATGCTTCTG | 68 | 472995 | 96 | 2249 | 2262 | kkddddddddddkk |
| 15194 | 15209 | TGCATGGTGATGCTTC | 69 | 473294 | 91 | 2251 | 2266 | kkkddddddddddeee |
| 15194 | 15209 | TGCATGGTGATGCTTC | 69 | 473597 | 94 | 2251 | 2266 | kdkdkdddddddddee |
| 15195 | 15208 | GCATGGTGATGCTT | 70 | 472996 | 94 | 2252 | 2265 | kkddddddddddkk |
| 15195 | 15210 | ATGCATGGTGATGCTT | 71 | 473295 | 92 | 2252 | 2267 | kkkddddddddddeee |
| 15262 | 15277 | CTGTGCAGCCCGGCAC | 73 | 473296 | 98 | 2319 | 2334 | kkkddddddddddeee |
| 15262 | 15277 | CTGTGCAGCCCGGCAC | 73 | 473450 | 95 | 2319 | 2334 | kkkddddkdddddeee |
| 15263 | 15276 | TGTGCAGCCCGGCA | 74 | 472998 | 97 | 2320 | 2333 | kkddddddddddkk |

### Example 2

### Modified Antisense Oligonucleotides Comprising cEt, MOE, and 3′-Fluoro-HNA Modifications Targeting Human Coagulation Factor VII

Additional antisense oligonucleotides were designed targeting a Factor VII nucleic acid and were tested for their effects on Factor VII mRNA in vitro. ISIS 407939 was also tested.

The newly designed modified antisense oligonucleotides and their motifs are described in Table 2. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, ‘e’ indicates a 2′-O-methoxyethyl nucleoside, and ‘g’ indicates a 3′-fluoro-HNA nucleoside. The ‘Sequence’ column provides the nucleobase sequence for each SEQ ID NO.

Each oligonucleotide listed in Table 2 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted human gene sequence. ‘n/a.’ indicates that the antisense oligonucleotide is not 100% complementary with that particular gene sequence. Oligonucleotides having multiple start and stop sites target a region that is repeated within a Factor VII sequence (e.g., within SEQ ID NO: 1).

Activity of the newly designed oligonucleotides was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (descried in Example 1) was used to measure mRNA levels. Factor VII mRNA levels were adjusted according to total RNA content, as measured by RIBOGREEN®. Results are presented as percent inhibition of Factor VII expression, relative to untreated control cells. A total of 765 oligonucleotides were tested. Only those oligonucleotides which were selected for further studies are shown in Table 2. All but one of the newly designed antisense oligonucleotides provided in Table 2 achieved greater than 30% inhibition and, therefore, are more active than ISIS 407939.

TABLE 2
Percent inhibition of human Factor VII mRNA levels by modified
antisense oligonucleotides targeted to SEQ ID NO: 1 and SEQ ID NO: 2

| Start | Stop | | | | | Start | Stop | |
|---|---|---|---|---|---|---|---|---|
| Site on | Site on | | SEQ | | | Site on | Site on | |
| SEQ ID | SEQ ID | | ID | ISIS | % | SEQ ID | SEQ ID | |
| NO: 1 | NO: 1 | Sequence | NO | No | inhibition | NO: 2 | NO: 2 | Sugar Chemistry |
| 15255 | 15274 | TGCAGCCCGGCACCCA | 72 | 407939 | 30 | 2312 | 2331 | eeeeeddddddddddeeeee |
| | | GCGA | | | | | | |
| 1384 | 1397 | GCCTGGATGCTGGT | 25 | 482838 | 81 | n/a | n/a | ggddddddddddgg |
| 4871 | 4886 | CTGCAGGAGCGGCCTA | 28 | 482992 | 93 | n/a | n/a | gggddddddddddggg |
| 6642 | 6657 | GAGGTGACCCGTGAGC | 30 | 482996 | 97 | n/a | n/a | gggddddddddddggg |
| 1382 | 1397 | GCCTGGATGCTGGTTT | 23 | 483284 | 82 | n/a | n/a | gdgdgdddddddddee |
| 4717 | 4732 | AGGAGGCTGAGGATGC | 27 | 483289 | 70 | n/a | n/a | gdgdgdddddddddee |
| 4871 | 4886 | CTGCAGGAGCGGCCTA | 28 | 483290 | 80 | n/a | n/a | gdgdgdddddddddee |
| 6642 | 6657 | GAGGTGACCCGTGAGC | 30 | 483294 | 69 | n/a | n/a | gdgdgdddddddddee |
| 1382 | 1397 | GCCTGGATGCTGGTTT | 23 | 483438 | 81 | n/a | n/a | ggddddddddddeeee |
| 4871 | 4886 | CTGCAGGAGCGGCCTA | 28 | 483444 | 84 | n/a | n/a | ggddddddddddeeee |
| 6642 | 6657 | GAGGTGACCCGTGAGC | 30 | 483448 | 77 | n/a | n/a | ggddddddddddeeee |
| 6643 | 6656 | AGGTGACCCGTGAG | 31 | 482847 | 79 | n/a | n/a | ggddddddddddgg |
| 6765 | 6778 | | | | | | | |
| 6887 | 6900 | | | | | | | |
| 6953 | 6966 | | | | | | | |
| 7071 | 7084 | | | | | | | |
| 7189 | 7202 | | | | | | | |
| 7243 | 7256 | | | | | | | |
| 11017 | 11030 | CTGCTCACAGCCGC | 32 | 482747 | 85 | 452 | 465 | ggddddddddddgg |
| 12634 | 12647 | GCTGCTCAGACCTG | 40 | 482873 | 81 | n/a | n/a | ggddddddddddgg |
| 12635 | 12648 | AGCTGCTCAGACCT | 75 | 482874 | 82 | n/a | n/a | ggddddddddddgg |
| 12636 | 12649 | AAGCTGCTCAGACC | 76 | 482875 | 82 | n/a | n/a | ggddddddddddgg |
| 11016 | 11031 | ACTGCTCACAGCCGCC | 77 | 482896 | 95 | 451 | 466 | gggddddddddddggg |
| 12629 | 12644 | GCTCAGACCTGGCTCT | 38 | 483019 | 89 | n/a | n/a | gggddddddddddggg |
| 11016 | 11031 | ACTGCTCACAGCCGCC | 77 | 483045 | 92 | 451 | 466 | gdgddddddddddgdg |
| 11016 | 11031 | ACTGCTCACAGCCGCC | 77 | 483194 | 64 | 451 | 466 | gdgdgdddddddddee |
| 12629 | 12644 | GCTCAGACCTGGCTCT | 38 | 483317 | 79 | n/a | n/a | gdgdgdddddddddee |
| 11016 | 11031 | ACTGCTCACAGCCGCC | 57 | 483343 | 75 | 451 | 466 | ggddddddddddeeee |
| 12629 | 12644 | GCTCAGACCTGGCTCT | 38 | 483471 | 76 | n/a | n/a | ggddddddddddeeee |
| 12941 | 12956 | ACCCAGCACCGCGGTC | 78 | 483478 | 20 | n/a | n/a | ggddddddddddeeee |
| 12978 | 12993 | | | | | | | |
| 13015 | 13030 | | | | | | | |
| 13052 | 13067 | | | | | | | |
| 13089 | 13104 | | | | | | | |
| 14093 | 14106 | GAGTAGCCGGCACA | 79 | 482784 | 83 | 1150 | 1163 | ggddddddddddgg |
| 14613 | 14626 | CCCTCTGTCCAGCG | 54 | 482794 | 91 | 1670 | 1683 | ggddddddddddgg |
| 15098 | 15111 | TGGAGTCAGCATCG | 58 | 482804 | 80 | 2155 | 2168 | ggddddddddddgg |
| 15191 | 15204 | GGTGATGCTTCTGA | 66 | 482812 | 81 | 2248 | 2261 | ggddddddddddgg |
| 15192 | 15205 | TGGTGATGCTTCTG | 68 | 482813 | 92 | 2249 | 2262 | ggddddddddddgg |
| 15195 | 15208 | GCATGGTGATGCTT | 70 | 482814 | 94 | 2252 | 2265 | ggddddddddddgg |
| 15196 | 15209 | TGCATGGTGATGCT | 80 | 482815 | 81 | 2253 | 2266 | ggddddddddddgg |
| 15263 | 15276 | TGTGCAGCCCGGCA | 74 | 482816 | 71 | 2320 | 2333 | ggddddddddddgg |
| 13741 | 13756 | GTGCTCGCTGAGGTCG | 44 | 482916 | 90 | 798 | 813 | gggddddddddddggg |
| 14079 | 14094 | CAGAACATGTACTCCG | 48 | 482932 | 89 | 1136 | 1151 | gggddddddddddggg |
| 15097 | 15112 | ATGGAGTCAGCATCGG | 57 | 482953 | 93 | 2154 | 2169 | gggddddddddddggg |
| 15191 | 15206 | ATGGTGATGCTTCTGA | 67 | 482962 | 97 | 2248 | 2263 | gggddddddddddggg |
| 15194 | 15209 | TGCATGGTGATGCTTC | 69 | 482963 | 96 | 2251 | 2266 | gggddddddddddggg |
| 15262 | 15277 | CTGTGCAGCCCGGCAC | 73 | 482965 | 89 | 2319 | 2334 | gggddddddddddggg |
| 13741 | 13756 | GTGCTCGCTGAGGTCG | 44 | 483065 | 69 | 798 | 813 | gdgddddddddddgdg |
| 14612 | 14627 | CCCCTCTGTCCAGCGC | 53 | 483092 | 89 | 1669 | 1684 | gdgddddddddddgdg |
| 14612 | 14627 | CCCCTCTGTCCAGCGC | 53 | 483241 | 79 | 1669 | 1684 | gdgdgdddddddddee |
| 15128 | 15143 | GCTAAACAACCGCCTT | 59 | 483253 | 76 | 2185 | 2200 | gdgdgdddddddddee |
| 15188 | 15203 | GTGATGCTTCTGAATT | 64 | 483258 | 70 | 2245 | 2260 | gdgdgdddddddddee |
| 15191 | 15206 | ATGGTGATGCTTCTGA | 67 | 483260 | 62 | 2248 | 2263 | gdgdgdddddddddee |
| 15194 | 15209 | TGCATGGTGATGCTTC | 69 | 483261 | 76 | 2251 | 2266 | gdgdgdddddddddee |
| 15195 | 15210 | ATGCATGGTGATGCTT | 71 | 483262 | 75 | 2252 | 2267 | gdgdgdddddddddee |
| 15262 | 15277 | CTGTGCAGCCCGGCAC | 73 | 483263 | 73 | 2319 | 2334 | gdgdgdddddddddee |
| 13760 | 13775 | GGCTCTGCTCATCCCC | 81 | 483364 | 78 | 817 | 832 | ggddddddddddeeee |
| 14612 | 14627 | CCCCTCTGTCCAGCGC | 53 | 483395 | 86 | 1669 | 1684 | ggddddddddddeeee |
| 15190 | 15205 | TGGTGATGCTTCTGAA | 65 | 483413 | 83 | 2247 | 2262 | ggddddddddddeeee |
| 15191 | 15206 | ATGGTGATGCTTCTGA | 67 | 483414 | 76 | 2248 | 2263 | ggddddddddddeeee |
| 15194 | 15209 | TGCATGGTGATGCTTC | 69 | 483415 | 85 | 2251 | 2266 | ggddddddddddeeee |
| 15195 | 15210 | ATGCATGGTGATGCTT | 71 | 483416 | 77 | 2252 | 2267 | ggddddddddddeeee |
| 15262 | 15277 | CTGTGCAGCCCGGCAC | 73 | 483417 | 83 | 2319 | 2334 | ggddddddddddeeee |

### Example 3

### Modified Oligonucleotides Comprising MOE, and/or cEt Modifications Targeting Human Coagulation Factor VII

Additional antisense oligonucleotides were designed targeting a Factor VII nucleic acid and were tested for their effects on Factor VII mRNA in vitro. Also tested were ISIS 403052, ISIS 407594, ISIS 407606, ISIS 407939, and ISIS 416438, which are 5-10-5 MOE gapmers described in an earlier publication (WO 2009/061851).

The newly designed modified antisense oligonucleotides and their motifs are described in Table 3. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO.

Each oligonucleotide listed in Table 3 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted human gene sequence. ‘n/a.’ indicates that the antisense oligonucleotide is not 100% complementary with that particular gene sequence.

Activity of the newly designed gapmers was compared to ISIS 403052, ISIS 407594, ISIS 407606, ISIS 407939, and ISIS 416438. Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells. A total of 380 oligonucleotides were tested. Only those oligonucleotides which were selected for further studies are shown in Table 3. Each of the newly designed antisense oligonucleotides provided in Table 3 achieved greater than 64% inhibition and, therefore, are more active than each of ISIS 403052, ISIS 407594, ISIS 407606, ISIS 407939, and ISIS 416438.

TABLE 3
Percent inhibition of human Factor VII mRNA levels by modified
antisense oligonucleotides targeted to SEQ ID NO: 1 and SEQ ID NO: 2

| Start | Stop | | | | | Start | Stop | |
|---|---|---|---|---|---|---|---|---|
| Site on | Site on | | SEQ | | | Site on | Site on | |
| SEQ ID | SEQ ID | | ID | | % | SEQ ID | SEQ ID | |
| NO: 1 | NO: 1 | Sequence | NO | ISIS No | inhibition | NO: 2 | NO: 2 | Sugar Chemistry |
| n/a | n/a | GGCACACTGGTCCCCATCAC | 82 | 403052 | 64 | 299 | 318 | eeeeeddddddddddeeeee |
| 1208 | 1227 | CCTGCAGCCAGGCAGCCCTG | 83 | 407594 | 40 | n/a | n/a | eeeeeddddddddddeeeee |
| 9204 | 9223 | CTGGTCCTTGCAGGAGCCCC | 84 | 407606 | 39 | 338 | 357 | eeeeeddddddddddeeeee |
| 15255 | 15274 | TGCAGCCCGGCACCCAGCGA | 72 | 407939 | 57 | 2312 | 2331 | eeeeeddddddddddeeeee |
| 9194 | 9213 | CAGGAGCCCCCATTCTGGCA | 85 | 416438 | 62 | 328 | 347 | eeeeeddddddddddeeeee |
| 11016 | 11031 | ACTGCTCACAGCCGCC | 77 | 484487 | 91 | 451 | 466 | kdkddddddddddkdk |
| 14612 | 14627 | CCCCTCTGTCCAGCGC | 53 | 484539 | 92 | 1669 | 1684 | kdkddddddddddkdk |
| 14708 | 14723 | GAGGCCAGCAGATCAC | 86 | 484546 | 92 | 1765 | 1780 | kdkddddddddddkdk |
| 14713 | 14728 | AGCCTGAGGCCAGCAG | 87 | 484547 | 89 | 1770 | 1785 | kdkddddddddddkdk |
| 15097 | 15112 | ATGGAGTCAGCATCGG | 57 | 484549 | 91 | 2154 | 2169 | kdkddddddddddkdk |
| 15190 | 15205 | TGGTGATGCTTCTGAA | 65 | 484557 | 92 | 2247 | 2262 | kdkddddddddddkdk |
| 15191 | 15206 | ATGGTGATGCTTCTGA | 67 | 484558 | 94 | 2248 | 2263 | kdkddddddddddkdk |
| 15194 | 15209 | TGCATGGTGATGCTTC | 69 | 484559 | 90 | 2251 | 2266 | kdkddddddddddkdk |
| 1382 | 1397 | GCCTGGATGCTGGTTT | 23 | 484582 | 88 | n/a | n/a | kdkddddddddddkdk |
| 9170 | 9185 | AGGCACACTGGTCCCC | 88 | 484632 | 90 | 304 | 319 | kkddddddddddeeee |
| 11016 | 11031 | ACTGCTCACAGCCGCC | 77 | 484641 | 91 | 451 | 466 | kkddddddddddeeee |
| 14092 | 14107 | CGAGTAGCCGGCACAG | 49 | 484679 | 90 | 1149 | 1164 | kkddddddddddeeee |
| 14612 | 14627 | CCCCTCTGTCCAGCGC | 53 | 484693 | 93 | 1669 | 1684 | kkddddddddddeeee |
| 15190 | 15205 | TGGTGATGCTTCTGAA | 65 | 484711 | 92 | 2247 | 2262 | kkddddddddddeeee |
| 15191 | 15206 | ATGGTGATGCTTCTGA | 67 | 484712 | 92 | 2248 | 2263 | kkddddddddddeeee |
| 15194 | 15209 | TGCATGGTGATGCTTC | 69 | 484713 | 85 | 2251 | 2266 | kkddddddddddeeee |
| 15195 | 15210 | ATGCATGGTGATGCTT | 71 | 484714 | 83 | 2252 | 2267 | kkddddddddddeeee |
| 15262 | 15277 | CTGTGCAGCCCGGCAC | 73 | 484715 | 93 | 2319 | 2334 | kkddddddddddeeee |
| 1382 | 1397 | GCCTGGATGCTGGTTT | 23 | 484736 | 89 | n/a | n/a | kkddddddddddeeee |
| 4871 | 4886 | CTGCAGGAGCGGCCTA | 28 | 484742 | 93 | n/a | n/a | kkddddddddddeeee |
| 6642 | 6657 | GAGGTGACCCGTGAGC | 30 | 484746 | 88 | n/a | n/a | kkddddddddddeeee |
| 12631 | 12646 | CTGCTCAGACCTGGCT | 89 | 484771 | 89 | n/a | n/a | kkddddddddddeeee |

### Example 4

### Modified Antisense Oligonucleotides Comprising MOE Modifications Targeting Human Coagulation Factor VII

Additional antisense oligonucleotides were designed targeting a Factor VII nucleic acid and were tested for their effects on Factor VII mRNA in vitro. Also tested were ISIS 403094, ISIS 407641, ISIS 407643, ISIS 407662, ISIS 407900, ISIS 407910, ISIS 407935, ISIS 407936, ISIS 407939, ISIS 416446, ISIS 416449, ISIS 416455, ISIS 416472, ISIS 416477, ISIS 416507, ISIS 416508, ISIS 422086, ISIS 422087, ISIS 422140, and ISIS 422142, which are 5-10-5 MOE gapmers targeting human Factor VII and are described in an earlier publication (WO 2009/061851).

The newly designed modified antisense oligonucleotides in Table 4 were designed as 5-10-5 MOE gapmers. The 5-10-5 MOE gapmers are 20 nucleosides in length, wherein the central gap segment comprises ten 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each oligonucleotide are phosphorothioate linkages. All cytosine residues throughout each oligonucleotide are 5-methylcytosines.

Each oligonucleotide listed in Table 4 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. Each oligonucleotide listed in Table 5 is targeted to human Factor VII gene sequence DB184141.1, designated herein as SEQ ID NO: 3. “Start site” indicates the 5′-most nucleoside to which the oligonucleotide is targeted in the human gene sequence. “Stop site” indicates the 3′-most nucleoside to which the oligonucleotide is targeted human gene sequence. ‘n/a’ indicates that the antisense oligonucleotide is not 100% complementary with that particular gene sequence.

Activity of the newly designed oligonucleotides was compared to ISIS 403094, ISIS 407641, ISIS 407643, ISIS 407662, ISIS 407900, ISIS 407910, ISIS 407935, ISIS 407936, ISIS 407939, ISIS 416446, ISIS 416449, ISIS 416455, ISIS 416472, ISIS 416477, ISIS 416507, ISIS 416508, ISIS 422086, ISIS 422087, ISIS 422140, and ISIS 422142. Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells. A total of 916 oligonucleotides were tested. Only those oligonucleotides which were selected for further studies are shown in Tables 4 and 5.

TABLE 4
Percent inhibition of human Factor VII mRNA levels by modified
antisense oligonucleotides targeted to SEQ ID NO: 1 and SEQ ID NO: 2

| Start | Stop | | | | Start | Stop | |
|---|---|---|---|---|---|---|---|
| Site on | Site on | | | | Site on | Site on | SEQ |
| SEQ ID | SEQ ID | | | % | SEQ ID | SEQ ID | ID |
| NO: 1 | NO: 1 | Sequence | ISIS No | inhibition | NO: 2 | NO: 2 | NO |
| 14240 | 14259 | ATGTACTGGGAGACCCTGGT | 403094 | 60 | 1297 | 1316 | 110 |
| 14707 | 14726 | CCTGAGGCCAGCAGATCACG | 407641 | 64 | 1764 | 1783 | 112 |
| 15098 | 15117 | CACACATGGAGTCAGCATCG | 407643 | 78 | 2155 | 2174 | 114 |
| 12141 | 12160 | CCCCACAATTCGGCCTTGGG | 407900 | 66 | 611 | 630 | 105 |
| 14104 | 14123 | AGTCCTTGCTGCCATCCGAG | 407910 | 25 | 1161 | 1180 | 108 |
| 15191 | 15210 | ATGCATGGTGATGCTTCTGA | 407935 | 91 | 2248 | 2267 | 120 |
| 15204 | 15223 | GGCATTCGCCACCATGCATG | 407936 | 80 | 2261 | 2280 | 122 |
| 15255 | 15274 | TGCAGCCCGGCACCCAGCGA | 407939 | 67 | 2312 | 2331 | 72 |
| 11024 | 11043 | GGTCACTGCAGTACTGCTCA | 416446 | 73 | 459 | 478 | 103 |
| 12094 | 12113 | TAGGTATTTTTCCACATGGA | 416449 | 33 | 564 | 583 | 104 |
| 13760 | 13779 | CGCCGGCTCTGCTCATCCCC | 416455 | 42 | 817 | 836 | 107 |
| 14348 | 14367 | CAGCCTTGGCTTTCTCTCCA | 416472 | 78 | 1405 | 1424 | 111 |
| 14710 | 14729 | CAGCCTGAGGCCAGCAGATC | 416477 | 25 | 1767 | 1786 | 113 |
| 4847 | 4866 | GGTTACTGAGCGCGGAAGAA | 416507 | 73 | n/a | n/a | 97 |
| 4873 | 4892 | CGAGTTCTGCAGGAGCGGCC | 416508 | 75 | n/a | n/a | 100 |
| 15190 | 15209 | TGCATGGTGATGCTTCTGAA | 422086 | 90 | 2247 | 2266 | 119 |
| 15192 | 15211 | CATGCATGGTGATGCTTCTG | 422087 | 89 | 2249 | 2268 | 121 |
| 4870 | 4889 | GTTCTGCAGGAGCGGCCTAA | 422140 | 59 | n/a | n/a | 98 |
| 4872 | 4891 | GAGTTCTGCAGGAGCGGCCT | 422142 | 73 | n/a | n/a | 99 |
| 1383 | 1402 | CCTGTGCCTGGATGCTGGTT | 490275 | 35 | n/a | n/a | 90 |
| 1385 | 1404 | CTCCTGTGCCTGGATGCTGG | 490277 | 73 | n/a | n/a | 91 |
| 1386 | 1405 | CCTCCTGTGCCTGGATGCTG | 490278 | 78 | n/a | n/a | 92 |
| 1387 | 1406 | CCCTCCTGTGCCTGGATGCT | 490279 | 66 | n/a | n/a | 93 |
| 2228 | 2247 | GGCAGTCCCTGCTCACCTCT | 490323 | 65 | n/a | n/a | 94 |
| 2487 | 2506 | GCATCAGAAAAGCTCTCAAG | 490368 | 78 | n/a | n/a | 95 |
| 4725 | 4744 | GTCTGGTTTGGAAGGAGGCT | 490396 | 76 | n/a | n/a | 96 |
| 4939 | 4958 | GGAGGGACGACCTTTGCTGG | 490424 | 57 | n/a | n/a | 101 |
| 10676 | 10695 | GACCACTCTTCCGAGCAGCT | 490803 | 70 | n/a | n/a | 102 |
| 12801 | 12820 | CTGAGCTCCATTCACCAACA | 490103 | 87 | 674 | 693 | 106 |
| 14232 | 14251 | GGAGACCCTGGTGTACACCC | 490149 | 82 | 1289 | 1308 | 109 |
| 15129 | 15148 | TGAGAGCTAAACAACCGCCT | 490196 | 81 | 2186 | 2205 | 115 |
| 15130 | 15149 | GTGAGAGCTAAACAACCGCC | 490197 | 85 | 2187 | 2206 | 116 |
| 15183 | 15202 | TGATGCTTCTGAATTGTCTG | 490208 | 89 | 2240 | 2259 | 117 |
| 15184 | 15203 | GTGATGCTTCTGAATTGTCT | 490209 | 81 | 2241 | 2260 | 118 |

TABLE 5
Percent inhibition of human Factor VII mRNA levels
by modified antisense oligonucleotides targeted to
SEQ ID NO: 3

| Start Site | Stop Site | | | | SEQ |
|---|---|---|---|---|---|
| on SEQ ID | on SEQ ID | | ISIS | % | ID |
| NO: 3 | NO: 3 | Sequence | No | inhibition | NO |
| 50 | 69 | TCCTGCAGCCAGGCAGCCCT | 407662 | 76 | 123 |
| 444 | 463 | GGTCACTGCAGTACTGCTCA | 416446 | 73 | 103 |

### Example 5

### Modified Antisense Oligonucleotides Comprising cEt Modifications Targeting Human Coagulation Factor VII

Additional antisense oligonucleotides were designed targeting a Factor VII nucleic acid and were tested for their effects on Factor VII mRNA in vitro. Also tested was ISIS 407939, a 5-10-5 MOE gapmer targeting human Factor VII, which was described in an earlier publication (WO 2009/061851). ISIS 457851, ISIS 472925, ISIS 472926, ISIS 472935, ISIS 472942, ISIS 472958, ISIS 472959, ISIS 472970, ISIS 472976, ISIS 472983, ISIS 472984, ISIS 472988, ISIS 472991, ISIS 472994, ISIS 472995, ISIS 472996, ISIS 472998, and ISIS 473020, described in the Examples above were also included in the screen.

The newly designed modified antisense oligonucleotides in Table 6 were designed as 2-10-2 cEt gapmers. The 2-10-2 cEt gapmers are 14 nucleosides in length, wherein the central gap segment comprises ten 2′-deoxynucleosides and is flanked by wing segments on the 5′ direction and the 3′ direction comprising two nucleosides each. Each nucleoside in the 5′ wing segment and each nucleoside in the 3′ wing segment has a cEt modification. The internucleoside linkages throughout each gapmer are phosphorothioate linkages. All cytosine residues throughout each olignucleotide are 5-methylcytosines.

Each oligonucleotide listed in Table 6 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted human gene sequence. ‘n/a.’ indicates that the antisense oligonucleotide is not 100% complementary with that particular gene sequence.

Activity of the newly designed oligonucleotides was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells. A total of 614 oligonucleotides were tested. Only those oligonucleotides which were selected for further studies are shown in Table 6. Many of the newly designed antisense oligonucleotides provided in Table 6 achieved greater than 72% inhibition and, therefore, are more active than ISIS 407939.

TABLE 6
Percent inhibition of human Factor VII mRNA levels by modified
antisense oligonucleotides targeted to SEQ ID NO: 1 and SEQ ID NO: 2

| Start | Stop | | | | | Start | Stop | |
|---|---|---|---|---|---|---|---|---|
| Site on | Site on | | | | | Site on | Site on | |
| SEQ ID | SEQ ID | | | % | | SEQ ID | SEQ ID | SEQ |
| NO: 1 | NO: 1 | Sequence | ISIS No | inhibition | Motif | NO: 2 | NO: 2 | ID NO |
| 15255 | 15274 | TGCAGCCCGGCACCCAGCGA | 407939 | 72 | 5-10-5 | 2312 | 2331 | 72 |
| 1384 | 1397 | GCCTGGATGCTGGT | 473020 | 90 | 2-10-2 | n/a | n/a | 25 |
| 1386 | 1399 | GTGCCTGGATGCTG | 492465 | 83 | 2-10-2 | n/a | n/a | 124 |
| 1388 | 1401 | CTGTGCCTGGATGC | 492467 | 74 | 2-10-2 | n/a | n/a | 125 |
| 2237 | 2250 | AGTGGCAGTCCCTG | 492492 | 84 | 2-10-2 | n/a | n/a | 126 |
| 2239 | 2252 | CCAGTGGCAGTCCC | 492494 | 91 | 2-10-2 | n/a | n/a | 127 |
| 2274 | 2287 | GGTGATGTTGGCCC | 492503 | 89 | 2-10-2 | n/a | n/a | 128 |
| 2482 | 2495 | GCTCTCAAGAACTG | 492530 | 91 | 2-10-2 | n/a | n/a | 129 |
| 2493 | 2506 | GCATCAGAAAAGCT | 492534 | 91 | 2-10-2 | n/a | n/a | 130 |
| 2499 | 2512 | AGATTTGCATCAGA | 492536 | 90 | 2-10-2 | n/a | n/a | 131 |
| 4711 | 4724 | GAGGATGCAGGCGG | 492541 | 84 | 2-10-2 | n/a | n/a | 132 |
| 4730 | 4743 | TCTGGTTTGGAAGG | 492545 | 89 | 2-10-2 | n/a | n/a | 133 |
| 4852 | 4865 | GTTACTGAGCGCGG | 492566 | 90 | 2-10-2 | n/a | n/a | 134 |
| 4874 | 4887 | TCTGCAGGAGCGGC | 492571 | 82 | 2-10-2 | n/a | n/a | 135 |
| 4875 | 4888 | TTCTGCAGGAGCGG | 492572 | 89 | 2-10-2 | n/a | n/a | 136 |
| 4876 | 4889 | GTTCTGCAGGAGCG | 492573 | 90 | 2-10-2 | n/a | n/a | 137 |
| 4877 | 4890 | AGTTCTGCAGGAGC | 492574 | 92 | 2-10-2 | n/a | n/a | 138 |
| 4878 | 4891 | GAGTTCTGCAGGAG | 492575 | 88 | 2-10-2 | n/a | n/a | 139 |
| 4923 | 4936 | GGACGAGGCCTCAG | 492593 | 83 | 2-10-2 | n/a | n/a | 140 |
| 5133 | 5146 | GCTGTGGGCACCAC | 492617 | 91 | 2-10-2 | n/a | n/a | 141 |
| 5134 | 5147 | AGCTGTGGGCACCA | 492618 | 92 | 2-10-2 | n/a | n/a | 142 |
| 5135 | 5148 | GAGCTGTGGGCACC | 492619 | 90 | 2-10-2 | n/a | n/a | 143 |
| 5199 | 5212 | GCTCCGAGCAGGCC | 492621 | 75 | 2-10-2 | n/a | n/a | 144 |
| 6077 | 6090 | CGGCCGCAGCTCCT | 492104 | 89 | 2-10-2 | 182 | 195 | 145 |
| 6078 | 6091 | CCGGCCGCAGCTCC | 492105 | 86 | 2-10-2 | 183 | 196 | 146 |
| 10988 | 11001 | AGATCAGCTGGTCA | 492189 | 88 | 2-10-2 | 423 | 436 | 147 |
| 11015 | 11028 | GCTCACAGCCGCCG | 492194 | 92 | 2-10-2 | 450 | 463 | 148 |
| 11016 | 11029 | TGCTCACAGCCGCC | 492195 | 90 | 2-10-2 | 451 | 464 | 149 |
| 11017 | 11030 | CTGCTCACAGCCGC | 472925 | 87 | 2-10-2 | 452 | 465 | 32 |
| 11018 | 11031 | ACTGCTCACAGCCG | 492196 | 91 | 2-10-2 | 453 | 466 | 150 |
| 11023 | 11036 | GCAGTACTGCTCAC | 472926 | 88 | 2-10-2 | 458 | 471 | 33 |
| 11030 | 11043 | GGTCACTGCAGTAC | 492205 | 92 | 2-10-2 | 465 | 478 | 151 |
| 12083 | 12096 | GGATATTCAACTGT | 492215 | 77 | 2-10-2 | 553 | 566 | 152 |
| 12099 | 12112 | AGGTATTTTTCCAC | 492221 | 79 | 2-10-2 | 569 | 582 | 153 |
| 12128 | 12141 | GGTTTGCTGGCATT | 472935 | 82 | 2-10-2 | 598 | 611 | 36 |
| 12796 | 12809 | TCACCAACAACAGG | 492234 | 86 | 2-10-2 | 669 | 682 | 154 |
| 12864 | 12877 | GAAACAGTGGGCCG | 472942 | 85 | 2-10-2 | 737 | 750 | 43 |
| 13778 | 13791 | ACCTGCGCCACCCG | 492276 | 75 | 2-10-2 | 835 | 848 | 155 |
| 13779 | 13792 | GACCTGCGCCACCC | 492277 | 75 | 2-10-2 | 836 | 849 | 156 |
| 13893 | 13906 | CCGTTCGGGCAGGC | 492306 | 85 | 2-10-2 | 950 | 963 | 157 |
| 14018 | 14031 | TGGGTCATCAGCCG | 492317 | 93 | 2-10-2 | 1075 | 1088 | 158 |
| 14019 | 14032 | CTGGGTCATCAGCC | 472958 | 92 | 2-10-2 | 1076 | 1089 | 46 |
| 14022 | 14035 | GTCCTGGGTCATCA | 472959 | 88 | 2-10-2 | 1079 | 1092 | 47 |
| 14077 | 14090 | ACATGTACTCCGTG | 492329 | 88 | 2-10-2 | 1134 | 1147 | 159 |
| 14079 | 14092 | GAACATGTACTCCG | 492331 | 95 | 2-10-2 | 1136 | 1149 | 160 |
| 14094 | 14107 | CGAGTAGCCGGCAC | 492333 | 85 | 2-10-2 | 1151 | 1164 | 161 |
| 14095 | 14108 | CCGAGTAGCCGGCA | 492334 | 88 | 2-10-2 | 1152 | 1165 | 162 |
| 14232 | 14245 | CCTGGTGTACACCC | 457851 | 89 | 2-10-2 | 1289 | 1302 | 51 |
| 14244 | 14257 | GTACTGGGAGACCC | 472970 | 92 | 2-10-2 | 1301 | 1314 | 52 |
| 14265 | 14278 | GAGCTTTTGCAGCC | 492365 | 69 | 2-10-2 | 1322 | 1335 | 163 |
| 14613 | 14626 | CCCTCTGTCCAGCG | 472976 | 94 | 2-10-2 | 1670 | 1683 | 54 |
| 14709 | 14722 | AGGCCAGCAGATCA | 472983 | 76 | 2-10-2 | 1766 | 1779 | 55 |
| 14714 | 14727 | GCCTGAGGCCAGCA | 472984 | 72 | 2-10-2 | 1771 | 1784 | 56 |
| 14741 | 14754 | GTCTCCAGCAATGA | 492377 | 70 | 2-10-2 | 1798 | 1811 | 164 |
| 15101 | 15114 | ACATGGAGTCAGCA | 492380 | 80 | 2-10-2 | 2158 | 2171 | 165 |
| 15105 | 15118 | GCACACATGGAGTC | 492384 | 61 | 2-10-2 | 2162 | 2175 | 166 |
| 15129 | 15142 | CTAAACAACCGCCT | 472988 | 59 | 2-10-2 | 2186 | 2199 | 60 |
| 15130 | 15143 | GCTAAACAACCGCC | 492388 | 70 | 2-10-2 | 2187 | 2200 | 167 |
| 15131 | 15144 | AGCTAAACAACCGC | 492389 | 70 | 2-10-2 | 2188 | 2201 | 168 |
| 15132 | 15145 | GAGCTAAACAACCG | 492390 | 89 | 2-10-2 | 2189 | 2202 | 169 |
| 15133 | 15146 | AGAGCTAAACAACC | 492391 | 80 | 2-10-2 | 2190 | 2203 | 170 |
| 15165 | 15178 | GAAGATGATAATGG | 472991 | 84 | 2-10-2 | 2222 | 2235 | 62 |
| 15184 | 15197 | CTTCTGAATTGTCT | 492398 | 88 | 2-10-2 | 2241 | 2254 | 171 |
| 15185 | 15198 | GCTTCTGAATTGTC | 492399 | 94 | 2-10-2 | 2242 | 2255 | 172 |
| 15187 | 15200 | ATGCTTCTGAATTG | 492401 | 91 | 2-10-2 | 2244 | 2257 | 173 |
| 15190 | 15203 | GTGATGCTTCTGAA | 492403 | 78 | 2-10-2 | 2247 | 2260 | 174 |
| 15191 | 15204 | GGTGATGCTTCTGA | 472994 | 95 | 2-10-2 | 2248 | 2261 | 66 |
| 15192 | 15205 | TGGTGATGCTTCTG | 472995 | 91 | 2-10-2 | 2249 | 2262 | 68 |
| 15193 | 15206 | ATGGTGATGCTTCT | 492404 | 84 | 2-10-2 | 2250 | 2263 | 175 |
| 15194 | 15207 | CATGGTGATGCTTC | 492405 | 87 | 2-10-2 | 2251 | 2264 | 176 |
| 15195 | 15208 | GCATGGTGATGCTT | 472996 | 85 | 2-10-2 | 2252 | 2265 | 70 |
| 15197 | 15210 | ATGCATGGTGATGC | 492406 | 43 | 2-10-2 | 2254 | 2267 | 177 |
| 15263 | 15276 | TGTGCAGCCCGGCA | 472998 | 92 | 2-10-2 | 2320 | 2333 | 74 |
| 15807 | 15820 | GGTGCCCAGGACGG | 492440 | 89 | 2-10-2 | 2864 | 2877 | 178 |

### Example 6

### Modified Antisense Oligonucleotides Comprising cEt Modifications Targeting Human Coagulation Factor VII

Additional antisense oligonucleotides were designed targeting a Factor VII nucleic acid and were tested for their effects on Factor VII mRNA in vitro. Also tested was ISIS 407939, a 5-10-5 MOE gapmer targeting human Factor VII, which was described in an earlier publication (WO 2009/061851). ISIS 472998 and ISIS 473046, described in the Examples above were also included in the screen.

The newly designed modified antisense oligonucleotides in Table 7 were designed as 2-10-2 cEt gapmers. The 2-10-2 cEt gapmers are 14 nucleosides in length, wherein the central gap segment comprises of ten 2′-deoxynucleosides and is flanked by wing segments on the 5′ direction and the 3′ direction comprising two nucleosides each. Each nucleoside in the 5′ wing segment and each nucleoside in the 3′ wing segment has a cEt sugar modification. The internucleoside linkages throughout each oligonucleotide are phosphorothioate linkages. All cytosine residues throughout each oligonucleotide are 5-methylcytosines.

Each oligonucleotide listed in Table 7 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotiode is targeted in the human gene sequence. “Stop site” indicates the 3′-most nucleoside to which the oligonucleotide is targeted human gene sequence. ‘n/a’ indicates that the antisense oligonucleotide is not 100% complementary with that particular gene sequence.

Activity of the newly designed gapmers was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells. A total of 757 oligonucleotides were tested. Only those oligonucleotides which were selected for further studies are shown in Table 7. Each of the newly designed antisense oligonucleotides provided in Table 7 achieved greater than 67% inhibition and, therefore, are more active than 407939.

TABLE 7
Percent inhibition of human Factor VII mRNA levels by modified
antisense oligonucleotides targeted to SEQ ID NO: 1 and SEQ ID NO: 2

| Start | Stop | | | | | Start | Stop | |
|---|---|---|---|---|---|---|---|---|
| Site on | Site on | | | | | Site on | Site on | SEQ |
| SEQ ID | SEQ ID | | | % | | SEQ ID | SEQ ID | ID |
| NO: 1 | NO: 1 | Sequence | ISIS No | inhibition | Motif | NO: 2 | NO: 2 | NO |
| 15255 | 15274 | TGCAGCCCGGCACCCAGCGA | 407939 | 67 | 5-10-5 | 2312 | 2331 | 72 |
| 15263 | 15276 | TGTGCAGCCCGGCA | 472998 | 85 | 2-10-2 | 2320 | 2333 | 74 |
| 11840 | 11853 | ATGGTCAGGGCTGG | 473046 | 79 | 2-10-2 | n/a | n/a | 35 |
| 5513 | 5526 | CGAGGCGCGGCCCC | 492651 | 77 | 2-10-2 | n/a | n/a | 179 |
| 5514 | 5527 | CCGAGGCGCGGCCC | 492652 | 84 | 2-10-2 | n/a | n/a | 180 |
| 5558 | 5571 | GTCTCCGGCGGCCA | 492658 | 87 | 2-10-2 | n/a | n/a | 181 |
| 8608 | 8621 | GCTGTGAGAATACA | 492725 | 74 | 2-10-2 | n/a | n/a | 182 |
| 8644 | 8657 | GAAACTGTTGGCCA | 492730 | 78 | 2-10-2 | n/a | n/a | 183 |
| 8645 | 8658 | AGAAACTGTTGGCC | 492731 | 72 | 2-10-2 | n/a | n/a | 184 |
| 8862 | 8875 | TGGGTGACCACACA | 492784 | 72 | 2-10-2 | n/a | n/a | 185 |
| 9358 | 9371 | GGTTGTGCACCCTG | 492816 | 70 | 2-10-2 | n/a | n/a | 186 |
| 9360 | 9373 | CAGGTTGTGCACCC | 492818 | 73 | 2-10-2 | n/a | n/a | 187 |
| 9599 | 9612 | AGTTTACCAAGCGG | 492877 | 83 | 2-10-2 | n/a | n/a | 188 |
| 9600 | 9613 | AAGTTTACCAAGCG | 492878 | 79 | 2-10-2 | n/a | n/a | 189 |
| 9940 | 9953 | CCTCTGGACACCGG | 492913 | 73 | 2-10-2 | n/a | n/a | 190 |
| 9941 | 9954 | ACCTCTGGACACCG | 492914 | 82 | 2-10-2 | n/a | n/a | 191 |
| 9960 | 9973 | GTGATTGAGCCCTG | 492928 | 76 | 5-10-5 | n/a | n/a | 192 |
| 10069 | 10082 | GGTCTAGCTGACAA | 492938 | 80 | 2-10-2 | n/a | n/a | 193 |
| 10385 | 10398 | GGATGCACACCAGG | 492991 | 91 | 2-10-2 | n/a | n/a | 194 |
| 10386 | 10399 | AGGATGCACACCAG | 492992 | 73 | 2-10-2 | n/a | n/a | 195 |
| 11144 | 11157 | GGTGTCATCTGGGA | 493087 | 81 | 2-10-2 | n/a | n/a | 196 |
| 11283 | 11296 | CTGTCGCTCTGGCC | 493114 | 80 | 2-10-2 | n/a | n/a | 197 |
| 11545 | 11558 | GGAAGTGCAGCCCA | 493178 | 86 | 2-10-2 | n/a | n/a | 198 |
| 11546 | 11559 | TGGAAGTGCAGCCC | 493179 | 69 | 2-10-2 | n/a | n/a | 199 |
| 11703 | 11716 | GTTGTTTTGATCCC | 493182 | 79 | 2-10-2 | n/a | n/a | 200 |
| 11838 | 11851 | GGTCAGGGCTGGTT | 493195 | 71 | 2-10-2 | n/a | n/a | 201 |
| 11847 | 11860 | GGAGACAATGGTCA | 493201 | 86 | 2-10-2 | n/a | n/a | 202 |
| 11848 | 11861 | AGGAGACAATGGTC | 493202 | 76 | 2-10-2 | n/a | n/a | 203 |
| 12406 | 12419 | TCTCTGCACAGGGT | 493255 | 80 | 2-10-2 | n/a | n/a | 204 |
| 12506 | 12519 | GATCCAATGCTCCT | 493291 | 84 | 2-10-2 | n/a | n/a | 205 |
| 12507 | 12520 | TGATCCAATGCTCC | 493292 | 90 | 2-10-2 | n/a | n/a | 206 |
| 12511 | 12524 | GCTTTGATCCAATG | 493296 | 82 | 2-10-2 | n/a | n/a | 207 |
| 12513 | 12526 | TAGCTTTGATCCAA | 493298 | 77 | 2-10-2 | n/a | n/a | 208 |
| 12514 | 12527 | ATAGCTTTGATCCA | 493299 | 76 | 2-10-2 | n/a | n/a | 209 |
| 12521 | 12534 | TCTTCACATAGCTT | 493304 | 77 | 2-10-2 | n/a | n/a | 210 |
| 12554 | 12567 | TCGCTGTGAGATTT | 493312 | 75 | 2-10-2 | n/a | n/a | 211 |
| 12692 | 12705 | GGCATTGCACAATT | 493333 | 76 | 2-10-2 | n/a | n/a | 212 |

### Example 7

### Dose-Dependent Antisense Inhibition of Human Factor VII in Hep3B Cells

Antisense oligonucleotides from the studies above, exhibiting in vitro inhibition of Factor VII mRNA were selected and tested at various doses in Hep3B cells. Also tested was ISIS 407939, a 5-10-5 MOE gapmer, which was described in an earlier publication (WO 2009/061851).

Cells were plated at a density of 20,000 cells per well and transfected using electroporation with 0.67 μM, 2.00 μM, 1.11 μM, and 6.00 μM concentrations of antisense oligonucleotide, as specified in Table 8. After a treatment period of approximately 16 hours, RNA was isolated from the cells and Factor VII mRNA levels were measured by quantitative real-time PCR. Human Factor VII primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells.

The half maximal inhibitory concentration (IC₅₀) of each oligonucleotide is also presented in Table 8. As illustrated in Table 8, Factor VII mRNA levels were reduced in a dose-dependent manner in antisense oligonucleotide treated cells. The data also confirms that many of the newly designed oligonucleotides achieved an IC₅₀ of less than 0.7 μM and, therefore, are more potent than ISIS 407939.

TABLE 8
Dose-dependent antisense inhibition (%) of human Factor VII
in Hep3B cells using electroporation

| | 666.6667 | 2000.0 | 6000.0 | IC₅₀ |
|---|---|---|---|---|
| ISIS No | nM | nM | nM | (μM) |
| 407939 | 47 | 68 | 85 | 0.7 |
| 457851 | 60 | 80 | 93 | <0.6 |
| 472925 | 62 | 86 | 95 | <0.6 |
| 472926 | 66 | 77 | 85 | <0.6 |
| 472935 | 54 | 84 | 94 | <0.6 |
| 472958 | 66 | 82 | 88 | <0.6 |
| 472959 | 64 | 81 | 93 | <0.6 |
| 472970 | 72 | 87 | 86 | <0.6 |
| 472976 | 78 | 92 | 97 | <0.6 |
| 472994 | 79 | 92 | 96 | <0.6 |
| 472995 | 61 | 82 | 93 | <0.6 |
| 472996 | 73 | 91 | 95 | <0.6 |
| 472998 | 63 | 90 | 95 | <0.6 |
| 473019 | 55 | 80 | 86 | <0.6 |
| 473020 | 61 | 76 | 85 | <0.6 |
| 473046 | 61 | 80 | 94 | <0.6 |
| 473055 | 55 | 84 | 94 | <0.6 |
| 492104 | 53 | 76 | 88 | <0.6 |
| 492105 | 62 | 80 | 90 | <0.6 |
| 492189 | 57 | 80 | 92 | <0.6 |
| 492194 | 57 | 83 | 91 | <0.6 |
| 492195 | 58 | 81 | 95 | <0.6 |
| 492196 | 62 | 86 | 95 | <0.6 |
| 492205 | 62 | 87 | 95 | <0.6 |
| 492215 | 60 | 78 | 89 | <0.6 |
| 492221 | 63 | 76 | 92 | <0.6 |
| 492234 | 51 | 74 | 91 | 0.5 |
| 492276 | 50 | 56 | 95 | 0.8 |
| 492277 | 58 | 73 | 81 | <0.6 |
| 492306 | 61 | 75 | 84 | <0.6 |
| 492317 | 59 | 80 | 93 | <0.6 |
| 492329 | 59 | 70 | 89 | <0.6 |
| 492331 | 69 | 87 | 95 | <0.6 |
| 492333 | 47 | 70 | 85 | 0.7 |
| 492334 | 57 | 77 | 90 | <0.6 |
| 492390 | 72 | 88 | 95 | <0.6 |
| 492399 | 68 | 91 | 96 | <0.6 |
| 492401 | 68 | 89 | 95 | <0.6 |
| 492404 | 65 | 87 | 94 | <0.6 |
| 492405 | 44 | 81 | 90 | 0.7 |
| 492406 | 65 | 82 | 92 | <0.6 |
| 492440 | 50 | 70 | 89 | 0.6 |
| 492465 | 16 | 80 | 79 | 1.4 |
| 492467 | 58 | 77 | 92 | <0.6 |
| 492492 | 45 | 80 | 94 | 0.7 |
| 492494 | 63 | 82 | 93 | <0.6 |
| 492503 | 55 | 81 | 93 | <0.6 |
| 492530 | 70 | 86 | 90 | <0.6 |
| 492534 | 67 | 85 | 91 | <0.6 |
| 492536 | 54 | 81 | 89 | <0.6 |
| 492541 | 54 | 71 | 85 | <0.6 |
| 492545 | 59 | 78 | 89 | <0.6 |
| 492566 | 59 | 84 | 85 | <0.6 |
| 492571 | 52 | 81 | 89 | <0.6 |
| 492572 | 67 | 83 | 90 | <0.6 |
| 492573 | 69 | 83 | 92 | <0.6 |
| 492574 | 65 | 82 | 91 | <0.6 |
| 492575 | 72 | 83 | 91 | <0.6 |
| 492593 | 61 | 78 | 90 | <0.6 |
| 492617 | 62 | 80 | 93 | <0.6 |
| 492618 | 47 | 79 | 94 | 0.6 |
| 492619 | 54 | 82 | 95 | <0.6 |
| 492621 | 44 | 85 | 92 | 0.6 |
| 492651 | 53 | 66 | 91 | 0.6 |
| 492652 | 61 | 78 | 88 | <0.6 |
| 492658 | 59 | 79 | 88 | <0.6 |
| 492725 | 43 | 84 | 89 | 0.6 |
| 492730 | 51 | 87 | 93 | 0.4 |
| 492731 | 46 | 82 | 90 | 0.6 |
| 492784 | 56 | 88 | 96 | <0.6 |
| 492816 | 68 | 89 | 97 | <0.6 |
| 492818 | 64 | 84 | 96 | <0.6 |
| 492877 | 67 | 91 | 93 | <0.6 |
| 492878 | 80 | 89 | 93 | <0.6 |
| 492913 | 53 | 87 | 92 | <0.6 |
| 492914 | 75 | 89 | 96 | <0.6 |
| 492928 | 60 | 83 | 94 | <0.6 |
| 492938 | 70 | 90 | 92 | <0.6 |
| 492991 | 67 | 93 | 99 | <0.6 |
| 492992 | 0 | 82 | 95 | 2.1 |
| 493087 | 54 | 81 | 90 | <0.6 |
| 493114 | 50 | 73 | 90 | 0.6 |
| 493178 | 71 | 88 | 96 | <0.6 |
| 493179 | 47 | 82 | 95 | 0.6 |
| 493182 | 79 | 87 | 91 | <0.6 |
| 493195 | 55 | 78 | 90 | <0.6 |
| 493201 | 87 | 93 | 96 | <0.6 |
| 493202 | 68 | 89 | 94 | <0.6 |
| 493255 | 57 | 79 | 93 | <0.6 |
| 493291 | 57 | 87 | 93 | <0.6 |
| 493292 | 70 | 89 | 93 | <0.6 |
| 493296 | 35 | 84 | 91 | 0.9 |
| 493298 | 57 | 84 | 92 | <0.6 |
| 493299 | 65 | 84 | 93 | <0.6 |
| 493304 | 68 | 86 | 94 | <0.6 |
| 493312 | 53 | 82 | 91 | <0.6 |
| 493333 | 66 | 84 | 87 | <0.6 |

### Example 8

### Dose-Dependent Antisense Inhibition of Human Factor VII in Hep3B Cells

Additional antisense oligonucleotides from the studies above, exhibiting in vitro inhibition of Factor VII mRNA were selected and tested at various doses in Hep3B cells. Also tested was ISIS 407939, a 5-10-5 MOE gapmer, which was described in an earlier publication (WO 2009/061851).

Cells were plated at a density of 20,000 cells per well and transfected using electroporation with 0.67 μM, 2.00 μM, 1.11 μM, and 6.00 μM concentrations of antisense oligonucleotide, as specified in Table 9. After a treatment period of approximately 16 hours, RNA was isolated from the cells and Factor VII mRNA levels were measured by quantitative real-time PCR. Human Factor VII primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells.

The half maximal inhibitory concentration (IC₅₀) of each oligonucleotide is also presented in Table 9. As illustrated in Table 9, Factor VII mRNA levels were reduced in a dose-dependent manner in antisense oligonucleotide treated cells. The data also confirms that each of the newly designed oligonucleotides achieved an IC₅₀ of less than 0.6 μM and, therefore, are more potent than ISIS 407939.

TABLE 9
Dose-dependent antisense inhibition (%) of human Factor VII
in Hep3B cells using electroporation

| | 0.67 | 2.00 | 6.00 | IC₅₀ |
|---|---|---|---|---|
| ISIS No | μM | μM | μM | (μM) |
| 407939 | 52 | 71 | 86 | 0.6 |
| 472983 | 49 | 83 | 97 | 0.5 |
| 472984 | 51 | 82 | 95 | 0.5 |
| 472991 | 49 | 82 | 95 | 0.5 |
| 472998 | 59 | 88 | 96 | <0.6 |
| 492365 | 74 | 91 | 96 | <0.6 |
| 492377 | 56 | 76 | 91 | <0.6 |
| 492380 | 63 | 79 | 95 | <0.6 |
| 492384 | 67 | 84 | 94 | <0.6 |
| 492388 | 69 | 87 | 97 | <0.6 |
| 492389 | 62 | 90 | 96 | <0.6 |
| 492391 | 56 | 84 | 94 | <0.6 |
| 492398 | 63 | 80 | 95 | <0.6 |
| 492403 | 58 | 81 | 91 | <0.6 |

### Example 9

### Modified Antisense Oligonucleotides Comprising MOE Modifications Targeting Human Coagulation Factor VII

Additional antisense oligonucleotides were designed targeting a Factor VII nucleic acid and were tested for their effects on Factor VII mRNA in vitro. Also tested were ISIS 403052, ISIS 407939, ISIS 416446, ISIS 416472, ISIS 416507, ISIS 416508, ISIS 422087, ISIS 422096, ISIS 422130, and ISIS 422142 which were described in an earlier publication (WO 2009/061851), incorporated herein by reference. ISIS 490149, ISIS 490197, ISIS 490209, ISIS 490275, ISIS 490277, and ISIS 490424, described in the Examples above, were also included in the screen.

The newly designed modified antisense oligonucleotides in Table 10 were designed as 3-10-4 MOE gapmers. These gapmers are 17 nucleosides in length, wherein the central gap segment comprises of ten 2′-deoxynucleosides and is flanked on both sides (in the 5′ and 3′ directions) with wing segments. The 5′ wing segment comprises three MOE nucleosides and the 3′ wing comprises four MOE nucleosides. The internucleoside linkages throughout each oligonucleotide are phosphorothioate linkages. All cytosine residues throughout each gapmer are 5-methylcytosines.

Each gapmer listed in Table 10 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted human gene sequence. ‘n/a.’ indicates that the antisense oligonucleotide is not 100% complementary with that particular gene sequence.

Activity of the newly designed oligonucleotides was compared to ISIS 403052, ISIS 407939, ISIS 416446, ISIS 416472, ISIS 416507, ISIS 416508, ISIS 422087, ISIS 422096, ISIS 422130, and ISIS 422142. Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells. A total of 272 oligonucleotides were tested. Only those oligonucleotides which were selected for further studies are shown in Table 10. Several of the newly designed antisense oligonucleotides provided in Table 10 are more active than antisense oligonucleotides from the previous publication.

TABLE 10
Percent inhibition of human Factor VII mRNA levels by modified
antisense oligonucleotides targeted to SEQ ID NO: 1 and SEQ ID NO: 2

| Start | Stop | | | | | Start | Stop | |
|---|---|---|---|---|---|---|---|---|
| Site on | Site on | | | | | Site on | Site on | SEQ |
| SEQ ID | SEQ ID | | ISIS | % | | SEQ ID | SEQ ID | ID |
| NO: 1 | NO: 1 | Sequence | No | inhibition | Motif | NO: 2 | NO: 2 | NO |
| n/a | n/a | GGCACACTGGTCCCCATCAC | 403052 | 51 | 5-10-5 | 299 | 318 | 82 |
| 15255 | 15274 | TGCAGCCCGGCACCCAGCGA | 407939 | 78 | 5-10-5 | 2312 | 2331 | 72 |
| 11024 | 11043 | GGTCACTGCAGTACTGCTCA | 416446 | 70 | 5-10-5 | 459 | 478 | 103 |
| 14348 | 14367 | CAGCCTTGGCTTTCTCTCCA | 416472 | 79 | 5-10-5 | 1405 | 1424 | 111 |
| 4847 | 4866 | GGTTACTGAGCGCGGAAGAA | 416507 | 84 | 5-10-5 | n/a | n/a | 97 |
| 4873 | 4892 | CGAGTTCTGCAGGAGCGGCC | 416508 | 80 | 5-10-5 | n/a | n/a | 100 |
| 15192 | 15211 | CATGCATGGTGATGCTTCTG | 422087 | 89 | 5-10-5 | 2249 | 2268 | 121 |
| 11839 | 11858 | AGACAATGGTCAGGGCTGGT | 422096 | 78 | 5-10-5 | n/a | n/a | 219 |
| 14708 | 14727 | GCCTGAGGCCAGCAGATCAC | 422130 | 81 | 5-10-5 | 1765 | 1784 | 225 |
| 4872 | 4891 | GAGTTCTGCAGGAGCGGCCT | 422142 | 84 | 5-10-5 | n/a | n/a | 99 |
| 1383 | 1402 | CCTGTGCCTGGATGCTGGTT | 490275 | 77 | 5-10-5 | n/a | n/a | 90 |
| 1383 | 1399 | GTGCCTGGATGCTGGTT | 513462 | 79 | 3-10-4 | n/a | n/a | 213 |
| 1384 | 1400 | TGTGCCTGGATGCTGGT | 513463 | 81 | 3-10-4 | n/a | n/a | 214 |
| 1385 | 1404 | CTCCTGTGCCTGGATGCTGG | 490277 | 74 | 5-10-5 | n/a | n/a | 91 |
| 2490 | 2506 | GCATCAGAAAAGCTCTC | 513487 | 83 | 3-10-4 | n/a | n/a | 215 |
| 4850 | 4866 | GGTTACTGAGCGCGGAA | 513504 | 81 | 3-10-4 | n/a | n/a | 216 |
| 4873 | 4889 | GTTCTGCAGGAGCGGCC | 513507 | 86 | 3-10-4 | n/a | n/a | 217 |
| 4874 | 4890 | AGTTCTGCAGGAGCGGC | 513508 | 85 | 3-10-4 | n/a | n/a | 218 |
| 4939 | 4958 | GGAGGGACGACCTTTGCTGG | 490424 | 69 | 5-10-5 | n/a | n/a | 101 |
| 12505 | 12524 | GCTTTGATCCAATGCTCCTG | 491122 | 87 | 5-10-5 | n/a | n/a | 220 |
| 12631 | 12647 | GCTGCTCAGACCTGGCT | 513642 | 79 | 3-10-4 | n/a | n/a | 221 |
| 14232 | 14251 | GGAGACCCTGGTGTACACCC | 490149 | 71 | 5-10-5 | 1289 | 1308 | 109 |
| 14612 | 14628 | GCCCCTCTGTCCAGCGC | 513419 | 90 | 3-10-4 | 1669 | 1685 | 222 |
| 14613 | 14629 | TGCCCCTCTGTCCAGCG | 513420 | 89 | 3-10-4 | 1670 | 1686 | 223 |
| 14614 | 14630 | CTGCCCCTCTGTCCAGC | 513421 | 88 | 3-10-4 | 1671 | 1687 | 224 |
| 15130 | 15149 | GTGAGAGCTAAACAACCGCC | 490197 | 77 | 5-10-5 | 2187 | 2206 | 116 |
| 15182 | 15198 | GCTTCTGAATTGTCTGA | 513446 | 89 | 3-10-4 | 2239 | 2255 | 226 |
| 15183 | 15199 | TGCTTCTGAATTGTCTG | 513447 | 83 | 3-10-4 | 2240 | 2256 | 227 |
| 15184 | 15203 | GTGATGCTTCTGAATTGTCT | 490209 | 79 | 5-10-5 | 2241 | 2260 | 118 |
| 15191 | 15207 | CATGGTGATGCTTCTGA | 513454 | 84 | 3-10-4 | 2248 | 2264 | 228 |
| 15192 | 15208 | GCATGGTGATGCTTCTG | 513455 | 92 | 3-10-4 | 2249 | 2265 | 229 |
| 15193 | 15209 | TGCATGGTGATGCTTCT | 513456 | 89 | 3-10-4 | 2250 | 2266 | 230 |
| 15194 | 15210 | ATGCATGGTGATGCTTC | 513457 | 83 | 3-10-4 | 2251 | 2267 | 231 |

### Example 10

### Dose-Dependent Antisense Inhibition of Human Factor VII in Hep3B Cells

Antisense oligonucleotides from the studies above, exhibiting in vitro inhibition of Factor VII mRNA, were selected and tested at various doses in Hep3B cells. Also tested were ISIS 403052, ISIS 407643, ISIS 407935, ISIS 407936, ISIS 407939, ISIS 416446, ISIS 416459, ISIS 416472, ISIS 416507, ISIS 416508, ISIS 416549, ISIS 422086, ISIS 422087, ISIS 422130, ISIS and 422142, 5-10-5 MOE gapmers targeting human Factor VII, which were described in an earlier publication (WO 2009/061851).

Cells were plated at a density of 20,000 cells per well and transfected using electroporation with 0.625 μM, 1.25 μM, 2.50 μM, 5.00 μM and 10.00 μM concentrations of antisense oligonucleotide, as specified in Table 11. After a treatment period of approximately 16 hours, RNA was isolated from the cells and Factor VII mRNA levels were measured by quantitative real-time PCR. Human Factor VII primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells.

The half maximal inhibitory concentration (IC₅₀) of each oligonucleotide is also presented in Table 11. As illustrated in Table 11, Factor VII mRNA levels were reduced in a dose-dependent manner in antisense oligonucleotide treated cells. The data also confirms that several of the newly designed oligonucleotides are more potent than oligonucleotides from the previous publication.

TABLE 11
Dose-dependent antisense inhibition (%) of human Factor VII
in Hep3B cells using electroporation

| | 0.625 | 1.25 | 2.50 | 5.00 | 10.00 | IC₅₀ |
|---|---|---|---|---|---|---|
| ISIS No | μM | μM | μM | μM | μM | (μM) |
| 403052 | 21 | 35 | 63 | 82 | 89 | 1.9 |
| 407643 | 29 | 46 | 67 | 83 | 90 | 1.4 |
| 407935 | 52 | 68 | 80 | 89 | 91 | <0.6 |
| 407936 | 31 | 51 | 62 | 78 | 84 | 1.4 |
| 407939 | 30 | 61 | 74 | 83 | 88 | 1.0 |
| 416446 | 37 | 53 | 64 | 76 | 83 | 1.2 |
| 416459 | 51 | 76 | 83 | 90 | 92 | <0.6 |
| 416472 | 37 | 52 | 66 | 78 | 85 | 1.2 |
| 416507 | 45 | 68 | 82 | 87 | 90 | 0.7 |
| 416508 | 33 | 56 | 74 | 84 | 89 | 1.1 |
| 416549 | 57 | 71 | 78 | 82 | 85 | <0.6 |
| 422086 | 46 | 67 | 77 | 89 | 92 | 0.7 |
| 422087 | 50 | 69 | 74 | 86 | 91 | 0.6 |
| 422130 | 32 | 65 | 78 | 92 | 93 | 0.9 |
| 422142 | 59 | 73 | 84 | 86 | 88 | <0.6 |
| 490103 | 52 | 57 | 66 | 83 | 88 | 0.9 |
| 490149 | 34 | 58 | 71 | 85 | 91 | 1.0 |
| 490196 | 26 | 59 | 66 | 79 | 84 | 1.3 |
| 490197 | 39 | 63 | 74 | 81 | 90 | 0.8 |
| 490208 | 44 | 70 | 76 | 83 | 88 | 0.6 |
| 490275 | 36 | 58 | 76 | 85 | 89 | 1.0 |
| 490277 | 37 | 63 | 73 | 87 | 87 | 0.8 |
| 490279 | 40 | 54 | 72 | 83 | 89 | 1.0 |
| 490323 | 49 | 68 | 79 | 86 | 90 | <0.6 |
| 490368 | 39 | 62 | 76 | 86 | 91 | 0.8 |
| 490396 | 36 | 53 | 69 | 80 | 87 | 1.1 |
| 490424 | 45 | 65 | 69 | 76 | 82 | 0.6 |
| 490803 | 57 | 74 | 85 | 89 | 92 | <0.6 |
| 513419 | 60 | 71 | 85 | 95 | 96 | <0.6 |
| 513420 | 37 | 69 | 79 | 94 | 96 | 0.7 |
| 513421 | 46 | 64 | 84 | 95 | 97 | 0.6 |
| 513446 | 47 | 81 | 88 | 95 | 96 | <0.6 |
| 513447 | 56 | 74 | 81 | 92 | 96 | <0.6 |
| 513454 | 50 | 77 | 82 | 93 | 95 | <0.6 |
| 513455 | 74 | 82 | 91 | 96 | 96 | <0.6 |
| 513456 | 66 | 80 | 88 | 94 | 95 | <0.6 |
| 513457 | 54 | 67 | 80 | 87 | 89 | <0.6 |
| 513462 | 49 | 72 | 84 | 87 | 89 | <0.6 |
| 513463 | 36 | 62 | 76 | 85 | 89 | 0.9 |
| 513487 | 42 | 56 | 73 | 87 | 93 | 0.9 |
| 513504 | 47 | 65 | 81 | 90 | 91 | 0.6 |
| 513505 | 39 | 50 | 78 | 85 | 92 | 1.0 |
| 513507 | 52 | 73 | 83 | 89 | 93 | <0.6 |
| 513508 | 56 | 78 | 85 | 91 | 94 | <0.6 |

### Example 11

### Dose-Dependent Antisense Inhibition of Human Factor VII in Hep3B Cells

Additional antisense oligonucleotides from the studies above, exhibiting in vitro inhibition of Factor VII mRNA, were tested at various doses in Hep3B cells. Also tested were ISIS 407935, ISIS 407939, ISIS 416446, ISIS 416472, ISIS 416507, ISIS 416549, ISIS 422086, ISIS 422087, ISIS 422096, and ISIS 422142, 5-10-5 MOE gapmers targeting human Factor VII, which were described in an earlier publication (WO 2009/061851).

Cells were plated at a density of 20,000 cells per well and transfected using electroporation with 0.3125 μM, 0.625 μM, 1.25 μM, 2.50 μM, 5.00 μM and 10.00 μM concentrations of antisense oligonucleotide, as specified in Table 12. After a treatment period of approximately 16 hours, RNA was isolated from the cells and Factor VII mRNA levels were measured by quantitative real-time PCR. Human Factor VII primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells.

The half maximal inhibitory concentration (IC₅₀) of each oligonucleotide is also presented in Table 12. As illustrated in Table 12, Factor VII mRNA levels were reduced in a dose-dependent manner in antisense oligonucleotide treated cells. The data also confirms that several of the newly designed oligonucleotides are more potent than oligonucleotides from the previous publication.

TABLE 12
Dose-dependent antisense inhibition (%) of
human Factor VII in Hep3B cells using electroporation

| | 0.3125 | 0.625 | 1.250 | 2.500 | | | IC₅₀ |
|---|---|---|---|---|---|---|---|
| ISIS No | μM | μM | μM | μM | 5.000 μM | 10.000 μM | (μM) |
| 407935 | 30 | 49 | 75 | 86 | 91 | 94 | 0.6 |
| 407939 | 30 | 48 | 61 | 78 | 85 | 90 | 0.8 |
| 416446 | 27 | 52 | 63 | 75 | 85 | 90 | 0.7 |
| 416472 | 38 | 51 | 72 | 83 | 88 | 94 | 0.5 |
| 416507 | 58 | 81 | 76 | 84 | 89 | 92 | <0.3 |
| 416549 | 52 | 67 | 75 | 81 | 88 | 89 | 0.3 |
| 422086 | 48 | 49 | 68 | 78 | 86 | 91 | 0.5 |
| 422087 | 30 | 56 | 66 | 83 | 72 | 92 | 0.6 |
| 422096 | 47 | 63 | 70 | 77 | 83 | 85 | <0.3 |
| 422142 | 69 | 85 | 87 | 85 | 89 | 91 | <0.3 |
| 490103 | 52 | 57 | 68 | 78 | 87 | 93 | 0.4 |
| 490149 | 33 | 64 | 62 | 77 | 86 | 93 | 0.5 |
| 490197 | 38 | 46 | 60 | 75 | 87 | 93 | 0.7 |
| 490208 | 46 | 62 | 73 | 83 | 88 | 91 | 0.4 |
| 490209 | 40 | 54 | 72 | 79 | 85 | 94 | 0.5 |
| 490275 | 52 | 61 | 67 | 78 | 85 | 91 | 0.3 |
| 490277 | 33 | 59 | 77 | 79 | 91 | 94 | 0.5 |
| 490323 | 43 | 61 | 72 | 69 | 84 | 87 | 0.4 |
| 490368 | 50 | 64 | 78 | 83 | 90 | 92 | <0.3 |
| 490396 | 46 | 64 | 68 | 84 | 84 | 90 | 0.3 |
| 490424 | 24 | 47 | 58 | 72 | 76 | 82 | 1.0 |
| 490803 | 45 | 60 | 70 | 84 | 88 | 89 | 0.3 |
| 513419 | 32 | 53 | 76 | 88 | 93 | 95 | 0.5 |
| 513420 | 35 | 59 | 72 | 82 | 94 | 97 | 0.5 |
| 513421 | 46 | 67 | 78 | 86 | 94 | 96 | <0.3 |
| 513446 | 26 | 61 | 77 | 89 | 91 | 97 | 0.5 |
| 513447 | 22 | 48 | 60 | 82 | 91 | 95 | 0.8 |
| 513454 | 25 | 59 | 76 | 86 | 94 | 96 | 0.5 |
| 513455 | 60 | 73 | 85 | 89 | 95 | 96 | <0.3 |
| 513456 | 49 | 60 | 81 | 88 | 94 | 95 | <0.3 |
| 513457 | 43 | 50 | 72 | 77 | 87 | 92 | 0.5 |
| 513462 | 25 | 48 | 58 | 76 | 83 | 88 | 0.8 |
| 513463 | 22 | 45 | 66 | 73 | 85 | 88 | 0.9 |
| 513487 | 41 | 56 | 65 | 79 | 86 | 90 | 0.4 |
| 513504 | 19 | 48 | 63 | 76 | 87 | 92 | 0.9 |
| 513505 | 11 | 21 | 54 | 73 | 85 | 90 | 1.4 |
| 513507 | 47 | 55 | 72 | 82 | 90 | 91 | 0.3 |
| 513508 | 31 | 59 | 74 | 85 | 92 | 93 | 0.5 |
| 513642 | 43 | 55 | 67 | 80 | 88 | 92 | 0.4 |

### Example 12

### Tolerability of MOE Gapmers Targeting Human Factor VII in BALB/c Mice

BALB/c mice are a multipurpose mice model, frequently utilized for safety and efficacy testing. The mice were treated with ISIS antisense oligonucleotides selected from studies described above and evaluated for changes in the levels of various plasma chemistry markers.

### Treatment

Groups of male BALB/c mice were injected subcutaneously twice a week for 3 weeks with 50 mg/kg of ISIS 407935, ISIS 416472, ISIS 416549, ISIS 422086, ISIS 422087, ISIS 422096, ISIS 422142, ISIS 490103, ISIS 490149, ISIS 490196, ISIS 490208, ISIS 490209, ISIS 513419, ISIS 513420, ISIS 513421, ISIS 513454, ISIS 513455, ISIS 513456, ISIS 513457, ISIS 513462, ISIS 513463, ISIS 513487, ISIS 513504, ISIS 513508, and ISIS 513642. One group of male BALB/c mice was injected subcutaneously twice a week for 3 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, N.Y.).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 407935, ISIS 416472, ISIS 416549, ISIS 422087, ISIS 422096, ISIS 490103, ISIS 490196, ISIS 490208, ISIS 513454, ISIS 513455, ISIS 513456, ISIS 513457, ISIS 513487, ISIS 513504, and ISIS 513508 were considered very tolerable in terms of liver function. Based on these criteria, ISIS 422086, ISIS 490209, ISIS 513419, ISIS 513420, and ISIS 513463 were considered tolerable in terms of liver function.

### Example 13

### Dose-Dependent Antisense Inhibition of Human Factor VII in Hep3B Cells

Additional antisense oligonucleotides from the studies above, exhibiting in vitro inhibition of Factor VII mRNA were selected and tested at various doses in Hep3B cells. Also tested was ISIS 407939, a 5-10-5 MOE gapmer, which was described in an earlier publication (WO 2009/061851).

Cells were plated at a density of 20,000 cells per well and transfected using electroporation with 0.074 μM, 0.222 μM, 0.667 μM, 2.000 μM, and 6.000 μM concentrations of antisense oligonucleotide, as specified in Table 13. After a treatment period of approximately 16 hours, RNA was isolated from the cells and Factor VII mRNA levels were measured by quantitative real-time PCR. Human Factor VII primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells.

The half maximal inhibitory concentration (IC₅₀) of each oligonucleotide is also presented in Table 13. As illustrated in Table 13, Factor VII mRNA levels were reduced in a dose-dependent manner in antisense oligonucleotide treated cells. Many of the newly designed antisense oligonucleotides provided in Table 13 achieved an IC₅₀ of less than 0.9 μM and, therefore, are more potent than ISIS 407939.

TABLE 13
Dose-dependent antisense inhibition (%) of human
Factor VII in Hep3B cells using electroporation

| | 0.074 | | | | | IC₅₀ |
|---|---|---|---|---|---|---|
| ISIS No | μM | 0.222 μM | 0.667 μM | 2.000 μM | 6.000 μM | (μM) |
| 407939 | 2 | 17 | 53 | 70 | 87 | 0.9 |
| 472970 | 17 | 47 | 75 | 92 | 95 | 0.3 |
| 472988 | 0 | 8 | 21 | 54 | 92 | 1.4 |
| 472996 | 18 | 59 | 74 | 93 | 95 | 0.2 |
| 473244 | 91 | 95 | 97 | 99 | 99 | <0.07 |
| 473286 | 6 | 53 | 85 | 92 | 98 | 0.3 |
| 473359 | 2 | 3 | 20 | 47 | 67 | 2.6 |
| 473392 | 71 | 85 | 88 | 92 | 96 | <0.07 |
| 473393 | 91 | 96 | 97 | 98 | 99 | <0.07 |
| 473547 | 85 | 88 | 93 | 97 | 98 | <0.07 |
| 473567 | 0 | 25 | 66 | 88 | 95 | 0.7 |
| 473589 | 8 | 47 | 79 | 94 | 99 | 0.3 |
| 482814 | 23 | 68 | 86 | 93 | 96 | 0.1 |
| 482815 | 6 | 48 | 65 | 90 | 96 | 0.4 |
| 482963 | 3 | 68 | 85 | 94 | 96 | 0.2 |
| 483241 | 14 | 33 | 44 | 76 | 93 | 0.6 |
| 483261 | 14 | 21 | 41 | 72 | 88 | 0.7 |
| 483290 | 0 | 1 | 41 | 69 | 92 | 1.0 |
| 483414 | 8 | 1 | 36 | 76 | 91 | 0.9 |
| 483415 | 0 | 40 | 52 | 84 | 94 | 0.6 |
| 484559 | 26 | 51 | 78 | 87 | 97 | 0.2 |
| 484713 | 6 | 5 | 53 | 64 | 88 | 0.9 |

### Example 14

### Modified Antisense Oligonucleotides Comprising cEt and MOE Modifications Targeting Human Coagulation Factor VII

Additional antisense oligonucleotides were designed targeting a Factor VII nucleic acid and were tested for their effects on Factor VII mRNA in vitro. Also tested was ISIS 407939, a 5-10-5 MOE gapmer targeting human Factor VII, which was described in an earlier publication (WO 2009/061851). ISIS 472998, ISIS 492878, ISIS 493201, and 493182, which are 2-10-2 cEt gapmers described in the Examples above, were also included in the screen.

The newly designed modified antisense oligonucleotides and their motifs are described in Table 14. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO.

Each oligonucleotide listed in Table 14 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted human gene sequence. ‘n/a’ indicates that the antisense oligonucleotide is not 100% complementary with that particular gene sequence.

Activity of newly designed oligonucleotides was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells. A total of 685 oligonucleotides were tested. Only those oligonucleotides which were selected for further studies are shown in Table 14. Many of the newly designed antisense oligonucleotides provided in Table 14 achieved greater than 68% inhibition and, therefore, are more active than ISISI 407939.

TABLE 14
Percent inhibition of human Factor VII mRNA levels by modified
antisense oligonucleotides targeted to SEQ ID NO: 1 and SEQ ID NO: 2

| Start | Stop | | | | | | | |
|---|---|---|---|---|---|---|---|---|
| Site on | Site on | | SEQ | | | Start Site | Stop Site | |
| SEQ ID | SEQ ID | | ID | | % | on SEQ | on SEQ | |
| NO: 1 | NO: 1 | Sequence | NO | ISIS No | inhibition | ID NO: 2 | ID NO: 2 | Sugar Chemistry |
| 15255 | 15274 | TGCAGCCCGGCACCCAGCGA | 72 | 407939 | 68 | 2312 | 2331 | eeeeeddddddddddeeeee |
| 9600 | 9613 | AAGTTTACCAAGCG | 189 | 492878 | 73 | n/a | n/a | kkddddddddddkk |
| 11703 | 11716 | GTTGTTTTGATCCC | 200 | 493182 | 80 | n/a | n/a | kkddddddddddkk |
| 11847 | 11860 | GGAGACAATGGTCA | 202 | 493201 | 84 | n/a | n/a | kkddddddddddkk |
| 15263 | 15276 | TGTGCAGCCCGGCA | 74 | 472998 | 91 | 2320 | 2333 | kkddddddddddkk |
| 1382 | 1397 | GCCTGGATGCTGGTTT | 23 | 515640 | 75 | n/a | n/a | eeeddddddddddkkk |
| 1383 | 1398 | TGCCTGGATGCTGGTT | 232 | 515637 | 77 | n/a | n/a | eeeddddddddddkkk |
| 2499 | 2514 | GCAGATTTGCATCAGA | 233 | 515554 | 72 | n/a | n/a | eeeddddddddddkkk |
| 4851 | 4866 | GGTTACTGAGCGCGGA | 234 | 515406 | 80 | n/a | n/a | kkkddddddddddeee |
| 4851 | 4866 | GGTTACTGAGCGCGGA | 234 | 515558 | 81 | n/a | n/a | eeeddddddddddkkk |
| 4872 | 4887 | TCTGCAGGAGCGGCCT | 235 | 515407 | 88 | n/a | n/a | kkkddddddddddeee |
| 4873 | 4888 | TTCTGCAGGAGCGGCC | 236 | 515408 | 85 | n/a | n/a | kkkddddddddddeee |
| 5374 | 5389 | GACCTCGCGCGGATCC | 237 | 515422 | 86 | n/a | n/a | kkkddddddddddeee |
| 5512 | 5527 | CCGAGGCGCGGCCCCT | 238 | 515423 | 90 | n/a | n/a | kkkddddddddddeee |
| 5512 | 5527 | CCGAGGCGCGGCCCCT | 238 | 515575 | 84 | n/a | n/a | eeeddddddddddkkk |
| 5513 | 5528 | TCCGAGGCGCGGCCCC | 239 | 515424 | 87 | n/a | n/a | kkkddddddddddeee |
| 8643 | 8658 | AGAAACTGTTGGCCAC | 240 | 515432 | 78 | n/a | n/a | kkkddddddddddeee |
| 8644 | 8659 | AAGAAACTGTTGGCCA | 241 | 515433 | 71 | n/a | n/a | kkkddddddddddeee |
| 8655 | 8670 | AGTGATTGCTGAAGAA | 242 | 515434 | 76 | n/a | n/a | kkkddddddddddeee |
| 9169 | 9184 | GGCACACTGGTCCCCA | 243 | 515334 | 85 | 303 | 318 | kkkddddddddddeee |
| 9170 | 9185 | AGGCACACTGGTCCCC | 88 | 515649 | 61 | 304 | 319 | eeeddddddddddkkk |
| 9225 | 9240 | CAGATATAGGACTGGA | 244 | 515338 | 86 | 359 | 374 | kkkddddddddddeee |
| 9359 | 9374 | CCAGGTTGTGCACCCT | 245 | 515438 | 76 | n/a | n/a | kkkddddddddddeee |
| 9453 | 9468 | CCTGTCAAAGACCTCA | 246 | 515439 | 75 | n/a | n/a | kkkddddddddddeee |
| 10383 | 10398 | GGATGCACACCAGGGC | 247 | 516003 | 87 | n/a | n/a | kkddddddddddeeee |
| 11016 | 11031 | ACTGCTCACAGCCGCC | 77 | 515647 | 60 | 451 | 466 | eeeddddddddddkkk |
| 11839 | 11854 | AATGGTCAGGGCTGGT | 34 | 515639 | 78 | n/a | n/a | eeeddddddddddkkk |
| 12127 | 12142 | GGGTTTGCTGGCATTT | 248 | 515648 | 36 | 597 | 612 | eeeddddddddddkkk |
| 12633 | 12648 | AGCTGCTCAGACCTGG | 39 | 515641 | 69 | n/a | n/a | eeeddddddddddkkk |
| 13741 | 13756 | GTGCTCGCTGAGGTCG | 44 | 515650 | 76 | 798 | 813 | eeeddddddddddkkk |
| 13742 | 13757 | CGTGCTCGCTGAGGTC | 249 | 515354 | 87 | 799 | 814 | kkkddddddddddeee |
| 14077 | 14092 | GAACATGTACTCCGTG | 250 | 515926 | 87 | 1134 | 1149 | kkddddddddddeeee |
| 14094 | 14109 | TCCGAGTAGCCGGCAC | 251 | 515366 | 87 | 1151 | 1166 | kkkddddddddddeee |
| 14243 | 14258 | TGTACTGGGAGACCCT | 252 | 515642 | 58 | 1300 | 1315 | eeeddddddddddkkk |
| 14612 | 14627 | CCCCTCTGTCCAGCGC | 53 | 515643 | 81 | 1669 | 1684 | eeeddddddddddkkk |
| 15130 | 15145 | GAGCTAAACAACCGCC | 253 | 515944 | 84 | 2187 | 2202 | kkddddddddddeeee |
| 15131 | 15146 | AGAGCTAAACAACCGC | 254 | 515380 | 90 | 2188 | 2203 | kkkddddddddddeee |
| 15131 | 15146 | AGAGCTAAACAACCGC | 254 | 515532 | 83 | 2188 | 2203 | eeeddddddddddkkk |
| 15131 | 15146 | AGAGCTAAACAACCGC | 254 | 515945 | 85 | 2188 | 2203 | kkddddddddddeeee |
| 15132 | 15147 | GAGAGCTAAACAACCG | 255 | 515381 | 82 | 2189 | 2204 | kkkddddddddddeee |
| 15183 | 15198 | GCTTCTGAATTGTCTG | 256 | 515382 | 95 | 2240 | 2255 | kkkddddddddddeee |
| 15183 | 15198 | GCTTCTGAATTGTCTG | 256 | 515948 | 94 | 2240 | 2255 | kkddddddddddeeee |
| 15185 | 15200 | ATGCTTCTGAATTGTC | 257 | 515949 | 87 | 2242 | 2257 | kkddddddddddeeee |
| 15186 | 15201 | GATGCTTCTGAATTGT | 258 | 515384 | 89 | 2243 | 2258 | kkkddddddddddeee |
| 15190 | 15205 | TGGTGATGCTTCTGAA | 65 | 515635 | 82 | 2247 | 2262 | eeeddddddddddkkk |
| 15191 | 15206 | ATGGTGATGCTTCTGA | 67 | 515638 | 90 | 2248 | 2263 | eeeddddddddddkkk |
| 15192 | 15207 | CATGGTGATGCTTCTG | 259 | 515386 | 92 | 2249 | 2264 | kkkddddddddddeee |
| 15192 | 15207 | CATGGTGATGCTTCTG | 259 | 515951 | 84 | 2249 | 2264 | kkddddddddddeeee |
| 15193 | 15208 | GCATGGTGATGCTTCT | 260 | 515387 | 78 | 2250 | 2265 | kkkddddddddddeee |
| 15193 | 15208 | GCATGGTGATGCTTCT | 260 | 515952 | 89 | 2250 | 2265 | kkddddddddddeeee |
| 15194 | 15209 | TGCATGGTGATGCTTC | 69 | 515636 | 90 | 2251 | 2266 | eeeddddddddddkkk |
| 15196 | 15211 | CATGCATGGTGATGCT | 261 | 515388 | 84 | 2253 | 2268 | kkkddddddddddeee |

### Example 15

### Tolerability of Modified Oligonucleotides Having Deoxy, MOE, and cEt Modifications Targeting Human Factor VII in BALB/c Mice

BALB/c mice were treated with ISIS antisense oligonucleotides selected from studies described above and evaluated for changes in the levels of various plasma chemistry markers.

Additionally, newly designed antisense oligonucleotides were also added to this screen. The newly designed modified antisense oligonucleotides and their motifs are described in Table 15. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO.

Each oligonucleotide listed in Table 15 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted human gene sequence. ‘n/a.’ indicates that the antisense oligonucleotide is not 100% complementary with that particular gene sequence.

TABLE 15
Modified antisense oligonucleotides targeted to
SEQ ID NO: 1 and SEQ ID NO: 2

| Start | Stop | | | | | | |
|---|---|---|---|---|---|---|---|
| Site on | Site on | | SEQ | | Start Site | Stop Site | |
| SEQ ID | SEQ ID | | ID | | on SEQ | on SEQ | |
| NO: 1 | NO: 1 | Sequence | NO | ISIS No | ID NO: 2 | ID NO: 2 | Sugar Chemistry |
| 1147 | 1162 | GATGAAATCTCTGCAG | 21 | 516044 | 36 | 51 | eeeddddddddddkkk |
| 1154 | 1169 | AGACCATGATGAAATC | 22 | 516045 | 43 | 58 | eeeddddddddddkkk |
| 2369 | 2384 | TGGAGCGGTCACTTCC | 26 | 516058 | n/a | n/a | eeeddddddddddkkk |
| 4717 | 4732 | AGGAGGCTGAGGATGC | 27 | 516059 | n/a | n/a | eeeddddddddddkkk |
| 4871 | 4886 | CTGCAGGAGCGGCCTA | 28 | 516060 | n/a | n/a | eeeddddddddddkkk |
| 6411 | 6426 | CGTATTTTCTGATGTG | 29 | 516061 | n/a | n/a | eeeddddddddddkkk |
| 6642 | 6657 | GAGGTGACCCGTGAGC | 30 | 516062 | n/a | n/a | eeeddddddddddkkk |
| 12141 | 12156 | ACAATTCGGCCTTGGG | 37 | 516046 | 611 | 626 | eeeddddddddddkkk |
| 12629 | 12644 | GCTCAGACCTGGCTCT | 38 | 516063 | n/a | n/a | eeeddddddddddkkk |
| 12631 | 12646 | CTGCTCAGACCTGGCT | 89 | 516064 | n/a | n/a | eeeddddddddddkkk |
| 12634 | 12649 | AAGCTGCTCAGACCTG | 262 | 516065 | n/a | n/a | eeeddddddddddkkk |
| 12635 | 12650 | AAAGCTGCTCAGACCT | 263 | 516066 | n/a | n/a | eeeddddddddddkkk |
| 12842 | 12857 | CCACCCAGATGGTGTT | 41 | 516047 | 715 | 730 | eeeddddddddddkkk |
| 12863 | 12878 | CGAAACAGTGGGCCGC | 42 | 516048 | 736 | 751 | eeeddddddddddkkk |
| 13760 | 13775 | GGCTCTGCTCATCCCC | 81 | 516049 | 817 | 832 | eeeddddddddddkkk |
| 13988 | 14003 | CCATGAGCTCCAGGGC | 45 | 516050 | 1045 | 1060 | eeeddddddddddkkk |
| 14079 | 14094 | CAGAACATGTACTCCG | 48 | 516051 | 1136 | 1151 | eeeddddddddddkkk |
| 14092 | 14107 | CGAGTAGCCGGCACAG | 49 | 516052 | 1149 | 1164 | eeeddddddddddkkk |
| 14128 | 14143 | TCCACTGTCCCCCTTG | 50 | 515652 | 1185 | 1200 | eeeddddddddddkkk |
| 14231 | 14246 | CCCTGGTGTACACCCC | 264 | 508039 | 1288 | 1303 | eeeddddddddddkkk |
| 14232 | 14247 | ACCCTGGTGTACACCC | 265 | 516053 | 1289 | 1304 | eeeddddddddddkkk |
| 14708 | 14723 | GAGGCCAGCAGATCAC | 76 | 515654 | 1765 | 1780 | eeeddddddddddkkk |
| 14713 | 14728 | AGCCTGAGGCCAGCAG | 77 | 515656 | 1770 | 1785 | eeeddddddddddkkk |
| 15097 | 15112 | ATGGAGTCAGCATCGG | 57 | 516054 | 2154 | 2169 | eeeddddddddddkkk |
| 15128 | 15143 | GCTAAACAACCGCCTT | 59 | 516055 | 2185 | 2200 | eeeddddddddddkkk |
| 15164 | 15179 | TGAAGATGATAATGGA | 61 | 515655 | 2221 | 2236 | eeeddddddddddkkk |
| 15181 | 15196 | TTCTGAATTGTCTGAA | 63 | 516056 | 2238 | 2253 | eeeddddddddddkkk |
| 15188 | 15203 | GTGATGCTTCTGAATT | 64 | 516057 | 2245 | 2260 | eeeddddddddddkkk |
| 15195 | 15210 | ATGCATGGTGATGCTT | 71 | 515653 | 2252 | 2267 | eeeddddddddddkkk |
| 15262 | 15277 | CTGTGCAGCCCGGCAC | 73 | 515657 | 2319 | 2334 | eeeddddddddddkkk |

### Treatment

Groups of 4-6-week old male BALB/c mice were injected subcutaneously twice a week for 3 weeks with 25 mg/kg of ISIS 457851, ISIS 515635, ISIS 515636, ISIS 515637, ISIS 515638, ISIS 515639, ISIS 515640, ISIS 515641, ISIS 515642, ISIS 515643, ISIS 515647, ISIS 515648, ISIS 515649, ISSI 515650, ISIS 515652, ISIS 515653, ISIS 515654, ISIS 515655, ISIS 515656, ISIS 515657, ISIS 516044, ISIS 516045, ISIS 516046, ISIS 516047, ISIS 516048, ISIS 516049, ISIS 516050, ISIS 516051, ISIS 516052, ISIS 516053, ISIS 516054, ISIS 516055, ISIS 516056, ISIS 516057, ISIS 516058, ISIS 516059, ISIS 516060, ISIS 516061, ISIS 516062, ISIS 516063, ISIS 516064, ISIS 516065, or ISIS 516066. One group of 4-6-week old male BALB/c mice was injected subcutaneously twice a week for 3 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, N.Y.).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 515636, ISIS 515639, ISIS 515641, ISIS 515642, ISIS 515648, ISIS 515650, ISIS 515652, ISIS 515653, ISIS 515655, ISIS 515657, ISIS 516044, ISIS 516045, ISIS 516047, ISIS 516048, ISIS 516051, ISIS 516052, ISIS 516053, ISIS 516055, ISIS 516056, ISIS 516058, ISIS 516059, ISIS 516060, ISIS 516061, ISIS 516062, ISIS 516063, ISIS 516064, ISIS 516065, and ISIS 516066 were considered very tolerable in terms of liver function. Based on these criteria, ISIS 457851, ISIS 515635, ISIS 515637, ISIS 515638, ISIS 515643, ISIS 515647, ISIS 515649, ISIS 515650, ISIS 515652, ISIS 515654, ISIS 515656, ISIS 516056, and ISIS 516057 were considered tolerable in terms of liver function.

### Example 16

### Efficacy of Modified Antisense Oligonucleotides Comprising MOE and cEt Modifications Targeting Human Factor VII in Transgenic Mice

Transgenic mice were developed at Taconic Farms Inc. harboring a Factor VII genomic DNA fragment. The mice were treated with ISIS antisense oligonucleotides selected from studies described above and evaluated for efficacy.

### Treatment

Groups of 3-4 male and female transgenic mice were injected subcutaneously twice a week for 3 weeks with 10 mg/kg of ISIS 457851, ISIS 515636, ISIS 515639, ISIS 515653, ISIS 516053, ISIS 516065, or ISIS 516066. One group of mice was injected subcutaneously twice a week for 3 weeks with control oligonucleotide, ISIS141923 (CCTTCCCTGAAGGTTCCTCC, 5-10-5 MOE gapmer with no known murine target, SEQ ID NO: 266). One group of mice was injected subcutaneously twice a week for 3 weeks with PBS. Mice were euthanized 48 hours after the last dose, and organs and plasma were harvested for further analysis.

### RNA Analysis

RNA was extracted from plasma for real-time PCR analysis of Factor VII, using primer probe set RTS2927 (described hereinabove in Example 1). The mRNA levels were normalized using RIBOGREEN®. Results are presented as percent inhibition of Factor VII expression, relative to control. As shown in Table 16, each of the antisense oligonucleotides achieved reduction of human Factor VII mRNA expression over the PBS control. Treatment with the control oligonucleotide did not achieve reduction in Factor VII levels, as expected.

TABLE 16
Percent inhibition of Factor VII mRNA in transgenic mice

| ISIS No | % inhibition |
|---|---|
| 141923 | 0 |
| 457851 | 76 |
| 515636 | 66 |
| 515639 | 49 |
| 515653 | 78 |
| 516053 | 72 |
| 516065 | 59 |
| 516066 | 39 |

### Protein Analysis

Plasma protein levels of Factor VII were estimated using a Zymutest FVII ELISA kit (Hyphen Bio-Med cat#ARK036A). Results are presented as percent inhibition of Factor VII, relative to control. As shown in Table 17, several antisense oligonucleotides achieved reduction of human Factor VII protein expression over the PBS control.

TABLE 17
Percent inhibition of Factor VII protein levels in transgenic mice

| ISIS No | % inhibition |
|---|---|
| 141923 | 0 |
| 457851 | 64 |
| 515636 | 68 |
| 515639 | 46 |
| 515653 | 0 |
| 516053 | 19 |
| 516065 | 0 |
| 516066 | 7 |

### Example 17

### Efficacy of Modified Antisense Oligonucleotides Comprising MOE and cEt Modifications Targeting Human Factor VII in Transgenic Mice

Transgenic mice were treated with ISIS antisense oligonucleotides selected from studies described above and evaluated for efficacy.

### Treatment

Groups of 2-4 male and female transgenic mice were injected subcutaneously twice a week for 3 weeks with 5 mg/kg of ISIS 407935, ISIS 416472, ISIS 416549, ISIS 422087, ISIS 422096, ISIS 473137, ISIS 473244, ISIS 473326, ISIS 473327, ISIS 473359, ISIS 473392, ISIS 473393, ISIS 473547, ISIS 473567, ISIS 473589, ISIS 473630, ISIS 484559, ISIS 484713, ISIS 490103, ISIS 490196, ISIS 490208, ISIS 513419, ISIS 513454, ISIS 513455, ISIS 513456, ISIS 513457, ISIS 513487, ISIS 513508, ISIS 515640, ISIS 515641, ISIS 515642, ISIS 515648, ISIS 515655, ISIS 515657, ISIS 516045, ISIS 516046, ISIS 516047, ISIS 516048, ISIS 516051, ISIS 516052, ISIS 516055, ISIS 516056, ISIS 516059, ISIS 516061, ISIS 516062, or ISIS 516063. One group of mice was injected subcutaneously twice a week for 3 weeks with PBS. Mice were euthanized 48 hours after the last dose, and organs and plasma were harvested for further analysis.

### Protein Analysis

Plasma protein levels of Factor VII were estimated using a Zymutest FVII ELISA kit (Hyphen Bio-Med cat#ARK036A). Results are presented as percent inhibition of Factor VII, relative to control. As shown in Table 18, several antisense oligonucleotides achieved reduction of human Factor VII relative to the PBS control.

TABLE 18
Percent inhibition of Factor VII plasma protein levels
in transgenic mice

| ISIS No | % inhibition |
|---|---|
| 407935 | 80 |
| 416472 | 49 |
| 416549 | 29 |
| 422087 | 12 |
| 422096 | 21 |
| 473137 | 57 |
| 473244 | 67 |
| 473326 | 42 |
| 473327 | 100 |
| 473359 | 0 |
| 473392 | 22 |
| 473393 | 32 |
| 473547 | 73 |
| 473567 | 77 |
| 473589 | 96 |
| 473630 | 75 |
| 484559 | 75 |
| 484713 | 56 |
| 490103 | 0 |
| 490196 | 74 |
| 490208 | 90 |
| 513419 | 90 |
| 513454 | 83 |
| 513455 | 91 |
| 513456 | 81 |
| 513457 | 12 |
| 513487 | 74 |
| 513508 | 77 |
| 515640 | 83 |
| 515641 | 87 |
| 515642 | 23 |
| 515648 | 32 |
| 515655 | 79 |
| 515657 | 81 |
| 516045 | 52 |
| 516046 | 79 |
| 516047 | 65 |
| 516048 | 79 |
| 516051 | 84 |
| 516052 | 72 |
| 516055 | 70 |
| 516056 | 0 |
| 516059 | 39 |
| 516061 | 64 |
| 516062 | 96 |
| 516063 | 24 |

### Example 18

### Dose-Dependent Antisense Inhibition of Human Factor VII in Hep3B Cells

Antisense oligonucleotides exhibiting in vitro inhibition of Factor VII mRNA were selected and tested at various doses in Hep3B cells. Also tested was ISIS 407939, a 5-10-5 MOE gapmer targeting human Factor VII, which was described in an earlier publication (WO 2009/061851).

Cells were plated at a density of 20,000 cells per well and transfected using electroporation with 0.074 μM, 0.222 μM, 0.667 μM, 2.000 μM, and 6.000 μM concentrations of antisense oligonucleotide, as specified in Table 19. After a treatment period of approximately 16 hours, RNA was isolated from the cells and Factor VII mRNA levels were measured by quantitative real-time PCR. Human Factor VII primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells.

The half maximal inhibitory concentration (IC₅₀) of each oligonucleotide is also presented in Table 19. As illustrated in Table 19, Factor VII mRNA levels were reduced in a dose-dependent manner in antisense oligonucleotide treated cells. Many of the newly designed antisense oligonucleotides provided in Table 19 achieved an IC₅₀ of less than 2.0 μM and, therefore, are more potent than ISIS 407939.

TABLE 19
Dose-dependent antisense inhibition (%) of human
Factor VII in Hep3B cells using electroporation

| | 0.074 | | | | | IC₅₀ |
|---|---|---|---|---|---|---|
| ISIS No | μM | 0.222 μM | 0.667 μM | 2.000 μM | 6.000 μM | (μM) |
| 407939 | 0 | 9 | 21 | 58 | 76 | 2.0 |
| 515636 | 14 | 32 | 50 | 62 | 81 | 0.7 |
| 515639 | 10 | 24 | 41 | 61 | 67 | 1.3 |
| 515640 | 4 | 16 | 35 | 52 | 63 | 2.0 |
| 515641 | 0 | 21 | 27 | 55 | 66 | 1.9 |
| 515642 | 3 | 13 | 36 | 44 | 66 | 2.2 |
| 515648 | 8 | 10 | 10 | 5 | 16 | >6.0 |
| 515653 | 9 | 35 | 26 | 55 | 71 | 1.5 |
| 515655 | 0 | 0 | 6 | 13 | 42 | >6.0 |
| 515657 | 0 | 13 | 17 | 38 | 51 | 6.0 |
| 516045 | 0 | 6 | 15 | 19 | 40 | >6.0 |
| 516046 | 0 | 7 | 32 | 48 | 69 | 2.1 |
| 516047 | 12 | 27 | 41 | 50 | 63 | 1.8 |
| 516051 | 9 | 8 | 34 | 52 | 66 | 2.0 |
| 516052 | 17 | 42 | 27 | 53 | 75 | 1.2 |
| 516053 | 9 | 7 | 28 | 63 | 77 | 1.3 |
| 516055 | 0 | 3 | 27 | 54 | 75 | 2.0 |
| 516056 | 0 | 4 | 14 | 52 | 66 | 2.6 |
| 516057 | 0 | 34 | 33 | 51 | 70 | 1.6 |
| 516058 | 13 | 12 | 25 | 47 | 74 | 2.0 |
| 516059 | 4 | 15 | 36 | 47 | 68 | 1.9 |
| 516060 | 0 | 1 | 39 | 29 | 63 | 3.2 |
| 516061 | 0 | 0 | 24 | 0 | 3 | <6.0 |
| 516062 | 0 | 20 | 43 | 65 | 78 | 1.0 |
| 516063 | 0 | 8 | 10 | 37 | 61 | 3.8 |
| 516064 | 0 | 3 | 13 | 45 | 69 | 2.7 |
| 516065 | 0 | 14 | 38 | 63 | 76 | 1.3 |
| 516066 | 0 | 3 | 30 | 55 | 75 | 1.7 |

### Example 19

### Modified Antisense Oligonucleotides Comprising cEt and MOE Modifications Targeting Human Coagulation Factor VII

Additional antisense oligonucleotides were designed targeting a Factor VII nucleic acid and were tested for their effects on Factor VII mRNA in vitro. ISIS 472998, ISIS 515652, ISIS 515653, ISIS 515654, ISIS 515655, ISIS 515656, and ISIS 515657, described in the Examples above were also included in the screen.

The newly designed modified antisense oligonucleotides and their motifs are described in Table 20. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO.

Each oligonucleotide listed in Table 20 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted in the human gene sequence. ‘n/a.’ indicates that the antisense oligonucleotide is not 100% complementary with that particular gene sequence.

Activity of newly designed oligonucleotides was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells.

TABLE 20
Percent inhibition of human Factor VII mRNA levels by modified antisense
oligonucleotides targeted to SEQ ID NO: 1 and SEQ ID NO: 2

| Start | Stop | | | | | | | |
|---|---|---|---|---|---|---|---|---|
| Site on | Site on | | | | | Start | Stop | |
| SEQ | SEQ | | SEQ | | | Site on | Site on | |
| ID | ID | | ID | ISIS | % | SEQ ID | SEQ | |
| NO: 1 | NO: 1 | Sequence | NO | No | inhibition | NO: 2 | ID NO: 2 | Sugar Chemistry |
| 15263 | 15276 | TGTGCAGCCCGGCA | 74 | 472998 | 85 | 2320 | 2333 | kkddddddddddkk |
| 14128 | 14143 | TCCACTGTCCCCCTTG | 50 | 515652 | 63 | 1185 | 1200 | eeeddddddddddkkk |
| 15195 | 15210 | ATGCATGGTGATGCTT | 71 | 515653 | 67 | 2252 | 2267 | eeeddddddddddkkk |
| 14708 | 14723 | GAGGCCAGCAGATCAC | 86 | 515654 | 78 | 1765 | 1780 | eeeddddddddddkkk |
| 15164 | 15179 | TGAAGATGATAATGGA | 61 | 515655 | 41 | 2221 | 2236 | eeeddddddddddkkk |
| 14713 | 14728 | AGCCTGAGGCCAGCAG | 87 | 515656 | 74 | 1770 | 1785 | eeeddddddddddkkk |
| 15262 | 15277 | CTGTGCAGCCCGGCAC | 73 | 515657 | 49 | 2319 | 2334 | eeeddddddddddkkk |
| n/a | n/a | TGGATATTCAACTGTGG | 267 | 529265 | 52 | 551 | 567 | eekddddddddddkeke |
| 1381 | 1397 | GCCTGGATGCTGGTTTC | 268 | 529332 | 82 | n/a | n/a | eekddddddddddkeke |
| 1382 | 1398 | TGCCTGGATGCTGGTTT | 269 | 529334 | 78 | n/a | n/a | eekddddddddddkeke |
| 1383 | 1399 | GTGCCTGGATGCTGGTT | 213 | 529186 | 85 | n/a | n/a | eekddddddddddkeke |
| 1383 | 1399 | GTGCCTGGATGCTGGTT | 213 | 529223 | 81 | n/a | n/a | eekddddddddddkkke |
| 1384 | 1399 | GTGCCTGGATGCTGGT | 270 | 529129 | 75 | n/a | n/a | eeeddddddddddkkk |
| 1384 | 1399 | GTGCCTGGATGCTGGT | 270 | 529149 | 82 | n/a | n/a | kkkddddddddddeee |
| 1384 | 1400 | TGTGCCTGGATGCTGGT | 214 | 529177 | 77 | n/a | n/a | eekddddddddddkeke |
| 1384 | 1400 | TGTGCCTGGATGCTGGT | 214 | 529214 | 78 | n/a | n/a | eekddddddddddkkke |
| 1386 | 1402 | CCTGTGCCTGGATGCTG | 271 | 529178 | 79 | n/a | n/a | eekddddddddddkeke |
| 1386 | 1402 | CCTGTGCCTGGATGCTG | 271 | 529215 | 82 | n/a | n/a | eekddddddddddkkke |
| 1387 | 1403 | TCCTGTGCCTGGATGCT | 272 | 529179 | 71 | n/a | n/a | eekddddddddddkeke |
| 1387 | 1403 | TCCTGTGCCTGGATGCT | 272 | 529216 | 77 | n/a | n/a | eekddddddddddkkke |
| 1388 | 1404 | CTCCTGTGCCTGGATGC | 273 | 529193 | 69 | n/a | n/a | eekddddddddddkeke |
| 1388 | 1404 | CTCCTGTGCCTGGATGC | 273 | 529230 | 70 | n/a | n/a | eekddddddddddkkke |
| 1389 | 1404 | CTCCTGTGCCTGGATG | 274 | 529136 | 48 | n/a | n/a | eeeddddddddddkkk |
| 1389 | 1404 | CTCCTGTGCCTGGATG | 274 | 529156 | 68 | n/a | n/a | kkkddddddddddeee |
| 2229 | 2245 | CAGTCCCTGCTCACCTC | 275 | 529194 | 44 | n/a | n/a | eekddddddddddkeke |
| 2229 | 2245 | CAGTCCCTGCTCACCTC | 275 | 529231 | 56 | n/a | n/a | eekddddddddddkkke |
| 2230 | 2245 | CAGTCCCTGCTCACCT | 276 | 529137 | 34 | n/a | n/a | eeeddddddddddkkk |
| 2230 | 2245 | CAGTCCCTGCTCACCT | 276 | 529157 | 79 | n/a | n/a | kkkddddddddddeee |
| 2235 | 2251 | CAGTGGCAGTCCCTGCT | 277 | 529336 | 57 | n/a | n/a | eekddddddddddkeke |
| 2237 | 2253 | ACCAGTGGCAGTCCCTG | 278 | 529338 | 73 | n/a | n/a | eekddddddddddkeke |
| 2248 | 2264 | CCCAGGACAAAACCAGT | 279 | 529195 | 55 | n/a | n/a | eekddddddddddkeke |
| 2248 | 2264 | CCCAGGACAAAACCAGT | 279 | 529232 | 68 | n/a | n/a | eekddddddddddkkke |
| 2272 | 2288 | AGGTGATGTTGGCCCCC | 280 | 529340 | 65 | n/a | n/a | eekddddddddddkeke |
| 2347 | 2363 | AGCAGGGAACACCCTCC | 281 | 529342 | 69 | n/a | n/a | eekddddddddddkeke |
| 2367 | 2382 | GAGCGGTCACTTCCTC | 282 | 529812 | 69 | n/a | n/a | kddddddddddkekee |
| 2367 | 2382 | GAGCGGTCACTTCCTC | 282 | 529831 | 62 | n/a | n/a | kddddddddddkdkee |
| 2368 | 2383 | GGAGCGGTCACTTCCT | 283 | 529733 | 64 | n/a | n/a | keddddddddddkeke |
| 2368 | 2383 | GGAGCGGTCACTTCCT | 283 | 529753 | 52 | n/a | n/a | ekddddddddddkeke |
| 2368 | 2383 | GGAGCGGTCACTTCCT | 283 | 529773 | 57 | n/a | n/a | keddddddddddkdke |
| 2368 | 2383 | GGAGCGGTCACTTCCT | 283 | 529793 | 36 | n/a | n/a | ekddddddddddkdke |
| 2368 | 2384 | TGGAGCGGTCACTTCCT | 284 | 529862 | 48 | n/a | n/a | kdeddddddddddkdke |
| 2368 | 2384 | TGGAGCGGTCACTTCCT | 284 | 529882 | 35 | n/a | n/a | edkddddddddddkdke |
| 2368 | 2384 | TGGAGCGGTCACTTCCT | 284 | 529902 | 44 | n/a | n/a | kddddkddddkddddke |
| 2369 | 2384 | TGGAGCGGTCACTTCC | 26 | 529559 | 71 | n/a | n/a | eekddddddddddkke |
| 2369 | 2384 | TGGAGCGGTCACTTCC | 26 | 529584 | 57 | n/a | n/a | keeddddddddddkke |
| 2369 | 2384 | TGGAGCGGTCACTTCC | 26 | 529609 | 58 | n/a | n/a | edkddddddddddkke |
| 2369 | 2384 | TGGAGCGGTCACTTCC | 26 | 529634 | 49 | n/a | n/a | kdeddddddddddkke |
| 2369 | 2384 | TGGAGCGGTCACTTCC | 26 | 529659 | 52 | n/a | n/a | kddkdddddddddkke |
| 2369 | 2384 | TGGAGCGGTCACTTCC | 26 | 529684 | 48 | n/a | n/a | kddedddddddddkke |
| 2369 | 2384 | TGGAGCGGTCACTTCC | 26 | 529709 | 61 | n/a | n/a | eddkdddddddddkke |
| 2369 | 2384 | TGGAGCGGTCACTTCC | 26 | 529922 | 52 | n/a | n/a | eeeedddddddddkke |
| 2480 | 2496 | AGCTCTCAAGAACTGAG | 285 | 529344 | 50 | n/a | n/a | eekddddddddddkeke |
| 2489 | 2504 | ATCAGAAAAGCTCTCA | 286 | 529138 | 32 | n/a | n/a | eeeddddddddddkkk |
| 2489 | 2504 | ATCAGAAAAGCTCTCA | 286 | 529158 | 75 | n/a | n/a | kkkddddddddddeee |
| 2490 | 2506 | GCATCAGAAAAGCTCTC | 215 | 529184 | 75 | n/a | n/a | eekddddddddddkeke |
| 2490 | 2506 | GCATCAGAAAAGCTCTC | 215 | 529221 | 78 | n/a | n/a | eekddddddddddkkke |
| 2491 | 2506 | GCATCAGAAAAGCTCT | 287 | 529127 | 67 | n/a | n/a | eeeddddddddddkkk |
| 2491 | 2506 | GCATCAGAAAAGCTCT | 287 | 529147 | 79 | n/a | n/a | kkkddddddddddeee |
| 2491 | 2507 | TGCATCAGAAAAGCTCT | 288 | 529346 | 58 | n/a | n/a | eekddddddddddkeke |
| 2497 | 2513 | CAGATTTGCATCAGAAA | 289 | 529348 | 65 | n/a | n/a | eekddddddddddkeke |
| 2498 | 2514 | GCAGATTTGCATCAGAA | 290 | 529350 | 77 | n/a | n/a | eekddddddddddkeke |
| 4715 | 4730 | GAGGCTGAGGATGCAG | 291 | 529813 | 20 | n/a | n/a | kddddddddddkekee |
| 4715 | 4730 | GAGGCTGAGGATGCAG | 291 | 529832 | 47 | n/a | n/a | kddddddddddkdkee |
| 4716 | 4731 | GGAGGCTGAGGATGCA | 292 | 529734 | 63 | n/a | n/a | keddddddddddkeke |
| 4716 | 4731 | GGAGGCTGAGGATGCA | 292 | 529754 | 58 | n/a | n/a | ekddddddddddkeke |
| 4716 | 4731 | GGAGGCTGAGGATGCA | 292 | 529774 | 49 | n/a | n/a | keddddddddddkdke |
| 4716 | 4731 | GGAGGCTGAGGATGCA | 292 | 529794 | 51 | n/a | n/a | ekddddddddddkdke |
| 4716 | 4732 | AGGAGGCTGAGGATGCA | 293 | 529863 | 64 | n/a | n/a | kdeddddddddddkdke |
| 4716 | 4732 | AGGAGGCTGAGGATGCA | 293 | 529883 | 78 | n/a | n/a | edkddddddddddkdke |
| 4716 | 4732 | AGGAGGCTGAGGATGCA | 293 | 529903 | 36 | n/a | n/a | kddddkddddkddddke |
| 4717 | 4732 | AGGAGGCTGAGGATGC | 27 | 529560 | 71 | n/a | n/a | eekddddddddddkke |
| 4717 | 4732 | AGGAGGCTGAGGATGC | 27 | 529585 | 70 | n/a | n/a | keeddddddddddkke |
| 4717 | 4732 | AGGAGGCTGAGGATGC | 27 | 529610 | 66 | n/a | n/a | edkddddddddddkke |
| 4717 | 4732 | AGGAGGCTGAGGATGC | 27 | 529635 | 45 | n/a | n/a | kdeddddddddddkke |
| 4717 | 4732 | AGGAGGCTGAGGATGC | 27 | 529660 | 53 | n/a | n/a | kddkdddddddddkke |
| 4717 | 4732 | AGGAGGCTGAGGATGC | 27 | 529685 | 42 | n/a | n/a | kddedddddddddkke |
| 4717 | 4732 | AGGAGGCTGAGGATGC | 27 | 529710 | 60 | n/a | n/a | eddkdddddddddkke |
| 4717 | 4732 | AGGAGGCTGAGGATGC | 27 | 529923 | 63 | n/a | n/a | eeeedddddddddkke |
| 4726 | 4742 | CTGGTTTGGAAGGAGGC | 294 | 529196 | 74 | n/a | n/a | eekddddddddddkeke |
| 4726 | 4742 | CTGGTTTGGAAGGAGGC | 294 | 529233 | 80 | n/a | n/a | eekddddddddddkkke |
| 4727 | 4742 | CTGGTTTGGAAGGAGG | 295 | 529139 | 75 | n/a | n/a | eeeddddddddddkkk |
| 4727 | 4742 | CTGGTTTGGAAGGAGG | 295 | 529159 | 62 | n/a | n/a | kkkddddddddddeee |
| 4728 | 4744 | GTCTGGTTTGGAAGGAG | 296 | 529352 | 74 | n/a | n/a | eekddddddddddkeke |
| 4816 | 4832 | GCGCTACTGGGCCACGT | 297 | 529354 | 67 | n/a | n/a | eekddddddddddkeke |
| 4848 | 4864 | TTACTGAGCGCGGAAGA | 298 | 529197 | 43 | n/a | n/a | eekddddddddddkeke |
| 4848 | 4864 | TTACTGAGCGCGGAAGA | 298 | 529234 | 58 | n/a | n/a | eekddddddddddkkke |
| 4849 | 4864 | TTACTGAGCGCGGAAG | 299 | 529140 | 29 | n/a | n/a | eeeddddddddddkkk |
| 4849 | 4864 | TTACTGAGCGCGGAAG | 299 | 529160 | 59 | n/a | n/a | kkkddddddddddeee |
| 4850 | 4866 | GGTTACTGAGCGCGGAA | 216 | 529180 | 80 | n/a | n/a | eekddddddddddkeke |
| 4850 | 4866 | GGTTACTGAGCGCGGAA | 216 | 529217 | 79 | n/a | n/a | eekddddddddddkkke |
| 4869 | 4884 | GCAGGAGCGGCCTAAA | 300 | 529814 | 51 | n/a | n/a | kddddddddddkekee |
| 4869 | 4884 | GCAGGAGCGGCCTAAA | 300 | 529833 | 52 | n/a | n/a | kddddddddddkdkee |
| 4870 | 4885 | TGCAGGAGCGGCCTAA | 301 | 529735 | 43 | n/a | n/a | keddddddddddkeke |
| 4870 | 4885 | TGCAGGAGCGGCCTAA | 301 | 529755 | 60 | n/a | n/a | ekddddddddddkeke |
| 4870 | 4885 | TGCAGGAGCGGCCTAA | 301 | 529775 | 38 | n/a | n/a | keddddddddddkdke |
| 4870 | 4885 | TGCAGGAGCGGCCTAA | 301 | 529795 | 58 | n/a | n/a | ekddddddddddkdke |
| 4870 | 4886 | CTGCAGGAGCGGCCTAA | 302 | 529864 | 41 | n/a | n/a | kdeddddddddddkdke |
| 4870 | 4886 | CTGCAGGAGCGGCCTAA | 302 | 529884 | 48 | n/a | n/a | edkddddddddddkdke |
| 4870 | 4886 | CTGCAGGAGCGGCCTAA | 302 | 529904 | 44 | n/a | n/a | kddddkddddkddddke |
| 4870 | 4886 | CTGCAGGAGCGGCCTAA | 302 | 529934 | 61 | n/a | n/a | eekddddddddddkeke |
| 4871 | 4887 | TCTGCAGGAGCGGCCTA | 303 | 529356 | 71 | n/a | n/a | eekddddddddddkeke |
| 4871 | 4886 | CTGCAGGAGCGGCCTA | 28 | 529561 | 75 | n/a | n/a | eekddddddddddkke |
| 4871 | 4886 | CTGCAGGAGCGGCCTA | 28 | 529586 | 65 | n/a | n/a | keeddddddddddkke |
| 4871 | 4886 | CTGCAGGAGCGGCCTA | 28 | 529611 | 54 | n/a | n/a | edkddddddddddkke |
| 4871 | 4886 | CTGCAGGAGCGGCCTA | 28 | 529636 | 39 | n/a | n/a | kdeddddddddddkke |
| 4871 | 4886 | CTGCAGGAGCGGCCTA | 28 | 529661 | 67 | n/a | n/a | kddkdddddddddkke |
| 4871 | 4886 | CTGCAGGAGCGGCCTA | 28 | 529686 | 66 | n/a | n/a | kddedddddddddkke |
| 4871 | 4886 | CTGCAGGAGCGGCCTA | 28 | 529711 | 60 | n/a | n/a | eddkdddddddddkke |
| 4871 | 4886 | CTGCAGGAGCGGCCTA | 28 | 529924 | 62 | n/a | n/a | eeeedddddddddkke |
| 4872 | 4888 | TTCTGCAGGAGCGGCCT | 304 | 529358 | 82 | n/a | n/a | eekddddddddddkeke |
| 4873 | 4889 | GTTCTGCAGGAGCGGCC | 217 | 529181 | 79 | n/a | n/a | eekddddddddddkeke |
| 4873 | 4889 | GTTCTGCAGGAGCGGCC | 217 | 529218 | 73 | n/a | n/a | eekddddddddddkkke |
| 4874 | 4890 | AGTTCTGCAGGAGCGGC | 218 | 529182 | 85 | n/a | n/a | eekddddddddddkeke |
| 4874 | 4890 | AGTTCTGCAGGAGCGGC | 218 | 529219 | 84 | n/a | n/a | eekddddddddddkkke |
| 4875 | 4891 | GAGTTCTGCAGGAGCGG | 305 | 529360 | 84 | n/a | n/a | eekddddddddddkeke |
| 4876 | 4892 | CGAGTTCTGCAGGAGCG | 306 | 529362 | 87 | n/a | n/a | eekddddddddddkeke |
| 4877 | 4893 | CCGAGTTCTGCAGGAGC | 307 | 529364 | 81 | n/a | n/a | eekddddddddddkeke |
| 4921 | 4937 | AGGACGAGGCCTCAGGT | 308 | 529366 | 77 | n/a | n/a | eekddddddddddkeke |
| 4940 | 4956 | AGGGACGACCTTTGCTG | 309 | 529198 | 28 | n/a | n/a | eekddddddddddkeke |
| 4940 | 4956 | AGGGACGACCTTTGCTG | 309 | 529235 | 8 | n/a | n/a | eekddddddddddkkke |
| 4941 | 4956 | AGGGACGACCTTTGCT | 310 | 529141 | 34 | n/a | n/a | eeeddddddddddkkk |
| 4941 | 4956 | AGGGACGACCTTTGCT | 310 | 529161 | 66 | n/a | n/a | kkkddddddddddeee |
| 5127 | 5143 | GTGGGCACCACGCGGTG | 311 | 529368 | 27 | n/a | n/a | eekddddddddddkeke |
| 5128 | 5144 | TGTGGGCACCACGCGGT | 312 | 529370 | 44 | n/a | n/a | eekddddddddddkeke |
| 5131 | 5147 | AGCTGTGGGCACCACGC | 313 | 529372 | 61 | n/a | n/a | eekddddddddddkeke |
| 5132 | 5148 | GAGCTGTGGGCACCACG | 314 | 529374 | 71 | n/a | n/a | eekddddddddddkeke |
| 5133 | 5149 | TGAGCTGTGGGCACCAC | 315 | 529376 | 63 | n/a | n/a | eekddddddddddkeke |
| 5373 | 5389 | GACCTCGCGCGGATCCT | 316 | 529378 | 68 | n/a | n/a | eekddddddddddkeke |
| 5511 | 5527 | CCGAGGCGCGGCCCCTG | 317 | 529380 | 79 | n/a | n/a | eekddddddddddkeke |
| 5512 | 5528 | TCCGAGGCGCGGCCCCT | 318 | 529382 | 77 | n/a | n/a | eekddddddddddkeke |
| 5556 | 5572 | CGTCTCCGGCGGCCAGA | 319 | 529384 | 75 | n/a | n/a | eekddddddddddkeke |
| 5601 | 5617 | ACAGCCGCCCGCGGAAA | 320 | 529386 | 40 | n/a | n/a | eekddddddddddkeke |
| 6075 | 6091 | CCGGCCGCAGCTCCTCC | 321 | 529240 | 73 | 180 | 196 | eekddddddddddkeke |
| 6076 | 6092 | CCCGGCCGCAGCTCCTC | 322 | 529241 | 67 | 181 | 197 | eekddddddddddkeke |
| 6100 | 6116 | TCCTTGCACTCCCTCTC | 323 | 529242 | 42 | 205 | 221 | eekddddddddddkeke |
| 6132 | 6148 | TCTCCCGGGCCTCCTCG | 324 | 529243 | 60 | 237 | 253 | eekddddddddddkeke |
| 6401 | 6417 | TGATGTGAAAACCGGCA | 325 | 529388 | 65 | n/a | n/a | eekddddddddddkeke |
| 6409 | 6424 | TATTTTCTGATGTGAA | 326 | 529815 | 37 | n/a | n/a | kddddddddddkekee |
| 6409 | 6424 | TATTTTCTGATGTGAA | 326 | 529834 | 44 | n/a | n/a | kddddddddddkdkee |
| 6410 | 6425 | GTATTTTCTGATGTGA | 327 | 529736 | 47 | n/a | n/a | keddddddddddkeke |
| 6410 | 6425 | GTATTTTCTGATGTGA | 327 | 529756 | 78 | n/a | n/a | ekddddddddddkeke |
| 6410 | 6425 | GTATTTTCTGATGTGA | 327 | 529776 | 37 | n/a | n/a | keddddddddddkdke |
| 6410 | 6425 | GTATTTTCTGATGTGA | 327 | 529796 | 71 | n/a | n/a | ekddddddddddkdke |
| 6410 | 6426 | CGTATTTTCTGATGTGA | 328 | 529865 | 70 | n/a | n/a | kdeddddddddddkdke |
| 6410 | 6426 | CGTATTTTCTGATGTGA | 328 | 529885 | 59 | n/a | n/a | edkddddddddddkdke |
| 6410 | 6426 | CGTATTTTCTGATGTGA | 328 | 529905 | 54 | n/a | n/a | kddddkddddkddddke |
| 6410 | 6426 | CGTATTTTCTGATGTGA | 328 | 529935 | 70 | n/a | n/a | eekddddddddddkeke |
| 6411 | 6426 | CGTATTTTCTGATGTG | 29 | 529562 | 87 | n/a | n/a | eekddddddddddkke |
| 6411 | 6426 | CGTATTTTCTGATGTG | 29 | 529587 | 68 | n/a | n/a | keeddddddddddkke |
| 6411 | 6426 | CGTATTTTCTGATGTG | 29 | 529612 | 67 | n/a | n/a | edkddddddddddkke |
| 6411 | 6426 | CGTATTTTCTGATGTG | 29 | 529637 | 64 | n/a | n/a | kdeddddddddddkke |
| 6411 | 6426 | CGTATTTTCTGATGTG | 29 | 529662 | 62 | n/a | n/a | kddkdddddddddkke |
| 6411 | 6426 | CGTATTTTCTGATGTG | 29 | 529687 | 63 | n/a | n/a | kddedddddddddkke |
| 6411 | 6426 | CGTATTTTCTGATGTG | 29 | 529712 | 61 | n/a | n/a | eddkdddddddddkke |
| 6411 | 6426 | CGTATTTTCTGATGTG | 29 | 529925 | 61 | n/a | n/a | eeeedddddddddkke |
| 6640 | 6655 | GGTGACCCGTGAGCGT | 329 | 529816 | 77 | n/a | n/a | kddddddddddkekee |
| 6640 | 6655 | GGTGACCCGTGAGCGT | 329 | 529835 | 80 | n/a | n/a | kddddddddddkdkee |
| 6641 | 6656 | AGGTGACCCGTGAGCG | 330 | 529737 | 82 | n/a | n/a | keddddddddddkeke |
| 6641 | 6656 | AGGTGACCCGTGAGCG | 330 | 529757 | 83 | n/a | n/a | ekddddddddddkeke |
| 6641 | 6656 | AGGTGACCCGTGAGCG | 330 | 529777 | 68 | n/a | n/a | keddddddddddkdke |
| 6641 | 6656 | AGGTGACCCGTGAGCG | 330 | 529797 | 77 | n/a | n/a | ekddddddddddkdke |
| 6641 | 6657 | GAGGTGACCCGTGAGCG | 331 | 529866 | 15 | n/a | n/a | kdeddddddddddkdke |
| 6641 | 6657 | GAGGTGACCCGTGAGCG | 331 | 529886 | 71 | n/a | n/a | edkddddddddddkdke |
| 6641 | 6657 | GAGGTGACCCGTGAGCG | 331 | 529906 | 63 | n/a | n/a | kddddkddddkddddke |
| 6641 | 6657 | GAGGTGACCCGTGAGCG | 331 | 529936 | 78 | n/a | n/a | eekddddddddddkeke |
| 6642 | 6657 | GAGGTGACCCGTGAGC | 30 | 529563 | 89 | n/a | n/a | eekddddddddddkke |
| 6642 | 6657 | GAGGTGACCCGTGAGC | 30 | 529588 | 84 | n/a | n/a | keeddddddddddkke |
| 6642 | 6657 | GAGGTGACCCGTGAGC | 30 | 529613 | 80 | n/a | n/a | edkddddddddddkke |
| 6642 | 6657 | GAGGTGACCCGTGAGC | 30 | 529638 | 48 | n/a | n/a | kdeddddddddddkke |
| 6642 | 6657 | GAGGTGACCCGTGAGC | 30 | 529663 | 85 | n/a | n/a | kddkdddddddddkke |
| 6642 | 6657 | GAGGTGACCCGTGAGC | 30 | 529688 | 42 | n/a | n/a | kddedddddddddkke |
| 6642 | 6657 | GAGGTGACCCGTGAGC | 30 | 529713 | 81 | n/a | n/a | eddkdddddddddkke |
| 6642 | 6657 | GAGGTGACCCGTGAGC | 30 | 529926 | 67 | n/a | n/a | eeeedddddddddkke |
| 8548 | 8564 | GGCATGACCATCCTCAA | 332 | 529390 | 53 | n/a | n/a | eekddddddddddkeke |
| 8553 | 8569 | TGCTAGGCATGACCATC | 333 | 529392 | 63 | n/a | n/a | eekddddddddddkeke |
| 8606 | 8622 | TGCTGTGAGAATACAAC | 334 | 529394 | 58 | n/a | n/a | eekddddddddddkeke |
| 8608 | 8624 | GGTGCTGTGAGAATACA | 335 | 529396 | 56 | n/a | n/a | eekddddddddddkeke |
| 8642 | 8658 | AGAAACTGTTGGCCACC | 336 | 529398 | 62 | n/a | n/a | eekddddddddddkeke |
| 8643 | 8659 | AAGAAACTGTTGGCCAC | 337 | 529400 | 44 | n/a | n/a | eekddddddddddkeke |
| 8654 | 8670 | AGTGATTGCTGAAGAAA | 338 | 529402 | 39 | n/a | n/a | eekddddddddddkeke |
| 8707 | 8723 | GCAGTAGCAGATGCAAA | 339 | 529404 | 46 | n/a | n/a | eekddddddddddkeke |
| 8860 | 8876 | ATGGGTGACCACACATT | 340 | 529406 | 63 | n/a | n/a | eekddddddddddkeke |
| 9168 | 9184 | GGCACACTGGTCCCCAT | 341 | 529244 | 58 | 302 | 318 | eekddddddddddkeke |
| 9169 | 9185 | AGGCACACTGGTCCCCA | 342 | 529245 | 68 | 303 | 319 | eekddddddddddkeke |
| 9171 | 9187 | TGAGGCACACTGGTCCC | 343 | 529246 | 60 | 305 | 321 | eekddddddddddkeke |
| 9199 | 9215 | TGCAGGAGCCCCCATTC | 344 | 529247 | 36 | 333 | 349 | eekddddddddddkeke |
| 9212 | 9228 | TGGAGCTGGTCCTTGCA | 345 | 529248 | 43 | 346 | 362 | eekddddddddddkeke |
| 9217 | 9233 | AGGACTGGAGCTGGTCC | 346 | 529249 | 23 | 351 | 367 | eekddddddddddkeke |
| 9224 | 9240 | CAGATATAGGACTGGAG | 347 | 529250 | 69 | 358 | 374 | eekddddddddddkeke |
| 9226 | 9242 | AGCAGATATAGGACTGG | 348 | 529251 | 15 | 360 | 376 | eekddddddddddkeke |
| 9258 | 9274 | ACAGTTCCGGCCCTCGA | 349 | 529252 | 44 | 392 | 408 | eekddddddddddkeke |
| 9261 | 9277 | CTCACAGTTCCGGCCCT | 350 | 529253 | 42 | 395 | 411 | eekddddddddddkeke |
| 9356 | 9372 | AGGTTGTGCACCCTGCA | 351 | 529408 | 67 | n/a | n/a | eekddddddddddkeke |
| 9358 | 9374 | CCAGGTTGTGCACCCTG | 352 | 529410 | 19 | n/a | n/a | eekddddddddddkeke |
| 9452 | 9468 | CCTGTCAAAGACCTCAG | 353 | 529412 | 57 | n/a | n/a | eekddddddddddkeke |
| 9598 | 9614 | GAAGTTTACCAAGCGGT | 354 | 529414 | 80 | n/a | n/a | eekddddddddddkeke |
| 9939 | 9955 | AACCTCTGGACACCGGG | 355 | 529416 | 85 | n/a | n/a | eekddddddddddkeke |
| 9958 | 9974 | TGTGATTGAGCCCTGAT | 356 | 529418 | 70 | n/a | n/a | eekddddddddddkeke |
| 10067 | 10083 | TGGTCTAGCTGACAATG | 357 | 529420 | 78 | n/a | n/a | eekddddddddddkeke |
| 10074 | 10090 | TGCTGGATGGTCTAGCT | 358 | 529422 | 19 | n/a | n/a | eekddddddddddkeke |
| 10334 | 10350 | GGCATTCTGGACCCAGA | 359 | 529424 | 48 | n/a | n/a | eekddddddddddkeke |
| 10383 | 10399 | AGGATGCACACCAGGGC | 360 | 529426 | 66 | n/a | n/a | eekddddddddddkeke |
| 10384 | 10400 | CAGGATGCACACCAGGG | 361 | 529428 | 59 | n/a | n/a | eekddddddddddkeke |
| 10417 | 10433 | AAGTCCAGGACTCCGGC | 362 | 529430 | 83 | n/a | n/a | eekddddddddddkeke |
| 10669 | 10685 | CCGAGCAGCTGATGGGA | 363 | 529432 | 84 | n/a | n/a | eekddddddddddkeke |
| 10677 | 10693 | CCACTCTTCCGAGCAGC | 364 | 529199 | 71 | n/a | n/a | eekddddddddddkeke |
| 10677 | 10693 | CCACTCTTCCGAGCAGC | 364 | 529236 | 76 | n/a | n/a | eekddddddddddkkke |
| 10678 | 10693 | CCACTCTTCCGAGCAG | 365 | 529142 | 64 | n/a | n/a | eeeddddddddddkkk |
| 10678 | 10693 | CCACTCTTCCGAGCAG | 365 | 529162 | 60 | n/a | n/a | kkkddddddddddeee |
| 10983 | 10999 | ATCAGCTGGTCATCCTT | 366 | 529254 | 46 | 418 | 434 | eekddddddddddkeke |
| 10986 | 11002 | CAGATCAGCTGGTCATC | 367 | 529255 | 52 | 421 | 437 | eekddddddddddkeke |
| 11013 | 11029 | TGCTCACAGCCGCCGTT | 368 | 529256 | 57 | 448 | 464 | eekddddddddddkeke |
| 11014 | 11030 | CTGCTCACAGCCGCCGT | 369 | 529257 | 55 | 449 | 465 | eekddddddddddkeke |
| 11015 | 11031 | ACTGCTCACAGCCGCCG | 370 | 529258 | 3 | 450 | 466 | eekddddddddddkeke |
| 11016 | 11032 | TACTGCTCACAGCCGCC | 371 | 529259 | 71 | 451 | 467 | eekddddddddddkeke |
| 11018 | 11034 | AGTACTGCTCACAGCCG | 372 | 529260 | 72 | 453 | 469 | eekddddddddddkeke |
| 11021 | 11037 | TGCAGTACTGCTCACAG | 373 | 529261 | 56 | 456 | 472 | eekddddddddddkeke |
| 11025 | 11041 | TCACTGCAGTACTGCTC | 374 | 529262 | 56 | 460 | 476 | eekddddddddddkeke |
| 11028 | 11044 | TGGTCACTGCAGTACTG | 375 | 529263 | 59 | 463 | 479 | eekddddddddddkeke |
| 11089 | 11105 | CACCCCGTCTGCCAGCA | 376 | 529264 | 49 | 524 | 540 | eekddddddddddkeke |
| 11142 | 11158 | TGGTGTCATCTGGGACT | 377 | 529434 | 83 | n/a | n/a | eekddddddddddkeke |
| 11162 | 11178 | ATCCGTAGTGGGACAGG | 378 | 529436 | 80 | n/a | n/a | eekddddddddddkeke |
| 11279 | 11295 | TGTCGCTCTGGCCTGTG | 379 | 529438 | 79 | n/a | n/a | eekddddddddddkeke |
| 11281 | 11297 | ACTGTCGCTCTGGCCTG | 380 | 529440 | 87 | n/a | n/a | eekddddddddddkeke |
| 11284 | 11300 | GTCACTGTCGCTCTGGC | 381 | 529442 | 68 | n/a | n/a | eekddddddddddkeke |
| 11401 | 11417 | AGGTCCTGCGAGTGGGA | 382 | 529443 | 72 | n/a | n/a | eekddddddddddkeke |
| 11403 | 11419 | GGAGGTCCTGCGAGTGG | 383 | 529444 | 68 | n/a | n/a | eekddddddddddkeke |
| 11454 | 11470 | AGCAGTCAGTACAGACA | 384 | 529445 | 85 | n/a | n/a | eekddddddddddkeke |
| 11543 | 11559 | TGGAAGTGCAGCCCATT | 385 | 529446 | 72 | n/a | n/a | eekddddddddddkeke |
| 11544 | 11560 | TTGGAAGTGCAGCCCAT | 386 | 529447 | 60 | n/a | n/a | eekddddddddddkeke |
| 11836 | 11852 | TGGTCAGGGCTGGTTTT | 387 | 529448 | 77 | n/a | n/a | eekddddddddddkeke |
| 11837 | 11852 | TGGTCAGGGCTGGTTT | 388 | 529807 | 78 | n/a | n/a | kddddddddddkekee |
| 11837 | 11852 | TGGTCAGGGCTGGTTT | 388 | 529826 | 61 | n/a | n/a | kddddddddddkdkee |
| 11838 | 11854 | AATGGTCAGGGCTGGTT | 389 | 529449 | 81 | n/a | n/a | eekddddddddddkeke |
| 11838 | 11853 | ATGGTCAGGGCTGGTT | 390 | 529728 | 75 | n/a | n/a | keddddddddddkeke |
| 11838 | 11853 | ATGGTCAGGGCTGGTT | 390 | 529748 | 80 | n/a | n/a | ekddddddddddkeke |
| 11838 | 11853 | ATGGTCAGGGCTGGTT | 390 | 529768 | 68 | n/a | n/a | keddddddddddkdke |
| 11838 | 11853 | ATGGTCAGGGCTGGTT | 390 | 529788 | 74 | n/a | n/a | ekddddddddddkdke |
| 11838 | 11854 | AATGGTCAGGGCTGGTT | 389 | 529857 | 67 | n/a | n/a | kdeddddddddddkdke |
| 11838 | 11854 | AATGGTCAGGGCTGGTT | 389 | 529877 | 77 | n/a | n/a | edkddddddddddkdke |
| 11838 | 11854 | AATGGTCAGGGCTGGTT | 389 | 529897 | 26 | n/a | n/a | kddddkddddkddddke |
| 11839 | 11855 | CAATGGTCAGGGCTGGT | 391 | 529200 | 78 | n/a | n/a | eekddddddddddkeke |
| 11839 | 11855 | CAATGGTCAGGGCTGGT | 391 | 529237 | 84 | n/a | n/a | eekddddddddddkkke |
| 11839 | 11854 | AATGGTCAGGGCTGGT | 34 | 529564 | 90 | n/a | n/a | eekddddddddddkke |
| 11839 | 11854 | AATGGTCAGGGCTGGT | 34 | 529589 | 86 | n/a | n/a | keeddddddddddkke |
| 11839 | 11854 | AATGGTCAGGGCTGGT | 34 | 529614 | 82 | n/a | n/a | edkddddddddddkke |
| 11839 | 11854 | AATGGTCAGGGCTGGT | 34 | 529639 | 80 | n/a | n/a | kdeddddddddddkke |
| 11839 | 11854 | AATGGTCAGGGCTGGT | 34 | 529664 | 69 | n/a | n/a | kddkdddddddddkke |
| 11839 | 11854 | AATGGTCAGGGCTGGT | 34 | 529689 | 71 | n/a | n/a | kddedddddddddkke |
| 11839 | 11854 | AATGGTCAGGGCTGGT | 34 | 529714 | 73 | n/a | n/a | eddkdddddddddkke |
| 11839 | 11854 | AATGGTCAGGGCTGGT | 34 | 529917 | 73 | n/a | n/a | eeeedddddddddkke |
| 11840 | 11855 | CAATGGTCAGGGCTGG | 392 | 529143 | 68 | n/a | n/a | eeeddddddddddkkk |
| 11840 | 11855 | CAATGGTCAGGGCTGG | 392 | 529163 | 50 | n/a | n/a | kkkddddddddddeee |
| 11840 | 11856 | ACAATGGTCAGGGCTGG | 393 | 529201 | 76 | n/a | n/a | eekddddddddddkeke |
| 11840 | 11856 | ACAATGGTCAGGGCTGG | 393 | 529238 | 72 | n/a | n/a | eekddddddddddkkke |
| 11841 | 11856 | ACAATGGTCAGGGCTG | 394 | 529144 | 57 | n/a | n/a | eeeddddddddddkkk |
| 11841 | 11856 | ACAATGGTCAGGGCTG | 394 | 529164 | 71 | n/a | n/a | kkkddddddddddeee |
| 11845 | 11861 | AGGAGACAATGGTCAGG | 395 | 529450 | 91 | n/a | n/a | eekddddddddddkeke |
| 11846 | 11862 | GAGGAGACAATGGTCAG | 396 | 529451 | 85 | n/a | n/a | eekddddddddddkeke |
| 12097 | 12113 | TAGGTATTTTTCCACAT | 397 | 529266 | 63 | 567 | 583 | eekddddddddddkeke |
| 12125 | 12140 | GTTTGCTGGCATTTCT | 398 | 529806 | 52 | 595 | 610 | kddddddddddkekee |
| 12125 | 12140 | GTTTGCTGGCATTTCT | 398 | 529825 | 44 | 595 | 610 | kddddddddddkdkee |
| 12126 | 12142 | GGGTTTGCTGGCATTTC | 399 | 529267 | 56 | 596 | 612 | eekddddddddddkeke |
| 12126 | 12141 | GGTTTGCTGGCATTTC | 400 | 529727 | 67 | 596 | 611 | keddddddddddkeke |
| 12126 | 12141 | GGTTTGCTGGCATTTC | 400 | 529747 | 63 | 596 | 611 | ekddddddddddkeke |
| 12126 | 12141 | GGTTTGCTGGCATTTC | 400 | 529767 | 67 | 596 | 611 | keddddddddddkdke |
| 12126 | 12141 | GGTTTGCTGGCATTTC | 400 | 529787 | 68 | 596 | 611 | ekddddddddddkdke |
| 12126 | 12142 | GGGTTTGCTGGCATTTC | 399 | 529856 | 42 | 596 | 612 | kdeddddddddddkdke |
| 12126 | 12142 | GGGTTTGCTGGCATTTC | 399 | 529876 | 36 | 596 | 612 | edkddddddddddkdke |
| 12126 | 12142 | GGGTTTGCTGGCATTTC | 399 | 529896 | 56 | 596 | 612 | kddddkddddkddddke |
| 12127 | 12142 | GGGTTTGCTGGCATTT | 248 | 529546 | 65 | 597 | 612 | eekddddddddddkke |
| 12127 | 12142 | GGGTTTGCTGGCATTT | 248 | 529571 | 80 | 597 | 612 | keeddddddddddkke |
| 12127 | 12142 | GGGTTTGCTGGCATTT | 248 | 529596 | 43 | 597 | 612 | edkddddddddddkke |
| 12127 | 12142 | GGGTTTGCTGGCATTT | 248 | 529621 | 38 | 597 | 612 | kdeddddddddddkke |
| 12127 | 12142 | GGGTTTGCTGGCATTT | 248 | 529646 | 68 | 597 | 612 | kddkdddddddddkke |
| 12127 | 12142 | GGGTTTGCTGGCATTT | 248 | 529671 | 50 | 597 | 612 | kddedddddddddkke |
| 12127 | 12142 | GGGTTTGCTGGCATTT | 248 | 529696 | 53 | 597 | 612 | eddkdddddddddkke |
| 12127 | 12142 | GGGTTTGCTGGCATTT | 248 | 529916 | 22 | 597 | 612 | eeeedddddddddkke |
| 12141 | 12156 | ACAATTCGGCCTTGGG | 37 | 529547 | 86 | 611 | 626 | eekddddddddddkke |
| 12141 | 12156 | ACAATTCGGCCTTGGG | 37 | 529572 | 75 | 611 | 626 | keeddddddddddkke |
| 12141 | 12156 | ACAATTCGGCCTTGGG | 37 | 529597 | 58 | 611 | 626 | edkddddddddddkke |
| 12141 | 12156 | ACAATTCGGCCTTGGG | 37 | 529622 | 58 | 611 | 626 | kdeddddddddddkke |
| 12141 | 12156 | ACAATTCGGCCTTGGG | 37 | 529647 | 18 | 611 | 626 | kddkdddddddddkke |
| 12141 | 12156 | ACAATTCGGCCTTGGG | 37 | 529672 | 23 | 611 | 626 | kddedddddddddkke |
| 12141 | 12156 | ACAATTCGGCCTTGGG | 37 | 529697 | 28 | 611 | 626 | eddkdddddddddkke |
| 12141 | 12156 | ACAATTCGGCCTTGGG | 37 | 529928 | 36 | 611 | 626 | eeeedddddddddkke |
| 12302 | 12318 | TGACTTGGAGCCTGGTG | 401 | 529452 | 63 | n/a | n/a | eekddddddddddkeke |
| 12404 | 12420 | TTCTCTGCACAGGGTAG | 402 | 529453 | 73 | n/a | n/a | eekddddddddddkeke |
| 12504 | 12520 | TGATCCAATGCTCCTGA | 403 | 529454 | 82 | n/a | n/a | eekddddddddddkeke |
| 12505 | 12521 | TTGATCCAATGCTCCTG | 404 | 529455 | 84 | n/a | n/a | eekddddddddddkeke |
| 12506 | 12522 | TTTGATCCAATGCTCCT | 405 | 529202 | 61 | n/a | n/a | eekddddddddddkeke |
| 12506 | 12522 | TTTGATCCAATGCTCCT | 405 | 529239 | 59 | n/a | n/a | eekddddddddddkkke |
| 12507 | 12522 | TTTGATCCAATGCTCC | 406 | 529145 | 54 | n/a | n/a | eeeddddddddddkkk |
| 12507 | 12522 | TTTGATCCAATGCTCC | 406 | 529165 | 77 | n/a | n/a | kkkddddddddddeee |
| 12509 | 12525 | AGCTTTGATCCAATGCT | 407 | 529456 | 69 | n/a | n/a | eekddddddddddkeke |
| 12511 | 12527 | ATAGCTTTGATCCAATG | 408 | 529457 | 81 | n/a | n/a | eekddddddddddkeke |
| 12512 | 12528 | CATAGCTTTGATCCAAT | 409 | 529458 | 72 | n/a | n/a | eekddddddddddkeke |
| 12519 | 12535 | ATCTTCACATAGCTTTG | 410 | 529459 | 86 | n/a | n/a | eekddddddddddkeke |
| 12552 | 12568 | GTCGCTGTGAGATTTCA | 411 | 529460 | 88 | n/a | n/a | eekddddddddddkeke |
| 12627 | 12642 | TCAGACCTGGCTCTGG | 412 | 529817 | 46 | n/a | n/a | kddddddddddkekee |
| 12627 | 12642 | TCAGACCTGGCTCTGG | 412 | 529836 | 49 | n/a | n/a | kddddddddddkdkee |
| 12628 | 12643 | CTCAGACCTGGCTCTG | 413 | 529738 | 51 | n/a | n/a | keddddddddddkeke |
| 12628 | 12643 | CTCAGACCTGGCTCTG | 413 | 529758 | 53 | n/a | n/a | ekddddddddddkeke |
| 12628 | 12643 | CTCAGACCTGGCTCTG | 413 | 529778 | 39 | n/a | n/a | keddddddddddkdke |
| 12628 | 12643 | CTCAGACCTGGCTCTG | 413 | 529798 | 52 | n/a | n/a | ekddddddddddkdke |
| 12628 | 12644 | GCTCAGACCTGGCTCTG | 414 | 529867 | 56 | n/a | n/a | kdeddddddddddkdke |
| 12628 | 12644 | GCTCAGACCTGGCTCTG | 414 | 529887 | 68 | n/a | n/a | edkddddddddddkdke |
| 12628 | 12644 | GCTCAGACCTGGCTCTG | 414 | 529907 | 28 | n/a | n/a | kddddkddddkddddke |
| 12628 | 12644 | GCTCAGACCTGGCTCTG | 414 | 529938 | 64 | n/a | n/a | eekddddddddddkeke |
| 12629 | 12644 | GCTCAGACCTGGCTCT | 38 | 529565 | 81 | n/a | n/a | eekddddddddddkke |
| 12629 | 12644 | GCTCAGACCTGGCTCT | 38 | 529590 | 49 | n/a | n/a | keeddddddddddkke |
| 12629 | 12644 | GCTCAGACCTGGCTCT | 38 | 529615 | 65 | n/a | n/a | edkddddddddddkke |
| 12629 | 12644 | GCTCAGACCTGGCTCT | 38 | 529640 | 54 | n/a | n/a | kdeddddddddddkke |
| 12629 | 12644 | GCTCAGACCTGGCTCT | 38 | 529665 | 77 | n/a | n/a | kddkdddddddddkke |
| 12629 | 12644 | GCTCAGACCTGGCTCT | 38 | 529690 | 77 | n/a | n/a | kddedddddddddkke |
| 12629 | 12644 | GCTCAGACCTGGCTCT | 38 | 529715 | 63 | n/a | n/a | eddkdddddddddkke |
| 12629 | 12644 | GCTCAGACCTGGCTCT | 38 | 529927 | 62 | n/a | n/a | eeeedddddddddkke |
| 12631 | 12647 | GCTGCTCAGACCTGGCT | 221 | 529185 | 66 | n/a | n/a | eekddddddddddkeke |
| 12631 | 12647 | GCTGCTCAGACCTGGCT | 221 | 529222 | 62 | n/a | n/a | eekddddddddddkkke |
| 12631 | 12646 | CTGCTCAGACCTGGCT | 89 | 529808 | 75 | n/a | n/a | kddddddddddkekee |
| 12631 | 12646 | CTGCTCAGACCTGGCT | 89 | 529827 | 67 | n/a | n/a | kddddddddddkdkee |
| 12632 | 12647 | GCTGCTCAGACCTGGC | 415 | 529128 | 64 | n/a | n/a | eeeddddddddddkkk |
| 12632 | 12647 | GCTGCTCAGACCTGGC | 415 | 529148 | 78 | n/a | n/a | kkkddddddddddeee |
| 12632 | 12648 | AGCTGCTCAGACCTGGC | 416 | 529461 | 87 | n/a | n/a | eekddddddddddkeke |
| 12632 | 12647 | GCTGCTCAGACCTGGC | 415 | 529729 | 71 | n/a | n/a | keddddddddddkeke |
| 12632 | 12647 | GCTGCTCAGACCTGGC | 415 | 529749 | 83 | n/a | n/a | ekddddddddddkeke |
| 12632 | 12647 | GCTGCTCAGACCTGGC | 415 | 529769 | 63 | n/a | n/a | keddddddddddkdke |
| 12632 | 12647 | GCTGCTCAGACCTGGC | 415 | 529789 | 10 | n/a | n/a | ekddddddddddkdke |
| 12632 | 12647 | GCTGCTCAGACCTGGC | 415 | 529800 | 69 | n/a | n/a | kddddddddddkekee |
| 12632 | 12647 | GCTGCTCAGACCTGGC | 415 | 529819 | 78 | n/a | n/a | kddddddddddkdkee |
| 12632 | 12648 | AGCTGCTCAGACCTGGC | 416 | 529858 | 60 | n/a | n/a | kdeddddddddddkdke |
| 12632 | 12648 | AGCTGCTCAGACCTGGC | 416 | 529878 | 75 | n/a | n/a | edkddddddddddkdke |
| 12632 | 12648 | AGCTGCTCAGACCTGGC | 416 | 529898 | 34 | n/a | n/a | kddddkddddkddddke |
| 12633 | 12648 | AGCTGCTCAGACCTGG | 39 | 529566 | 61 | n/a | n/a | eekddddddddddkke |
| 12633 | 12648 | AGCTGCTCAGACCTGG | 39 | 529591 | 71 | n/a | n/a | keeddddddddddkke |
| 12633 | 12648 | AGCTGCTCAGACCTGG | 39 | 529616 | 71 | n/a | n/a | edkddddddddddkke |
| 12633 | 12648 | AGCTGCTCAGACCTGG | 39 | 529641 | 65 | n/a | n/a | kdeddddddddddkke |
| 12633 | 12648 | AGCTGCTCAGACCTGG | 39 | 529666 | 70 | n/a | n/a | kddkdddddddddkke |
| 12633 | 12648 | AGCTGCTCAGACCTGG | 39 | 529691 | 67 | n/a | n/a | kddedddddddddkke |
| 12633 | 12648 | AGCTGCTCAGACCTGG | 39 | 529716 | 75 | n/a | n/a | eddkdddddddddkke |
| 12633 | 12648 | AGCTGCTCAGACCTGG | 39 | 529721 | 71 | n/a | n/a | keddddddddddkeke |
| 12633 | 12648 | AGCTGCTCAGACCTGG | 39 | 529741 | 81 | n/a | n/a | ekddddddddddkeke |
| 12633 | 12648 | AGCTGCTCAGACCTGG | 39 | 529761 | 66 | n/a | n/a | keddddddddddkdke |
| 12633 | 12648 | AGCTGCTCAGACCTGG | 39 | 529781 | 65 | n/a | n/a | ekddddddddddkdke |
| 12633 | 12648 | AGCTGCTCAGACCTGG | 39 | 529801 | 71 | n/a | n/a | kddddddddddkekee |
| 12633 | 12648 | AGCTGCTCAGACCTGG | 39 | 529820 | 74 | n/a | n/a | kddddddddddkdkee |
| 12633 | 12649 | AAGCTGCTCAGACCTGG | 417 | 529850 | 63 | n/a | n/a | kdeddddddddddkdke |
| 12633 | 12649 | AAGCTGCTCAGACCTGG | 417 | 529870 | 72 | n/a | n/a | edkddddddddddkdke |
| 12633 | 12649 | AAGCTGCTCAGACCTGG | 417 | 529890 | 23 | n/a | n/a | kddddkddddkddddke |
| 12633 | 12648 | AGCTGCTCAGACCTGG | 39 | 529918 | 54 | n/a | n/a | eeeedddddddddkke |
| 12634 | 12649 | AAGCTGCTCAGACCTG | 262 | 529567 | 75 | n/a | n/a | eekddddddddddkke |
| 12634 | 12649 | AAGCTGCTCAGACCTG | 262 | 529592 | 80 | n/a | n/a | keeddddddddddkke |
| 12634 | 12649 | AAGCTGCTCAGACCTG | 262 | 529617 | 65 | n/a | n/a | edkddddddddddkke |
| 12634 | 12649 | AAGCTGCTCAGACCTG | 262 | 529642 | 62 | n/a | n/a | kdeddddddddddkke |
| 12634 | 12649 | AAGCTGCTCAGACCTG | 262 | 529667 | 75 | n/a | n/a | kddkdddddddddkke |
| 12634 | 12649 | AAGCTGCTCAGACCTG | 262 | 529692 | 53 | n/a | n/a | kddedddddddddkke |
| 12634 | 12649 | AAGCTGCTCAGACCTG | 262 | 529717 | 69 | n/a | n/a | eddkdddddddddkke |
| 12634 | 12649 | AAGCTGCTCAGACCTG | 262 | 529722 | 74 | n/a | n/a | keddddddddddkeke |
| 12634 | 12649 | AAGCTGCTCAGACCTG | 262 | 529742 | 81 | n/a | n/a | ekddddddddddkeke |
| 12634 | 12649 | AAGCTGCTCAGACCTG | 262 | 529762 | 66 | n/a | n/a | keddddddddddkdke |
| 12634 | 12649 | AAGCTGCTCAGACCTG | 262 | 529782 | 68 | n/a | n/a | ekddddddddddkdke |
| 12634 | 12650 | AAAGCTGCTCAGACCTG | 418 | 529851 | 68 | n/a | n/a | kdeddddddddddkdke |
| 12634 | 12650 | AAAGCTGCTCAGACCTG | 418 | 529871 | 77 | n/a | n/a | edkddddddddddkdke |
| 12634 | 12650 | AAAGCTGCTCAGACCTG | 418 | 529891 | 36 | n/a | n/a | kddddkddddkddddke |
| 12634 | 12649 | AAGCTGCTCAGACCTG | 262 | 529910 | 60 | n/a | n/a | eeeedddddddddkke |
| 12635 | 12650 | AAAGCTGCTCAGACCT | 263 | 529568 | 79 | n/a | n/a | eekddddddddddkke |
| 12635 | 12650 | AAAGCTGCTCAGACCT | 263 | 529593 | 70 | n/a | n/a | keeddddddddddkke |
| 12635 | 12650 | AAAGCTGCTCAGACCT | 263 | 529618 | 77 | n/a | n/a | edkddddddddddkke |
| 12635 | 12650 | AAAGCTGCTCAGACCT | 263 | 529643 | 72 | n/a | n/a | kdeddddddddddkke |
| 12635 | 12650 | AAAGCTGCTCAGACCT | 263 | 529668 | 73 | n/a | n/a | kddkdddddddddkke |
| 12635 | 12650 | AAAGCTGCTCAGACCT | 263 | 529693 | 62 | n/a | n/a | kddedddddddddkke |
| 12635 | 12650 | AAAGCTGCTCAGACCT | 263 | 529718 | 69 | n/a | n/a | eddkdddddddddkke |
| 12635 | 12650 | AAAGCTGCTCAGACCT | 263 | 529911 | 66 | n/a | n/a | eeeedddddddddkke |
| 12693 | 12709 | CTCTGGCATTGCACAAT | 419 | 529462 | 76 | n/a | n/a | eekddddddddddkeke |
| 12794 | 12810 | TTCACCAACAACAGGAC | 420 | 529268 | 18 | 667 | 683 | eekddddddddddkeke |
| 12802 | 12818 | GAGCTCCATTCACCAAC | 421 | 529187 | 46 | 675 | 691 | eekddddddddddkeke |
| 12802 | 12818 | GAGCTCCATTCACCAAC | 421 | 529224 | 48 | 675 | 691 | eekddddddddddkkke |
| 12803 | 12818 | GAGCTCCATTCACCAA | 422 | 529130 | 34 | 676 | 691 | eeeddddddddddkkk |
| 12803 | 12818 | GAGCTCCATTCACCAA | 422 | 529150 | 51 | 676 | 691 | kkkddddddddddeee |
| 12863 | 12878 | CGAAACAGTGGGCCGC | 42 | 529549 | 85 | 736 | 751 | eekddddddddddkke |
| 12863 | 12878 | CGAAACAGTGGGCCGC | 42 | 529574 | 81 | 736 | 751 | keeddddddddddkke |
| 12863 | 12878 | CGAAACAGTGGGCCGC | 42 | 529599 | 64 | 736 | 751 | edkddddddddddkke |
| 12863 | 12878 | CGAAACAGTGGGCCGC | 42 | 529624 | 68 | 736 | 751 | kdeddddddddddkke |
| 12863 | 12878 | CGAAACAGTGGGCCGC | 42 | 529649 | 77 | 736 | 751 | kddkdddddddddkke |
| 12863 | 12878 | CGAAACAGTGGGCCGC | 42 | 529674 | 65 | 736 | 751 | kddedddddddddkke |
| 12863 | 12878 | CGAAACAGTGGGCCGC | 42 | 529699 | 63 | 736 | 751 | eddkdddddddddkke |
| 12863 | 12878 | CGAAACAGTGGGCCGC | 42 | 529931 | 59 | 736 | 751 | eeeedddddddddkke |
| 13739 | 13754 | GCTCGCTGAGGTCGTG | 423 | 529810 | 80 | 796 | 811 | kddddddddddkekee |
| 13739 | 13754 | GCTCGCTGAGGTCGTG | 423 | 529829 | 67 | 796 | 811 | kddddddddddkdkee |
| 13740 | 13756 | GTGCTCGCTGAGGTCGT | 424 | 529269 | 65 | 797 | 813 | eekddddddddddkeke |
| 13740 | 13755 | TGCTCGCTGAGGTCGT | 425 | 529731 | 66 | 797 | 812 | keddddddddddkeke |
| 13740 | 13755 | TGCTCGCTGAGGTCGT | 425 | 529751 | 76 | 797 | 812 | ekddddddddddkeke |
| 13740 | 13755 | TGCTCGCTGAGGTCGT | 425 | 529771 | 73 | 797 | 812 | keddddddddddkdke |
| 13740 | 13755 | TGCTCGCTGAGGTCGT | 425 | 529791 | 65 | 797 | 812 | ekddddddddddkdke |
| 13740 | 13756 | GTGCTCGCTGAGGTCGT | 424 | 529860 | 73 | 797 | 813 | kdeddddddddddkdke |
| 13740 | 13756 | GTGCTCGCTGAGGTCGT | 424 | 529880 | 74 | 797 | 813 | edkddddddddddkdke |
| 13740 | 13756 | GTGCTCGCTGAGGTCGT | 424 | 529900 | 62 | 797 | 813 | kddddkddddkddddke |
| 13741 | 13757 | CGTGCTCGCTGAGGTCG | 480 | 529270 | 69 | 798 | 814 | eekddddddddddkeke |
| 13741 | 13756 | GTGCTCGCTGAGGTCG | 44 | 529550 | 81 | 798 | 813 | eekddddddddddkke |
| 13741 | 13756 | GTGCTCGCTGAGGTCG | 44 | 529575 | 88 | 798 | 813 | keeddddddddddkke |
| 13741 | 13756 | GTGCTCGCTGAGGTCG | 44 | 529600 | 78 | 798 | 813 | edkddddddddddkke |
| 13741 | 13756 | GTGCTCGCTGAGGTCG | 44 | 529625 | 74 | 798 | 813 | kdeddddddddddkke |
| 13741 | 13756 | GTGCTCGCTGAGGTCG | 44 | 529650 | 81 | 798 | 813 | kddkdddddddddkke |
| 13741 | 13756 | GTGCTCGCTGAGGTCG | 44 | 529675 | 76 | 798 | 813 | kddedddddddddkke |
| 13741 | 13756 | GTGCTCGCTGAGGTCG | 44 | 529700 | 73 | 798 | 813 | eddkdddddddddkke |
| 13741 | 13756 | GTGCTCGCTGAGGTCG | 44 | 529920 | 67 | 798 | 813 | eeeedddddddddkke |
| 13762 | 13778 | GCCGGCTCTGCTCATCC | 427 | 529271 | 43 | 819 | 835 | eekddddddddddkeke |
| 13772 | 13788 | TGCGCCACCCGCCGGCT | 428 | 529272 | 0 | 829 | 845 | eekddddddddddkeke |
| 13776 | 13792 | GACCTGCGCCACCCGCC | 429 | 529273 | 62 | 833 | 849 | eekddddddddddkeke |
| 13777 | 13793 | TGACCTGCGCCACCCGC | 430 | 529274 | 78 | 834 | 850 | eekddddddddddkeke |
| 13836 | 13852 | CAGGCGGAGCAGCGCGA | 431 | 529275 | 70 | 893 | 909 | eekddddddddddkeke |
| 13891 | 13907 | TCCGTTCGGGCAGGCAG | 432 | 529276 | 73 | 948 | 964 | eekddddddddddkeke |
| 14017 | 14033 | CCTGGGTCATCAGCCGG | 433 | 529277 | 71 | 1074 | 1090 | eekddddddddddkeke |
| 14020 | 14036 | AGTCCTGGGTCATCAGC | 434 | 529278 | 72 | 1077 | 1093 | eekddddddddddkeke |
| 14023 | 14039 | GGCAGTCCTGGGTCATC | 435 | 529279 | 10 | 1080 | 1096 | eekddddddddddkeke |
| 14074 | 14090 | ACATGTACTCCGTGATA | 436 | 529280 | 11 | 1131 | 1147 | eekddddddddddkeke |
| 14075 | 14091 | AACATGTACTCCGTGAT | 437 | 529281 | 82 | 1132 | 1148 | eekddddddddddkeke |
| 14077 | 14093 | AGAACATGTACTCCGTG | 438 | 529282 | 87 | 1134 | 1150 | eekddddddddddkeke |
| 14077 | 14092 | GAACATGTACTCCGTG | 250 | 529803 | 71 | 1134 | 1149 | kddddddddddkekee |
| 14077 | 14092 | GAACATGTACTCCGTG | 250 | 529822 | 72 | 1134 | 1149 | kddddddddddkdkee |
| 14078 | 14093 | AGAACATGTACTCCGT | 439 | 529724 | 76 | 1135 | 1150 | keddddddddddkeke |
| 14078 | 14093 | AGAACATGTACTCCGT | 439 | 529744 | 81 | 1135 | 1150 | ekddddddddddkeke |
| 14078 | 14093 | AGAACATGTACTCCGT | 439 | 529764 | 65 | 1135 | 1150 | keddddddddddkdke |
| 14078 | 14093 | AGAACATGTACTCCGT | 439 | 529784 | 68 | 1135 | 1150 | ekddddddddddkdke |
| 14078 | 14094 | CAGAACATGTACTCCGT | 440 | 529853 | 64 | 1135 | 1151 | kdeddddddddddkdke |
| 14078 | 14094 | CAGAACATGTACTCCGT | 440 | 529873 | 69 | 1135 | 1151 | edkddddddddddkdke |
| 14078 | 14094 | CAGAACATGTACTCCGT | 440 | 529893 | 45 | 1135 | 1151 | kddddkddddkddddke |
| 14078 | 14094 | CAGAACATGTACTCCGT | 440 | 529937 | 81 | 1135 | 1151 | eekddddddddddkeke |
| 14079 | 14094 | CAGAACATGTACTCCG | 48 | 529551 | 88 | 1136 | 1151 | eekddddddddddkke |
| 14079 | 14094 | CAGAACATGTACTCCG | 48 | 529576 | 71 | 1136 | 1151 | keeddddddddddkke |
| 14079 | 14094 | CAGAACATGTACTCCG | 48 | 529601 | 74 | 1136 | 1151 | edkddddddddddkke |
| 14079 | 14094 | CAGAACATGTACTCCG | 48 | 529626 | 72 | 1136 | 1151 | kdeddddddddddkke |
| 14079 | 14094 | CAGAACATGTACTCCG | 48 | 529651 | 85 | 1136 | 1151 | kddkdddddddddkke |
| 14079 | 14094 | CAGAACATGTACTCCG | 48 | 529676 | 67 | 1136 | 1151 | kddedddddddddkke |
| 14079 | 14094 | CAGAACATGTACTCCG | 48 | 529701 | 82 | 1136 | 1151 | eddkdddddddddkke |
| 14079 | 14094 | CAGAACATGTACTCCG | 48 | 529913 | 76 | 1136 | 1151 | eeeedddddddddkke |
| 14090 | 14105 | AGTAGCCGGCACAGAA | 441 | 529811 | 56 | 1147 | 1162 | kddddddddddkekee |
| 14090 | 14105 | AGTAGCCGGCACAGAA | 441 | 529830 | 46 | 1147 | 1162 | kddddddddddkdkee |
| 14091 | 14106 | GAGTAGCCGGCACAGA | 442 | 529732 | 63 | 1148 | 1163 | keddddddddddkeke |
| 14091 | 14106 | GAGTAGCCGGCACAGA | 442 | 529752 | 72 | 1148 | 1163 | ekddddddddddkeke |
| 14091 | 14106 | GAGTAGCCGGCACAGA | 442 | 529772 | 61 | 1148 | 1163 | keddddddddddkdke |
| 14091 | 14106 | GAGTAGCCGGCACAGA | 442 | 529792 | 68 | 1148 | 1163 | ekddddddddddkdke |
| 14091 | 14107 | CGAGTAGCCGGCACAGA | 443 | 529861 | 54 | 1148 | 1164 | kdeddddddddddkdke |
| 14091 | 14107 | CGAGTAGCCGGCACAGA | 443 | 529881 | 78 | 1148 | 1164 | edkddddddddddkdke |
| 14091 | 14107 | CGAGTAGCCGGCACAGA | 443 | 529901 | 29 | 1148 | 1164 | kddddkddddkddddke |
| 14091 | 14107 | CGAGTAGCCGGCACAGA | 443 | 529939 | 67 | 1148 | 1164 | eekddddddddddkeke |
| 14092 | 14108 | CCGAGTAGCCGGCACAG | 444 | 529283 | 70 | 1149 | 1165 | eekddddddddddkeke |
| 14092 | 14107 | CGAGTAGCCGGCACAG | 49 | 529552 | 72 | 1149 | 1164 | eekddddddddddkke |
| 14092 | 14107 | CGAGTAGCCGGCACAG | 49 | 529577 | 80 | 1149 | 1164 | keeddddddddddkke |
| 14092 | 14107 | CGAGTAGCCGGCACAG | 49 | 529602 | 64 | 1149 | 1164 | edkddddddddddkke |
| 14092 | 14107 | CGAGTAGCCGGCACAG | 49 | 529627 | 56 | 1149 | 1164 | kdeddddddddddkke |
| 14092 | 14107 | CGAGTAGCCGGCACAG | 49 | 529652 | 57 | 1149 | 1164 | kddkdddddddddkke |
| 14092 | 14107 | CGAGTAGCCGGCACAG | 49 | 529677 | 43 | 1149 | 1164 | kddedddddddddkke |
| 14092 | 14107 | CGAGTAGCCGGCACAG | 49 | 529702 | 54 | 1149 | 1164 | eddkdddddddddkke |
| 14092 | 14107 | CGAGTAGCCGGCACAG | 49 | 529921 | 42 | 1149 | 1164 | eeeedddddddddkke |
| 14093 | 14109 | TCCGAGTAGCCGGCACA | 445 | 529284 | 76 | 1150 | 1166 | eekddddddddddkeke |
| 14119 | 14135 | CCCCCTTGCAGGAGTCC | 446 | 529285 | 77 | 1176 | 1192 | eekddddddddddkeke |
| 14120 | 14136 | TCCCCCTTGCAGGAGTC | 447 | 529286 | 68 | 1177 | 1193 | eekddddddddddkeke |
| 14127 | 14143 | TCCACTGTCCCCCTTGC | 448 | 529287 | 65 | 1184 | 1200 | eekddddddddddkeke |
| 14231 | 14246 | CCCTGGTGTACACCCC | 264 | 529719 | 73 | 1288 | 1303 | keddddddddddkeke |
| 14231 | 14246 | CCCTGGTGTACACCCC | 264 | 529739 | 83 | 1288 | 1303 | ekddddddddddkeke |
| 14231 | 14246 | CCCTGGTGTACACCCC | 264 | 529759 | 63 | 1288 | 1303 | keddddddddddkdke |
| 14231 | 14246 | CCCTGGTGTACACCCC | 244 | 529779 | 70 | 1288 | 1303 | ekddddddddddkdke |
| 14231 | 14247 | ACCCTGGTGTACACCCC | 449 | 529848 | 60 | 1288 | 1304 | kdeddddddddddkdke |
| 14231 | 14247 | ACCCTGGTGTACACCCC | 449 | 529868 | 63 | 1288 | 1304 | edkddddddddddkdke |
| 14231 | 14247 | ACCCTGGTGTACACCCC | 449 | 529888 | 53 | 1288 | 1304 | kddddkddddkddddke |
| 14232 | 14247 | ACCCTGGTGTACACCC | 265 | 529553 | 81 | 1289 | 1304 | eekddddddddddkke |
| 14232 | 14247 | ACCCTGGTGTACACCC | 265 | 529578 | 65 | 1289 | 1304 | keeddddddddddkke |
| 14232 | 14247 | ACCCTGGTGTACACCC | 265 | 529603 | 60 | 1289 | 1304 | edkddddddddddkke |
| 14232 | 14247 | ACCCTGGTGTACACCC | 265 | 529628 | 59 | 1289 | 1304 | kdeddddddddddkke |
| 14232 | 14247 | ACCCTGGTGTACACCC | 265 | 529653 | 76 | 1289 | 1304 | kddkdddddddddkke |
| 14232 | 14247 | ACCCTGGTGTACACCC | 265 | 529678 | 56 | 1289 | 1304 | kddedddddddddkke |
| 14232 | 14247 | ACCCTGGTGTACACCC | 265 | 529703 | 68 | 1289 | 1304 | eddkdddddddddkke |
| 14232 | 14247 | ACCCTGGTGTACACCC | 265 | 529908 | 69 | 1289 | 1304 | eeeedddddddddkke |
| 14233 | 14249 | AGACCCTGGTGTACACC | 450 | 529168 | 64 | 1290 | 1306 | eekddddddddddkeke |
| 14233 | 14249 | AGACCCTGGTGTACACC | 450 | 529205 | 62 | 1290 | 1306 | eekddddddddddkkke |
| 14240 | 14256 | TACTGGGAGACCCTGGT | 451 | 529290 | 53 | 1297 | 1313 | eekddddddddddkeke |
| 14241 | 14256 | TACTGGGAGACCCTGG | 452 | 529802 | 57 | 1298 | 1313 | kddddddddddkekee |
| 14241 | 14256 | TACTGGGAGACCCTGG | 452 | 529821 | 61 | 1298 | 1313 | kddddddddddkdkee |
| 14242 | 14258 | TGTACTGGGAGACCCTG | 453 | 529292 | 74 | 1299 | 1315 | eekddddddddddkeke |
| 14242 | 14257 | GTACTGGGAGACCCTG | 454 | 529723 | 68 | 1299 | 1314 | keddddddddddkeke |
| 14242 | 14257 | GTACTGGGAGACCCTG | 454 | 529743 | 84 | 1299 | 1314 | ekddddddddddkeke |
| 14242 | 14257 | GTACTGGGAGACCCTG | 454 | 529763 | 64 | 1299 | 1314 | keddddddddddkdke |
| 14242 | 14257 | GTACTGGGAGACCCTG | 454 | 529783 | 72 | 1299 | 1314 | ekddddddddddkdke |
| 14242 | 14258 | TGTACTGGGAGACCCTG | 453 | 529852 | 66 | 1299 | 1315 | kdeddddddddddkdke |
| 14242 | 14258 | TGTACTGGGAGACCCTG | 453 | 529872 | 62 | 1299 | 1315 | edkddddddddddkdke |
| 14242 | 14258 | TGTACTGGGAGACCCTG | 453 | 529892 | 43 | 1299 | 1315 | kddddkddddkddddke |
| 14243 | 14258 | TGTACTGGGAGACCCT | 252 | 529554 | 80 | 1300 | 1315 | eekddddddddddkke |
| 14243 | 14258 | TGTACTGGGAGACCCT | 252 | 529579 | 83 | 1300 | 1315 | keeddddddddddkke |
| 14243 | 14258 | TGTACTGGGAGACCCT | 252 | 529604 | 73 | 1300 | 1315 | edkddddddddddkke |
| 14243 | 14258 | TGTACTGGGAGACCCT | 252 | 529629 | 64 | 1300 | 1315 | kdeddddddddddkke |
| 14243 | 14258 | TGTACTGGGAGACCCT | 252 | 529654 | 69 | 1300 | 1315 | kddkdddddddddkke |
| 14243 | 14258 | TGTACTGGGAGACCCT | 252 | 529679 | 52 | 1300 | 1315 | kddedddddddddkke |
| 14243 | 14258 | TGTACTGGGAGACCCT | 252 | 529704 | 63 | 1300 | 1315 | eddkdddddddddkke |
| 14243 | 14258 | TGTACTGGGAGACCCT | 252 | 529912 | 64 | 1300 | 1315 | eeeedddddddddkke |
| 14246 | 14262 | TCGATGTACTGGGAGAC | 455 | 529294 | 74 | 1303 | 1319 | eekddddddddddkeke |
| 14247 | 14263 | CTCGATGTACTGGGAGA | 456 | 529296 | 52 | 1304 | 1320 | eekddddddddddkeke |
| 14262 | 14278 | GAGCTTTTGCAGCCACT | 457 | 529298 | 60 | 1319 | 1335 | eekddddddddddkeke |
| 14263 | 14279 | TGAGCTTTTGCAGCCAC | 458 | 529300 | 71 | 1320 | 1336 | eekddddddddddkeke |
| 14349 | 14365 | GCCTTGGCTTTCTCTCC | 459 | 529188 | 79 | 1406 | 1422 | eekddddddddddkeke |
| 14349 | 14365 | GCCTTGGCTTTCTCTCC | 459 | 529225 | 78 | 1406 | 1422 | eekddddddddddkkke |
| 14350 | 14365 | GCCTTGGCTTTCTCTC | 460 | 529131 | 58 | 1407 | 1422 | eeeddddddddddkkk |
| 14350 | 14365 | GCCTTGGCTTTCTCTC | 460 | 529151 | 71 | 1407 | 1422 | kkkddddddddddeee |
| 14611 | 14627 | CCCCTCTGTCCAGCGCC | 461 | 529302 | 74 | 1668 | 1684 | eekddddddddddkeke |
| 14612 | 14628 | GCCCCTCTGTCCAGCGC | 222 | 529189 | 64 | 1669 | 1685 | eekddddddddddkeke |
| 14612 | 14628 | GCCCCTCTGTCCAGCGC | 222 | 529226 | 50 | 1669 | 1685 | eekddddddddddkkke |
| 14613 | 14628 | GCCCCTCTGTCCAGCG | 462 | 529132 | 78 | 1670 | 1685 | eeeddddddddddkkk |
| 14613 | 14628 | GCCCCTCTGTCCAGCG | 462 | 529152 | 62 | 1670 | 1685 | kkkddddddddddeee |
| 14613 | 14629 | TGCCCCTCTGTCCAGCG | 223 | 529190 | 76 | 1670 | 1686 | eekddddddddddkeke |
| 14613 | 14629 | TGCCCCTCTGTCCAGCG | 250 | 529227 | 88 | 1670 | 1686 | eekddddddddddkkke |
| 14614 | 14629 | TGCCCCTCTGTCCAGC | 463 | 529133 | 81 | 1671 | 1686 | eeeddddddddddkkk |
| 14614 | 14629 | TGCCCCTCTGTCCAGC | 463 | 529153 | 68 | 1671 | 1686 | kkkddddddddddeee |
| 14614 | 14630 | CTGCCCCTCTGTCCAGC | 224 | 529191 | 78 | 1671 | 1687 | eekddddddddddkeke |
| 14614 | 14630 | CTGCCCCTCTGTCCAGC | 224 | 529228 | 85 | 1671 | 1687 | eekddddddddddkkke |
| 14615 | 14630 | CTGCCCCTCTGTCCAG | 464 | 529134 | 75 | 1672 | 1687 | eeeddddddddddkkk |
| 14615 | 14630 | CTGCCCCTCTGTCCAG | 464 | 529154 | 61 | 1672 | 1687 | kkkddddddddddeee |
| 14707 | 14723 | GAGGCCAGCAGATCACG | 465 | 529304 | 89 | 1764 | 1780 | eekddddddddddkeke |
| 14712 | 14728 | AGCCTGAGGCCAGCAGA | 466 | 529306 | 84 | 1769 | 1785 | eekddddddddddkeke |
| 14735 | 14751 | TCCAGCAATGAAGGCAG | 467 | 529308 | 68 | 1792 | 1808 | eekddddddddddkeke |
| 14739 | 14755 | TGTCTCCAGCAATGAAG | 468 | 529310 | 59 | 1796 | 1812 | eekddddddddddkeke |
| 15099 | 15115 | CACATGGAGTCAGCATC | 469 | 529169 | 79 | 2156 | 2172 | eekddddddddddkeke |
| 15099 | 15115 | CACATGGAGTCAGCATC | 469 | 529206 | 82 | 2156 | 2172 | eekddddddddddkkke |
| 15100 | 15116 | ACACATGGAGTCAGCAT | 470 | 529312 | 68 | 2157 | 2173 | eekddddddddddkeke |
| 15109 | 15125 | GAGGACAGCACACATGG | 471 | 529314 | 61 | 2166 | 2182 | eekddddddddddkeke |
| 15128 | 15144 | AGCTAAACAACCGCCTT | 472 | 529316 | 62 | 2185 | 2201 | eekddddddddddkeke |
| 15128 | 15143 | GCTAAACAACCGCCTT | 59 | 529555 | 78 | 2185 | 2200 | eekddddddddddkke |
| 15128 | 15143 | GCTAAACAACCGCCTT | 59 | 529580 | 73 | 2185 | 2200 | keeddddddddddkke |
| 15128 | 15143 | GCTAAACAACCGCCTT | 59 | 529605 | 71 | 2185 | 2200 | edkddddddddddkke |
| 15128 | 15143 | GCTAAACAACCGCCTT | 59 | 529630 | 64 | 2185 | 2200 | kdeddddddddddkke |
| 15128 | 15143 | GCTAAACAACCGCCTT | 59 | 529655 | 63 | 2185 | 2200 | kddkdddddddddkke |
| 15128 | 15143 | GCTAAACAACCGCCTT | 59 | 529680 | 43 | 2185 | 2200 | kddedddddddddkke |
| 15128 | 15143 | GCTAAACAACCGCCTT | 59 | 529705 | 63 | 2185 | 2200 | eddkdddddddddkke |
| 15128 | 15143 | GCTAAACAACCGCCTT | 59 | 529932 | 60 | 2185 | 2200 | eeeedddddddddkke |
| 15129 | 15145 | GAGCTAAACAACCGCCT | 473 | 529318 | 82 | 2186 | 2202 | eekddddddddddkeke |
| 15130 | 15146 | AGAGCTAAACAACCGCC | 474 | 529170 | 85 | 2187 | 2203 | eekddddddddddkeke |
| 15130 | 15146 | AGAGCTAAACAACCGCC | 474 | 529207 | 88 | 2187 | 2203 | eekddddddddddkkke |
| 15131 | 15147 | GAGAGCTAAACAACCGC | 475 | 529171 | 81 | 2188 | 2204 | eekddddddddddkeke |
| 15131 | 15147 | GAGAGCTAAACAACCGC | 475 | 529208 | 84 | 2188 | 2204 | eekddddddddddkkke |
| 15162 | 15177 | AAGATGATAATGGATA | 476 | 529805 | 40 | 2219 | 2234 | kddddddddddkekee |
| 15162 | 15177 | AAGATGATAATGGATA | 476 | 529824 | 32 | 2219 | 2234 | kddddddddddkdkee |
| 15163 | 15179 | TGAAGATGATAATGGAT | 477 | 529320 | 74 | 2220 | 2236 | eekddddddddddkeke |
| 15163 | 15178 | GAAGATGATAATGGAT | 478 | 529726 | 80 | 2220 | 2235 | keddddddddddkeke |
| 15163 | 15178 | GAAGATGATAATGGAT | 478 | 529746 | 82 | 2220 | 2235 | ekddddddddddkeke |
| 15163 | 15178 | GAAGATGATAATGGAT | 478 | 529766 | 63 | 2220 | 2235 | keddddddddddkdke |
| 15163 | 15178 | GAAGATGATAATGGAT | 478 | 529786 | 69 | 2220 | 2235 | ekddddddddddkdke |
| 15163 | 15179 | TGAAGATGATAATGGAT | 477 | 529855 | 39 | 2220 | 2236 | kdeddddddddddkdke |
| 15163 | 15179 | TGAAGATGATAATGGAT | 477 | 529875 | 40 | 2220 | 2236 | edkddddddddddkdke |
| 15163 | 15179 | TGAAGATGATAATGGAT | 477 | 529895 | 27 | 2220 | 2236 | kddddkddddkddddke |
| 15164 | 15179 | TGAAGATGATAATGGA | 61 | 529556 | 72 | 2221 | 2236 | eekddddddddddkke |
| 15164 | 15179 | TGAAGATGATAATGGA | 61 | 529581 | 68 | 2221 | 2236 | keeddddddddddkke |
| 15164 | 15179 | TGAAGATGATAATGGA | 61 | 529606 | 54 | 2221 | 2236 | edkddddddddddkke |
| 15164 | 15179 | TGAAGATGATAATGGA | 61 | 529631 | 29 | 2221 | 2236 | kdeddddddddddkke |
| 15164 | 15179 | TGAAGATGATAATGGA | 61 | 529656 | 74 | 2221 | 2236 | kddkdddddddddkke |
| 15164 | 15179 | TGAAGATGATAATGGA | 61 | 529681 | 32 | 2221 | 2236 | kddedddddddddkke |
| 15164 | 15179 | TGAAGATGATAATGGA | 61 | 529706 | 41 | 2221 | 2236 | eddkdddddddddkke |
| 15164 | 15179 | TGAAGATGATAATGGA | 61 | 529915 | 51 | 2221 | 2236 | eeeedddddddddkke |
| 15182 | 15198 | GCTTCTGAATTGTCTGA | 226 | 529172 | 88 | 2239 | 2255 | eekddddddddddkeke |
| 15182 | 15198 | GCTTCTGAATTGTCTGA | 226 | 529209 | 87 | 2239 | 2255 | eekddddddddddkkke |
| 15183 | 15199 | TGCTTCTGAATTGTCTG | 227 | 529173 | 92 | 2240 | 2256 | eekddddddddddkeke |
| 15183 | 15199 | TGCTTCTGAATTGTCTG | 227 | 529210 | 89 | 2240 | 2256 | eekddddddddddkkke |
| 15184 | 15200 | ATGCTTCTGAATTGTCT | 479 | 529183 | 85 | 2241 | 2257 | eekddddddddddkeke |
| 15184 | 15200 | ATGCTTCTGAATTGTCT | 479 | 529220 | 92 | 2241 | 2257 | eekddddddddddkkke |
| 15185 | 15200 | ATGCTTCTGAATTGTC | 257 | 529126 | 83 | 2242 | 2257 | eeeddddddddddkkk |
| 15185 | 15200 | ATGCTTCTGAATTGTC | 257 | 529146 | 84 | 2242 | 2257 | kkkddddddddddeee |
| 15185 | 15201 | GATGCTTCTGAATTGTC | 480 | 529174 | 85 | 2242 | 2258 | eekddddddddddkeke |
| 15185 | 15201 | GATGCTTCTGAATTGTC | 480 | 529211 | 86 | 2242 | 2258 | eekddddddddddkkke |
| 15188 | 15204 | GGTGATGCTTCTGAATT | 481 | 529322 | 71 | 2245 | 2261 | eekddddddddddkeke |
| 15189 | 15205 | TGGTGATGCTTCTGAAT | 482 | 529324 | 79 | 2246 | 2262 | eekddddddddddkeke |
| 15190 | 15206 | ATGGTGATGCTTCTGAA | 483 | 529326 | 85 | 2247 | 2263 | eekddddddddddkeke |
| 15191 | 15207 | CATGGTGATGCTTCTGA | 228 | 529175 | 92 | 2248 | 2264 | eekddddddddddkeke |
| 15191 | 15207 | CATGGTGATGCTTCTGA | 228 | 529212 | 92 | 2248 | 2264 | eekddddddddddkkke |
| 15192 | 15208 | GCATGGTGATGCTTCTG | 229 | 529176 | 89 | 2249 | 2265 | eekddddddddddkeke |
| 15192 | 15208 | GCATGGTGATGCTTCTG | 229 | 529213 | 90 | 2249 | 2265 | eekddddddddddkkke |
| 15192 | 15207 | CATGGTGATGCTTCTG | 259 | 529804 | 89 | 2249 | 2264 | kddddddddddkekee |
| 15192 | 15207 | CATGGTGATGCTTCTG | 259 | 529823 | 89 | 2249 | 2264 | kddddddddddkdkee |
| 15193 | 15209 | TGCATGGTGATGCTTCT | 230 | 529166 | 83 | 2250 | 2266 | eekddddddddddkeke |
| 15193 | 15209 | TGCATGGTGATGCTTCT | 230 | 529203 | 86 | 2250 | 2266 | eekddddddddddkkke |
| 15193 | 15208 | GCATGGTGATGCTTCT | 260 | 529725 | 92 | 2250 | 2265 | keddddddddddkeke |
| 15193 | 15208 | GCATGGTGATGCTTCT | 260 | 529745 | 91 | 2250 | 2265 | ekddddddddddkeke |
| 15193 | 15208 | GCATGGTGATGCTTCT | 260 | 529765 | 88 | 2250 | 2265 | keddddddddddkdke |
| 15193 | 15208 | GCATGGTGATGCTTCT | 260 | 529785 | 91 | 2250 | 2265 | ekddddddddddkdke |
| 15193 | 15208 | GCATGGTGATGCTTCT | 260 | 529799 | 89 | 2250 | 2265 | kddddddddddkekee |
| 15193 | 15208 | GCATGGTGATGCTTCT | 260 | 529818 | 88 | 2250 | 2265 | kddddddddddkdkee |
| 15193 | 15209 | TGCATGGTGATGCTTCT | 230 | 529854 | 90 | 2250 | 2266 | kdeddddddddddkdke |
| 15193 | 15209 | TGCATGGTGATGCTTCT | 230 | 529874 | 81 | 2250 | 2266 | edkddddddddddkdke |
| 15193 | 15209 | TGCATGGTGATGCTTCT | 230 | 529894 | 60 | 2250 | 2266 | kddddkddddkddddke |
| 15194 | 15210 | ATGCATGGTGATGCTTC | 231 | 529167 | 71 | 2251 | 2267 | eekddddddddddkeke |
| 15194 | 15210 | ATGCATGGTGATGCTTC | 231 | 529204 | 70 | 2251 | 2267 | eekddddddddddkkke |
| 15194 | 15209 | TGCATGGTGATGCTTC | 69 | 529557 | 86 | 2251 | 2266 | eekddddddddddkke |
| 15194 | 15209 | TGCATGGTGATGCTTC | 69 | 529582 | 86 | 2251 | 2266 | keeddddddddddkke |
| 15194 | 15209 | TGCATGGTGATGCTTC | 69 | 529607 | 84 | 2251 | 2266 | edkddddddddddkke |
| 15194 | 15209 | TGCATGGTGATGCTTC | 69 | 529632 | 81 | 2251 | 2266 | kdeddddddddddkke |
| 15194 | 15209 | TGCATGGTGATGCTTC | 69 | 529657 | 85 | 2251 | 2266 | kddkdddddddddkke |
| 15194 | 15209 | TGCATGGTGATGCTTC | 69 | 529682 | 78 | 2251 | 2266 | kddedddddddddkke |
| 15194 | 15209 | TGCATGGTGATGCTTC | 69 | 529707 | 79 | 2251 | 2266 | eddkdddddddddkke |
| 15194 | 15209 | TGCATGGTGATGCTTC | 69 | 529720 | 75 | 2251 | 2266 | keddddddddddkeke |
| 15194 | 15209 | TGCATGGTGATGCTTC | 69 | 529740 | 70 | 2251 | 2266 | ekddddddddddkeke |
| 15194 | 15209 | TGCATGGTGATGCTTC | 69 | 529760 | 78 | 2251 | 2266 | keddddddddddkdke |
| 15194 | 15209 | TGCATGGTGATGCTTC | 69 | 529780 | 83 | 2251 | 2266 | ekddddddddddkdke |
| 15194 | 15210 | ATGCATGGTGATGCTTC | 231 | 529849 | 80 | 2251 | 2267 | kdeddddddddddkdke |
| 15194 | 15210 | ATGCATGGTGATGCTTC | 231 | 529869 | 72 | 2251 | 2267 | edkddddddddddkdke |
| 15194 | 15210 | ATGCATGGTGATGCTTC | 231 | 529889 | 49 | 2251 | 2267 | kddddkddddkddddke |
| 15194 | 15209 | TGCATGGTGATGCTTC | 69 | 529914 | 69 | 2251 | 2266 | eeeedddddddddkke |
| 15195 | 15211 | CATGCATGGTGATGCTT | 484 | 529328 | 68 | 2252 | 2268 | eekddddddddddkeke |
| 15195 | 15210 | ATGCATGGTGATGCTT | 71 | 529558 | 71 | 2252 | 2267 | eekddddddddddkke |
| 15195 | 15210 | ATGCATGGTGATGCTT | 71 | 529583 | 81 | 2252 | 2267 | keeddddddddddkke |
| 15195 | 15210 | ATGCATGGTGATGCTT | 71 | 529608 | 68 | 2252 | 2267 | edkddddddddddkke |
| 15195 | 15210 | ATGCATGGTGATGCTT | 71 | 529633 | 73 | 2252 | 2267 | kdeddddddddddkke |
| 15195 | 15210 | ATGCATGGTGATGCTT | 71 | 529658 | 63 | 2252 | 2267 | kddkdddddddddkke |
| 15195 | 15210 | ATGCATGGTGATGCTT | 71 | 529683 | 74 | 2252 | 2267 | kddedddddddddkke |
| 15195 | 15210 | ATGCATGGTGATGCTT | 71 | 529708 | 70 | 2252 | 2267 | eddkdddddddddkke |
| 15195 | 15210 | ATGCATGGTGATGCTT | 71 | 529909 | 59 | 2252 | 2267 | eeeedddddddddkke |
| 15205 | 15221 | CATTCGCCACCATGCAT | 485 | 529192 | 51 | 2262 | 2278 | eekddddddddddkeke |
| 15205 | 15221 | CATTCGCCACCATGCAT | 485 | 529229 | 69 | 2262 | 2278 | eekddddddddddkkke |
| 15206 | 15221 | CATTCGCCACCATGCA | 486 | 529135 | 54 | 2263 | 2278 | eeeddddddddddkkk |
| 15206 | 15221 | CATTCGCCACCATGCA | 486 | 529155 | 56 | 2263 | 2278 | kkkddddddddddeee |
| 15805 | 15821 | TGGTGCCCAGGACGGCC | 487 | 529330 | 37 | 2862 | 2878 | eekddddddddddkeke |

### Example 20

### Design of Modified Antisense Oligonucleotides Comprising MOE and cEt Modifications Targeting Human Coagulation Factor VII

Based on the activity of the antisense oligonucleotides listed above, additional antisense oligonucleotides were designed targeting a Factor VII nucleic acid at start positions 1147, 1154, or 12842 of SEQ ID NO: 1. The newly designed modified antisense oligonucleotides and their motifs are described in Table 21. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO.

Each oligonucleotide listed in Table 21 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted human gene sequence. ‘n/a.’ indicates that the antisense oligonucleotide is not 100% complementary with that particular gene sequence.

TABLE 21
Chimeric antisense oligonucleotides targeted to SEQ ID NO: 1 and SEQ ID NO: 2

| Start | Stop | | | | Start | Stop | |
|---|---|---|---|---|---|---|---|
| Site on | Site on | | SEQ | | Site on | Site on | |
| SEQ ID | SEQ ID | | ID | | SEQ ID | SEQ ID | |
| NO: 1 | NO: 1 | Sequence | NO | ISIS No | NO: 2 | NO: 2 | Sugar Chemistry |
| 1147 | 1162 | GATGAAATCTCTGCAG | 21 | 529544 | 36 | 51 | eekddddddddddkke |
| 1147 | 1162 | GATGAAATCTCTGCAG | 21 | 529569 | 36 | 51 | keeddddddddddkke |
| 1147 | 1162 | GATGAAATCTCTGCAG | 21 | 529594 | 36 | 51 | edkddddddddddkke |
| 1147 | 1162 | GATGAAATCTCTGCAG | 21 | 529619 | 36 | 51 | kdeddddddddddkke |
| 1147 | 1162 | GATGAAATCTCTGCAG | 21 | 529644 | 36 | 51 | kddkdddddddddkke |
| 1147 | 1162 | GATGAAATCTCTGCAG | 21 | 529669 | 36 | 51 | kddedddddddddkke |
| 1147 | 1162 | GATGAAATCTCTGCAG | 21 | 529694 | 36 | 51 | eddkdddddddddkke |
| 1147 | 1162 | GATGAAATCTCTGCAG | 21 | 529929 | 36 | 51 | eeeedddddddddkke |
| 1152 | 1167 | ACCATGATGAAATCTC | 488 | 529809 | 41 | 56 | kddddddddddkekee |
| 1152 | 1167 | ACCATGATGAAATCTC | 488 | 529828 | 41 | 56 | kddddddddddkdkee |
| 1153 | 1168 | GACCATGATGAAATCT | 489 | 529730 | 42 | 57 | keddddddddddkeke |
| 1153 | 1168 | GACCATGATGAAATCT | 489 | 529750 | 42 | 57 | ekddddddddddkeke |
| 1153 | 1168 | GACCATGATGAAATCT | 489 | 529770 | 42 | 57 | keddddddddddkdke |
| 1153 | 1168 | GACCATGATGAAATCT | 489 | 529790 | 42 | 57 | ekddddddddddkdke |
| 1153 | 1169 | AGACCATGATGAAATCT | 490 | 529859 | 42 | 58 | kdeddddddddddkdke |
| 1153 | 1169 | AGACCATGATGAAATCT | 490 | 529879 | 42 | 58 | edkddddddddddkdke |
| 1153 | 1169 | AGACCATGATGAAATCT | 490 | 529899 | 42 | 58 | kddddkddddkddddke |
| 1154 | 1169 | AGACCATGATGAAATC | 22 | 529545 | 43 | 58 | eekddddddddddkke |
| 1154 | 1169 | AGACCATGATGAAATC | 22 | 529570 | 43 | 58 | keeddddddddddkke |
| 1154 | 1169 | AGACCATGATGAAATC | 22 | 529595 | 43 | 58 | edkddddddddddkke |
| 1154 | 1169 | AGACCATGATGAAATC | 22 | 529620 | 43 | 58 | kdeddddddddddkke |
| 1154 | 1169 | AGACCATGATGAAATC | 22 | 529645 | 43 | 58 | kddkdddddddddkke |
| 1154 | 1169 | AGACCATGATGAAATC | 22 | 529670 | 43 | 58 | kddedddddddddkke |
| 1154 | 1169 | AGACCATGATGAAATC | 22 | 529695 | 43 | 58 | eddkdddddddddkke |
| 1154 | 1169 | AGACCATGATGAAATC | 22 | 529919 | 43 | 58 | eeeedddddddddkke |
| 12842 | 12857 | CCACCCAGATGGTGTT | 41 | 529548 | 715 | 730 | eekddddddddddkke |
| 12842 | 12857 | CCACCCAGATGGTGTT | 41 | 529573 | 715 | 730 | keeddddddddddkke |
| 12842 | 12857 | CCACCCAGATGGTGTT | 41 | 529598 | 715 | 730 | edkddddddddddkke |
| 12842 | 12857 | CCACCCAGATGGTGTT | 41 | 529623 | 715 | 730 | kdeddddddddddkke |
| 12842 | 12857 | CCACCCAGATGGTGTT | 41 | 529648 | 715 | 730 | kddkdddddddddkke |
| 12842 | 12857 | CCACCCAGATGGTGTT | 41 | 529673 | 715 | 730 | kddedddddddddkke |
| 12842 | 12857 | CCACCCAGATGGTGTT | 41 | 529698 | 715 | 730 | eddkdddddddddkke |
| 12842 | 12857 | CCACCCAGATGGTGTT | 41 | 529930 | 715 | 730 | eeeedddddddddkke |

### Example 21

### Modified Antisense Oligonucleotides Comprising cEt and MOE Modifications Targeting Human Coagulation Factor VII

Additional antisense oligonucleotides were designed targeting a Factor VII nucleic acid and were tested for their effects on Factor VII mRNA in vitro. ISIS 472998, a 2-10-2 cEt gapmer, and ISIS 515554, a deoxy, MOE, and cEt oligonucleotide, described in the Examples above were also included in the screen.

The newly designed modified antisense oligonucleotides and their motifs are described in Table 22. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO.

Each oligonucleotide listed in Table 22 is targeted to either the human Factor VII genomic sequence, designated herein as SEQ ID NO: 1 (GENBANK Accession No. NT_{—}027140.6 truncated from nucleotides 1255000 to 1273000) or the human Factor VII mRNA sequence, designated herein as SEQ ID NO: 2 (GENBANK Accession No. NM_{—}019616.2), or both. “Start site” indicates the 5′-most nucleoside to which the oligonucleotide is targeted in the human gene sequence. “Stop site” indicates the 3′-most nucleoside to which the oligonucleotide is targeted in the human gene sequence. ‘n/a.’ indicates that the antisense oligonucleotide is not 100% complementary with that particular gene sequence.

Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 (described hereinabove in Example 1) was used to measure mRNA levels. Factor VII mRNA levels were adjusted according to total RNA content, as measured by RIBOGREEN®. Results are presented as percent inhibition of Factor VII expression, relative to untreated control cells.

TABLE 22
Percent inhibition of human Factor VII mRNA levels by modified antisense
oligonucleotides targeted to SEQ ID NO: 1 and SEQ ID NO: 2

| Start | Stop | | | | | Start | Stop | |
|---|---|---|---|---|---|---|---|---|
| Site on | Site on | | SEQ | | | Site on | Site on | |
| SEQ ID | SEQ ID | | ID | | % | SEQ ID | SEQ ID | |
| NO: 1 | NO: 1 | Sequence | NO | ISIS No | inhibition | NO: 2 | NO: 2 | Sugar Chemistry |
| 15263 | 15276 | TGTGCAGCCCGGCA | 74 | 472998 | 88 | 2320 | 2333 | kkddddddddddkk |
| 2499 | 2514 | GCAGATTTGCATCAGA | 493 | 515554 | 75 | n/a | n/a | eeeddddddddddkkk |
| 2238 | 2253 | ACCAGTGGCAGTCCCT | 491 | 534530 | 92 | n/a | n/a | kekedddddddddkek |
| 2238 | 2253 | ACCAGTGGCAGTCCCT | 491 | 534563 | 92 | n/a | n/a | kekdddddddddekek |
| 2238 | 2253 | ACCAGTGGCAGTCCCT | 491 | 534596 | 88 | n/a | n/a | ekeedddddddddkke |
| 2238 | 2253 | ACCAGTGGCAGTCCCT | 491 | 534629 | 89 | n/a | n/a | ekedddddddddekke |
| 2238 | 2253 | ACCAGTGGCAGTCCCT | 491 | 534662 | 87 | n/a | n/a | eekkdddddddddeke |
| 2238 | 2253 | ACCAGTGGCAGTCCCT | 491 | 534695 | 92 | n/a | n/a | eekdddddddddkeke |
| 2238 | 2253 | ACCAGTGGCAGTCCCT | 491 | 534732 | 90 | n/a | n/a | ekekddddddddkeke |
| 2238 | 2253 | ACCAGTGGCAGTCCCT | 491 | 534767 | 92 | n/a | n/a | keekddddddddkeek |
| 2238 | 2253 | ACCAGTGGCAGTCCCT | 491 | 534802 | 93 | n/a | n/a | ekkddddddddddkke |
| 2238 | 2253 | ACCAGTGGCAGTCCCT | 491 | 534832 | 83 | n/a | n/a | edkddddddddddkke |
| 2238 | 2253 | ACCAGTGGCAGTCCCT | 491 | 534862 | 72 | n/a | n/a | kdeddddddddddkke |
| 2238 | 2253 | ACCAGTGGCAGTCCCT | 491 | 534892 | 82 | n/a | n/a | eekddddddddddkke |
| 2238 | 2253 | ACCAGTGGCAGTCCCT | 491 | 534922 | 80 | n/a | n/a | kddkdddddddddkke |
| 2238 | 2253 | ACCAGTGGCAGTCCCT | 491 | 534952 | 72 | n/a | n/a | kddedddddddddkke |
| 2238 | 2253 | ACCAGTGGCAGTCCCT | 491 | 534982 | 77 | n/a | n/a | eddkdddddddddkke |
| 2238 | 2253 | ACCAGTGGCAGTCCCT | 491 | 535012 | 70 | n/a | n/a | eeeedddddddddkke |
| 2238 | 2253 | ACCAGTGGCAGTCCCT | 491 | 535045 | 84 | n/a | n/a | eeeedddddddddkkk |
| 2238 | 2253 | ACCAGTGGCAGTCCCT | 491 | 535078 | 87 | n/a | n/a | eeekdddddddddkke |
| 2238 | 2253 | ACCAGTGGCAGTCCCT | 491 | 535111 | 63 | n/a | n/a | eeeeeddddddddkke |
| 2238 | 2253 | ACCAGTGGCAGTCCCT | 491 | 535144 | 69 | n/a | n/a | ededkddddddddkke |
| 2238 | 2253 | ACCAGTGGCAGTCCCT | 491 | 535177 | 68 | n/a | n/a | edkdeddddddddkke |
| 2492 | 2507 | TGCATCAGAAAAGCTC | 492 | 534531 | 61 | n/a | n/a | kekedddddddddkek |
| 2492 | 2507 | TGCATCAGAAAAGCTC | 492 | 534564 | 30 | n/a | n/a | kekdddddddddekek |
| 2492 | 2507 | TGCATCAGAAAAGCTC | 492 | 534597 | 67 | n/a | n/a | ekeedddddddddkke |
| 2492 | 2507 | TGCATCAGAAAAGCTC | 492 | 534630 | 54 | n/a | n/a | ekedddddddddekke |
| 2492 | 2507 | TGCATCAGAAAAGCTC | 492 | 534663 | 94 | n/a | n/a | eekkdddddddddeke |
| 2492 | 2507 | TGCATCAGAAAAGCTC | 492 | 534696 | 68 | n/a | n/a | eekdddddddddkeke |
| 2492 | 2507 | TGCATCAGAAAAGCTC | 492 | 534733 | 44 | n/a | n/a | ekekddddddddkeke |
| 2492 | 2507 | TGCATCAGAAAAGCTC | 492 | 534768 | 55 | n/a | n/a | keekddddddddkeek |
| 2492 | 2507 | TGCATCAGAAAAGCTC | 492 | 534803 | 73 | n/a | n/a | ekkddddddddddkke |
| 2492 | 2507 | TGCATCAGAAAAGCTC | 492 | 534833 | 65 | n/a | n/a | edkddddddddddkke |
| 2492 | 2507 | TGCATCAGAAAAGCTC | 492 | 534863 | 53 | n/a | n/a | kdeddddddddddkke |
| 2492 | 2507 | TGCATCAGAAAAGCTC | 492 | 534893 | 61 | n/a | n/a | eekddddddddddkke |
| 2492 | 2507 | TGCATCAGAAAAGCTC | 492 | 534923 | 70 | n/a | n/a | kddkdddddddddkke |
| 2492 | 2507 | TGCATCAGAAAAGCTC | 492 | 534953 | 54 | n/a | n/a | kddedddddddddkke |
| 2492 | 2507 | TGCATCAGAAAAGCTC | 492 | 534983 | 58 | n/a | n/a | eddkdddddddddkke |
| 2492 | 2507 | TGCATCAGAAAAGCTC | 492 | 535013 | 52 | n/a | n/a | eeeedddddddddkke |
| 2492 | 2507 | TGCATCAGAAAAGCTC | 492 | 535046 | 67 | n/a | n/a | eeeedddddddddkkk |
| 2492 | 2507 | TGCATCAGAAAAGCTC | 492 | 535079 | 57 | n/a | n/a | eeekdddddddddkke |
| 2492 | 2507 | TGCATCAGAAAAGCTC | 492 | 535112 | 42 | n/a | n/a | eeeeeddddddddkke |
| 2492 | 2507 | TGCATCAGAAAAGCTC | 492 | 535145 | 41 | n/a | n/a | ededkddddddddkke |
| 2492 | 2507 | TGCATCAGAAAAGCTC | 492 | 535178 | 35 | n/a | n/a | edkdeddddddddkke |
| 2498 | 2513 | CAGATTTGCATCAGAA | 493 | 534565 | 87 | n/a | n/a | kekdddddddddekek |
| 2498 | 2513 | CAGATTTGCATCAGAA | 493 | 534598 | 72 | n/a | n/a | ekeedddddddddkke |
| 2498 | 2513 | CAGATTTGCATCAGAA | 493 | 534631 | 70 | n/a | n/a | ekedddddddddekke |
| 2498 | 2513 | CAGATTTGCATCAGAA | 493 | 534664 | 94 | n/a | n/a | eekkdddddddddeke |
| 2498 | 2513 | CAGATTTGCATCAGAA | 493 | 534697 | 90 | n/a | n/a | eekdddddddddkeke |
| 2498 | 2513 | CAGATTTGCATCAGAA | 493 | 534734 | 74 | n/a | n/a | ekekddddddddkeke |
| 2498 | 2513 | CAGATTTGCATCAGAA | 493 | 534769 | 80 | n/a | n/a | keekddddddddkeek |
| 2498 | 2513 | CAGATTTGCATCAGAA | 493 | 534804 | 87 | n/a | n/a | ekkddddddddddkke |
| 2498 | 2513 | CAGATTTGCATCAGAA | 493 | 534834 | 76 | n/a | n/a | edkddddddddddkke |
| 2498 | 2513 | CAGATTTGCATCAGAA | 493 | 534864 | 56 | n/a | n/a | kdeddddddddddkke |
| 2498 | 2513 | CAGATTTGCATCAGAA | 493 | 534894 | 67 | n/a | n/a | eekddddddddddkke |
| 2498 | 2513 | CAGATTTGCATCAGAA | 493 | 534924 | 71 | n/a | n/a | kddkdddddddddkke |
| 2498 | 2513 | CAGATTTGCATCAGAA | 493 | 534954 | 54 | n/a | n/a | kddedddddddddkke |
| 2498 | 2513 | CAGATTTGCATCAGAA | 493 | 534984 | 48 | n/a | n/a | eddkdddddddddkke |
| 2498 | 2513 | CAGATTTGCATCAGAA | 493 | 535014 | 43 | n/a | n/a | eeeedddddddddkke |
| 2498 | 2513 | CAGATTTGCATCAGAA | 493 | 535047 | 60 | n/a | n/a | eeeedddddddddkkk |
| 2498 | 2513 | CAGATTTGCATCAGAA | 493 | 535080 | 64 | n/a | n/a | eeekdddddddddkke |
| 2498 | 2513 | CAGATTTGCATCAGAA | 493 | 535113 | 32 | n/a | n/a | eeeeeddddddddkke |
| 2498 | 2513 | CAGATTTGCATCAGAA | 493 | 535146 | 31 | n/a | n/a | ededkddddddddkke |
| 2498 | 2513 | CAGATTTGCATCAGAA | 493 | 535179 | 28 | n/a | n/a | edkdeddddddddkke |
| 4729 | 4744 | GTCTGGTTTGGAAGGA | 494 | 534533 | 82 | n/a | n/a | kekedddddddddkek |
| 4729 | 4744 | GTCTGGTTTGGAAGGA | 494 | 534566 | 88 | n/a | n/a | kekdddddddddekek |
| 4729 | 4744 | GTCTGGTTTGGAAGGA | 494 | 534599 | 65 | n/a | n/a | ekeedddddddddkke |
| 4729 | 4744 | GTCTGGTTTGGAAGGA | 494 | 534632 | 69 | n/a | n/a | ekedddddddddekke |
| 4729 | 4744 | GTCTGGTTTGGAAGGA | 494 | 534665 | 87 | n/a | n/a | eekkdddddddddeke |
| 4729 | 4744 | GTCTGGTTTGGAAGGA | 494 | 534698 | 64 | n/a | n/a | eekdddddddddkeke |
| 4729 | 4744 | GTCTGGTTTGGAAGGA | 494 | 534735 | 63 | n/a | n/a | ekekddddddddkeke |
| 4729 | 4744 | GTCTGGTTTGGAAGGA | 494 | 534770 | 66 | n/a | n/a | keekddddddddkeek |
| 4729 | 4744 | GTCTGGTTTGGAAGGA | 494 | 534805 | 87 | n/a | n/a | ekkddddddddddkke |
| 4729 | 4744 | GTCTGGTTTGGAAGGA | 494 | 534835 | 68 | n/a | n/a | edkddddddddddkke |
| 4729 | 4744 | GTCTGGTTTGGAAGGA | 494 | 534865 | 66 | n/a | n/a | kdeddddddddddkke |
| 4729 | 4744 | GTCTGGTTTGGAAGGA | 494 | 534895 | 57 | n/a | n/a | eekddddddddddkke |
| 4729 | 4744 | GTCTGGTTTGGAAGGA | 494 | 534925 | 82 | n/a | n/a | kddkdddddddddkke |
| 4729 | 4744 | GTCTGGTTTGGAAGGA | 494 | 534955 | 76 | n/a | n/a | kddedddddddddkke |
| 4729 | 4744 | GTCTGGTTTGGAAGGA | 494 | 534985 | 71 | n/a | n/a | eddkdddddddddkke |
| 4729 | 4744 | GTCTGGTTTGGAAGGA | 494 | 535015 | 59 | n/a | n/a | eeeedddddddddkke |
| 4729 | 4744 | GTCTGGTTTGGAAGGA | 494 | 535048 | 69 | n/a | n/a | eeeedddddddddkkk |
| 4729 | 4744 | GTCTGGTTTGGAAGGA | 494 | 535081 | 67 | n/a | n/a | eeekdddddddddkke |
| 4729 | 4744 | GTCTGGTTTGGAAGGA | 494 | 535114 | 37 | n/a | n/a | eeeeeddddddddkke |
| 4729 | 4744 | GTCTGGTTTGGAAGGA | 494 | 535147 | 32 | n/a | n/a | ededkddddddddkke |
| 4729 | 4744 | GTCTGGTTTGGAAGGA | 494 | 535180 | 31 | n/a | n/a | edkdeddddddddkke |
| 4851 | 4866 | GGTTACTGAGCGCGGA | 234 | 534534 | 94 | n/a | n/a | kekedddddddddkek |
| 4851 | 4866 | GGTTACTGAGCGCGGA | 234 | 534567 | 92 | n/a | n/a | kekdddddddddekek |
| 4851 | 4866 | GGTTACTGAGCGCGGA | 234 | 534600 | 92 | n/a | n/a | ekeedddddddddkke |
| 4851 | 4866 | GGTTACTGAGCGCGGA | 234 | 534633 | 91 | n/a | n/a | ekedddddddddekke |
| 4851 | 4866 | GGTTACTGAGCGCGGA | 234 | 534666 | 89 | n/a | n/a | eekkdddddddddeke |
| 4851 | 4866 | GGTTACTGAGCGCGGA | 234 | 534699 | 91 | n/a | n/a | eekdddddddddkeke |
| 4851 | 4866 | GGTTACTGAGCGCGGA | 234 | 534736 | 83 | n/a | n/a | ekekddddddddkeke |
| 4851 | 4866 | GGTTACTGAGCGCGGA | 234 | 534771 | 80 | n/a | n/a | keekddddddddkeek |
| 4851 | 4866 | GGTTACTGAGCGCGGA | 234 | 534806 | 96 | n/a | n/a | ekkddddddddddkke |
| 4851 | 4866 | GGTTACTGAGCGCGGA | 234 | 534836 | 86 | n/a | n/a | edkddddddddddkke |
| 4851 | 4866 | GGTTACTGAGCGCGGA | 234 | 534866 | 82 | n/a | n/a | kdeddddddddddkke |
| 4851 | 4866 | GGTTACTGAGCGCGGA | 234 | 534896 | 82 | n/a | n/a | eekddddddddddkke |
| 4851 | 4866 | GGTTACTGAGCGCGGA | 234 | 534926 | 89 | n/a | n/a | kddkdddddddddkke |
| 4851 | 4866 | GGTTACTGAGCGCGGA | 234 | 534956 | 91 | n/a | n/a | kddedddddddddkke |
| 4851 | 4866 | GGTTACTGAGCGCGGA | 234 | 534986 | 87 | n/a | n/a | eddkdddddddddkke |
| 4851 | 4866 | GGTTACTGAGCGCGGA | 234 | 535016 | 83 | n/a | n/a | eeeedddddddddkke |
| 4851 | 4866 | GGTTACTGAGCGCGGA | 234 | 535049 | 87 | n/a | n/a | eeeedddddddddkkk |
| 4851 | 4866 | GGTTACTGAGCGCGGA | 234 | 535082 | 87 | n/a | n/a | eeekdddddddddkke |
| 4851 | 4866 | GGTTACTGAGCGCGGA | 234 | 535115 | 77 | n/a | n/a | eeeeeddddddddkke |
| 4851 | 4866 | GGTTACTGAGCGCGGA | 234 | 535148 | 73 | n/a | n/a | ededkddddddddkke |
| 4851 | 4866 | GGTTACTGAGCGCGGA | 234 | 535181 | 68 | n/a | n/a | edkdeddddddddkke |
| 4873 | 4888 | TTCTGCAGGAGCGGCC | 236 | 534535 | 66 | n/a | n/a | kekedddddddddkek |
| 4873 | 4888 | TTCTGCAGGAGCGGCC | 236 | 534568 | 85 | n/a | n/a | kekdddddddddekek |
| 4873 | 4888 | TTCTGCAGGAGCGGCC | 236 | 534601 | 51 | n/a | n/a | ekeedddddddddkke |
| 4873 | 4888 | TTCTGCAGGAGCGGCC | 236 | 534634 | 80 | n/a | n/a | ekedddddddddekke |
| 4873 | 4888 | TTCTGCAGGAGCGGCC | 236 | 534667 | 90 | n/a | n/a | eekkdddddddddeke |
| 4873 | 4888 | TTCTGCAGGAGCGGCC | 236 | 534700 | 88 | n/a | n/a | eekdddddddddkeke |
| 4873 | 4888 | TTCTGCAGGAGCGGCC | 236 | 534737 | 65 | n/a | n/a | ekekddddddddkeke |
| 4873 | 4888 | TTCTGCAGGAGCGGCC | 236 | 534772 | 77 | n/a | n/a | keekddddddddkeek |
| 4873 | 4888 | TTCTGCAGGAGCGGCC | 236 | 534807 | 84 | n/a | n/a | ekkddddddddddkke |
| 4873 | 4888 | TTCTGCAGGAGCGGCC | 236 | 534837 | 78 | n/a | n/a | edkddddddddddkke |
| 4873 | 4888 | TTCTGCAGGAGCGGCC | 236 | 534867 | 44 | n/a | n/a | kdeddddddddddkke |
| 4873 | 4888 | TTCTGCAGGAGCGGCC | 236 | 534897 | 82 | n/a | n/a | eekddddddddddkke |
| 4873 | 4888 | TTCTGCAGGAGCGGCC | 236 | 534927 | 61 | n/a | n/a | kddkdddddddddkke |
| 4873 | 4888 | TTCTGCAGGAGCGGCC | 236 | 534957 | 58 | n/a | n/a | kddedddddddddkke |
| 4873 | 4888 | TTCTGCAGGAGCGGCC | 236 | 534987 | 49 | n/a | n/a | eddkdddddddddkke |
| 4873 | 4888 | TTCTGCAGGAGCGGCC | 236 | 535017 | 38 | n/a | n/a | eeeedddddddddkke |
| 4873 | 4888 | TTCTGCAGGAGCGGCC | 236 | 535050 | 32 | n/a | n/a | eeeedddddddddkkk |
| 4873 | 4888 | TTCTGCAGGAGCGGCC | 236 | 535083 | 43 | n/a | n/a | eeekdddddddddkke |
| 4873 | 4888 | TTCTGCAGGAGCGGCC | 236 | 535116 | 9 | n/a | n/a | eeeeeddddddddkke |
| 4873 | 4888 | TTCTGCAGGAGCGGCC | 236 | 535149 | 23 | n/a | n/a | ededkddddddddkke |
| 4873 | 4888 | TTCTGCAGGAGCGGCC | 236 | 535182 | 18 | n/a | n/a | edkdeddddddddkke |
| 5512 | 5527 | CCGAGGCGCGGCCCCT | 238 | 534536 | 89 | n/a | n/a | kekedddddddddkek |
| 5512 | 5527 | CCGAGGCGCGGCCCCT | 238 | 534569 | 90 | n/a | n/a | kekdddddddddekek |
| 5512 | 5527 | CCGAGGCGCGGCCCCT | 238 | 534602 | 85 | n/a | n/a | ekeedddddddddkke |
| 5512 | 5527 | CCGAGGCGCGGCCCCT | 238 | 534635 | 87 | n/a | n/a | ekedddddddddekke |
| 5512 | 5527 | CCGAGGCGCGGCCCCT | 238 | 534668 | 90 | n/a | n/a | eekkdddddddddeke |
| 5512 | 5527 | CCGAGGCGCGGCCCCT | 238 | 534701 | 92 | n/a | n/a | eekdddddddddkeke |
| 5512 | 5527 | CCGAGGCGCGGCCCCT | 238 | 534738 | 81 | n/a | n/a | ekekddddddddkeke |
| 5512 | 5527 | CCGAGGCGCGGCCCCT | 238 | 534773 | 79 | n/a | n/a | keekddddddddkeek |
| 5512 | 5527 | CCGAGGCGCGGCCCCT | 238 | 534808 | 90 | n/a | n/a | ekkddddddddddkke |
| 5512 | 5527 | CCGAGGCGCGGCCCCT | 238 | 534838 | 88 | n/a | n/a | edkddddddddddkke |
| 5512 | 5527 | CCGAGGCGCGGCCCCT | 238 | 534868 | 67 | n/a | n/a | kdeddddddddddkke |
| 5512 | 5527 | CCGAGGCGCGGCCCCT | 238 | 534898 | 89 | n/a | n/a | eekddddddddddkke |
| 5512 | 5527 | CCGAGGCGCGGCCCCT | 238 | 534928 | 81 | n/a | n/a | kddkdddddddddkke |
| 5512 | 5527 | CCGAGGCGCGGCCCCT | 238 | 534958 | 78 | n/a | n/a | kddedddddddddkke |
| 5512 | 5527 | CCGAGGCGCGGCCCCT | 238 | 534988 | 66 | n/a | n/a | eddkdddddddddkke |
| 5512 | 5527 | CCGAGGCGCGGCCCCT | 238 | 535018 | 78 | n/a | n/a | eeeedddddddddkke |
| 5512 | 5527 | CCGAGGCGCGGCCCCT | 238 | 535051 | 76 | n/a | n/a | eeeedddddddddkkk |
| 5512 | 5527 | CCGAGGCGCGGCCCCT | 238 | 535084 | 80 | n/a | n/a | eeekdddddddddkke |
| 5512 | 5527 | CCGAGGCGCGGCCCCT | 238 | 535117 | 58 | n/a | n/a | eeeeeddddddddkke |
| 5512 | 5527 | CCGAGGCGCGGCCCCT | 238 | 535150 | 51 | n/a | n/a | ededkddddddddkke |
| 5512 | 5527 | CCGAGGCGCGGCCCCT | 238 | 535183 | 53 | n/a | n/a | edkdeddddddddkke |
| 5513 | 5528 | TCCGAGGCGCGGCCCC | 239 | 534537 | 91 | n/a | n/a | kekedddddddddkek |
| 5513 | 5528 | TCCGAGGCGCGGCCCC | 239 | 534570 | 85 | n/a | n/a | kekdddddddddekek |
| 5513 | 5528 | TCCGAGGCGCGGCCCC | 239 | 534603 | 79 | n/a | n/a | ekeedddddddddkke |
| 5513 | 5528 | TCCGAGGCGCGGCCCC | 239 | 534636 | 72 | n/a | n/a | ekedddddddddekke |
| 5513 | 5528 | TCCGAGGCGCGGCCCC | 239 | 534669 | 85 | n/a | n/a | eekkdddddddddeke |
| 5513 | 5528 | TCCGAGGCGCGGCCCC | 239 | 534702 | 85 | n/a | n/a | eekdddddddddkeke |
| 5513 | 5528 | TCCGAGGCGCGGCCCC | 239 | 534739 | 73 | n/a | n/a | ekekddddddddkeke |
| 5513 | 5528 | TCCGAGGCGCGGCCCC | 239 | 534774 | 77 | n/a | n/a | keekddddddddkeek |
| 5513 | 5528 | TCCGAGGCGCGGCCCC | 239 | 534809 | 91 | n/a | n/a | ekkddddddddddkke |
| 5513 | 5528 | TCCGAGGCGCGGCCCC | 239 | 534839 | 86 | n/a | n/a | edkddddddddddkke |
| 5513 | 5528 | TCCGAGGCGCGGCCCC | 239 | 534869 | 71 | n/a | n/a | kdeddddddddddkke |
| 5513 | 5528 | TCCGAGGCGCGGCCCC | 239 | 534899 | 82 | n/a | n/a | eekddddddddddkke |
| 5513 | 5528 | TCCGAGGCGCGGCCCC | 239 | 534929 | 83 | n/a | n/a | kddkdddddddddkke |
| 5513 | 5528 | TCCGAGGCGCGGCCCC | 239 | 534959 | 80 | n/a | n/a | kddedddddddddkke |
| 5513 | 5528 | TCCGAGGCGCGGCCCC | 239 | 534989 | 79 | n/a | n/a | eddkdddddddddkke |
| 5513 | 5528 | TCCGAGGCGCGGCCCC | 239 | 535019 | 76 | n/a | n/a | eeeedddddddddkke |
| 5513 | 5528 | TCCGAGGCGCGGCCCC | 239 | 535052 | 79 | n/a | n/a | eeeedddddddddkkk |
| 5513 | 5528 | TCCGAGGCGCGGCCCC | 239 | 535085 | 81 | n/a | n/a | eeekdddddddddkke |
| 5513 | 5528 | TCCGAGGCGCGGCCCC | 239 | 535118 | 58 | n/a | n/a | eeeeeddddddddkke |
| 5513 | 5528 | TCCGAGGCGCGGCCCC | 239 | 535151 | 65 | n/a | n/a | ededkddddddddkke |
| 5513 | 5528 | TCCGAGGCGCGGCCCC | 239 | 535184 | 60 | n/a | n/a | edkdeddddddddkke |
| 6077 | 6092 | CCCGGCCGCAGCTCCT | 495 | 534516 | 77 | 182 | 197 | kekedddddddddkek |
| 6077 | 6092 | CCCGGCCGCAGCTCCT | 495 | 534549 | 80 | 182 | 197 | kekdddddddddekek |
| 6077 | 6092 | CCCGGCCGCAGCTCCT | 495 | 534582 | 73 | 182 | 197 | ekeedddddddddkke |
| 6077 | 6092 | CCCGGCCGCAGCTCCT | 495 | 534615 | 79 | 182 | 197 | ekedddddddddekke |
| 6077 | 6092 | CCCGGCCGCAGCTCCT | 495 | 534648 | 67 | 182 | 197 | eekkdddddddddeke |
| 6077 | 6092 | CCCGGCCGCAGCTCCT | 495 | 534681 | 87 | 182 | 197 | eekdddddddddkeke |
| 6077 | 6092 | CCCGGCCGCAGCTCCT | 495 | 534718 | 46 | 182 | 197 | ekekddddddddkeke |
| 6077 | 6092 | CCCGGCCGCAGCTCCT | 495 | 534753 | 68 | 182 | 197 | keekddddddddkeek |
| 6077 | 6092 | CCCGGCCGCAGCTCCT | 495 | 534788 | 84 | 182 | 197 | ekkddddddddddkke |
| 6077 | 6092 | CCCGGCCGCAGCTCCT | 495 | 534818 | 82 | 182 | 197 | edkddddddddddkke |
| 6077 | 6092 | CCCGGCCGCAGCTCCT | 495 | 534848 | 75 | 182 | 197 | kdeddddddddddkke |
| 6077 | 6092 | CCCGGCCGCAGCTCCT | 495 | 534878 | 72 | 182 | 197 | eekddddddddddkke |
| 6077 | 6092 | CCCGGCCGCAGCTCCT | 495 | 534908 | 81 | 182 | 197 | kddkdddddddddkke |
| 6077 | 6092 | CCCGGCCGCAGCTCCT | 495 | 534938 | 69 | 182 | 197 | kddedddddddddkke |
| 6077 | 6092 | CCCGGCCGCAGCTCCT | 495 | 534968 | 77 | 182 | 197 | eddkdddddddddkke |
| 6077 | 6092 | CCCGGCCGCAGCTCCT | 495 | 534998 | 76 | 182 | 197 | eeeedddddddddkke |
| 6077 | 6092 | CCCGGCCGCAGCTCCT | 495 | 535031 | 76 | 182 | 197 | eeeedddddddddkkk |
| 6077 | 6092 | CCCGGCCGCAGCTCCT | 495 | 535064 | 70 | 182 | 197 | eeekdddddddddkke |
| 6077 | 6092 | CCCGGCCGCAGCTCCT | 495 | 535097 | 57 | 182 | 197 | eeeeeddddddddkke |
| 6077 | 6092 | CCCGGCCGCAGCTCCT | 495 | 535130 | 69 | 182 | 197 | ededkddddddddkke |
| 6077 | 6092 | CCCGGCCGCAGCTCCT | 495 | 535163 | 58 | 182 | 197 | edkdeddddddddkke |
| 8644 | 8659 | AAGAAACTGTTGGCCA | 241 | 534538 | 71 | n/a | n/a | kekedddddddddkek |
| 8644 | 8659 | AAGAAACTGTTGGCCA | 241 | 534571 | 64 | n/a | n/a | kekdddddddddekek |
| 8644 | 8659 | AAGAAACTGTTGGCCA | 241 | 534604 | 66 | n/a | n/a | ekeedddddddddkke |
| 8644 | 8659 | AAGAAACTGTTGGCCA | 241 | 534637 | 74 | n/a | n/a | ekedddddddddekke |
| 8644 | 8659 | AAGAAACTGTTGGCCA | 241 | 534670 | 87 | n/a | n/a | eekkdddddddddeke |
| 8644 | 8659 | AAGAAACTGTTGGCCA | 241 | 534703 | 72 | n/a | n/a | eekdddddddddkeke |
| 8644 | 8659 | AAGAAACTGTTGGCCA | 241 | 534740 | 56 | n/a | n/a | ekekddddddddkeke |
| 8644 | 8659 | AAGAAACTGTTGGCCA | 241 | 534775 | 53 | n/a | n/a | keekddddddddkeek |
| 8644 | 8659 | AAGAAACTGTTGGCCA | 241 | 534810 | 78 | n/a | n/a | ekkddddddddddkke |
| 8644 | 8659 | AAGAAACTGTTGGCCA | 241 | 534840 | 73 | n/a | n/a | edkddddddddddkke |
| 8644 | 8659 | AAGAAACTGTTGGCCA | 241 | 534870 | 65 | n/a | n/a | kdeddddddddddkke |
| 8644 | 8659 | AAGAAACTGTTGGCCA | 241 | 534900 | 69 | n/a | n/a | eekddddddddddkke |
| 8644 | 8659 | AAGAAACTGTTGGCCA | 241 | 534930 | 67 | n/a | n/a | kddkdddddddddkke |
| 8644 | 8659 | AAGAAACTGTTGGCCA | 241 | 534960 | 62 | n/a | n/a | kddedddddddddkke |
| 8644 | 8659 | AAGAAACTGTTGGCCA | 241 | 534990 | 66 | n/a | n/a | eddkdddddddddkke |
| 8644 | 8659 | AAGAAACTGTTGGCCA | 241 | 535020 | 61 | n/a | n/a | eeeedddddddddkke |
| 8644 | 8659 | AAGAAACTGTTGGCCA | 241 | 535053 | 47 | n/a | n/a | eeeedddddddddkkk |
| 8644 | 8659 | AAGAAACTGTTGGCCA | 241 | 535086 | 61 | n/a | n/a | eeekdddddddddkke |
| 8644 | 8659 | AAGAAACTGTTGGCCA | 241 | 535119 | 49 | n/a | n/a | eeeeeddddddddkke |
| 8644 | 8659 | AAGAAACTGTTGGCCA | 241 | 535152 | 48 | n/a | n/a | ededkddddddddkke |
| 8644 | 8659 | AAGAAACTGTTGGCCA | 241 | 535185 | 57 | n/a | n/a | edkdeddddddddkke |
| 8861 | 8876 | ATGGGTGACCACACAT | 496 | 534539 | 70 | n/a | n/a | kekedddddddddkek |
| 8861 | 8876 | ATGGGTGACCACACAT | 496 | 534572 | 82 | n/a | n/a | kekdddddddddekek |
| 8861 | 8876 | ATGGGTGACCACACAT | 496 | 534605 | 59 | n/a | n/a | ekeedddddddddkke |
| 8861 | 8876 | ATGGGTGACCACACAT | 496 | 534638 | 69 | n/a | n/a | ekedddddddddekke |
| 8861 | 8876 | ATGGGTGACCACACAT | 496 | 534671 | 89 | n/a | n/a | eekkdddddddddeke |
| 8861 | 8876 | ATGGGTGACCACACAT | 496 | 534704 | 83 | n/a | n/a | eekdddddddddkeke |
| 8861 | 8876 | ATGGGTGACCACACAT | 496 | 534741 | 47 | n/a | n/a | ekekddddddddkeke |
| 8861 | 8876 | ATGGGTGACCACACAT | 496 | 534776 | 46 | n/a | n/a | keekddddddddkeek |
| 8861 | 8876 | ATGGGTGACCACACAT | 496 | 534811 | 71 | n/a | n/a | ekkddddddddddkke |
| 8861 | 8876 | ATGGGTGACCACACAT | 496 | 534841 | 61 | n/a | n/a | edkddddddddddkke |
| 8861 | 8876 | ATGGGTGACCACACAT | 496 | 534871 | 53 | n/a | n/a | kdeddddddddddkke |
| 8861 | 8876 | ATGGGTGACCACACAT | 496 | 534901 | 55 | n/a | n/a | eekddddddddddkke |
| 8861 | 8876 | ATGGGTGACCACACAT | 496 | 534931 | 73 | n/a | n/a | kddkdddddddddkke |
| 8861 | 8876 | ATGGGTGACCACACAT | 496 | 534961 | 53 | n/a | n/a | kddedddddddddkke |
| 8861 | 8876 | ATGGGTGACCACACAT | 496 | 534991 | 56 | n/a | n/a | eddkdddddddddkke |
| 8861 | 8876 | ATGGGTGACCACACAT | 496 | 535021 | 58 | n/a | n/a | eeeedddddddddkke |
| 8861 | 8876 | ATGGGTGACCACACAT | 496 | 535054 | 59 | n/a | n/a | eeeedddddddddkkk |
| 8861 | 8876 | ATGGGTGACCACACAT | 496 | 535087 | 0 | n/a | n/a | eeekdddddddddkke |
| 8861 | 8876 | ATGGGTGACCACACAT | 496 | 535120 | 41 | n/a | n/a | eeeeeddddddddkke |
| 8861 | 8876 | ATGGGTGACCACACAT | 496 | 535153 | 44 | n/a | n/a | ededkddddddddkke |
| 8861 | 8876 | ATGGGTGACCACACAT | 496 | 535186 | 35 | n/a | n/a | edkdeddddddddkke |
| 9598 | 9613 | AAGTTTACCAAGCGGT | 497 | 534573 | 76 | n/a | n/a | kekdddddddddekek |
| 9598 | 9613 | AAGTTTACCAAGCGGT | 497 | 534606 | 55 | n/a | n/a | ekeedddddddddkke |
| 9598 | 9613 | AAGTTTACCAAGCGGT | 497 | 534639 | 72 | n/a | n/a | ekedddddddddekke |
| 9598 | 9613 | AAGTTTACCAAGCGGT | 497 | 534672 | 89 | n/a | n/a | eekkdddddddddeke |
| 9598 | 9613 | AAGTTTACCAAGCGGT | 497 | 534705 | 87 | n/a | n/a | eekdddddddddkeke |
| 9598 | 9613 | AAGTTTACCAAGCGGT | 497 | 534742 | 84 | n/a | n/a | ekekddddddddkeke |
| 9598 | 9613 | AAGTTTACCAAGCGGT | 497 | 534777 | 79 | n/a | n/a | keekddddddddkeek |
| 9598 | 9613 | AAGTTTACCAAGCGGT | 497 | 534812 | 76 | n/a | n/a | ekkddddddddddkke |
| 9598 | 9613 | AAGTTTACCAAGCGGT | 497 | 534842 | 74 | n/a | n/a | edkddddddddddkke |
| 9598 | 9613 | AAGTTTACCAAGCGGT | 497 | 534872 | 53 | n/a | n/a | kdeddddddddddkke |
| 9598 | 9613 | AAGTTTACCAAGCGGT | 497 | 534902 | 70 | n/a | n/a | eekddddddddddkke |
| 9598 | 9613 | AAGTTTACCAAGCGGT | 497 | 534932 | 73 | n/a | n/a | kddkdddddddddkke |
| 9598 | 9613 | AAGTTTACCAAGCGGT | 497 | 534962 | 60 | n/a | n/a | kddedddddddddkke |
| 9598 | 9613 | AAGTTTACCAAGCGGT | 497 | 534992 | 61 | n/a | n/a | eddkdddddddddkke |
| 9598 | 9613 | AAGTTTACCAAGCGGT | 497 | 535022 | 38 | n/a | n/a | eeeedddddddddkke |
| 9598 | 9613 | AAGTTTACCAAGCGGT | 497 | 535055 | 42 | n/a | n/a | eeeedddddddddkkk |
| 9598 | 9613 | AAGTTTACCAAGCGGT | 497 | 535088 | 56 | n/a | n/a | eeekdddddddddkke |
| 9598 | 9613 | AAGTTTACCAAGCGGT | 497 | 535121 | 5 | n/a | n/a | eeeeeddddddddkke |
| 9598 | 9613 | AAGTTTACCAAGCGGT | 497 | 535154 | 22 | n/a | n/a | ededkddddddddkke |
| 9598 | 9613 | AAGTTTACCAAGCGGT | 497 | 535187 | 16 | n/a | n/a | edkdeddddddddkke |
| 9599 | 9614 | GAAGTTTACCAAGCGG | 498 | 534541 | 86 | n/a | n/a | kekedddddddddkek |
| 9599 | 9614 | GAAGTTTACCAAGCGG | 498 | 534574 | 89 | n/a | n/a | kekdddddddddekek |
| 9599 | 9614 | GAAGTTTACCAAGCGG | 498 | 534607 | 59 | n/a | n/a | ekeedddddddddkke |
| 9599 | 9614 | GAAGTTTACCAAGCGG | 498 | 534640 | 76 | n/a | n/a | ekedddddddddekke |
| 9599 | 9614 | GAAGTTTACCAAGCGG | 498 | 534673 | 89 | n/a | n/a | eekkdddddddddeke |
| 9599 | 9614 | GAAGTTTACCAAGCGG | 498 | 534706 | 86 | n/a | n/a | eekdddddddddkeke |
| 9599 | 9614 | GAAGTTTACCAAGCGG | 498 | 534743 | 79 | n/a | n/a | ekekddddddddkeke |
| 9599 | 9614 | GAAGTTTACCAAGCGG | 498 | 534778 | 80 | n/a | n/a | keekddddddddkeek |
| 9599 | 9614 | GAAGTTTACCAAGCGG | 498 | 534813 | 83 | n/a | n/a | ekkddddddddddkke |
| 9599 | 9614 | GAAGTTTACCAAGCGG | 498 | 534843 | 82 | n/a | n/a | edkddddddddddkke |
| 9599 | 9614 | GAAGTTTACCAAGCGG | 498 | 534873 | 83 | n/a | n/a | kdeddddddddddkke |
| 9599 | 9614 | GAAGTTTACCAAGCGG | 498 | 534903 | 78 | n/a | n/a | eekddddddddddkke |
| 9599 | 9614 | GAAGTTTACCAAGCGG | 498 | 534933 | 83 | n/a | n/a | kddkdddddddddkke |
| 9599 | 9614 | GAAGTTTACCAAGCGG | 498 | 534963 | 70 | n/a | n/a | kddedddddddddkke |
| 9599 | 9614 | GAAGTTTACCAAGCGG | 498 | 534993 | 78 | n/a | n/a | eddkdddddddddkke |
| 9599 | 9614 | GAAGTTTACCAAGCGG | 498 | 535023 | 56 | n/a | n/a | eeeedddddddddkke |
| 9599 | 9614 | GAAGTTTACCAAGCGG | 498 | 535056 | 59 | n/a | n/a | eeeedddddddddkkk |
| 9599 | 9614 | GAAGTTTACCAAGCGG | 498 | 535089 | 73 | n/a | n/a | eeekdddddddddkke |
| 9599 | 9614 | GAAGTTTACCAAGCGG | 498 | 535122 | 39 | n/a | n/a | eeeeeddddddddkke |
| 9599 | 9614 | GAAGTTTACCAAGCGG | 498 | 535155 | 60 | n/a | n/a | ededkddddddddkke |
| 9599 | 9614 | GAAGTTTACCAAGCGG | 498 | 535188 | 41 | n/a | n/a | edkdeddddddddkke |
| 9939 | 9954 | ACCTCTGGACACCGGG | 499 | 534542 | 75 | n/a | n/a | kekedddddddddkek |
| 9939 | 9954 | ACCTCTGGACACCGGG | 499 | 534575 | 82 | n/a | n/a | kekdddddddddekek |
| 9939 | 9954 | ACCTCTGGACACCGGG | 499 | 534608 | 72 | n/a | n/a | ekeedddddddddkke |
| 9939 | 9954 | ACCTCTGGACACCGGG | 499 | 534641 | 69 | n/a | n/a | ekedddddddddekke |
| 9939 | 9954 | ACCTCTGGACACCGGG | 499 | 534674 | 84 | n/a | n/a | eekkdddddddddeke |
| 9939 | 9954 | ACCTCTGGACACCGGG | 499 | 534707 | 78 | n/a | n/a | eekdddddddddkeke |
| 9939 | 9954 | ACCTCTGGACACCGGG | 499 | 534744 | 72 | n/a | n/a | ekekddddddddkeke |
| 9939 | 9954 | ACCTCTGGACACCGGG | 499 | 534779 | 75 | n/a | n/a | keekddddddddkeek |
| 9939 | 9954 | ACCTCTGGACACCGGG | 499 | 534814 | 81 | n/a | n/a | ekkddddddddddkke |
| 9939 | 9954 | ACCTCTGGACACCGGG | 499 | 534844 | 75 | n/a | n/a | edkddddddddddkke |
| 9939 | 9954 | ACCTCTGGACACCGGG | 499 | 534874 | 70 | n/a | n/a | kdeddddddddddkke |
| 9939 | 9954 | ACCTCTGGACACCGGG | 499 | 534904 | 71 | n/a | n/a | eekddddddddddkke |
| 9939 | 9954 | ACCTCTGGACACCGGG | 499 | 534934 | 73 | n/a | n/a | kddkdddddddddkke |
| 9939 | 9954 | ACCTCTGGACACCGGG | 499 | 534964 | 72 | n/a | n/a | kddedddddddddkke |
| 9939 | 9954 | ACCTCTGGACACCGGG | 499 | 534994 | 69 | n/a | n/a | eddkdddddddddkke |
| 9939 | 9954 | ACCTCTGGACACCGGG | 499 | 535024 | 56 | n/a | n/a | eeeedddddddddkke |
| 9939 | 9954 | ACCTCTGGACACCGGG | 499 | 535057 | 63 | n/a | n/a | eeeedddddddddkkk |
| 9939 | 9954 | ACCTCTGGACACCGGG | 499 | 535090 | 64 | n/a | n/a | eeekdddddddddkke |
| 9939 | 9954 | ACCTCTGGACACCGGG | 499 | 535123 | 40 | n/a | n/a | eeeeeddddddddkke |
| 9939 | 9954 | ACCTCTGGACACCGGG | 499 | 535156 | 47 | n/a | n/a | ededkddddddddkke |
| 9939 | 9954 | ACCTCTGGACACCGGG | 499 | 535189 | 48 | n/a | n/a | edkdeddddddddkke |
| 11839 | 11854 | AATGGTCAGGGCTGGT | 34 | 534515 | 52 | n/a | n/a | kekedddddddddkek |
| 11839 | 11854 | AATGGTCAGGGCTGGT | 34 | 534548 | 85 | n/a | n/a | kekdddddddddekek |
| 11839 | 11854 | AATGGTCAGGGCTGGT | 34 | 534581 | 75 | n/a | n/a | ekeedddddddddkke |
| 11839 | 11854 | AATGGTCAGGGCTGGT | 34 | 534614 | 83 | n/a | n/a | ekedddddddddekke |
| 11839 | 11854 | AATGGTCAGGGCTGGT | 34 | 534647 | 65 | n/a | n/a | eekkdddddddddeke |
| 11839 | 11854 | AATGGTCAGGGCTGGT | 34 | 534680 | 88 | n/a | n/a | eekdddddddddkeke |
| 11839 | 11854 | AATGGTCAGGGCTGGT | 34 | 534717 | 76 | n/a | n/a | ekekddddddddkeke |
| 11839 | 11854 | AATGGTCAGGGCTGGT | 34 | 534752 | 79 | n/a | n/a | keekddddddddkeek |
| 11839 | 11854 | AATGGTCAGGGCTGGT | 34 | 534787 | 90 | n/a | n/a | ekkddddddddddkke |
| 11839 | 11854 | AATGGTCAGGGCTGGT | 34 | 535030 | 77 | n/a | n/a | eeeedddddddddkkk |
| 11839 | 11854 | AATGGTCAGGGCTGGT | 34 | 535063 | 75 | n/a | n/a | eeekdddddddddkke |
| 11839 | 11854 | AATGGTCAGGGCTGGT | 34 | 535096 | 54 | n/a | n/a | eeeeeddddddddkke |
| 11839 | 11854 | AATGGTCAGGGCTGGT | 34 | 535129 | 66 | n/a | n/a | ededkddddddddkke |
| 11839 | 11854 | AATGGTCAGGGCTGGT | 34 | 535162 | 49 | n/a | n/a | edkdeddddddddkke |
| 11847 | 11862 | GAGGAGACAATGGTCA | 500 | 534543 | 66 | n/a | n/a | kekedddddddddkek |
| 11847 | 11862 | GAGGAGACAATGGTCA | 500 | 534576 | 69 | n/a | n/a | kekdddddddddekek |
| 11847 | 11862 | GAGGAGACAATGGTCA | 500 | 534609 | 77 | n/a | n/a | ekeedddddddddkke |
| 11847 | 11862 | GAGGAGACAATGGTCA | 500 | 534642 | 62 | n/a | n/a | ekedddddddddekke |
| 11847 | 11862 | GAGGAGACAATGGTCA | 500 | 534675 | 80 | n/a | n/a | eekkdddddddddeke |
| 11847 | 11862 | GAGGAGACAATGGTCA | 500 | 534708 | 81 | n/a | n/a | eekdddddddddkeke |
| 11847 | 11862 | GAGGAGACAATGGTCA | 500 | 534745 | 68 | n/a | n/a | ekekddddddddkeke |
| 11847 | 11862 | GAGGAGACAATGGTCA | 500 | 534780 | 69 | n/a | n/a | keekddddddddkeek |
| 11847 | 11862 | GAGGAGACAATGGTCA | 500 | 534815 | 85 | n/a | n/a | ekkddddddddddkke |
| 11847 | 11862 | GAGGAGACAATGGTCA | 500 | 534845 | 72 | n/a | n/a | edkddddddddddkke |
| 11847 | 11862 | GAGGAGACAATGGTCA | 500 | 534875 | 56 | n/a | n/a | kdeddddddddddkke |
| 11847 | 11862 | GAGGAGACAATGGTCA | 500 | 534905 | 65 | n/a | n/a | eekddddddddddkke |
| 11847 | 11862 | GAGGAGACAATGGTCA | 500 | 534935 | 78 | n/a | n/a | kddkdddddddddkke |
| 11847 | 11862 | GAGGAGACAATGGTCA | 500 | 534965 | 48 | n/a | n/a | kddedddddddddkke |
| 11847 | 11862 | GAGGAGACAATGGTCA | 500 | 534995 | 62 | n/a | n/a | eddkdddddddddkke |
| 11847 | 11862 | GAGGAGACAATGGTCA | 500 | 535025 | 58 | n/a | n/a | eeeedddddddddkke |
| 11847 | 11862 | GAGGAGACAATGGTCA | 500 | 535058 | 60 | n/a | n/a | eeeedddddddddkkk |
| 11847 | 11862 | GAGGAGACAATGGTCA | 500 | 535091 | 61 | n/a | n/a | eeekdddddddddkke |
| 11847 | 11862 | GAGGAGACAATGGTCA | 500 | 535124 | 51 | n/a | n/a | eeeeeddddddddkke |
| 11847 | 11862 | GAGGAGACAATGGTCA | 500 | 535157 | 55 | n/a | n/a | ededkddddddddkke |
| 11847 | 11862 | GAGGAGACAATGGTCA | 500 | 535190 | 47 | n/a | n/a | edkdeddddddddkke |
| 12082 | 12097 | TGGATATTCAACTGTG | 501 | 534517 | 71 | 552 | 567 | kekedddddddddkek |
| 12082 | 12097 | TGGATATTCAACTGTG | 501 | 534550 | 80 | 552 | 567 | kekdddddddddekek |
| 12082 | 12097 | TGGATATTCAACTGTG | 501 | 534583 | 70 | 552 | 567 | ekeedddddddddkke |
| 12082 | 12097 | TGGATATTCAACTGTG | 501 | 534616 | 84 | 552 | 567 | ekedddddddddekke |
| 12082 | 12097 | TGGATATTCAACTGTG | 501 | 534649 | 68 | 552 | 567 | eekkdddddddddeke |
| 12082 | 12097 | TGGATATTCAACTGTG | 501 | 534682 | 87 | 552 | 567 | eekdddddddddkeke |
| 12082 | 12097 | TGGATATTCAACTGTG | 501 | 534719 | 90 | 552 | 567 | ekekddddddddkeke |
| 12082 | 12097 | TGGATATTCAACTGTG | 501 | 534754 | 83 | 552 | 567 | keekddddddddkeek |
| 12082 | 12097 | TGGATATTCAACTGTG | 501 | 534789 | 86 | 552 | 567 | ekkddddddddddkke |
| 12082 | 12097 | TGGATATTCAACTGTG | 501 | 534819 | 69 | 552 | 567 | edkddddddddddkke |
| 12082 | 12097 | TGGATATTCAACTGTG | 501 | 534849 | 62 | 552 | 567 | kdeddddddddddkke |
| 12082 | 12097 | TGGATATTCAACTGTG | 501 | 534879 | 69 | 552 | 567 | eekddddddddddkke |
| 12082 | 12097 | TGGATATTCAACTGTG | 501 | 534909 | 73 | 552 | 567 | kddkdddddddddkke |
| 12082 | 12097 | TGGATATTCAACTGTG | 501 | 534939 | 49 | 552 | 567 | kddedddddddddkke |
| 12082 | 12097 | TGGATATTCAACTGTG | 501 | 534969 | 47 | 552 | 567 | eddkdddddddddkke |
| 12082 | 12097 | TGGATATTCAACTGTG | 501 | 534999 | 51 | 552 | 567 | eeeedddddddddkke |
| 12082 | 12097 | TGGATATTCAACTGTG | 501 | 535032 | 51 | 552 | 567 | eeeedddddddddkkk |
| 12082 | 12097 | TGGATATTCAACTGTG | 501 | 535065 | 64 | 552 | 567 | eeekdddddddddkke |
| 12082 | 12097 | TGGATATTCAACTGTG | 501 | 535098 | 31 | 552 | 567 | eeeeeddddddddkke |
| 12082 | 12097 | TGGATATTCAACTGTG | 501 | 535131 | 31 | 552 | 567 | ededkddddddddkke |
| 12082 | 12097 | TGGATATTCAACTGTG | 501 | 535164 | 40 | 552 | 567 | edkdeddddddddkke |
| 12098 | 12113 | TAGGTATTTTTCCACA | 502 | 534518 | 81 | 568 | 583 | kekedddddddddkek |
| 12098 | 12113 | TAGGTATTTTTCCACA | 502 | 534551 | 88 | 568 | 583 | kekdddddddddekek |
| 12098 | 12113 | TAGGTATTTTTCCACA | 502 | 534584 | 78 | 568 | 583 | ekeedddddddddkke |
| 12098 | 12113 | TAGGTATTTTTCCACA | 502 | 534617 | 80 | 568 | 583 | ekedddddddddekke |
| 12098 | 12113 | TAGGTATTTTTCCACA | 502 | 534650 | 83 | 568 | 583 | eekkdddddddddeke |
| 12098 | 12113 | TAGGTATTTTTCCACA | 502 | 534683 | 93 | 568 | 583 | eekdddddddddkeke |
| 12098 | 12113 | TAGGTATTTTTCCACA | 502 | 534720 | 87 | 568 | 583 | ekekddddddddkeke |
| 12098 | 12113 | TAGGTATTTTTCCACA | 502 | 534755 | 82 | 568 | 583 | keekddddddddkeek |
| 12098 | 12113 | TAGGTATTTTTCCACA | 502 | 534790 | 89 | 568 | 583 | ekkddddddddddkke |
| 12098 | 12113 | TAGGTATTTTTCCACA | 502 | 534820 | 64 | 568 | 583 | edkddddddddddkke |
| 12098 | 12113 | TAGGTATTTTTCCACA | 502 | 534850 | 38 | 568 | 583 | kdeddddddddddkke |
| 12098 | 12113 | TAGGTATTTTTCCACA | 502 | 534880 | 68 | 568 | 583 | eekddddddddddkke |
| 12098 | 12113 | TAGGTATTTTTCCACA | 502 | 534910 | 60 | 568 | 583 | kddkdddddddddkke |
| 12098 | 12113 | TAGGTATTTTTCCACA | 502 | 534940 | 37 | 568 | 583 | kddedddddddddkke |
| 12098 | 12113 | TAGGTATTTTTCCACA | 502 | 534970 | 59 | 568 | 583 | eddkdddddddddkke |
| 12098 | 12113 | TAGGTATTTTTCCACA | 502 | 535000 | 30 | 568 | 583 | eeeedddddddddkke |
| 12098 | 12113 | TAGGTATTTTTCCACA | 502 | 535033 | 44 | 568 | 583 | eeeedddddddddkkk |
| 12098 | 12113 | TAGGTATTTTTCCACA | 502 | 535066 | 64 | 568 | 583 | eeekdddddddddkke |
| 12098 | 12113 | TAGGTATTTTTCCACA | 502 | 535099 | 22 | 568 | 583 | eeeeeddddddddkke |
| 12098 | 12113 | TAGGTATTTTTCCACA | 502 | 535132 | 54 | 568 | 583 | ededkddddddddkke |
| 12098 | 12113 | TAGGTATTTTTCCACA | 502 | 535165 | 45 | 568 | 583 | edkdeddddddddkke |
| 12512 | 12527 | ATAGCTTTGATCCAAT | 503 | 534544 | 80 | n/a | n/a | kekedddddddddkek |
| 12512 | 12527 | ATAGCTTTGATCCAAT | 503 | 534577 | 83 | n/a | n/a | kekdddddddddekek |
| 12512 | 12527 | ATAGCTTTGATCCAAT | 503 | 534610 | 62 | n/a | n/a | ekeedddddddddkke |
| 12512 | 12527 | ATAGCTTTGATCCAAT | 503 | 534643 | 66 | n/a | n/a | ekedddddddddekke |
| 12512 | 12527 | ATAGCTTTGATCCAAT | 503 | 534676 | 95 | n/a | n/a | eekkdddddddddeke |
| 12512 | 12527 | ATAGCTTTGATCCAAT | 503 | 534709 | 86 | n/a | n/a | eekdddddddddkeke |
| 12512 | 12527 | ATAGCTTTGATCCAAT | 503 | 534746 | 73 | n/a | n/a | ekekddddddddkeke |
| 12512 | 12527 | ATAGCTTTGATCCAAT | 503 | 534781 | 71 | n/a | n/a | keekddddddddkeek |
| 12512 | 12527 | ATAGCTTTGATCCAAT | 503 | 534816 | 83 | n/a | n/a | ekkddddddddddkke |
| 12512 | 12527 | ATAGCTTTGATCCAAT | 503 | 534846 | 73 | n/a | n/a | edkddddddddddkke |
| 12512 | 12527 | ATAGCTTTGATCCAAT | 503 | 534876 | 39 | n/a | n/a | kdeddddddddddkke |
| 12512 | 12527 | ATAGCTTTGATCCAAT | 503 | 534906 | 67 | n/a | n/a | eekddddddddddkke |
| 12512 | 12527 | ATAGCTTTGATCCAAT | 503 | 534936 | 66 | n/a | n/a | kddkdddddddddkke |
| 12512 | 12527 | ATAGCTTTGATCCAAT | 503 | 534966 | 48 | n/a | n/a | kddedddddddddkke |
| 12512 | 12527 | ATAGCTTTGATCCAAT | 503 | 534996 | 56 | n/a | n/a | eddkdddddddddkke |
| 12512 | 12527 | ATAGCTTTGATCCAAT | 503 | 535026 | 39 | n/a | n/a | eeeedddddddddkke |
| 12512 | 12527 | ATAGCTTTGATCCAAT | 503 | 535059 | 45 | n/a | n/a | eeeedddddddddkkk |
| 12512 | 12527 | ATAGCTTTGATCCAAT | 503 | 535092 | 48 | n/a | n/a | eeekdddddddddkke |
| 12512 | 12527 | ATAGCTTTGATCCAAT | 503 | 535125 | 26 | n/a | n/a | eeeeeddddddddkke |
| 12512 | 12527 | ATAGCTTTGATCCAAT | 503 | 535158 | 44 | n/a | n/a | ededkddddddddkke |
| 12512 | 12527 | ATAGCTTTGATCCAAT | 503 | 535191 | 34 | n/a | n/a | edkdeddddddddkke |
| 12513 | 12528 | CATAGCTTTGATCCAA | 504 | 534545 | 83 | n/a | n/a | kekedddddddddkek |
| 12513 | 12528 | CATAGCTTTGATCCAA | 504 | 534578 | 81 | n/a | n/a | kekdddddddddekek |
| 12513 | 12528 | CATAGCTTTGATCCAA | 504 | 534611 | 78 | n/a | n/a | ekeedddddddddkke |
| 12513 | 12528 | CATAGCTTTGATCCAA | 504 | 534644 | 72 | n/a | n/a | ekedddddddddekke |
| 12513 | 12528 | CATAGCTTTGATCCAA | 504 | 534677 | 92 | n/a | n/a | eekkdddddddddeke |
| 12513 | 12528 | CATAGCTTTGATCCAA | 504 | 534710 | 78 | n/a | n/a | eekdddddddddkeke |
| 12513 | 12528 | CATAGCTTTGATCCAA | 504 | 534747 | 85 | n/a | n/a | ekekddddddddkeke |
| 12513 | 12528 | CATAGCTTTGATCCAA | 504 | 534782 | 85 | n/a | n/a | keekddddddddkeek |
| 12513 | 12528 | CATAGCTTTGATCCAA | 504 | 534817 | 88 | n/a | n/a | ekkddddddddddkke |
| 12513 | 12528 | CATAGCTTTGATCCAA | 504 | 534847 | 73 | n/a | n/a | edkddddddddddkke |
| 12513 | 12528 | CATAGCTTTGATCCAA | 504 | 534877 | 66 | n/a | n/a | kdeddddddddddkke |
| 12513 | 12528 | CATAGCTTTGATCCAA | 504 | 534907 | 73 | n/a | n/a | eekddddddddddkke |
| 12513 | 12528 | CATAGCTTTGATCCAA | 504 | 534937 | 85 | n/a | n/a | kddkdddddddddkke |
| 12513 | 12528 | CATAGCTTTGATCCAA | 504 | 534967 | 80 | n/a | n/a | kddedddddddddkke |
| 12513 | 12528 | CATAGCTTTGATCCAA | 504 | 534997 | 74 | n/a | n/a | eddkdddddddddkke |
| 12513 | 12528 | CATAGCTTTGATCCAA | 504 | 535027 | 64 | n/a | n/a | eeeedddddddddkke |
| 12513 | 12528 | CATAGCTTTGATCCAA | 504 | 535060 | 68 | n/a | n/a | eeeedddddddddkkk |
| 12513 | 12528 | CATAGCTTTGATCCAA | 504 | 535093 | 73 | n/a | n/a | eeekdddddddddkke |
| 12513 | 12528 | CATAGCTTTGATCCAA | 504 | 535126 | 42 | n/a | n/a | eeeeeddddddddkke |
| 12513 | 12528 | CATAGCTTTGATCCAA | 504 | 535159 | 49 | n/a | n/a | ededkddddddddkke |
| 12513 | 12528 | CATAGCTTTGATCCAA | 504 | 535192 | 51 | n/a | n/a | edkdeddddddddkke |
| 14076 | 14091 | AACATGTACTCCGTGA | 505 | 534519 | 87 | 1133 | 1148 | kekedddddddddkek |
| 14076 | 14091 | AACATGTACTCCGTGA | 505 | 534552 | 85 | 1133 | 1148 | kekdddddddddekek |
| 14076 | 14091 | AACATGTACTCCGTGA | 505 | 534585 | 76 | 1133 | 1148 | ekeedddddddddkke |
| 14076 | 14091 | AACATGTACTCCGTGA | 505 | 534618 | 78 | 1133 | 1148 | ekedddddddddekke |
| 14076 | 14091 | AACATGTACTCCGTGA | 505 | 534651 | 79 | 1133 | 1148 | eekkdddddddddeke |
| 14076 | 14091 | AACATGTACTCCGTGA | 505 | 534684 | 87 | 1133 | 1148 | eekdddddddddkeke |
| 14076 | 14091 | AACATGTACTCCGTGA | 505 | 534721 | 89 | 1133 | 1148 | ekekddddddddkeke |
| 14076 | 14091 | AACATGTACTCCGTGA | 505 | 534756 | 90 | 1133 | 1148 | keekddddddddkeek |
| 14076 | 14091 | AACATGTACTCCGTGA | 505 | 534791 | 84 | 1133 | 1148 | ekkddddddddddkke |
| 14076 | 14091 | AACATGTACTCCGTGA | 505 | 534821 | 79 | 1133 | 1148 | edkddddddddddkke |
| 14076 | 14091 | AACATGTACTCCGTGA | 505 | 534851 | 64 | 1133 | 1148 | kdeddddddddddkke |
| 14076 | 14091 | AACATGTACTCCGTGA | 505 | 534881 | 65 | 1133 | 1148 | eekddddddddddkke |
| 14076 | 14091 | AACATGTACTCCGTGA | 505 | 534911 | 85 | 1133 | 1148 | kddkdddddddddkke |
| 14076 | 14091 | AACATGTACTCCGTGA | 505 | 534941 | 66 | 1133 | 1148 | kddedddddddddkke |
| 14076 | 14091 | AACATGTACTCCGTGA | 505 | 534971 | 75 | 1133 | 1148 | eddkdddddddddkke |
| 14076 | 14091 | AACATGTACTCCGTGA | 505 | 535001 | 62 | 1133 | 1148 | eeeedddddddddkke |
| 14076 | 14091 | AACATGTACTCCGTGA | 505 | 535034 | 65 | 1133 | 1148 | eeeedddddddddkkk |
| 14076 | 14091 | AACATGTACTCCGTGA | 505 | 535067 | 76 | 1133 | 1148 | eeekdddddddddkke |
| 14076 | 14091 | AACATGTACTCCGTGA | 505 | 535100 | 5 | 1133 | 1148 | eeeeeddddddddkke |
| 14076 | 14091 | AACATGTACTCCGTGA | 505 | 535133 | 30 | 1133 | 1148 | ededkddddddddkke |
| 14076 | 14091 | AACATGTACTCCGTGA | 505 | 535166 | 23 | 1133 | 1148 | edkdeddddddddkke |
| 14094 | 14109 | TCCGAGTAGCCGGCAC | 251 | 534520 | 87 | 1151 | 1166 | kekedddddddddkek |
| 14094 | 14109 | TCCGAGTAGCCGGCAC | 251 | 534553 | 79 | 1151 | 1166 | kekdddddddddekek |
| 14094 | 14109 | TCCGAGTAGCCGGCAC | 251 | 534586 | 60 | 1151 | 1166 | ekeedddddddddkke |
| 14094 | 14109 | TCCGAGTAGCCGGCAC | 251 | 534619 | 62 | 1151 | 1166 | ekedddddddddekke |
| 14094 | 14109 | TCCGAGTAGCCGGCAC | 251 | 534652 | 84 | 1151 | 1166 | eekkdddddddddeke |
| 14094 | 14109 | TCCGAGTAGCCGGCAC | 251 | 534685 | 84 | 1151 | 1166 | eekdddddddddkeke |
| 14094 | 14109 | TCCGAGTAGCCGGCAC | 251 | 534722 | 75 | 1151 | 1166 | ekekddddddddkeke |
| 14094 | 14109 | TCCGAGTAGCCGGCAC | 251 | 534757 | 81 | 1151 | 1166 | keekddddddddkeek |
| 14094 | 14109 | TCCGAGTAGCCGGCAC | 251 | 534792 | 87 | 1151 | 1166 | ekkddddddddddkke |
| 14094 | 14109 | TCCGAGTAGCCGGCAC | 251 | 534822 | 80 | 1151 | 1166 | edkddddddddddkke |
| 14094 | 14109 | TCCGAGTAGCCGGCAC | 251 | 534852 | 38 | 1151 | 1166 | kdeddddddddddkke |
| 14094 | 14109 | TCCGAGTAGCCGGCAC | 251 | 534882 | 75 | 1151 | 1166 | eekddddddddddkke |
| 14094 | 14109 | TCCGAGTAGCCGGCAC | 251 | 534912 | 74 | 1151 | 1166 | kddkdddddddddkke |
| 14094 | 14109 | TCCGAGTAGCCGGCAC | 251 | 534942 | 58 | 1151 | 1166 | kddedddddddddkke |
| 14094 | 14109 | TCCGAGTAGCCGGCAC | 251 | 534972 | 59 | 1151 | 1166 | eddkdddddddddkke |
| 14094 | 14109 | TCCGAGTAGCCGGCAC | 251 | 535002 | 50 | 1151 | 1166 | eeeedddddddddkke |
| 14094 | 14109 | TCCGAGTAGCCGGCAC | 251 | 535035 | 57 | 1151 | 1166 | eeeedddddddddkkk |
| 14094 | 14109 | TCCGAGTAGCCGGCAC | 251 | 535068 | 67 | 1151 | 1166 | eeekdddddddddkke |
| 14094 | 14109 | TCCGAGTAGCCGGCAC | 251 | 535101 | 24 | 1151 | 1166 | eeeeeddddddddkke |
| 14094 | 14109 | TCCGAGTAGCCGGCAC | 251 | 535134 | 23 | 1151 | 1166 | ededkddddddddkke |
| 14094 | 14109 | TCCGAGTAGCCGGCAC | 251 | 535167 | 26 | 1151 | 1166 | edkdeddddddddkke |
| 14243 | 14258 | TGTACTGGGAGACCCT | 252 | 534513 | 90 | 1300 | 1315 | kekedddddddddkek |
| 14243 | 14258 | TGTACTGGGAGACCCT | 252 | 534546 | 92 | 1300 | 1315 | kekdddddddddekek |
| 14243 | 14258 | TGTACTGGGAGACCCT | 252 | 534579 | 78 | 1300 | 1315 | ekeedddddddddkke |
| 14243 | 14258 | TGTACTGGGAGACCCT | 252 | 534612 | 82 | 1300 | 1315 | ekedddddddddekke |
| 14243 | 14258 | TGTACTGGGAGACCCT | 252 | 534645 | 73 | 1300 | 1315 | eekkdddddddddeke |
| 14243 | 14258 | TGTACTGGGAGACCCT | 252 | 534678 | 91 | 1300 | 1315 | eekdddddddddkeke |
| 14243 | 14258 | TGTACTGGGAGACCCT | 252 | 534715 | 87 | 1300 | 1315 | ekekddddddddkeke |
| 14243 | 14258 | TGTACTGGGAGACCCT | 252 | 534750 | 88 | 1300 | 1315 | keekddddddddkeek |
| 14243 | 14258 | TGTACTGGGAGACCCT | 252 | 534785 | 89 | 1300 | 1315 | ekkddddddddddkke |
| 14243 | 14258 | TGTACTGGGAGACCCT | 252 | 535028 | 52 | 1300 | 1315 | eeeedddddddddkkk |
| 14243 | 14258 | TGTACTGGGAGACCCT | 252 | 535061 | 73 | 1300 | 1315 | eeekdddddddddkke |
| 14243 | 14258 | TGTACTGGGAGACCCT | 252 | 535094 | 61 | 1300 | 1315 | eeeeeddddddddkke |
| 14243 | 14258 | TGTACTGGGAGACCCT | 252 | 535127 | 59 | 1300 | 1315 | ededkddddddddkke |
| 14243 | 14258 | TGTACTGGGAGACCCT | 252 | 535160 | 62 | 1300 | 1315 | edkdeddddddddkke |
| 15100 | 15115 | CACATGGAGTCAGCAT | 506 | 534521 | 86 | 2157 | 2172 | kekedddddddddkek |
| 15100 | 15115 | CACATGGAGTCAGCAT | 506 | 534554 | 87 | 2157 | 2172 | kekdddddddddekek |
| 15100 | 15115 | CACATGGAGTCAGCAT | 506 | 534587 | 62 | 2157 | 2172 | ekeedddddddddkke |
| 15100 | 15115 | CACATGGAGTCAGCAT | 506 | 534620 | 68 | 2157 | 2172 | ekedddddddddekke |
| 15100 | 15115 | CACATGGAGTCAGCAT | 506 | 534653 | 77 | 2157 | 2172 | eekkdddddddddeke |
| 15100 | 15115 | CACATGGAGTCAGCAT | 506 | 534686 | 90 | 2157 | 2172 | eekdddddddddkeke |
| 15100 | 15115 | CACATGGAGTCAGCAT | 506 | 534723 | 88 | 2157 | 2172 | ekekddddddddkeke |
| 15100 | 15115 | CACATGGAGTCAGCAT | 506 | 534758 | 79 | 2157 | 2172 | keekddddddddkeek |
| 15100 | 15115 | CACATGGAGTCAGCAT | 506 | 534793 | 85 | 2157 | 2172 | ekkddddddddddkke |
| 15100 | 15115 | CACATGGAGTCAGCAT | 506 | 534823 | 81 | 2157 | 2172 | edkddddddddddkke |
| 15100 | 15115 | CACATGGAGTCAGCAT | 506 | 534853 | 59 | 2157 | 2172 | kdeddddddddddkke |
| 15100 | 15115 | CACATGGAGTCAGCAT | 506 | 534883 | 69 | 2157 | 2172 | eekddddddddddkke |
| 15100 | 15115 | CACATGGAGTCAGCAT | 506 | 534913 | 76 | 2157 | 2172 | kddkdddddddddkke |
| 15100 | 15115 | CACATGGAGTCAGCAT | 506 | 534943 | 53 | 2157 | 2172 | kddedddddddddkke |
| 15100 | 15115 | CACATGGAGTCAGCAT | 506 | 534973 | 61 | 2157 | 2172 | eddkdddddddddkke |
| 15100 | 15115 | CACATGGAGTCAGCAT | 506 | 535003 | 53 | 2157 | 2172 | eeeedddddddddkke |
| 15100 | 15115 | CACATGGAGTCAGCAT | 506 | 535036 | 35 | 2157 | 2172 | eeeedddddddddkkk |
| 15100 | 15115 | CACATGGAGTCAGCAT | 506 | 535069 | 62 | 2157 | 2172 | eeekdddddddddkke |
| 15100 | 15115 | CACATGGAGTCAGCAT | 506 | 535102 | 31 | 2157 | 2172 | eeeeeddddddddkke |
| 15100 | 15115 | CACATGGAGTCAGCAT | 506 | 535135 | 44 | 2157 | 2172 | ededkddddddddkke |
| 15100 | 15115 | CACATGGAGTCAGCAT | 506 | 535168 | 34 | 2157 | 2172 | edkdeddddddddkke |
| 15129 | 15144 | AGCTAAACAACCGCCT | 507 | 534522 | 83 | 2186 | 2201 | kekedddddddddkek |
| 15129 | 15144 | AGCTAAACAACCGCCT | 507 | 534555 | 81 | 2186 | 2201 | kekdddddddddekek |
| 15129 | 15144 | AGCTAAACAACCGCCT | 507 | 534588 | 72 | 2186 | 2201 | ekeedddddddddkke |
| 15129 | 15144 | AGCTAAACAACCGCCT | 507 | 534621 | 74 | 2186 | 2201 | ekedddddddddekke |
| 15129 | 15144 | AGCTAAACAACCGCCT | 507 | 534654 | 78 | 2186 | 2201 | eekkdddddddddeke |
| 15129 | 15144 | AGCTAAACAACCGCCT | 507 | 534687 | 91 | 2186 | 2201 | eekdddddddddkeke |
| 15129 | 15144 | AGCTAAACAACCGCCT | 507 | 534724 | 84 | 2186 | 2201 | ekekddddddddkeke |
| 15129 | 15144 | AGCTAAACAACCGCCT | 507 | 534759 | 86 | 2186 | 2201 | keekddddddddkeek |
| 15129 | 15144 | AGCTAAACAACCGCCT | 507 | 534794 | 78 | 2186 | 2201 | ekkddddddddddkke |
| 15129 | 15144 | AGCTAAACAACCGCCT | 507 | 534824 | 75 | 2186 | 2201 | edkddddddddddkke |
| 15129 | 15144 | AGCTAAACAACCGCCT | 507 | 534854 | 63 | 2186 | 2201 | kdeddddddddddkke |
| 15129 | 15144 | AGCTAAACAACCGCCT | 507 | 534884 | 60 | 2186 | 2201 | eekddddddddddkke |
| 15129 | 15144 | AGCTAAACAACCGCCT | 507 | 534914 | 75 | 2186 | 2201 | kddkdddddddddkke |
| 15129 | 15144 | AGCTAAACAACCGCCT | 507 | 534944 | 69 | 2186 | 2201 | kddedddddddddkke |
| 15129 | 15144 | AGCTAAACAACCGCCT | 507 | 534974 | 66 | 2186 | 2201 | eddkdddddddddkke |
| 15129 | 15144 | AGCTAAACAACCGCCT | 507 | 535004 | 56 | 2186 | 2201 | eeeedddddddddkke |
| 15129 | 15144 | AGCTAAACAACCGCCT | 507 | 535037 | 50 | 2186 | 2201 | eeeedddddddddkkk |
| 15129 | 15144 | AGCTAAACAACCGCCT | 507 | 535070 | 68 | 2186 | 2201 | eeekdddddddddkke |
| 15129 | 15144 | AGCTAAACAACCGCCT | 507 | 535103 | 55 | 2186 | 2201 | eeeeeddddddddkke |
| 15129 | 15144 | AGCTAAACAACCGCCT | 507 | 535136 | 51 | 2186 | 2201 | ededkddddddddkke |
| 15129 | 15144 | AGCTAAACAACCGCCT | 507 | 535169 | 54 | 2186 | 2201 | edkdeddddddddkke |
| 15130 | 15145 | GAGCTAAACAACCGCC | 253 | 534523 | 89 | 2187 | 2202 | kekedddddddddkek |
| 15130 | 15145 | GAGCTAAACAACCGCC | 253 | 534556 | 91 | 2187 | 2202 | kekdddddddddekek |
| 15130 | 15145 | GAGCTAAACAACCGCC | 253 | 534589 | 88 | 2187 | 2202 | ekeedddddddddkke |
| 15130 | 15145 | GAGCTAAACAACCGCC | 253 | 534622 | 93 | 2187 | 2202 | ekedddddddddekke |
| 15130 | 15145 | GAGCTAAACAACCGCC | 253 | 534655 | 72 | 2187 | 2202 | eekkdddddddddeke |
| 15130 | 15145 | GAGCTAAACAACCGCC | 253 | 534688 | 92 | 2187 | 2202 | eekdddddddddkeke |
| 15130 | 15145 | GAGCTAAACAACCGCC | 253 | 534725 | 87 | 2187 | 2202 | ekekddddddddkeke |
| 15130 | 15145 | GAGCTAAACAACCGCC | 253 | 534760 | 92 | 2187 | 2202 | keekddddddddkeek |
| 15130 | 15145 | GAGCTAAACAACCGCC | 253 | 534795 | 93 | 2187 | 2202 | ekkddddddddddkke |
| 15130 | 15145 | GAGCTAAACAACCGCC | 253 | 534825 | 82 | 2187 | 2202 | edkddddddddddkke |
| 15130 | 15145 | GAGCTAAACAACCGCC | 253 | 534855 | 73 | 2187 | 2202 | kdeddddddddddkke |
| 15130 | 15145 | GAGCTAAACAACCGCC | 253 | 534885 | 82 | 2187 | 2202 | eekddddddddddkke |
| 15130 | 15145 | GAGCTAAACAACCGCC | 253 | 534915 | 88 | 2187 | 2202 | kddkdddddddddkke |
| 15130 | 15145 | GAGCTAAACAACCGCC | 253 | 534945 | 82 | 2187 | 2202 | kddedddddddddkke |
| 15130 | 15145 | GAGCTAAACAACCGCC | 253 | 534975 | 68 | 2187 | 2202 | eddkdddddddddkke |
| 15130 | 15145 | GAGCTAAACAACCGCC | 253 | 535005 | 69 | 2187 | 2202 | eeeedddddddddkke |
| 15130 | 15145 | GAGCTAAACAACCGCC | 253 | 535038 | 72 | 2187 | 2202 | eeeedddddddddkkk |
| 15130 | 15145 | GAGCTAAACAACCGCC | 253 | 535071 | 74 | 2187 | 2202 | eeekdddddddddkke |
| 15130 | 15145 | GAGCTAAACAACCGCC | 253 | 535104 | 61 | 2187 | 2202 | eeeeeddddddddkke |
| 15130 | 15145 | GAGCTAAACAACCGCC | 253 | 535137 | 67 | 2187 | 2202 | ededkddddddddkke |
| 15130 | 15145 | GAGCTAAACAACCGCC | 253 | 535170 | 51 | 2187 | 2202 | edkdeddddddddkke |
| 15131 | 15146 | AGAGCTAAACAACCGC | 254 | 534524 | 95 | 2188 | 2203 | kekedddddddddkek |
| 15131 | 15146 | AGAGCTAAACAACCGC | 254 | 534557 | 98 | 2188 | 2203 | kekdddddddddekek |
| 15131 | 15146 | AGAGCTAAACAACCGC | 254 | 534590 | 91 | 2188 | 2203 | ekeedddddddddkke |
| 15131 | 15146 | AGAGCTAAACAACCGC | 254 | 534623 | 91 | 2188 | 2203 | ekedddddddddekke |
| 15131 | 15146 | AGAGCTAAACAACCGC | 254 | 534656 | 90 | 2188 | 2203 | eekkdddddddddeke |
| 15131 | 15146 | AGAGCTAAACAACCGC | 254 | 534689 | 92 | 2188 | 2203 | eekdddddddddkeke |
| 15131 | 15146 | AGAGCTAAACAACCGC | 254 | 534726 | 57 | 2188 | 2203 | ekekddddddddkeke |
| 15131 | 15146 | AGAGCTAAACAACCGC | 254 | 534761 | 89 | 2188 | 2203 | keekddddddddkeek |
| 15131 | 15146 | AGAGCTAAACAACCGC | 254 | 534796 | 93 | 2188 | 2203 | ekkddddddddddkke |
| 15131 | 15146 | AGAGCTAAACAACCGC | 254 | 534826 | 89 | 2188 | 2203 | edkddddddddddkke |
| 15131 | 15146 | AGAGCTAAACAACCGC | 254 | 534856 | 87 | 2188 | 2203 | kdeddddddddddkke |
| 15131 | 15146 | AGAGCTAAACAACCGC | 254 | 534886 | 85 | 2188 | 2203 | eekddddddddddkke |
| 15131 | 15146 | AGAGCTAAACAACCGC | 254 | 534916 | 87 | 2188 | 2203 | kddkdddddddddkke |
| 15131 | 15146 | AGAGCTAAACAACCGC | 254 | 534946 | 86 | 2188 | 2203 | kddedddddddddkke |
| 15131 | 15146 | AGAGCTAAACAACCGC | 254 | 534976 | 77 | 2188 | 2203 | eddkdddddddddkke |
| 15131 | 15146 | AGAGCTAAACAACCGC | 254 | 535006 | 83 | 2188 | 2203 | eeeedddddddddkke |
| 15131 | 15146 | AGAGCTAAACAACCGC | 254 | 535039 | 86 | 2188 | 2203 | eeeedddddddddkkk |
| 15131 | 15146 | AGAGCTAAACAACCGC | 254 | 535072 | 87 | 2188 | 2203 | eeekdddddddddkke |
| 15131 | 15146 | AGAGCTAAACAACCGC | 254 | 535105 | 68 | 2188 | 2203 | eeeeeddddddddkke |
| 15131 | 15146 | AGAGCTAAACAACCGC | 254 | 535138 | 70 | 2188 | 2203 | ededkddddddddkke |
| 15131 | 15146 | AGAGCTAAACAACCGC | 254 | 535171 | 65 | 2188 | 2203 | edkdeddddddddkke |
| 15132 | 15147 | GAGAGCTAAACAACCG | 255 | 534558 | 92 | 2189 | 2204 | kekdddddddddekek |
| 15132 | 15147 | GAGAGCTAAACAACCG | 255 | 534591 | 91 | 2189 | 2204 | ekeedddddddddkke |
| 15132 | 15147 | GAGAGCTAAACAACCG | 255 | 534624 | 86 | 2189 | 2204 | ekedddddddddekke |
| 15132 | 15147 | GAGAGCTAAACAACCG | 255 | 534657 | 90 | 2189 | 2204 | eekkdddddddddeke |
| 15132 | 15147 | GAGAGCTAAACAACCG | 255 | 534690 | 76 | 2189 | 2204 | eekdddddddddkeke |
| 15132 | 15147 | GAGAGCTAAACAACCG | 255 | 534727 | 92 | 2189 | 2204 | ekekddddddddkeke |
| 15132 | 15147 | GAGAGCTAAACAACCG | 255 | 534762 | 91 | 2189 | 2204 | keekddddddddkeek |
| 15132 | 15147 | GAGAGCTAAACAACCG | 255 | 534797 | 94 | 2189 | 2204 | ekkddddddddddkke |
| 15132 | 15147 | GAGAGCTAAACAACCG | 255 | 534827 | 90 | 2189 | 2204 | edkddddddddddkke |
| 15132 | 15147 | GAGAGCTAAACAACCG | 255 | 534857 | 80 | 2189 | 2204 | kdeddddddddddkke |
| 15132 | 15147 | GAGAGCTAAACAACCG | 255 | 534887 | 76 | 2189 | 2204 | eekddddddddddkke |
| 15132 | 15147 | GAGAGCTAAACAACCG | 255 | 534917 | 91 | 2189 | 2204 | kddkdddddddddkke |
| 15132 | 15147 | GAGAGCTAAACAACCG | 255 | 534947 | 91 | 2189 | 2204 | kddedddddddddkke |
| 15132 | 15147 | GAGAGCTAAACAACCG | 255 | 534977 | 86 | 2189 | 2204 | eddkdddddddddkke |
| 15132 | 15147 | GAGAGCTAAACAACCG | 255 | 535007 | 80 | 2189 | 2204 | eeeedddddddddkke |
| 15132 | 15147 | GAGAGCTAAACAACCG | 255 | 535040 | 86 | 2189 | 2204 | eeeedddddddddkkk |
| 15132 | 15147 | GAGAGCTAAACAACCG | 255 | 535073 | 87 | 2189 | 2204 | eeekdddddddddkke |
| 15132 | 15147 | GAGAGCTAAACAACCG | 255 | 535106 | 70 | 2189 | 2204 | eeeeeddddddddkke |
| 15132 | 15147 | GAGAGCTAAACAACCG | 255 | 535139 | 73 | 2189 | 2204 | ededkddddddddkke |
| 15132 | 15147 | GAGAGCTAAACAACCG | 255 | 535172 | 69 | 2189 | 2204 | edkdeddddddddkke |
| 15164 | 15179 | TGAAGATGATAATGGA | 61 | 534514 | 90 | 2221 | 2236 | kekedddddddddkek |
| 15164 | 15179 | TGAAGATGATAATGGA | 61 | 534547 | 92 | 2221 | 2236 | kekdddddddddekek |
| 15164 | 15179 | TGAAGATGATAATGGA | 61 | 534580 | 78 | 2221 | 2236 | ekeedddddddddkke |
| 15164 | 15179 | TGAAGATGATAATGGA | 61 | 534613 | 80 | 2221 | 2236 | ekedddddddddekke |
| 15164 | 15179 | TGAAGATGATAATGGA | 61 | 534646 | 79 | 2221 | 2236 | eekkdddddddddeke |
| 15164 | 15179 | TGAAGATGATAATGGA | 61 | 534679 | 93 | 2221 | 2236 | eekdddddddddkeke |
| 15164 | 15179 | TGAAGATGATAATGGA | 61 | 534716 | 94 | 2221 | 2236 | ekekddddddddkeke |
| 15164 | 15179 | TGAAGATGATAATGGA | 61 | 534751 | 86 | 2221 | 2236 | keekddddddddkeek |
| 15164 | 15179 | TGAAGATGATAATGGA | 61 | 534786 | 83 | 2221 | 2236 | ekkddddddddddkke |
| 15164 | 15179 | TGAAGATGATAATGGA | 61 | 535029 | 45 | 2221 | 2236 | eeeedddddddddkkk |
| 15164 | 15179 | TGAAGATGATAATGGA | 61 | 535062 | 81 | 2221 | 2236 | eeekdddddddddkke |
| 15164 | 15179 | TGAAGATGATAATGGA | 61 | 535095 | 57 | 2221 | 2236 | eeeeeddddddddkke |
| 15164 | 15179 | TGAAGATGATAATGGA | 61 | 535128 | 58 | 2221 | 2236 | ededkddddddddkke |
| 15164 | 15179 | TGAAGATGATAATGGA | 61 | 535161 | 49 | 2221 | 2236 | edkdeddddddddkke |
| 15183 | 15198 | GCTTCTGAATTGTCTG | 256 | 534526 | 94 | 2240 | 2255 | kekedddddddddkek |
| 15183 | 15198 | GCTTCTGAATTGTCTG | 256 | 534559 | 95 | 2240 | 2255 | kekdddddddddekek |
| 15183 | 15198 | GCTTCTGAATTGTCTG | 256 | 534592 | 93 | 2240 | 2255 | ekeedddddddddkke |
| 15183 | 15198 | GCTTCTGAATTGTCTG | 256 | 534625 | 93 | 2240 | 2255 | ekedddddddddekke |
| 15183 | 15198 | GCTTCTGAATTGTCTG | 256 | 534658 | 93 | 2240 | 2255 | eekkdddddddddeke |
| 15183 | 15198 | GCTTCTGAATTGTCTG | 256 | 534691 | 96 | 2240 | 2255 | eekdddddddddkeke |
| 15183 | 15198 | GCTTCTGAATTGTCTG | 256 | 534728 | 93 | 2240 | 2255 | ekekddddddddkeke |
| 15183 | 15198 | GCTTCTGAATTGTCTG | 256 | 534763 | 93 | 2240 | 2255 | keekddddddddkeek |
| 15183 | 15198 | GCTTCTGAATTGTCTG | 256 | 534798 | 97 | 2240 | 2255 | ekkddddddddddkke |
| 15183 | 15198 | GCTTCTGAATTGTCTG | 256 | 534828 | 94 | 2240 | 2255 | edkddddddddddkke |
| 15183 | 15198 | GCTTCTGAATTGTCTG | 256 | 534858 | 92 | 2240 | 2255 | kdeddddddddddkke |
| 15183 | 15198 | GCTTCTGAATTGTCTG | 256 | 534888 | 93 | 2240 | 2255 | eekddddddddddkke |
| 15183 | 15198 | GCTTCTGAATTGTCTG | 256 | 534918 | 95 | 2240 | 2255 | kddkdddddddddkke |
| 15183 | 15198 | GCTTCTGAATTGTCTG | 256 | 534948 | 93 | 2240 | 2255 | kddedddddddddkke |
| 15183 | 15198 | GCTTCTGAATTGTCTG | 256 | 534978 | 91 | 2240 | 2255 | eddkdddddddddkke |
| 15183 | 15198 | GCTTCTGAATTGTCTG | 256 | 535008 | 88 | 2240 | 2255 | eeeedddddddddkke |
| 15183 | 15198 | GCTTCTGAATTGTCTG | 256 | 535041 | 87 | 2240 | 2255 | eeeedddddddddkkk |
| 15183 | 15198 | GCTTCTGAATTGTCTG | 256 | 535074 | 90 | 2240 | 2255 | eeekdddddddddkke |
| 15183 | 15198 | GCTTCTGAATTGTCTG | 256 | 535107 | 78 | 2240 | 2255 | eeeeeddddddddkke |
| 15183 | 15198 | GCTTCTGAATTGTCTG | 256 | 535140 | 81 | 2240 | 2255 | ededkddddddddkke |
| 15183 | 15198 | GCTTCTGAATTGTCTG | 256 | 535173 | 81 | 2240 | 2255 | edkdeddddddddkke |
| 15186 | 15201 | GATGCTTCTGAATTGT | 258 | 534527 | 95 | 2243 | 2258 | kekedddddddddkek |
| 15186 | 15201 | GATGCTTCTGAATTGT | 258 | 534560 | 96 | 2243 | 2258 | kekdddddddddekek |
| 15186 | 15201 | GATGCTTCTGAATTGT | 258 | 534593 | 87 | 2243 | 2258 | ekeedddddddddkke |
| 15186 | 15201 | GATGCTTCTGAATTGT | 258 | 534626 | 85 | 2243 | 2258 | ekedddddddddekke |
| 15186 | 15201 | GATGCTTCTGAATTGT | 258 | 534659 | 90 | 2243 | 2258 | eekkdddddddddeke |
| 15186 | 15201 | GATGCTTCTGAATTGT | 258 | 534692 | 91 | 2243 | 2258 | eekdddddddddkeke |
| 15186 | 15201 | GATGCTTCTGAATTGT | 258 | 534729 | 91 | 2243 | 2258 | ekekddddddddkeke |
| 15186 | 15201 | GATGCTTCTGAATTGT | 258 | 534764 | 91 | 2243 | 2258 | keekddddddddkeek |
| 15186 | 15201 | GATGCTTCTGAATTGT | 258 | 534799 | 96 | 2243 | 2258 | ekkddddddddddkke |
| 15186 | 15201 | GATGCTTCTGAATTGT | 258 | 534829 | 91 | 2243 | 2258 | edkddddddddddkke |
| 15186 | 15201 | GATGCTTCTGAATTGT | 258 | 534859 | 87 | 2243 | 2258 | kdeddddddddddkke |
| 15186 | 15201 | GATGCTTCTGAATTGT | 258 | 534889 | 81 | 2243 | 2258 | eekddddddddddkke |
| 15186 | 15201 | GATGCTTCTGAATTGT | 258 | 534919 | 92 | 2243 | 2258 | kddkdddddddddkke |
| 15186 | 15201 | GATGCTTCTGAATTGT | 258 | 534949 | 91 | 2243 | 2258 | kddedddddddddkke |
| 15186 | 15201 | GATGCTTCTGAATTGT | 258 | 534979 | 84 | 2243 | 2258 | eddkdddddddddkke |
| 15186 | 15201 | GATGCTTCTGAATTGT | 258 | 535009 | 78 | 2243 | 2258 | eeeedddddddddkke |
| 15186 | 15201 | GATGCTTCTGAATTGT | 258 | 535042 | 76 | 2243 | 2258 | eeeedddddddddkkk |
| 15186 | 15201 | GATGCTTCTGAATTGT | 258 | 535075 | 83 | 2243 | 2258 | eeekdddddddddkke |
| 15186 | 15201 | GATGCTTCTGAATTGT | 258 | 535108 | 64 | 2243 | 2258 | eeeeeddddddddkke |
| 15186 | 15201 | GATGCTTCTGAATTGT | 258 | 535141 | 69 | 2243 | 2258 | ededkddddddddkke |
| 15186 | 15201 | GATGCTTCTGAATTGT | 258 | 535174 | 65 | 2243 | 2258 | edkdeddddddddkke |
| 15193 | 15208 | GCATGGTGATGCTTCT | 260 | 534528 | 94 | 2250 | 2265 | kekedddddddddkek |
| 15193 | 15208 | GCATGGTGATGCTTCT | 260 | 534561 | 0 | 2250 | 2265 | kekdddddddddekek |
| 15193 | 15208 | GCATGGTGATGCTTCT | 260 | 534594 | 92 | 2250 | 2265 | ekeedddddddddkke |
| 15193 | 15208 | GCATGGTGATGCTTCT | 260 | 534627 | 90 | 2250 | 2265 | ekedddddddddekke |
| 15193 | 15208 | GCATGGTGATGCTTCT | 260 | 534660 | 92 | 2250 | 2265 | eekkdddddddddeke |
| 15193 | 15208 | GCATGGTGATGCTTCT | 260 | 534693 | 95 | 2250 | 2265 | eekdddddddddkeke |
| 15193 | 15208 | GCATGGTGATGCTTCT | 260 | 534730 | 93 | 2250 | 2265 | ekekddddddddkeke |
| 15193 | 15208 | GCATGGTGATGCTTCT | 260 | 534765 | 92 | 2250 | 2265 | keekddddddddkeek |
| 15193 | 15208 | GCATGGTGATGCTTCT | 260 | 534800 | 93 | 2250 | 2265 | ekkddddddddddkke |
| 15193 | 15208 | GCATGGTGATGCTTCT | 260 | 534830 | 93 | 2250 | 2265 | edkddddddddddkke |
| 15193 | 15208 | GCATGGTGATGCTTCT | 260 | 534860 | 85 | 2250 | 2265 | kdeddddddddddkke |
| 15193 | 15208 | GCATGGTGATGCTTCT | 260 | 534890 | 91 | 2250 | 2265 | eekddddddddddkke |
| 15193 | 15208 | GCATGGTGATGCTTCT | 260 | 534920 | 93 | 2250 | 2265 | kddkdddddddddkke |
| 15193 | 15208 | GCATGGTGATGCTTCT | 260 | 534950 | 90 | 2250 | 2265 | kddedddddddddkke |
| 15193 | 15208 | GCATGGTGATGCTTCT | 260 | 534980 | 88 | 2250 | 2265 | eddkdddddddddkke |
| 15193 | 15208 | GCATGGTGATGCTTCT | 260 | 535010 | 88 | 2250 | 2265 | eeeedddddddddkke |
| 15193 | 15208 | GCATGGTGATGCTTCT | 260 | 535043 | 89 | 2250 | 2265 | eeeedddddddddkkk |
| 15193 | 15208 | GCATGGTGATGCTTCT | 260 | 535076 | 88 | 2250 | 2265 | eeekdddddddddkke |
| 15193 | 15208 | GCATGGTGATGCTTCT | 260 | 535109 | 76 | 2250 | 2265 | eeeeeddddddddkke |
| 15193 | 15208 | GCATGGTGATGCTTCT | 260 | 535142 | 86 | 2250 | 2265 | ededkddddddddkke |
| 15193 | 15208 | GCATGGTGATGCTTCT | 260 | 535175 | 71 | 2250 | 2265 | edkdeddddddddkke |
| 15196 | 15211 | CATGCATGGTGATGCT | 261 | 534529 | 70 | 2253 | 2268 | kekedddddddddkek |
| 15196 | 15211 | CATGCATGGTGATGCT | 261 | 534562 | 86 | 2253 | 2268 | kekdddddddddekek |
| 15196 | 15211 | CATGCATGGTGATGCT | 261 | 534595 | 56 | 2253 | 2268 | ekeedddddddddkke |
| 15196 | 15211 | CATGCATGGTGATGCT | 261 | 534628 | 73 | 2253 | 2268 | ekedddddddddekke |
| 15196 | 15211 | CATGCATGGTGATGCT | 261 | 534661 | 64 | 2253 | 2268 | eekkdddddddddeke |
| 15196 | 15211 | CATGCATGGTGATGCT | 261 | 534694 | 75 | 2253 | 2268 | eekdddddddddkeke |
| 15196 | 15211 | CATGCATGGTGATGCT | 261 | 534731 | 47 | 2253 | 2268 | ekekddddddddkeke |
| 15196 | 15211 | CATGCATGGTGATGCT | 261 | 534766 | 30 | 2253 | 2268 | keekddddddddkeek |
| 15196 | 15211 | CATGCATGGTGATGCT | 261 | 534801 | 83 | 2253 | 2268 | ekkddddddddddkke |
| 15196 | 15211 | CATGCATGGTGATGCT | 261 | 534831 | 84 | 2253 | 2268 | edkddddddddddkke |
| 15196 | 15211 | CATGCATGGTGATGCT | 261 | 534861 | 71 | 2253 | 2268 | kdeddddddddddkke |
| 15196 | 15211 | CATGCATGGTGATGCT | 261 | 534891 | 73 | 2253 | 2268 | eekddddddddddkke |
| 15196 | 15211 | CATGCATGGTGATGCT | 261 | 534921 | 55 | 2253 | 2268 | kddkdddddddddkke |
| 15196 | 15211 | CATGCATGGTGATGCT | 261 | 534951 | 61 | 2253 | 2268 | kddedddddddddkke |
| 15196 | 15211 | CATGCATGGTGATGCT | 261 | 534981 | 48 | 2253 | 2268 | eddkdddddddddkke |
| 15196 | 15211 | CATGCATGGTGATGCT | 261 | 535011 | 54 | 2253 | 2268 | eeeedddddddddkke |
| 15196 | 15211 | CATGCATGGTGATGCT | 261 | 535044 | 46 | 2253 | 2268 | eeeedddddddddkkk |
| 15196 | 15211 | CATGCATGGTGATGCT | 261 | 535077 | 29 | 2253 | 2268 | eeekdddddddddkke |
| 15196 | 15211 | CATGCATGGTGATGCT | 261 | 535110 | 19 | 2253 | 2269 | eeeeeddddddddkke |
| 15196 | 15211 | CATGCATGGTGATGCT | 261 | 535143 | 15 | 2253 | 2268 | ededkddddddddkke |
| 15196 | 15211 | CATGCATGGTGATGCT | 261 | 535176 | 37 | 2253 | 2268 | edkdeddddddddkke |

### Example 22

### Modified Antisense Oligonucleotides Comprising cEt and MOE Modifications Targeting Intronic Repeat Sequences of the Human Coagulation Factor VII Genomic Sequence

Additional antisense oligonucleotides were designed targeting intronic repeat regions of SEQ ID NO: 1. The newly designed modified antisense oligonucleotides and their motifs are described in Table 23. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO.

Each oligonucleotide listed in Table 23 is targeted to intronic regions of human Factor VII genomic sequence, designated herein as SEQ ID NO: 1 (GENBANK Accession No. NT_{—}027140.6 truncated from nucleotides 1255000 to 1273000). “Start site” indicates the 5′-most nucleoside to which the oligonucleotide is targeted in the human gene sequence. “Stop site” indicates the 3′-most nucleoside to which the oligonucleotide is targeted in the human gene sequence. Oligonucleotides having multiple start and stop sites target a region that is repeated within a Factor VII sequence (e.g., within SEQ ID NO: 1).

Cultured Hep3B cells at a density of 20,000 cells per well were transfected using electroporation with 2,000 nM antisense oligonucleotide. After a treatment period of approximately 24 hours, RNA was isolated from the cells and Factor VII mRNA levels were measured by quantitative real-time PCR. Human primer probe set RTS2927 was used to measure mRNA levels. Factor VII mRNA levels were adjusted according to total RNA content, as measured by RIBOGREEN®. Results are presented as percent inhibition of Factor VII expression, relative to untreated control cells.

TABLE 23
Percent inhibition of human Factor VII mRNA levels by modified antisense
oligonucleotides targeted to SEQ ID NO: 1

| Start Site | Stop Site | | | | | |
|---|---|---|---|---|---|---|
| on SEQ ID | on SEQ ID | | SEQ ID | | % | |
| NO: 1 | NO: 1 | Sequence | NO | ISIS No | inhibition | Sugar Chemistry |
| 15263 | 15276 | TGTGCAGCCCGGCA | 508 | 472998 | 90 | kkddddddddddkk |
| 6642 | 6657 | GAGGTGACCCGTGAGC | 30 | 473327 | 88 | kkkddddddddddeee |
| 6712 | 6727 | GTGTGAGGTGACCTGT | 509 | 537024 | 74 | eeeddddddddddkkk |
| 6834 | 6849 | | | | | |
| 7022 | 7037 | | | | | |
| 7140 | 7155 | | | | | |
| 7397 | 7412 | | | | | |
| 7463 | 7478 | | | | | |
| 7862 | 7877 | | | | | |
| 6713 | 6728 | AGTGTGAGGTGACCTG | 510 | 537025 | 79 | eeeddddddddddkkk |
| 6835 | 6850 | | | | | |
| 7398 | 7413 | | | | | |
| 7863 | 7878 | | | | | |
| 6714 | 6729 | GAGTGTGAGGTGACCT | 511 | 537026 | 76 | eeeddddddddddkkk |
| 6836 | 6851 | | | | | |
| 7399 | 7414 | | | | | |
| 7864 | 7879 | | | | | |
| 6716 | 6731 | GTGAGTGTGAGGTGAC | 512 | 537028 | 37 | eeeddddddddddkkk |
| 6838 | 6853 | | | | | |
| 6960 | 6975 | | | | | |
| 7078 | 7093 | | | | | |
| 7196 | 7211 | | | | | |
| 7401 | 7416 | | | | | |
| 7866 | 7881 | | | | | |
| 6717 | 6732 | TGTGAGTGTGAGGTGA | 513 | 537029 | 45 | eeeddddddddddkkk |
| 6839 | 6854 | | | | | |
| 6961 | 6976 | | | | | |
| 7079 | 7094 | | | | | |
| 7197 | 7212 | | | | | |
| 7338 | 7353 | | | | | |
| 7402 | 7417 | | | | | |
| 7867 | 7882 | | | | | |
| 6718 | 6733 | CTGTGAGTGTGAGGTG | 514 | 537030 | 67 | eeeddddddddddkkk |
| 6736 | 6751 | | | | | |
| 6840 | 6855 | | | | | |
| 6858 | 6873 | | | | | |
| 6962 | 6977 | | | | | |
| 7080 | 7095 | | | | | |
| 7198 | 7213 | | | | | |
| 7339 | 7354 | | | | | |
| 7403 | 7418 | | | | | |
| 7637 | 7652 | | | | | |
| 7868 | 7883 | | | | | |
| 8146 | 8161 | | | | | |
| 8393 | 8408 | | | | | |
| 6719 | 6734 | CCTGTGAGTGTGAGGT | 515 | 537031 | 59 | eeeddddddddddkkk |
| 6737 | 6752 | | | | | |
| 6841 | 6856 | | | | | |
| 6859 | 6874 | | | | | |
| 6963 | 6978 | | | | | |
| 7081 | 7096 | | | | | |
| 7199 | 7214 | | | | | |
| 7340 | 7355 | | | | | |
| 7404 | 7419 | | | | | |
| 7638 | 7653 | | | | | |
| 7869 | 7884 | | | | | |
| 8147 | 8162 | | | | | |
| 8394 | 8409 | | | | | |
| 6720 | 6735 | TCCTGTGAGTGTGAGG | 516 | 537032 | 9 | eeeddddddddddkkk |
| 6842 | 6857 | | | | | |
| 6964 | 6979 | | | | | |
| 7082 | 7097 | | | | | |
| 7200 | 7215 | | | | | |
| 7341 | 7356 | | | | | |
| 7405 | 7420 | | | | | |
| 7870 | 7885 | | | | | |
| 8395 | 8410 | | | | | |
| 8485 | 8500 | | | | | |
| 6721 | 6736 | GTCCTGTGAGTGTGAG | 517 | 537033 | 65 | eeeddddddddddkkk |
| 6843 | 6858 | | | | | |
| 6965 | 6980 | | | | | |
| 7083 | 7098 | | | | | |
| 7201 | 7216 | | | | | |
| 7342 | 7357 | | | | | |
| 7406 | 7421 | | | | | |
| 7871 | 7886 | | | | | |
| 6722 | 6737 | TGTCCTGTGAGTGTGA | 518 | 537034 | 71 | eeeddddddddddkkk |
| 6844 | 6859 | | | | | |
| 6966 | 6981 | | | | | |
| 7084 | 7099 | | | | | |
| 7202 | 7217 | | | | | |
| 7343 | 7358 | | | | | |
| 7407 | 7422 | | | | | |
| 7872 | 7887 | | | | | |
| 6723 | 6738 | GTGTCCTGTGAGTGTG | 519 | 537035 | 68 | eeeddddddddddkkk |
| 6845 | 6860 | | | | | |
| 6967 | 6982 | | | | | |
| 7085 | 7100 | | | | | |
| 7203 | 7218 | | | | | |
| 7344 | 7359 | | | | | |
| 7408 | 7423 | | | | | |
| 7873 | 7888 | | | | | |
| 6724 | 6739 | GGTGTCCTGTGAGTGT | 520 | 537036 | 74 | eeeddddddddddkkk |
| 6846 | 6861 | | | | | |
| 6968 | 6983 | | | | | |
| 7086 | 7101 | | | | | |
| 7204 | 7219 | | | | | |
| 7345 | 7360 | | | | | |
| 7409 | 7424 | | | | | |
| 7874 | 7889 | | | | | |
| 6726 | 6741 | GAGGTGTCCTGTGAGT | 521 | 537038 | 69 | eeeddddddddddkkk |
| 6848 | 6863 | | | | | |
| 6970 | 6985 | | | | | |
| 7088 | 7103 | | | | | |
| 7206 | 7221 | | | | | |
| 7347 | 7362 | | | | | |
| 7411 | 7426 | | | | | |
| 7876 | 7891 | | | | | |
| 6727 | 6742 | TGAGGTGTCCTGTGAG | 522 | 537039 | 67 | eeeddddddddddkkk |
| 6849 | 6864 | | | | | |
| 6971 | 6986 | | | | | |
| 7089 | 7104 | | | | | |
| 7207 | 7222 | | | | | |
| 7348 | 7363 | | | | | |
| 7412 | 7427 | | | | | |
| 7628 | 7643 | | | | | |
| 7664 | 7679 | | | | | |
| 7682 | 7697 | | | | | |
| 7877 | 7892 | | | | | |
| 8137 | 8152 | | | | | |
| 8173 | 8188 | | | | | |
| 6728 | 6743 | GTGAGGTGTCCTGTGA | 523 | 537040 | 68 | eeeddddddddddkkk |
| 6850 | 6865 | | | | | |
| 6972 | 6987 | | | | | |
| 7090 | 7105 | | | | | |
| 7208 | 7223 | | | | | |
| 7349 | 7364 | | | | | |
| 7413 | 7428 | | | | | |
| 7629 | 7644 | | | | | |
| 7665 | 7680 | | | | | |
| 7683 | 7698 | | | | | |
| 7878 | 7893 | | | | | |
| 8138 | 8153 | | | | | |
| 8174 | 8189 | | | | | |
| 6729 | 6744 | TGTGAGGTGTCCTGTG | 524 | 537041 | 76 | eeeddddddddddkkk |
| 6851 | 6866 | | | | | |
| 6973 | 6988 | | | | | |
| 7039 | 7054 | | | | | |
| 7091 | 7106 | | | | | |
| 7157 | 7172 | | | | | |
| 7209 | 7224 | | | | | |
| 7263 | 7278 | | | | | |
| 7291 | 7306 | | | | | |
| 7350 | 7365 | | | | | |
| 7414 | 7429 | | | | | |
| 7480 | 7495 | | | | | |
| 7512 | 7527 | | | | | |
| 7526 | 7541 | | | | | |
| 7558 | 7573 | | | | | |
| 7630 | 7645 | | | | | |
| 7684 | 7699 | | | | | |
| 7879 | 7894 | | | | | |
| 7911 | 7926 | | | | | |
| 7975 | 7990 | | | | | |
| 8035 | 8050 | | | | | |
| 8067 | 8082 | | | | | |
| 8139 | 8154 | | | | | |
| 8175 | 8190 | | | | | |
| 6730 | 6745 | GTGTGAGGTGTCCTGT | 525 | 537042 | 77 | eeeddddddddddkkk |
| 6852 | 6867 | | | | | |
| 6974 | 6989 | | | | | |
| 7040 | 7055 | | | | | |
| 7092 | 7107 | | | | | |
| 7158 | 7173 | | | | | |
| 7210 | 7225 | | | | | |
| 7292 | 7307 | | | | | |
| 7351 | 7366 | | | | | |
| 7513 | 7528 | | | | | |
| 7559 | 7574 | | | | | |
| 7631 | 7646 | | | | | |
| 7685 | 7700 | | | | | |
| 8068 | 8083 | | | | | |
| 8140 | 8155 | | | | | |
| 8176 | 8191 | | | | | |
| 6731 | 6746 | AGTGTGAGGTGTCCTG | 526 | 537043 | 70 | eeeddddddddddkkk |
| 6853 | 6868 | | | | | |
| 7211 | 7226 | | | | | |
| 7293 | 7308 | | | | | |
| 7560 | 7575 | | | | | |
| 7632 | 7647 | | | | | |
| 8069 | 8084 | | | | | |
| 8141 | 8156 | | | | | |
| 6732 | 6747 | GAGTGTGAGGTGTCCT | 527 | 537044 | 82 | eeeddddddddddkkk |
| 6854 | 6869 | | | | | |
| 7212 | 7227 | | | | | |
| 7294 | 7309 | | | | | |
| 7561 | 7576 | | | | | |
| 7633 | 7648 | | | | | |
| 8070 | 8085 | | | | | |
| 8142 | 8157 | | | | | |
| 6733 | 6748 | TGAGTGTGAGGTGTCC | 528 | 537045 | 69 | eeeddddddddddkkk |
| 6855 | 6870 | | | | | |
| 7213 | 7228 | | | | | |
| 7295 | 7310 | | | | | |
| 7562 | 7577 | | | | | |
| 7634 | 7649 | | | | | |
| 8071 | 8086 | | | | | |
| 8143 | 8158 | | | | | |
| 6735 | 6750 | TGTGAGTGTGAGGTGT | 529 | 537047 | 35 | eeeddddddddddkkk |
| 6857 | 6872 | | | | | |
| 7636 | 7651 | | | | | |
| 8145 | 8160 | | | | | |
| 8392 | 8407 | | | | | |
| 6739 | 6754 | GCCCTGTGAGTGTGAG | 530 | 537049 | 62 | eeeddddddddddkkk |
| 6861 | 6876 | | | | | |
| 6741 | 6756 | GTGCCCTGTGAGTGTG | 531 | 537051 | 62 | eeeddddddddddkkk |
| 6863 | 6878 | | | | | |
| 6755 | 6770 | CGTGAGTGTGAAGTGT | 532 | 537055 | 16 | eeeddddddddddkkk |
| 6877 | 6892 | | | | | |
| 6943 | 6958 | | | | | |
| 7061 | 7076 | | | | | |
| 7179 | 7194 | | | | | |
| 6756 | 6771 | CCGTGAGTGTGAAGTG | 533 | 537056 | 25 | eeeddddddddddkkk |
| 6878 | 6893 | | | | | |
| 6944 | 6959 | | | | | |
| 7062 | 7077 | | | | | |
| 7180 | 7195 | | | | | |
| 7234 | 7249 | | | | | |
| 6757 | 6772 | CCCGTGAGTGTGAAGT | 534 | 537057 | 49 | eeeddddddddddkkk |
| 6879 | 6894 | | | | | |
| 6945 | 6960 | | | | | |
| 7063 | 7078 | | | | | |
| 7181 | 7196 | | | | | |
| 7235 | 7250 | | | | | |
| 6758 | 6773 | ACCCGTGAGTGTGAAG | 535 | 537058 | 49 | eeeddddddddddkkk |
| 6880 | 6895 | | | | | |
| 6946 | 6961 | | | | | |
| 7064 | 7079 | | | | | |
| 7182 | 7197 | | | | | |
| 7236 | 7251 | | | | | |
| 6759 | 6774 | GACCCGTGAGTGTGAA | 536 | 537059 | 53 | eeeddddddddddkkk |
| 6881 | 6896 | | | | | |
| 6947 | 6962 | | | | | |
| 7065 | 7080 | | | | | |
| 7183 | 7198 | | | | | |
| 7237 | 7252 | | | | | |
| 6760 | 6775 | TGACCCGTGAGTGTGA | 537 | 537060 | 73 | eeeddddddddddkkk |
| 6882 | 6897 | | | | | |
| 6948 | 6963 | | | | | |
| 7066 | 7081 | | | | | |
| 7184 | 7199 | | | | | |
| 7238 | 7253 | | | | | |
| 6761 | 6776 | GTGACCCGTGAGTGTG | 538 | 537061 | 70 | eeeddddddddddkkk |
| 6883 | 6898 | | | | | |
| 6949 | 6964 | | | | | |
| 7067 | 7082 | | | | | |
| 7185 | 7200 | | | | | |
| 7239 | 7254 | | | | | |
| 6762 | 6777 | GGTGACCCGTGAGTGT | 539 | 537062 | 69 | eeeddddddddddkkk |
| 6884 | 6899 | | | | | |
| 6950 | 6965 | | | | | |
| 7068 | 7083 | | | | | |
| 7186 | 7201 | | | | | |
| 7240 | 7255 | | | | | |
| 6763 | 6778 | AGGTGACCCGTGAGTG | 540 | 537063 | 68 | eeeddddddddddkkk |
| 6885 | 6900 | | | | | |
| 6951 | 6966 | | | | | |
| 7069 | 7084 | | | | | |
| 7187 | 7202 | | | | | |
| 7241 | 7256 | | | | | |
| 6764 | 6779 | GAGGTGACCCGTGAGT | 541 | 537064 | 71 | eeeddddddddddkkk |
| 6886 | 6901 | | | | | |
| 6952 | 6967 | | | | | |
| 7070 | 7085 | | | | | |
| 7188 | 7203 | | | | | |
| 7242 | 7257 | | | | | |
| 6643 | 6658 | TGAGGTGACCCGTGAG | 542 | 537065 | 67 | eeeddddddddddkkk |
| 6765 | 6780 | | | | | |
| 6887 | 6902 | | | | | |
| 6953 | 6968 | | | | | |
| 7071 | 7086 | | | | | |
| 7189 | 7204 | | | | | |
| 7243 | 7258 | | | | | |
| 6644 | 6659 | GTGAGGTGACCCGTGA | 543 | 537066 | 68 | eeeddddddddddkkk |
| 6766 | 6781 | | | | | |
| 6888 | 6903 | | | | | |
| 6954 | 6969 | | | | | |
| 7072 | 7087 | | | | | |
| 7190 | 7205 | | | | | |
| 7244 | 7259 | | | | | |
| 6645 | 6660 | TGTGAGGTGACCCGTG | 544 | 537067 | 71 | eeeddddddddddkkk |
| 6767 | 6782 | | | | | |
| 6889 | 6904 | | | | | |
| 6955 | 6970 | | | | | |
| 7073 | 7088 | | | | | |
| 7191 | 7206 | | | | | |
| 7245 | 7260 | | | | | |
| 6646 | 6661 | GTGTGAGGTGACCCGT | 545 | 537068 | 86 | eeeddddddddddkkk |
| 6768 | 6783 | | | | | |
| 6890 | 6905 | | | | | |
| 6956 | 6971 | | | | | |
| 7074 | 7089 | | | | | |
| 7192 | 7207 | | | | | |
| 7246 | 7261 | | | | | |
| 6647 | 6662 | AGTGTGAGGTGACCCG | 546 | 537069 | 82 | eeeddddddddddkkk |
| 6769 | 6784 | | | | | |
| 6891 | 6906 | | | | | |
| 6957 | 6972 | | | | | |
| 7075 | 7090 | | | | | |
| 7193 | 7208 | | | | | |
| 6648 | 6663 | GAGTGTGAGGTGACCC | 547 | 537070 | 87 | eeeddddddddddkkk |
| 6770 | 6785 | | | | | |
| 6892 | 6907 | | | | | |
| 6958 | 6973 | | | | | |
| 7076 | 7091 | | | | | |
| 7194 | 7209 | | | | | |
| 6697 | 6712 | TGAGTGTGAAGTGTGC | 548 | 537792 | 36 | eeeddddddddddkkk |
| 6753 | 6768 | | | | | |
| 6819 | 6834 | | | | | |
| 6875 | 6890 | | | | | |
| 6941 | 6956 | | | | | |
| 7007 | 7022 | | | | | |
| 7059 | 7074 | | | | | |
| 7125 | 7140 | | | | | |
| 7177 | 7192 | | | | | |
| 7382 | 7397 | | | | | |
| 7448 | 7463 | | | | | |
| 7795 | 7810 | | | | | |
| 7945 | 7960 | | | | | |
| 8286 | 8301 | | | | | |
| 6698 | 6713 | GTGAGTGTGAAGTGTG | 549 | 537793 | 35 | eeeddddddddddkkk |
| 6754 | 6769 | | | | | |
| 6820 | 6835 | | | | | |
| 6876 | 6891 | | | | | |
| 6942 | 6957 | | | | | |
| 7008 | 7023 | | | | | |
| 7060 | 7075 | | | | | |
| 7126 | 7141 | | | | | |
| 7178 | 7193 | | | | | |
| 7383 | 7398 | | | | | |
| 7449 | 7464 | | | | | |
| 7796 | 7811 | | | | | |
| 8287 | 8302 | | | | | |
| 6699 | 6714 | TGTGAGTGTGAAGTGT | 550 | 537794 | 35 | eeeddddddddddkkk |
| 6821 | 6836 | | | | | |
| 7009 | 7024 | | | | | |
| 7127 | 7142 | | | | | |
| 7384 | 7399 | | | | | |
| 7450 | 7465 | | | | | |
| 7797 | 7812 | | | | | |
| 8288 | 8303 | | | | | |
| 6700 | 6715 | CTGTGAGTGTGAAGTG | 551 | 537795 | 33 | eeeddddddddddkkk |
| 6822 | 6837 | | | | | |
| 7010 | 7025 | | | | | |
| 7128 | 7143 | | | | | |
| 7385 | 7400 | | | | | |
| 7451 | 7466 | | | | | |
| 7798 | 7813 | | | | | |
| 8289 | 8304 | | | | | |
| 6701 | 6716 | CCTGTGAGTGTGAAGT | 552 | 537796 | 49 | eeeddddddddddkkk |
| 6823 | 6838 | | | | | |
| 7011 | 7026 | | | | | |
| 7129 | 7144 | | | | | |
| 7386 | 7401 | | | | | |
| 7452 | 7467 | | | | | |
| 7799 | 7814 | | | | | |
| 8290 | 8305 | | | | | |
| 6702 | 6717 | ACCTGTGAGTGTGAAG | 553 | 537797 | 54 | eeeddddddddddkkk |
| 6824 | 6839 | | | | | |
| 7012 | 7027 | | | | | |
| 7130 | 7145 | | | | | |
| 7387 | 7402 | | | | | |
| 7453 | 7468 | | | | | |
| 7800 | 7815 | | | | | |
| 8291 | 8306 | | | | | |
| 6703 | 6718 | GACCTGTGAGTGTGAA | 554 | 537798 | 68 | eeeddddddddddkkk |
| 6825 | 6840 | | | | | |
| 7013 | 7028 | | | | | |
| 7131 | 7146 | | | | | |
| 7388 | 7403 | | | | | |
| 7454 | 7469 | | | | | |
| 7801 | 7816 | | | | | |
| 8292 | 8307 | | | | | |
| 6704 | 6719 | TGACCTGTGAGTGTGA | 555 | 537799 | 72 | eeeddddddddddkkk |
| 6826 | 6841 | | | | | |
| 7014 | 7029 | | | | | |
| 7132 | 7147 | | | | | |
| 7389 | 7404 | | | | | |
| 7455 | 7470 | | | | | |
| 7605 | 7620 | | | | | |
| 7641 | 7656 | | | | | |
| 7802 | 7817 | | | | | |
| 8114 | 8129 | | | | | |
| 8150 | 8165 | | | | | |
| 8293 | 8308 | | | | | |
| 6705 | 6720 | GTGACCTGTGAGTGTG | 556 | 537800 | 69 | eeeddddddddddkkk |
| 6827 | 6842 | | | | | |
| 7015 | 7030 | | | | | |
| 7133 | 7148 | | | | | |
| 7390 | 7405 | | | | | |
| 7456 | 7471 | | | | | |
| 7606 | 7621 | | | | | |
| 7642 | 7657 | | | | | |
| 7803 | 7818 | | | | | |
| 8115 | 8130 | | | | | |
| 8151 | 8166 | | | | | |
| 8294 | 8309 | | | | | |
| 6706 | 6721 | GGTGACCTGTGAGTGT | 557 | 537801 | 82 | eeeddddddddddkkk |
| 6828 | 6843 | | | | | |
| 7016 | 7031 | | | | | |
| 7134 | 7149 | | | | | |
| 7391 | 7406 | | | | | |
| 7457 | 7472 | | | | | |
| 7607 | 7622 | | | | | |
| 7643 | 7658 | | | | | |
| 8116 | 8131 | | | | | |
| 8152 | 8167 | | | | | |
| 6707 | 6722 | AGGTGACCTGTGAGTG | 558 | 537802 | 72 | eeeddddddddddkkk |
| 6829 | 6844 | | | | | |
| 7017 | 7032 | | | | | |
| 7135 | 7150 | | | | | |
| 7392 | 7407 | | | | | |
| 7458 | 7473 | | | | | |
| 7608 | 7623 | | | | | |
| 7644 | 7659 | | | | | |
| 8117 | 8132 | | | | | |
| 8153 | 8168 | | | | | |
| 6708 | 6723 | GAGGTGACCTGTGAGT | 559 | 537803 | 72 | eeeddddddddddkkk |
| 6830 | 6845 | | | | | |
| 7018 | 7033 | | | | | |
| 7136 | 7151 | | | | | |
| 7393 | 7408 | | | | | |
| 7459 | 7474 | | | | | |
| 6709 | 6724 | TGAGGTGACCTGTGAG | 560 | 537804 | 67 | eeeddddddddddkkk |
| 6831 | 6846 | | | | | |
| 7019 | 7034 | | | | | |
| 7137 | 7152 | | | | | |
| 7394 | 7409 | | | | | |
| 7460 | 7475 | | | | | |
| 7859 | 7874 | | | | | |
| 6710 | 6725 | GTGAGGTGACCTGTGA | 561 | 537805 | 74 | eeeddddddddddkkk |
| 6832 | 6847 | | | | | |
| 7020 | 7035 | | | | | |
| 7138 | 7153 | | | | | |
| 7395 | 7410 | | | | | |
| 7461 | 7476 | | | | | |
| 7860 | 7875 | | | | | |
| 6711 | 6726 | TGTGAGGTGACCTGTG | 562 | 537806 | 70 | eeeddddddddddkkk |
| 6833 | 6848 | | | | | |
| 7021 | 7036 | | | | | |
| 7139 | 7154 | | | | | |
| 7396 | 7411 | | | | | |
| 7462 | 7477 | | | | | |
| 7861 | 7876 | | | | | |
| 6691 | 6706 | TGAAGTGTGCCCTGTG | 563 | 537809 | 60 | eeeddddddddddkkk |
| 6747 | 6762 | | | | | |
| 6813 | 6828 | | | | | |
| 6869 | 6884 | | | | | |
| 6935 | 6950 | | | | | |
| 7053 | 7068 | | | | | |
| 7171 | 7186 | | | | | |
| 7698 | 7713 | | | | | |
| 8189 | 8204 | | | | | |
| 6692 | 6707 | GTGAAGTGTGCCCTGT | 564 | 537810 | 71 | eeeddddddddddkkk |
| 6748 | 6763 | | | | | |
| 6814 | 6829 | | | | | |
| 6870 | 6885 | | | | | |
| 6936 | 6951 | | | | | |
| 7054 | 7069 | | | | | |
| 7172 | 7187 | | | | | |
| 7699 | 7714 | | | | | |
| 8190 | 8205 | | | | | |
| 6693 | 6708 | TGTGAAGTGTGCCCTG | 565 | 537811 | 69 | eeeddddddddddkkk |
| 6749 | 6764 | | | | | |
| 6815 | 6830 | | | | | |
| 6871 | 6886 | | | | | |
| 6937 | 6952 | | | | | |
| 7055 | 7070 | | | | | |
| 7173 | 7188 | | | | | |
| 7791 | 7806 | | | | | |
| 8282 | 8297 | | | | | |
| 6694 | 6709 | GTGTGAAGTGTGCCCT | 566 | 537812 | 80 | eeeddddddddddkkk |
| 6750 | 6765 | | | | | |
| 6816 | 6831 | | | | | |
| 6872 | 6887 | | | | | |
| 6938 | 6953 | | | | | |
| 7056 | 7071 | | | | | |
| 7174 | 7189 | | | | | |
| 7792 | 7807 | | | | | |
| 8283 | 8298 | | | | | |
| 6695 | 6710 | AGTGTGAAGTGTGCCC | 567 | 537813 | 74 | eeeddddddddddkkk |
| 6751 | 6766 | | | | | |
| 6817 | 6832 | | | | | |
| 6873 | 6888 | | | | | |
| 6939 | 6954 | | | | | |
| 7005 | 7020 | | | | | |
| 7057 | 7072 | | | | | |
| 7123 | 7138 | | | | | |
| 7175 | 7190 | | | | | |
| 7380 | 7395 | | | | | |
| 7446 | 7461 | | | | | |
| 7793 | 7808 | | | | | |
| 7943 | 7958 | | | | | |
| 8284 | 8299 | | | | | |
| 6696 | 6711 | GAGTGTGAAGTGTGCC | 568 | 537814 | 54 | eeeddddddddddkkk |
| 6752 | 6767 | | | | | |
| 6818 | 6833 | | | | | |
| 6874 | 6889 | | | | | |
| 6940 | 6955 | | | | | |
| 7006 | 7021 | | | | | |
| 7058 | 7073 | | | | | |
| 7124 | 7139 | | | | | |
| 7176 | 7191 | | | | | |
| 7381 | 7396 | | | | | |
| 7447 | 7462 | | | | | |
| 7794 | 7809 | | | | | |
| 7944 | 7959 | | | | | |
| 8285 | 8300 | | | | | |
| 6678 | 6693 | GTGTGAGGTGTCCTCT | 569 | 537837 | 70 | eeeddddddddddkkk |
| 6800 | 6815 | | | | | |
| 6922 | 6937 | | | | | |
| 6679 | 6694 | TGTGTGAGGTGTCCTC | 570 | 537838 | 76 | eeeddddddddddkkk |
| 6801 | 6816 | | | | | |
| 6923 | 6938 | | | | | |
| 6680 | 6695 | CTGTGTGAGGTGTCCT | 571 | 537839 | 76 | eeeddddddddddkkk |
| 6802 | 6817 | | | | | |
| 6924 | 6939 | | | | | |
| 7042 | 7057 | | | | | |
| 7160 | 7175 | | | | | |
| 7515 | 7530 | | | | | |
| 7687 | 7702 | | | | | |
| 8178 | 8193 | | | | | |
| 6681 | 6696 | CCTGTGTGAGGTGTCC | 572 | 537840 | 80 | eeeddddddddddkkk |
| 6803 | 6818 | | | | | |
| 6925 | 6940 | | | | | |
| 7043 | 7058 | | | | | |
| 7161 | 7176 | | | | | |
| 7516 | 7531 | | | | | |
| 7688 | 7703 | | | | | |
| 8179 | 8194 | | | | | |
| 6682 | 6697 | CCCTGTGTGAGGTGTC | 573 | 537841 | 81 | eeeddddddddddkkk |
| 6804 | 6819 | | | | | |
| 6926 | 6941 | | | | | |
| 7044 | 7059 | | | | | |
| 7162 | 7177 | | | | | |
| 7689 | 7704 | | | | | |
| 8180 | 8195 | | | | | |
| 6683 | 6698 | GCCCTGTGTGAGGTGT | 574 | 537842 | 75 | eeeddddddddddkkk |
| 6805 | 6820 | | | | | |
| 6927 | 6942 | | | | | |
| 7045 | 7060 | | | | | |
| 7163 | 7178 | | | | | |
| 7690 | 7705 | | | | | |
| 8181 | 8196 | | | | | |
| 6684 | 6699 | TGCCCTGTGTGAGGTG | 575 | 537843 | 70 | eeeddddddddddkkk |
| 6806 | 6821 | | | | | |
| 6928 | 6943 | | | | | |
| 7046 | 7061 | | | | | |
| 7164 | 7179 | | | | | |
| 7691 | 7706 | | | | | |
| 8182 | 8197 | | | | | |
| 6685 | 6700 | GTGCCCTGTGTGAGGT | 576 | 537844 | 73 | eeeddddddddddkkk |
| 6807 | 6822 | | | | | |
| 6929 | 6944 | | | | | |
| 7047 | 7062 | | | | | |
| 7165 | 7180 | | | | | |
| 7692 | 7707 | | | | | |
| 8183 | 8198 | | | | | |
| 6686 | 6701 | TGTGCCCTGTGTGAGG | 577 | 537845 | 59 | eeeddddddddddkkk |
| 6808 | 6823 | | | | | |
| 6930 | 6945 | | | | | |
| 7048 | 7063 | | | | | |
| 7166 | 7181 | | | | | |
| 7693 | 7708 | | | | | |
| 8184 | 8199 | | | | | |
| 6687 | 6702 | GTGTGCCCTGTGTGAG | 578 | 537846 | 51 | eeeddddddddddkkk |
| 6809 | 6824 | | | | | |
| 6931 | 6946 | | | | | |
| 7049 | 7064 | | | | | |
| 7167 | 7182 | | | | | |
| 7694 | 7709 | | | | | |
| 8185 | 8200 | | | | | |
| 6688 | 6703 | AGTGTGCCCTGTGTGA | 579 | 537847 | 52 | eeeddddddddddkkk |
| 6810 | 6825 | | | | | |
| 6932 | 6947 | | | | | |
| 7050 | 7065 | | | | | |
| 7168 | 7183 | | | | | |
| 7695 | 7710 | | | | | |
| 8186 | 8201 | | | | | |
| 6689 | 6704 | AAGTGTGCCCTGTGTG | 580 | 537848 | 41 | eeeddddddddddkkk |
| 6811 | 6826 | | | | | |
| 6933 | 6948 | | | | | |
| 7051 | 7066 | | | | | |
| 7169 | 7184 | | | | | |
| 7696 | 7711 | | | | | |
| 8187 | 8202 | | | | | |
| 6690 | 6705 | GAAGTGTGCCCTGTGT | 581 | 537849 | 44 | eeeddddddddddkkk |
| 6812 | 6827 | | | | | |
| 6934 | 6949 | | | | | |
| 7052 | 7067 | | | | | |
| 7170 | 7185 | | | | | |
| 7697 | 7712 | | | | | |
| 8188 | 8203 | | | | | |
| 6975 | 6990 | TGTGTGAGGTGTCCTG | 582 | 538160 | 69 | eeeddddddddddkkk |
| 7041 | 7056 | | | | | |
| 7093 | 7108 | | | | | |
| 7159 | 7174 | | | | | |
| 7352 | 7367 | | | | | |
| 7514 | 7529 | | | | | |
| 7686 | 7701 | | | | | |
| 8177 | 8192 | | | | | |
| 6987 | 7002 | TGTGAGGTGTCTTGTG | 583 | 538172 | 24 | eeeddddddddddkkk |
| 7105 | 7120 | | | | | |
| 8443 | 8458 | | | | | |
| 6988 | 7003 | GTGTGAGGTGTCTTGT | 584 | 538173 | 23 | eeeddddddddddkkk |
| 7106 | 7121 | | | | | |
| 8444 | 8459 | | | | | |
| 7000 | 7015 | GAAGTGTGCCCCGTGT | 585 | 538185 | 68 | eeeddddddddddkkk |
| 7118 | 7133 | | | | | |
| 7375 | 7390 | | | | | |
| 7441 | 7456 | | | | | |
| 7938 | 7953 | | | | | |
| 7002 | 7017 | GTGAAGTGTGCCCCGT | 585 | 538187 | 69 | eeeddddddddddkkk |
| 7120 | 7135 | | | | | |
| 7377 | 7392 | | | | | |
| 7443 | 7458 | | | | | |
| 7940 | 7955 | | | | | |
| 7004 | 7019 | GTGTGAAGTGTGCCCC | 587 | 538189 | 81 | eeeddddddddddkkk |
| 7122 | 7137 | | | | | |
| 7379 | 7394 | | | | | |
| 7445 | 7460 | | | | | |
| 7942 | 7957 | | | | | |
| 7024 | 7039 | GGGTGTGAGGTGACCT | 588 | 538191 | 66 | eeeddddddddddkkk |
| 7142 | 7157 | | | | | |
| 7465 | 7480 | | | | | |
| 7497 | 7512 | | | | | |
| 7543 | 7558 | | | | | |
| 7896 | 7911 | | | | | |
| 7960 | 7975 | | | | | |
| 7992 | 8007 | | | | | |
| 8052 | 8067 | | | | | |
| 7025 | 7040 | TGGGTGTGAGGTGACC | 589 | 538192 | 59 | eeeddddddddddkkk |
| 7143 | 7158 | | | | | |
| 7249 | 7264 | | | | | |
| 7466 | 7481 | | | | | |
| 7498 | 7513 | | | | | |
| 7544 | 7559 | | | | | |
| 7897 | 7912 | | | | | |
| 7961 | 7976 | | | | | |
| 7993 | 8008 | | | | | |
| 8053 | 8068 | | | | | |
| 7026 | 7041 | GTGGGTGTGAGGTGAC | 590 | 538193 | 16 | eeeddddddddddkkk |
| 7144 | 7159 | | | | | |
| 7250 | 7265 | | | | | |
| 7467 | 7482 | | | | | |
| 7499 | 7514 | | | | | |
| 7545 | 7560 | | | | | |
| 7898 | 7913 | | | | | |
| 7962 | 7977 | | | | | |
| 7994 | 8009 | | | | | |
| 8054 | 8069 | | | | | |
| 7027 | 7042 | TGTGGGTGTGAGGTGA | 591 | 538194 | 10 | eeeddddddddddkkk |
| 7145 | 7160 | | | | | |
| 7251 | 7266 | | | | | |
| 7468 | 7483 | | | | | |
| 7500 | 7515 | | | | | |
| 7546 | 7561 | | | | | |
| 7899 | 7914 | | | | | |
| 7963 | 7978 | | | | | |
| 7995 | 8010 | | | | | |
| 8055 | 8070 | | | | | |
| 7028 | 7043 | CTGTGGGTGTGAGGTG | 592 | 538195 | 15 | eeeddddddddddkkk |
| 7146 | 7161 | | | | | |
| 7252 | 7267 | | | | | |
| 7469 | 7484 | | | | | |
| 7501 | 7516 | | | | | |
| 7547 | 7562 | | | | | |
| 7900 | 7915 | | | | | |
| 7964 | 7979 | | | | | |
| 7996 | 8011 | | | | | |
| 8056 | 8071 | | | | | |
| 7029 | 7044 | CCTGTGGGTGTGAGGT | 593 | 538196 | 3 | eeeddddddddddkkk |
| 7147 | 7162 | | | | | |
| 7253 | 7268 | | | | | |
| 7470 | 7485 | | | | | |
| 7502 | 7517 | | | | | |
| 7548 | 7563 | | | | | |
| 7901 | 7916 | | | | | |
| 7965 | 7980 | | | | | |
| 7997 | 8012 | | | | | |
| 8057 | 8072 | | | | | |
| 7030 | 7045 | TCCTGTGGGTGTGAGG | 594 | 538197 | 36 | eeeddddddddddkkk |
| 7148 | 7163 | | | | | |
| 7254 | 7269 | | | | | |
| 7471 | 7486 | | | | | |
| 7503 | 7518 | | | | | |
| 7549 | 7564 | | | | | |
| 7902 | 7917 | | | | | |
| 7966 | 7981 | | | | | |
| 7998 | 8013 | | | | | |
| 8058 | 8073 | | | | | |
| 7031 | 7046 | GTCCTGTGGGTGTGAG | 595 | 538198 | 49 | eeeddddddddddkkk |
| 7149 | 7164 | | | | | |
| 7255 | 7270 | | | | | |
| 7472 | 7487 | | | | | |
| 7504 | 7519 | | | | | |
| 7550 | 7565 | | | | | |
| 7903 | 7918 | | | | | |
| 7967 | 7982 | | | | | |
| 7999 | 8014 | | | | | |
| 8059 | 8074 | | | | | |
| 7032 | 7047 | TGTCCTGTGGGTGTGA | 596 | 538199 | 47 | eeeddddddddddkkk |
| 7150 | 7165 | | | | | |
| 7256 | 7271 | | | | | |
| 7473 | 7488 | | | | | |
| 7505 | 7520 | | | | | |
| 7551 | 7566 | | | | | |
| 7904 | 7919 | | | | | |
| 7968 | 7983 | | | | | |
| 8000 | 8015 | | | | | |
| 8060 | 8075 | | | | | |
| 7033 | 7048 | GTGTCCTGTGGGTGTG | 597 | 538200 | 57 | eeeddddddddddkkk |
| 7151 | 7166 | | | | | |
| 7257 | 7272 | | | | | |
| 7474 | 7489 | | | | | |
| 7506 | 7521 | | | | | |
| 7552 | 7567 | | | | | |
| 7905 | 7920 | | | | | |
| 7969 | 7984 | | | | | |
| 8061 | 8076 | | | | | |
| 7034 | 7049 | GGTGTCCTGTGGGTGT | 598 | 538201 | 71 | eeeddddddddddkkk |
| 7152 | 7167 | | | | | |
| 7258 | 7273 | | | | | |
| 7475 | 7490 | | | | | |
| 7507 | 7522 | | | | | |
| 7553 | 7568 | | | | | |
| 7906 | 7921 | | | | | |
| 7970 | 7985 | | | | | |
| 8062 | 8077 | | | | | |
| 7035 | 7050 | AGGTGTCCTGTGGGTG | 599 | 538202 | 60 | eeeddddddddddkkk |
| 7153 | 7168 | | | | | |
| 7259 | 7274 | | | | | |
| 7476 | 7491 | | | | | |
| 7508 | 7523 | | | | | |
| 7554 | 7569 | | | | | |
| 7907 | 7922 | | | | | |
| 7971 | 7986 | | | | | |
| 8063 | 8078 | | | | | |
| 7036 | 7051 | GAGGTGTCCTGTGGGT | 600 | 538203 | 55 | eeeddddddddddkkk |
| 7154 | 7169 | | | | | |
| 7260 | 7275 | | | | | |
| 7477 | 7492 | | | | | |
| 7509 | 7524 | | | | | |
| 7555 | 7570 | | | | | |
| 7908 | 7923 | | | | | |
| 7972 | 798762 | | | | | |
| 8064 | 8079 | | | | | |
| 7037 | 7052 | TGAGGTGTCCTGTGGG | 601 | 538204 | 62 | eeeddddddddddkkk |
| 7155 | 7170 | | | | | |
| 7261 | 7276 | | | | | |
| 7478 | 7493 | | | | | |
| 7510 | 7525 | | | | | |
| 7556 | 7571 | | | | | |
| 7909 | 7924 | | | | | |
| 7973 | 7988 | | | | | |
| 8065 | 8080 | | | | | |
| 7038 | 7053 | GTGAGGTGTCCTGTGG | 602 | 538205 | 68 | eeeddddddddddkkk |
| 7156 | 7171 | | | | | |
| 7262 | 7277 | | | | | |
| 7479 | 7494 | | | | | |
| 7511 | 7526 | | | | | |
| 7557 | 7572 | | | | | |
| 7910 | 7925 | | | | | |
| 7974 | 7989 | | | | | |
| 8066 | 8081 | | | | | |
| 7264 | 7279 | CTGTGAGGTGTCCTGT | 603 | 538228 | 63 | eeeddddddddddkkk |
| 7415 | 7430 | | | | | |
| 7481 | 7496 | | | | | |
| 7527 | 7542 | | | | | |
| 7880 | 7895 | | | | | |
| 7912 | 7927 | | | | | |
| 7976 | 7991 | | | | | |
| 7265 | 7280 | TCTGTGAGGTGTCCTG | 604 | 538229 | 26 | eeeddddddddddkkk |
| 7416 | 7431 | | | | | |
| 7482 | 7497 | | | | | |
| 7528 | 7543 | | | | | |
| 7881 | 7896 | | | | | |
| 7913 | 7928 | | | | | |
| 7977 | 7992 | | | | | |
| 7266 | 7281 | CTCTGTGAGGTGTCCT | 605 | 538230 | 75 | eeeddddddddddkkk |
| 7417 | 7432 | | | | | |
| 7483 | 7498 | | | | | |
| 7529 | 7544 | | | | | |
| 7882 | 7897 | | | | | |
| 7914 | 7929 | | | | | |
| 7978 | 7993 | | | | | |
| 7267 | 7282 | CCTCTGTGAGGTGTCC | 606 | 538231 | 75 | eeeddddddddddkkk |
| 7418 | 7433 | | | | | |
| 7484 | 7499 | | | | | |
| 7530 | 7545 | | | | | |
| 7883 | 7898 | | | | | |
| 7915 | 7930 | | | | | |
| 7979 | 7994 | | | | | |
| 7269 | 7284 | GACCTCTGTGAGGTGT | 607 | 538233 | 52 | eeeddddddddddkkk |
| 7420 | 7435 | | | | | |
| 7486 | 7501 | | | | | |
| 7532 | 7547 | | | | | |
| 7885 | 7900 | | | | | |
| 7917 | 7932 | | | | | |
| 7981 | 7996 | | | | | |
| 7271 | 7286 | GTGACCTCTGTGAGGT | 608 | 538235 | 26 | eeeddddddddddkkk |
| 7422 | 7437 | | | | | |
| 7488 | 7503 | | | | | |
| 7534 | 7549 | | | | | |
| 7887 | 7902 | | | | | |
| 7919 | 7934 | | | | | |
| 7983 | 7998 | | | | | |
| 7273 | 7288 | AGGTGACCTCTGTGAG | 609 | 538237 | 28 | eeeddddddddddkkk |
| 7424 | 7439 | | | | | |
| 7490 | 7505 | | | | | |
| 7536 | 7551 | | | | | |
| 7889 | 7904 | | | | | |
| 7921 | 7936 | | | | | |
| 7985 | 8000 | | | | | |
| 8017 | 8032 | | | | | |
| 7275 | 7290 | TGAGGTGACCTCTGTG | 610 | 538239 | 54 | eeeddddddddddkkk |
| 7426 | 7441 | | | | | |
| 7492 | 7507 | | | | | |
| 7538 | 7553 | | | | | |
| 7891 | 7906 | | | | | |
| 7923 | 7938 | | | | | |
| 7987 | 8002 | | | | | |
| 8019 | 8034 | | | | | |
| 7277 | 7292 | TGTGAGGTGACCTCTG | 611 | 538241 | 73 | eeeddddddddddkkk |
| 7428 | 7443 | | | | | |
| 7494 | 7509 | | | | | |
| 7540 | 7555 | | | | | |
| 7893 | 7908 | | | | | |
| 7925 | 7940 | | | | | |
| 7989 | 8004 | | | | | |
| 8021 | 8036 | | | | | |
| 7278 | 7293 | GTGTGAGGTGACCTCT | 612 | 538242 | 68 | eeeddddddddddkkk |
| 7429 | 7444 | | | | | |
| 7495 | 7510 | | | | | |
| 7541 | 7556 | | | | | |
| 7894 | 7909 | | | | | |
| 7926 | 7941 | | | | | |
| 7990 | 8005 | | | | | |
| 8022 | 8037 | | | | | |
| 7279 | 7294 | TGTGTGAGGTGACCTC | 613 | 538243 | 61 | eeeddddddddddkkk |
| 8023 | 8038 | | | | | |
| 7281 | 7296 | CCTGTGTGAGGTGACC | 614 | 538245 | 75 | eeeddddddddddkkk |
| 8025 | 8040 | | | | | |
| 7289 | 7304 | TGAGGTGTCCTGTGTG | 615 | 538253 | 37 | eeeddddddddddkkk |
| 7524 | 7539 | | | | | |
| 8033 | 8048 | | | | | |
| 7290 | 7305 | GTGAGGTGTCCTGTGT | 616 | 538254 | 45 | eeeddddddddddkkk |
| 7525 | 7540 | | | | | |
| 8034 | 8049 | | | | | |
| 7604 | 7619 | GACCTGTGAGTGTGAG | 617 | 538361 | 56 | eeeddddddddddkkk |
| 7640 | 7655 | | | | | |
| 8113 | 8128 | | | | | |
| 8149 | 8164 | | | | | |
| 8373 | 8388 | | | | | |
| 7625 | 7640 | GGTGTCCTGTGAGAGT | 618 | 538378 | 70 | eeeddddddddddkkk |
| 7661 | 7676 | | | | | |
| 7679 | 7694 | | | | | |
| 8134 | 8149 | | | | | |
| 8170 | 8185 | | | | | |
| 7627 | 7642 | GAGGTGTCCTGTGAGA | 619 | 538380 | 68 | eeeddddddddddkkk |
| 7663 | 7678 | | | | | |
| 7681 | 7696 | | | | | |
| 7840 | 7855 | | | | | |
| 8136 | 8151 | | | | | |
| 8172 | 8187 | | | | | |
| 8331 | 8346 | | | | | |
| 7639 | 7654 | ACCTGTGAGTGTGAGG | 620 | 538381 | 57 | eeeddddddddddkkk |
| 8148 | 8163 | | | | | |
| 2560 | 2575 | CGGGACACCCACACCC | 621 | 540361 | 71 | eeeddddddddddkkk |
| 3257 | 3272 | | | | | |
| 3700 | 3715 | | | | | |
| 3717 | 3732 | | | | | |
| 4023 | 4038 | | | | | |
| 4109 | 4124 | | | | | |
| 4296 | 4311 | | | | | |
| 4551 | 4566 | | | | | |
| 2562 | 2577 | CCCGGGACACCCACAC | 622 | 540362 | 73 | eeeddddddddddkkk |
| 2579 | 2594 | | | | | |
| 2613 | 2628 | | | | | |
| 2647 | 2662 | | | | | |
| 2715 | 2730 | | | | | |
| 2783 | 2798 | | | | | |
| 2817 | 2832 | | | | | |
| 2885 | 2900 | | | | | |
| 2953 | 2968 | | | | | |
| 3021 | 3036 | | | | | |
| 3055 | 3070 | | | | | |
| 3089 | 3104 | | | | | |
| 3259 | 3274 | | | | | |
| 3361 | 3376 | | | | | |
| 3565 | 3580 | | | | | |
| 3685 | 3700 | | | | | |
| 3702 | 3717 | | | | | |
| 3719 | 3734 | | | | | |
| 3736 | 3751 | | | | | |
| 3872 | 3887 | | | | | |
| 3940 | 3955 | | | | | |
| 4025 | 4040 | | | | | |
| 4111 | 4126 | | | | | |
| 4145 | 4160 | | | | | |
| 4298 | 4313 | | | | | |
| 4332 | 4347 | | | | | |
| 4434 | 4449 | | | | | |
| 4468 | 4483 | | | | | |
| 4553 | 4568 | | | | | |
| 2564 | 2579 | CTCCCGGGACACCCAC | 623 | 540363 | 78 | eeeddddddddddkkk |
| 2632 | 2647 | | | | | |
| 2666 | 2681 | | | | | |
| 2734 | 2749 | | | | | |
| 2802 | 2817 | | | | | |
| 2836 | 2851 | | | | | |
| 2904 | 2919 | | | | | |
| 2972 | 2987 | | | | | |
| 3006 | 3021 | | | | | |
| 3040 | 3055 | | | | | |
| 3074 | 3089 | | | | | |
| 3091 | 3106 | | | | | |
| 3278 | 3293 | | | | | |
| 3380 | 3395 | | | | | |
| 3482 | 3497 | | | | | |
| 3602 | 3617 | | | | | |
| 3721 | 3736 | | | | | |
| 3755 | 3770 | | | | | |
| 3857 | 3872 | | | | | |
| 3891 | 3906 | | | | | |
| 3959 | 3974 | | | | | |
| 4045 | 4060 | | | | | |
| 4130 | 4145 | | | | | |
| 4164 | 4179 | | | | | |
| 4266 | 4281 | | | | | |
| 4317 | 4332 | | | | | |
| 4453 | 4468 | | | | | |
| 4573 | 4588 | | | | | |
| 2565 | 2580 | ACTCCCGGGACACCCA | 624 | 540364 | 89 | eeeddddddddddkkk |
| 2633 | 2648 | | | | | |
| 2667 | 2682 | | | | | |
| 2735 | 2750 | | | | | |
| 2803 | 2818 | | | | | |
| 2837 | 2852 | | | | | |
| 2905 | 2920 | | | | | |
| 3007 | 3022 | | | | | |
| 3041 | 3056 | | | | | |
| 3075 | 3090 | | | | | |
| 3092 | 3107 | | | | | |
| 3279 | 3294 | | | | | |
| 3381 | 3396 | | | | | |
| 3483 | 3498 | | | | | |
| 3603 | 3618 | | | | | |
| 3722 | 3737 | | | | | |
| 3756 | 3771 | | | | | |
| 3858 | 3873 | | | | | |
| 3892 | 3907 | | | | | |
| 3960 | 3975 | | | | | |
| 4046 | 4061 | | | | | |
| 4131 | 4146 | | | | | |
| 4165 | 4180 | | | | | |
| 4318 | 4333 | | | | | |
| 4454 | 4469 | | | | | |
| 2566 | 2581 | CACTCCCGGGACACCC | 625 | 540365 | 83 | eeeddddddddddkkk |
| 2634 | 2649 | | | | | |
| 2668 | 2683 | | | | | |
| 2702 | 2717 | | | | | |
| 2736 | 2751 | | | | | |
| 2770 | 2785 | | | | | |
| 2804 | 2819 | | | | | |
| 2838 | 2853 | | | | | |
| 2872 | 2887 | | | | | |
| 2906 | 2921 | | | | | |
| 2940 | 2955 | | | | | |
| 3008 | 3023 | | | | | |
| 3042 | 3057 | | | | | |
| 3076 | 3091 | | | | | |
| 3093 | 3108 | | | | | |
| 3127 | 3142 | | | | | |
| 3280 | 3295 | | | | | |
| 3314 | 3329 | | | | | |
| 3348 | 3363 | | | | | |
| 3382 | 3397 | | | | | |
| 3416 | 3431 | | | | | |
| 3450 | 3465 | | | | | |
| 3484 | 3499 | | | | | |
| 3518 | 3533 | | | | | |
| 3552 | 3567 | | | | | |
| 3604 | 3619 | | | | | |
| 3638 | 3653 | | | | | |
| 3672 | 3687 | | | | | |
| 3723 | 3738 | | | | | |
| 3757 | 3772 | | | | | |
| 3859 | 3874 | | | | | |
| 3893 | 3908 | | | | | |
| 3961 | 3976 | | | | | |
| 4047 | 4062 | | | | | |
| 4081 | 4096 | | | | | |
| 4132 | 4147 | | | | | |
| 4166 | 4181 | | | | | |
| 4200 | 4215 | | | | | |
| 4319 | 4334 | | | | | |
| 4387 | 4402 | | | | | |
| 4421 | 4436 | | | | | |
| 4455 | 4470 | | | | | |
| 2567 | 2582 | ACACTCCCGGGACACC | 626 | 540366 | 84 | eeeddddddddddkkk |
| 2635 | 2650 | | | | | |
| 2669 | 2684 | | | | | |
| 2703 | 2718 | | | | | |
| 2737 | 2752 | | | | | |
| 2771 | 2786 | | | | | |
| 2805 | 2820 | | | | | |
| 2839 | 2854 | | | | | |
| 2873 | 2888 | | | | | |
| 2907 | 2922 | | | | | |
| 2941 | 2956 | | | | | |
| 3009 | 3024 | | | | | |
| 3043 | 3058 | | | | | |
| 3077 | 3092 | | | | | |
| 3094 | 3109 | | | | | |
| 3128 | 3143 | | | | | |
| 3281 | 3296 | | | | | |
| 3315 | 3330 | | | | | |
| 3349 | 3364 | | | | | |
| 3383 | 3398 | | | | | |
| 3417 | 3432 | | | | | |
| 3451 | 3466 | | | | | |
| 3485 | 3500 | | | | | |
| 3519 | 3534 | | | | | |
| 3553 | 3568 | | | | | |
| 3605 | 3620 | | | | | |
| 3639 | 3654 | | | | | |
| 3673 | 3688 | | | | | |
| 3724 | 3739 | | | | | |
| 3758 | 3773 | | | | | |
| 3860 | 3875 | | | | | |
| 3894 | 3909 | | | | | |
| 3962 | 3977 | | | | | |
| 4048 | 4063 | | | | | |
| 4082 | 4097 | | | | | |
| 4133 | 4148 | | | | | |
| 4167 | 4182 | | | | | |
| 4201 | 4216 | | | | | |
| 4320 | 4335 | | | | | |
| 4388 | 4403 | | | | | |
| 4422 | 4437 | | | | | |
| 4456 | 4471 | | | | | |
| 2568 | 2583 | CACACTCCCGGGACAC | 627 | 540367 | 65 | eeeddddddddddkkk |
| 2636 | 2651 | | | | | |
| 2670 | 2685 | | | | | |
| 2704 | 2719 | | | | | |
| 2738 | 2753 | | | | | |
| 2772 | 2787 | | | | | |
| 2806 | 2821 | | | | | |
| 2840 | 2855 | | | | | |
| 2874 | 2889 | | | | | |
| 2908 | 2923 | | | | | |
| 2942 | 2957 | | | | | |
| 3010 | 3025 | | | | | |
| 3044 | 3059 | | | | | |
| 3078 | 3093 | | | | | |
| 3095 | 3110 | | | | | |
| 3129 | 3144 | | | | | |
| 3282 | 3297 | | | | | |
| 3316 | 3331 | | | | | |
| 3350 | 3365 | | | | | |
| 3384 | 3399 | | | | | |
| 3418 | 3433 | | | | | |
| 3452 | 3467 | | | | | |
| 3486 | 3501 | | | | | |
| 3520 | 3535 | | | | | |
| 3554 | 3569 | | | | | |
| 3606 | 3621 | | | | | |
| 3640 | 3655 | | | | | |
| 3674 | 3689 | | | | | |
| 3725 | 3740 | | | | | |
| 3759 | 3774 | | | | | |
| 3861 | 3876 | | | | | |
| 3895 | 3910 | | | | | |
| 3963 | 3978 | | | | | |
| 4049 | 4064 | | | | | |
| 4083 | 4098 | | | | | |
| 4134 | 4149 | | | | | |
| 4168 | 4183 | | | | | |
| 4202 | 4217 | | | | | |
| 4321 | 4336 | | | | | |
| 4389 | 4404 | | | | | |
| 4423 | 4438 | | | | | |
| 4457 | 4472 | | | | | |
| 2571 | 2586 | ACCCACACTCCCGGGA | 628 | 540368 | 55 | eeeddddddddddkkk |
| 2639 | 2654 | | | | | |
| 2673 | 2688 | | | | | |
| 2707 | 2722 | | | | | |
| 2741 | 2756 | | | | | |
| 2775 | 2790 | | | | | |
| 2809 | 2824 | | | | | |
| 2843 | 2858 | | | | | |
| 2877 | 2892 | | | | | |
| 2911 | 2926 | | | | | |
| 2945 | 2960 | | | | | |
| 3013 | 3028 | | | | | |
| 3047 | 3062 | | | | | |
| 3081 | 3096 | | | | | |
| 3098 | 3113 | | | | | |
| 3285 | 3300 | | | | | |
| 3319 | 3334 | | | | | |
| 3353 | 3368 | | | | | |
| 3387 | 3402 | | | | | |
| 3421 | 3436 | | | | | |
| 3455 | 3470 | | | | | |
| 3489 | 3504 | | | | | |
| 3523 | 3538 | | | | | |
| 3557 | 3572 | | | | | |
| 3609 | 3624 | | | | | |
| 3643 | 3658 | | | | | |
| 3677 | 3692 | | | | | |
| 3728 | 3743 | | | | | |
| 3762 | 3777 | | | | | |
| 3864 | 3879 | | | | | |
| 3898 | 3913 | | | | | |
| 3966 | 3981 | | | | | |
| 4052 | 4067 | | | | | |
| 4086 | 4101 | | | | | |
| 4137 | 4152 | | | | | |
| 4171 | 4186 | | | | | |
| 4205 | 4220 | | | | | |
| 4324 | 4339 | | | | | |
| 4358 | 4373 | | | | | |
| 4392 | 4407 | | | | | |
| 4426 | 4441 | | | | | |
| 4460 | 4475 | | | | | |
| 2573 | 2588 | ACACCCACACTCCCGG | 629 | 540369 | 82 | eeeddddddddddkkk |
| 2641 | 2656 | | | | | |
| 2675 | 2690 | | | | | |
| 2709 | 2724 | | | | | |
| 2743 | 2758 | | | | | |
| 2777 | 2792 | | | | | |
| 2811 | 2826 | | | | | |
| 2845 | 2860 | | | | | |
| 2879 | 2894 | | | | | |
| 2913 | 2928 | | | | | |
| 2947 | 2962 | | | | | |
| 3015 | 3030 | | | | | |
| 3049 | 3064 | | | | | |
| 3083 | 3098 | | | | | |
| 3100 | 3115 | | | | | |
| 3287 | 3302 | | | | | |
| 3321 | 3336 | | | | | |
| 3355 | 3370 | | | | | |
| 3389 | 3404 | | | | | |
| 3423 | 3438 | | | | | |
| 3457 | 3472 | | | | | |
| 3491 | 3506 | | | | | |
| 3525 | 3540 | | | | | |
| 3559 | 3574 | | | | | |
| 3611 | 3626 | | | | | |
| 3645 | 3660 | | | | | |
| 3679 | 3694 | | | | | |
| 3730 | 3745 | | | | | |
| 3764 | 3779 | | | | | |
| 3866 | 3881 | | | | | |
| 3900 | 3915 | | | | | |
| 3968 | 3983 | | | | | |
| 4054 | 4069 | | | | | |
| 4088 | 4103 | | | | | |
| 4139 | 4154 | | | | | |
| 4173 | 4188 | | | | | |
| 4207 | 4222 | | | | | |
| 4326 | 4341 | | | | | |
| 4360 | 4375 | | | | | |
| 4394 | 4409 | | | | | |
| 4428 | 4443 | | | | | |
| 4462 | 4477 | | | | | |
| 2576 | 2591 | GGGACACCCACACTCC | 630 | 540370 | 86 | eeeddddddddddkkk |
| 2610 | 2625 | | | | | |
| 2644 | 2659 | | | | | |
| 2678 | 2693 | | | | | |
| 2712 | 2727 | | | | | |
| 2746 | 2761 | | | | | |
| 2780 | 2795 | | | | | |
| 2814 | 2829 | | | | | |
| 2848 | 2863 | | | | | |
| 2882 | 2897 | | | | | |
| 2916 | 2931 | | | | | |
| 2950 | 2965 | | | | | |
| 3018 | 3033 | | | | | |
| 3052 | 3067 | | | | | |
| 3086 | 3101 | | | | | |
| 3358 | 3373 | | | | | |
| 3460 | 3475 | | | | | |
| 3562 | 3577 | | | | | |
| 3682 | 3697 | | | | | |
| 3733 | 3748 | | | | | |
| 3869 | 3884 | | | | | |
| 3903 | 3918 | | | | | |
| 3937 | 3952 | | | | | |
| 4091 | 4106 | | | | | |
| 4142 | 4157 | | | | | |
| 4329 | 4344 | | | | | |
| 4431 | 4446 | | | | | |
| 4465 | 4480 | | | | | |
| 2578 | 2593 | CCGGGACACCCACACT | 631 | 540371 | 74 | eeeddddddddddkkk |
| 2612 | 2627 | | | | | |
| 2646 | 2661 | | | | | |
| 2680 | 2695 | | | | | |
| 2714 | 2729 | | | | | |
| 2748 | 2763 | | | | | |
| 2782 | 2797 | | | | | |
| 2816 | 2831 | | | | | |
| 2850 | 2865 | | | | | |
| 2884 | 2899 | | | | | |
| 2918 | 2933 | | | | | |
| 2952 | 2967 | | | | | |
| 3020 | 3035 | | | | | |
| 3054 | 3069 | | | | | |
| 3088 | 3103 | | | | | |
| 3360 | 3375 | | | | | |
| 3564 | 3579 | | | | | |
| 3684 | 3699 | | | | | |
| 3735 | 3750 | | | | | |
| 3871 | 3886 | | | | | |
| 3905 | 3920 | | | | | |
| 3939 | 3954 | | | | | |
| 4144 | 4159 | | | | | |
| 4331 | 4346 | | | | | |
| 4433 | 4448 | | | | | |
| 4467 | 4482 | | | | | |
| 2580 | 2595 | CCCCGGGACACCCACA | 632 | 540372 | 82 | eeeddddddddddkkk |
| 2614 | 2629 | | | | | |
| 2648 | 2663 | | | | | |
| 2716 | 2731 | | | | | |
| 2784 | 2799 | | | | | |
| 2818 | 2833 | | | | | |
| 2886 | 2901 | | | | | |
| 2954 | 2969 | | | | | |
| 3022 | 3037 | | | | | |
| 3056 | 3071 | | | | | |
| 3260 | 3275 | | | | | |
| 3362 | 3377 | | | | | |
| 3566 | 3581 | | | | | |
| 3686 | 3701 | | | | | |
| 3703 | 3718 | | | | | |
| 3737 | 3752 | | | | | |
| 3873 | 3888 | | | | | |
| 3941 | 3956 | | | | | |
| 4026 | 4041 | | | | | |
| 4112 | 4127 | | | | | |
| 4146 | 4161 | | | | | |
| 4299 | 4314 | | | | | |
| 4333 | 4348 | | | | | |
| 4435 | 4450 | | | | | |
| 4469 | 4484 | | | | | |
| 4554 | 4569 | | | | | |
| 2581 | 2596 | CCCCCGGGACACCCAC | 633 | 540373 | 81 | eeeddddddddddkkk |
| 2615 | 2630 | | | | | |
| 2649 | 2664 | | | | | |
| 2717 | 2732 | | | | | |
| 2785 | 2800 | | | | | |
| 2819 | 2834 | | | | | |
| 2887 | 2902 | | | | | |
| 2955 | 2970 | | | | | |
| 2989 | 3004 | | | | | |
| 3023 | 3038 | | | | | |
| 3057 | 3072 | | | | | |
| 3159 | 3174 | | | | | |
| 3176 | 3191 | | | | | |
| 3244 | 3259 | | | | | |
| 3261 | 3276 | | | | | |
| 3363 | 3378 | | | | | |
| 3567 | 3582 | | | | | |
| 3584 | 3599 | | | | | |
| 3687 | 3702 | | | | | |
| 3704 | 3719 | | | | | |
| 3738 | 3753 | | | | | |
| 3840 | 3855 | | | | | |
| 3874 | 3889 | | | | | |
| 3942 | 3957 | | | | | |
| 4027 | 4042 | | | | | |
| 4113 | 4128 | | | | | |
| 4147 | 4162 | | | | | |
| 4249 | 4264 | | | | | |
| 4300 | 4315 | | | | | |
| 4334 | 4349 | | | | | |
| 4436 | 4451 | | | | | |
| 4470 | 4485 | | | | | |
| 4538 | 4553 | | | | | |
| 4555 | 4570 | | | | | |
| 2583 | 2598 | CGCCCCCGGGACACCC | 634 | 540374 | 87 | eeeddddddddddkkk |
| 2617 | 2632 | | | | | |
| 2651 | 2666 | | | | | |
| 2787 | 2802 | | | | | |
| 2957 | 2972 | | | | | |
| 2991 | 3006 | | | | | |
| 3025 | 3040 | | | | | |
| 3059 | 3074 | | | | | |
| 3161 | 3176 | | | | | |
| 3178 | 3193 | | | | | |
| 3263 | 3278 | | | | | |
| 3365 | 3380 | | | | | |
| 3569 | 3584 | | | | | |
| 3842 | 3857 | | | | | |
| 3944 | 3959 | | | | | |
| 4115 | 4130 | | | | | |
| 4251 | 4266 | | | | | |
| 4302 | 4317 | | | | | |
| 4438 | 4453 | | | | | |
| 4472 | 4487 | | | | | |
| 2586 | 2601 | CCACGCCCCCGGGACA | 635 | 540375 | 78 | eeeddddddddddkkk |
| 2620 | 2635 | | | | | |
| 2654 | 2669 | | | | | |
| 2790 | 2805 | | | | | |
| 2960 | 2975 | | | | | |
| 2994 | 3009 | | | | | |
| 3028 | 3043 | | | | | |
| 3062 | 3077 | | | | | |
| 3147 | 3162 | | | | | |
| 3164 | 3179 | | | | | |
| 3181 | 3196 | | | | | |
| 3266 | 3281 | | | | | |
| 3368 | 3383 | | | | | |
| 3572 | 3587 | | | | | |
| 3845 | 3860 | | | | | |
| 3947 | 3962 | | | | | |
| 4118 | 4133 | | | | | |
| 4254 | 4269 | | | | | |
| 4305 | 4320 | | | | | |
| 4441 | 4456 | | | | | |
| 4475 | 4490 | | | | | |
| 2589 | 2604 | CACCCACGCCCCCGGG | 636 | 540376 | 69 | eeeddddddddddkkk |
| 2623 | 2638 | | | | | |
| 2657 | 2672 | | | | | |
| 2793 | 2808 | | | | | |
| 2963 | 2978 | | | | | |
| 2997 | 3012 | | | | | |
| 3031 | 3046 | | | | | |
| 3065 | 3080 | | | | | |
| 3150 | 3165 | | | | | |
| 3167 | 3182 | | | | | |
| 3184 | 3199 | | | | | |
| 3269 | 3284 | | | | | |
| 3371 | 3386 | | | | | |
| 3575 | 3590 | | | | | |
| 3848 | 3863 | | | | | |
| 3950 | 3965 | | | | | |
| 4121 | 4136 | | | | | |
| 4257 | 4272 | | | | | |
| 4308 | 4323 | | | | | |
| 4444 | 4459 | | | | | |
| 2592 | 2607 | GGACACCCACGCCCCC | 637 | 540377 | 88 | eeeddddddddddkkk |
| 2626 | 2641 | | | | | |
| 2660 | 2675 | | | | | |
| 2796 | 2811 | | | | | |
| 2966 | 2981 | | | | | |
| 3000 | 3015 | | | | | |
| 3034 | 3049 | | | | | |
| 3068 | 3083 | | | | | |
| 3153 | 3168 | | | | | |
| 3170 | 3185 | | | | | |
| 3272 | 3287 | | | | | |
| 3374 | 3389 | | | | | |
| 3578 | 3593 | | | | | |
| 3851 | 3866 | | | | | |
| 3953 | 3968 | | | | | |
| 4124 | 4139 | | | | | |
| 4260 | 4275 | | | | | |
| 4311 | 4326 | | | | | |
| 4447 | 4462 | | | | | |
| 4532 | 4547 | | | | | |
| 2593 | 2608 | GGGACACCCACGCCCC | 638 | 540378 | 85 | eeeddddddddddkkk |
| 2627 | 2642 | | | | | |
| 2661 | 2676 | | | | | |
| 2797 | 2812 | | | | | |
| 2967 | 2982 | | | | | |
| 3001 | 3016 | | | | | |
| 3035 | 3050 | | | | | |
| 3069 | 3084 | | | | | |
| 3154 | 3169 | | | | | |
| 3171 | 3186 | | | | | |
| 3239 | 3254 | | | | | |
| 3273 | 3288 | | | | | |
| 3375 | 3390 | | | | | |
| 3477 | 3492 | | | | | |
| 3579 | 3594 | | | | | |
| 3852 | 3867 | | | | | |
| 3954 | 3969 | | | | | |
| 4125 | 4140 | | | | | |
| 4261 | 4276 | | | | | |
| 4312 | 4327 | | | | | |
| 4448 | 4463 | | | | | |
| 4533 | 4548 | | | | | |
| 2628 | 2643 | CGGGACACCCACGCCC | 639 | 540379 | 77 | eeeddddddddddkkk |
| 2662 | 2677 | | | | | |
| 2798 | 2813 | | | | | |
| 2968 | 2983 | | | | | |
| 3002 | 3017 | | | | | |
| 3036 | 3051 | | | | | |
| 3070 | 3085 | | | | | |
| 3155 | 3170 | | | | | |
| 3172 | 3187 | | | | | |
| 3240 | 3255 | | | | | |
| 3274 | 3289 | | | | | |
| 3376 | 3391 | | | | | |
| 3478 | 3493 | | | | | |
| 3580 | 3595 | | | | | |
| 3853 | 3868 | | | | | |
| 3955 | 3970 | | | | | |
| 4126 | 4141 | | | | | |
| 4262 | 4277 | | | | | |
| 4313 | 4328 | | | | | |
| 4449 | 4464 | | | | | |
| 4534 | 4549 | | | | | |
| 2629 | 2644 | CCGGGACACCCACGCC | 640 | 540380 | 84 | eeeddddddddddkkk |
| 2663 | 2678 | | | | | |
| 2799 | 2814 | | | | | |
| 2969 | 2984 | | | | | |
| 3003 | 3018 | | | | | |
| 3037 | 3052 | | | | | |
| 3071 | 3086 | | | | | |
| 3156 | 3171 | | | | | |
| 3173 | 3188 | | | | | |
| 3241 | 3256 | | | | | |
| 3275 | 3290 | | | | | |
| 3377 | 3392 | | | | | |
| 3479 | 3494 | | | | | |
| 3581 | 3596 | | | | | |
| 3854 | 3869 | | | | | |
| 3956 | 3971 | | | | | |
| 4127 | 4142 | | | | | |
| 4263 | 4278 | | | | | |
| 4314 | 4329 | | | | | |
| 4450 | 4465 | | | | | |
| 4535 | 4550 | | | | | |
| 2630 | 2645 | CCCGGGACACCCACGC | 641 | 540381 | 85 | eeeddddddddddkkk |
| 2664 | 2679 | | | | | |
| 2800 | 2815 | | | | | |
| 2970 | 2985 | | | | | |
| 2987 | 3002 | | | | | |
| 3004 | 3019 | | | | | |
| 3038 | 3053 | | | | | |
| 3072 | 3087 | | | | | |
| 3157 | 3172 | | | | | |
| 3174 | 3189 | | | | | |
| 3242 | 3257 | | | | | |
| 3276 | 3291 | | | | | |
| 3378 | 3393 | | | | | |
| 3480 | 3495 | | | | | |
| 3582 | 3597 | | | | | |
| 3838 | 3853 | | | | | |
| 3855 | 3870 | | | | | |
| 3957 | 3972 | | | | | |
| 4128 | 4143 | | | | | |
| 4247 | 4262 | | | | | |
| 4264 | 4279 | | | | | |
| 4315 | 4330 | | | | | |
| 4451 | 4466 | | | | | |
| 4536 | 4551 | | | | | |
| 2683 | 2698 | CCTCCGGGACACCCAC | 642 | 540382 | 69 | eeeddddddddddkkk |
| 2751 | 2766 | | | | | |
| 2853 | 2868 | | | | | |
| 2921 | 2936 | | | | | |
| 3806 | 3821 | | | | | |
| 3908 | 3923 | | | | | |
| 2684 | 2699 | GCCTCCGGGACACCCA | 643 | 540383 | 85 | eeeddddddddddkkk |
| 2752 | 2767 | | | | | |
| 2854 | 2869 | | | | | |
| 2922 | 2937 | | | | | |
| 3807 | 3822 | | | | | |
| 3909 | 3924 | | | | | |
| 2692 | 2707 | ACACCCTCGCCTCCGG | 644 | 540384 | 88 | eeeddddddddddkkk |
| 2760 | 2775 | | | | | |
| 2862 | 2877 | | | | | |
| 2930 | 2945 | | | | | |
| 3117 | 3132 | | | | | |
| 3338 | 3353 | | | | | |
| 3440 | 3455 | | | | | |
| 3508 | 3523 | | | | | |
| 3542 | 3557 | | | | | |
| 3628 | 3643 | | | | | |
| 3662 | 3677 | | | | | |
| 3781 | 3796 | | | | | |
| 3815 | 3830 | | | | | |
| 3917 | 3932 | | | | | |
| 4190 | 4205 | | | | | |
| 4224 | 4239 | | | | | |
| 4377 | 4392 | | | | | |
| 4411 | 4426 | | | | | |
| 2695 | 2710 | GGGACACCCTCGCCTC | 645 | 540385 | 87 | eeeddddddddddkkk |
| 2763 | 2778 | | | | | |
| 2865 | 2880 | | | | | |
| 2933 | 2948 | | | | | |
| 3120 | 3135 | | | | | |
| 3341 | 3356 | | | | | |
| 3443 | 3458 | | | | | |
| 3511 | 3526 | | | | | |
| 3545 | 3560 | | | | | |
| 3631 | 3646 | | | | | |
| 3665 | 3680 | | | | | |
| 3784 | 3799 | | | | | |
| 3818 | 3833 | | | | | |
| 3920 | 3935 | | | | | |
| 4074 | 4089 | | | | | |
| 4193 | 4208 | | | | | |
| 4227 | 4242 | | | | | |
| 4380 | 4395 | | | | | |
| 4414 | 4429 | | | | | |
| 2697 | 2712 | CCGGGACACCCTCGCC | 646 | 540386 | 86 | eeeddddddddddkkk |
| 2765 | 2780 | | | | | |
| 2867 | 2882 | | | | | |
| 2935 | 2950 | | | | | |
| 3122 | 3137 | | | | | |
| 3343 | 3358 | | | | | |
| 3445 | 3460 | | | | | |
| 3513 | 3528 | | | | | |
| 3547 | 3562 | | | | | |
| 3633 | 3648 | | | | | |
| 3667 | 3682 | | | | | |
| 3820 | 3835 | | | | | |
| 4076 | 4091 | | | | | |
| 4195 | 4210 | | | | | |
| 4229 | 4244 | | | | | |
| 4382 | 4397 | | | | | |
| 4416 | 4431 | | | | | |
| 2699 | 2714 | TCCCGGGACACCCTCG | 647 | 540387 | 77 | eeeddddddddddkkk |
| 2767 | 2782 | | | | | |
| 2869 | 2884 | | | | | |
| 2937 | 2952 | | | | | |
| 3124 | 3139 | | | | | |
| 3345 | 3360 | | | | | |
| 3447 | 3462 | | | | | |
| 3515 | 3530 | | | | | |
| 3549 | 3564 | | | | | |
| 3635 | 3650 | | | | | |
| 3669 | 3684 | | | | | |
| 3822 | 3837 | | | | | |
| 4078 | 4093 | | | | | |
| 4197 | 4212 | | | | | |
| 4231 | 4246 | | | | | |
| 4384 | 4399 | | | | | |
| 4418 | 4433 | | | | | |
| 2701 | 2716 | ACTCCCGGGACACCCT | 648 | 540388 | 86 | eeeddddddddddkkk |
| 2769 | 2784 | | | | | |
| 2871 | 2886 | | | | | |
| 2939 | 2954 | | | | | |
| 3126 | 3141 | | | | | |
| 3313 | 3328 | | | | | |
| 3347 | 3362 | | | | | |
| 3415 | 3430 | | | | | |
| 3449 | 3464 | | | | | |
| 3517 | 3532 | | | | | |
| 3551 | 3566 | | | | | |
| 3637 | 3652 | | | | | |
| 3671 | 3686 | | | | | |
| 4080 | 4095 | | | | | |
| 4199 | 4214 | | | | | |
| 4386 | 4401 | | | | | |
| 4420 | 4435 | | | | | |
| 2974 | 2989 | CGCTCCCGGGACACCC | 649 | 540389 | 86 | eeeddddddddddkkk |
| 3825 | 3840 | | | | | |
| 4234 | 4249 | | | | | |
| 4268 | 4283 | | | | | |
| 4575 | 4590 | | | | | |
| 2988 | 3003 | CCCCGGGACACCCACG | 650 | 540390 | 85 | eeeddddddddddkkk |
| 3158 | 3173 | | | | | |
| 3175 | 3190 | | | | | |
| 3243 | 3258 | | | | | |
| 3583 | 3598 | | | | | |
| 3839 | 3854 | | | | | |
| 4248 | 4263 | | | | | |
| 4537 | 4552 | | | | | |
| 3103 | 3118 | GGAACACCCACACTCC | 651 | 540391 | 83 | eeeddddddddddkkk |
| 3290 | 3305 | | | | | |
| 3324 | 3339 | | | | | |
| 3392 | 3407 | | | | | |
| 3426 | 3441 | | | | | |
| 3494 | 3509 | | | | | |
| 3528 | 3543 | | | | | |
| 3614 | 3629 | | | | | |
| 3648 | 3663 | | | | | |
| 3767 | 3782 | | | | | |
| 3971 | 3986 | | | | | |
| 4057 | 4072 | | | | | |
| 4176 | 4191 | | | | | |
| 4210 | 4225 | | | | | |
| 4363 | 4378 | | | | | |
| 4397 | 4412 | | | | | |
| 3106 | 3121 | TCCGGAACACCCACAC | 652 | 540392 | 43 | eeeddddddddddkkk |
| 3293 | 3308 | | | | | |
| 3327 | 3342 | | | | | |
| 3395 | 3410 | | | | | |
| 3429 | 3444 | | | | | |
| 3497 | 3512 | | | | | |
| 3531 | 3546 | | | | | |
| 3617 | 3632 | | | | | |
| 3651 | 3666 | | | | | |
| 3770 | 3785 | | | | | |
| 4179 | 4194 | | | | | |
| 4213 | 4228 | | | | | |
| 4366 | 4381 | | | | | |
| 4400 | 4415 | | | | | |
| 3109 | 3124 | GCCTCCGGAACACCCA | 653 | 540393 | 88 | eeeddddddddddkkk |
| 3194 | 3209 | | | | | |
| 3330 | 3345 | | | | | |
| 3432 | 3447 | | | | | |
| 3500 | 3515 | | | | | |
| 3534 | 3549 | | | | | |
| 3620 | 3635 | | | | | |
| 3654 | 3669 | | | | | |
| 3773 | 3788 | | | | | |
| 4182 | 4197 | | | | | |
| 4216 | 4231 | | | | | |
| 4369 | 4384 | | | | | |
| 4403 | 4418 | | | | | |
| 3112 | 3127 | CTCGCCTCCGGAACAC | 654 | 540394 | 68 | eeeddddddddddkkk |
| 3197 | 3212 | | | | | |
| 3333 | 3348 | | | | | |
| 3435 | 3450 | | | | | |
| 3503 | 3518 | | | | | |
| 3537 | 3552 | | | | | |
| 3623 | 3638 | | | | | |
| 3657 | 3672 | | | | | |
| 3776 | 3791 | | | | | |
| 4185 | 4200 | | | | | |
| 4219 | 4234 | | | | | |
| 4372 | 4387 | | | | | |
| 4406 | 4421 | | | | | |
| 3115 | 3130 | ACCCTCGCCTCCGGAA | 655 | 540395 | 87 | eeeddddddddddkkk |
| 3200 | 3215 | | | | | |
| 3336 | 3351 | | | | | |
| 3438 | 3453 | | | | | |
| 3506 | 3521 | | | | | |
| 3540 | 3555 | | | | | |
| 3626 | 3641 | | | | | |
| 3660 | 3675 | | | | | |
| 3779 | 3794 | | | | | |
| 4188 | 4203 | | | | | |
| 4222 | 4237 | | | | | |
| 4375 | 4390 | | | | | |
| 4409 | 4424 | | | | | |
| 3245 | 3260 | ACCCCCGGGACACCCA | 656 | 540396 | 87 | eeeddddddddddkkk |
| 3585 | 3600 | | | | | |
| 3688 | 3703 | | | | | |
| 3705 | 3720 | | | | | |
| 4028 | 4043 | | | | | |
| 4539 | 4554 | | | | | |
| 4556 | 4571 | | | | | |
| 3249 | 3264 | CCACACCCCCGGGACA | 657 | 540397 | 59 | eeeddddddddddkkk |
| 3692 | 3707 | | | | | |
| 3709 | 3724 | | | | | |
| 4015 | 4030 | | | | | |
| 4543 | 4558 | | | | | |
| 3252 | 3267 | CACCCACACCCCCGGG | 658 | 540398 | 36 | eeeddddddddddkkk |
| 3695 | 3710 | | | | | |
| 3712 | 3727 | | | | | |
| 4018 | 4033 | | | | | |
| 4546 | 4561 | | | | | |
| 14810 | 14825 | GTGTGTGCATATCTCT | 659 | 540399 | 81 | eeeddddddddddkkk |
| 14886 | 14901 | | | | | |
| 14976 | 14991 | | | | | |

### Example 23

### High Dose Tolerability of Modified Oligonucleotides Comprising MOE and cEt Modifications Targeting Human Factor VII in BALB/c Mice

BALB/c mice were treated at a high dose with ISIS antisense oligonucleotides selected from studies described above and evaluated for changes in the levels of various plasma chemistry markers.

Additionally, newly designed antisense oligonucleotides were also added to this screen. The newly designed modified antisense oligonucleotides are presented in Table 24 and were designed with the same sequences as antisense oligonucleotides from the study described above. The newly designed oligonucleotides are 16 nucleosides in length and target intronic repeat regions of SEQ ID NO: 1. The newly designed modified antisense oligonucleotides and their motifs are described in Table 24. The internucleoside linkages throughout each oligonucleotide are phosphorothioate linkages. All cytosines in the oligonucleotides are 5-methylcytosines. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO.

Each oligonucleotide listed in Table 24 is targeted to intronic regions of human Factor VII genomic sequence, designated herein as SEQ ID NO: 1 (GENBANK Accession No. NT_{—}027140.6 truncated from nucleotides 1255000 to 1273000). “Start site” indicates the 5′-most nucleoside to which the oligonucleotide is targeted in the human gene sequence. “Stop site” indicates the 3′-most nucleoside to which the oligonucleotide is targeted in the human gene sequence.

TABLE 24
Modified antisense oligonucleotides targeted to SEQ ID NO: 1

| Start Site | Stop Site | | | | |
|---|---|---|---|---|---|
| on SEQ | on SEQ | | SEQ ID | | |
| ID NO: 1 | ID NO: 1 | Sequence | NO | ISIS No | Sugar Chemistry |
| 6712 | 6727 | GTGTGAGGTGACCTGT | 509 | 537721 | kkkddddddddddeee |
| 6834 | 6849 | | | | |
| 7022 | 7037 | | | | |
| 7140 | 7155 | | | | |
| 7397 | 7412 | | | | |
| 7463 | 7478 | | | | |
| 7862 | 7877 | | | | |
| 6729 | 6744 | TGTGAGGTGTCCTGTG | 524 | 537738 | kkkddddddddddeee |
| 6851 | 6866 | | | | |
| 6973 | 6988 | | | | |
| 7039 | 7054 | | | | |
| 7091 | 7106 | | | | |
| 7157 | 7172 | | | | |
| 7209 | 7224 | | | | |
| 7263 | 7278 | | | | |
| 7291 | 7306 | | | | |
| 7350 | 7365 | | | | |
| 7414 | 7429 | | | | |
| 7480 | 7495 | | | | |
| 7512 | 7527 | | | | |
| 7526 | 7541 | | | | |
| 7558 | 7573 | | | | |
| 7630 | 7645 | | | | |
| 7684 | 7699 | | | | |
| 7879 | 7894 | | | | |
| 7911 | 7926 | | | | |
| 7975 | 7990 | | | | |
| 8035 | 8050 | | | | |
| 8067 | 8082 | | | | |
| 8139 | 8154 | | | | |
| 8175 | 8190 | | | | |
| 6762 | 6777 | GGTGACCCGTGAGTGT | 539 | 537759 | kkkddddddddddeee |
| 6884 | 6899 | | | | |
| 6950 | 6965 | | | | |
| 7068 | 7083 | | | | |
| 7186 | 7201 | | | | |
| 7240 | 7255 | | | | |
| 6764 | 6779 | GAGGTGACCCGTGAGT | 541 | 537761 | kkkddddddddddeee |
| 6886 | 6901 | | | | |
| 6952 | 6967 | | | | |
| 7070 | 7085 | | | | |
| 7188 | 7203 | | | | |
| 7242 | 7257 | | | | |
| 6644 | 6659 | GTGAGGTGACCCGTGA | 543 | 537763 | kkkddddddddddeee |
| 6766 | 6781 | | | | |
| 6888 | 6903 | | | | |
| 6954 | 6969 | | | | |
| 7072 | 7087 | | | | |
| 7190 | 7205 | | | | |
| 7244 | 7259 | | | | |
| 6697 | 6712 | TGAGTGTGAAGTGTGC | 548 | 537850 | kkkddddddddddeee |
| 6753 | 6768 | | | | |
| 6819 | 6834 | | | | |
| 6875 | 6890 | | | | |
| 6941 | 6956 | | | | |
| 7007 | 7022 | | | | |
| 7059 | 7074 | | | | |
| 7125 | 7140 | | | | |
| 7177 | 7192 | | | | |
| 7382 | 7397 | | | | |
| 7448 | 7463 | | | | |
| 7795 | 7810 | | | | |
| 7945 | 7960 | | | | |
| 8286 | 8301 | | | | |
| 6705 | 6720 | GTGACCTGTGAGTGTG | 556 | 537858 | kkkddddddddddeee |
| 6827 | 6842 | | | | |
| 7015 | 7030 | | | | |
| 7133 | 7148 | | | | |
| 7390 | 7405 | | | | |
| 7456 | 7471 | | | | |
| 7606 | 7621 | | | | |
| 7642 | 7657 | | | | |
| 7803 | 7818 | | | | |
| 8115 | 8130 | | | | |
| 8151 | 8166 | | | | |
| 8294 | 8309 | | | | |
| 6711 | 6726 | TGTGAGGTGACCTGTG | 562 | 537864 | kkkddddddddddeee |
| 6833 | 6848 | | | | |
| 7021 | 7036 | | | | |
| 7139 | 7154 | | | | |
| 7396 | 7411 | | | | |
| 7462 | 7477 | | | | |
| 7861 | 7876 | | | | |
| 6693 | 6708 | TGTGAAGTGTGCCCTG | 565 | 537869 | kkkddddddddddeee |
| 6749 | 6764 | | | | |
| 6815 | 6830 | | | | |
| 6871 | 6886 | | | | |
| 6937 | 6952 | | | | |
| 7055 | 7070 | | | | |
| 7173 | 7188 | | | | |
| 7791 | 7806 | | | | |
| 8282 | 8297 | | | | |
| 6696 | 6711 | GAGTGTGAAGTGTGCC | 568 | 537872 | kkkddddddddddeee |
| 6752 | 6767 | | | | |
| 6818 | 6833 | | | | |
| 6874 | 6889 | | | | |
| 6940 | 6955 | | | | |
| 7006 | 7021 | | | | |
| 7058 | 7073 | | | | |
| 7124 | 7139 | | | | |
| 7176 | 7191 | | | | |
| 7381 | 7396 | | | | |
| 7447 | 7462 | | | | |
| 7794 | 7809 | | | | |
| 7944 | 7959 | | | | |
| 8285 | 8300 | | | | |
| 6680 | 6695 | CTGTGTGAGGTGTCCT | 571 | 537897 | kkkddddddddddeee |
| 6802 | 6817 | | | | |
| 6924 | 6939 | | | | |
| 7042 | 7057 | | | | |
| 7160 | 7175 | | | | |
| 7515 | 7530 | | | | |
| 7687 | 7702 | | | | |
| 8178 | 8193 | | | | |
| 6975 | 6990 | TGTGTGAGGTGTCCTG | 582 | 540118 | kkkddddddddddeee |
| 7041 | 7056 | | | | |
| 7093 | 7108 | | | | |
| 7159 | 7174 | | | | |
| 7352 | 7367 | | | | |
| 7514 | 7529 | | | | |
| 7686 | 7701 | | | | |
| 8177 | 8192 | | | | |
| 7038 | 7053 | GTGAGGTGTCCTGTGG | 602 | 540138 | kkkddddddddddeee |
| 7156 | 7171 | | | | |
| 7262 | 7277 | | | | |
| 7479 | 7494 | | | | |
| 7511 | 7526 | | | | |
| 7557 | 7572 | | | | |
| 7910 | 7925 | | | | |
| 7974 | 7989 | | | | |
| 8066 | 8081 | | | | |
| 7264 | 7279 | CTGTGAGGTGTCCTGT | 603 | 540139 | kkkddddddddddeee |
| 7415 | 7430 | | | | |
| 7481 | 7496 | | | | |
| 7527 | 7542 | | | | |
| 7880 | 7895 | | | | |
| 7912 | 7927 | | | | |
| 7976 | 7991 | | | | |
| 7278 | 7293 | GTGTGAGGTGACCTCT | 612 | 540148 | kkkddddddddddeee |
| 7429 | 7444 | | | | |
| 7495 | 7510 | | | | |
| 7541 | 7556 | | | | |
| 7894 | 7909 | | | | |
| 7926 | 7941 | | | | |
| 7990 | 8005 | | | | |
| 8022 | 8037 | | | | |
| 7604 | 7619 | GACCTGTGAGTGTGAG | 617 | 540153 | kkkddddddddddeee |
| 7640 | 7655 | | | | |
| 8113 | 8128 | | | | |
| 8149 | 8164 | | | | |
| 8373 | 8388 | | | | |
| 7627 | 7642 | GAGGTGTCCTGTGAGA | 619 | 540155 | kkkddddddddddeee |
| 7663 | 7678 | | | | |
| 7681 | 7696 | | | | |
| 7840 | 7855 | | | | |
| 8136 | 8151 | | | | |
| 8172 | 8187 | | | | |
| 8331 | 8346 | | | | |
| 2565 | 2580 | ACTCCCGGGACACCCA | 624 | 540162 | eekddddddddddkke |
| 2633 | 2648 | | | | |
| 2667 | 2682 | | | | |
| 2735 | 2750 | | | | |
| 2803 | 2818 | | | | |
| 2837 | 2852 | | | | |
| 2905 | 2920 | | | | |
| 3007 | 3022 | | | | |
| 3041 | 3056 | | | | |
| 3075 | 3090 | | | | |
| 3092 | 3107 | | | | |
| 3279 | 3294 | | | | |
| 3381 | 3396 | | | | |
| 3483 | 3498 | | | | |
| 3603 | 3618 | | | | |
| 3722 | 3737 | | | | |
| 3756 | 3771 | | | | |
| 3858 | 3873 | | | | |
| 3892 | 3907 | | | | |
| 3960 | 3975 | | | | |
| 4046 | 4061 | | | | |
| 4131 | 4146 | | | | |
| 4165 | 4180 | | | | |
| 4318 | 4333 | | | | |
| 4454 | 4469 | | | | |
| 2567 | 2582 | ACACTCCCGGGACACC | 626 | 540164 | eekddddddddddkke |
| 2635 | 2650 | | | | |
| 2669 | 2684 | | | | |
| 2703 | 2718 | | | | |
| 2737 | 2752 | | | | |
| 2771 | 2786 | | | | |
| 2805 | 2820 | | | | |
| 2839 | 2854 | | | | |
| 2873 | 2888 | | | | |
| 2907 | 2922 | | | | |
| 2941 | 2956 | | | | |
| 3009 | 3024 | | | | |
| 3043 | 3058 | | | | |
| 3077 | 3092 | | | | |
| 3094 | 3109 | | | | |
| 3128 | 3143 | | | | |
| 3281 | 3296 | | | | |
| 3315 | 3330 | | | | |
| 3349 | 3364 | | | | |
| 3383 | 3398 | | | | |
| 3417 | 3432 | | | | |
| 3451 | 3466 | | | | |
| 3485 | 3500 | | | | |
| 3519 | 3534 | | | | |
| 3553 | 3568 | | | | |
| 3605 | 3620 | | | | |
| 3639 | 3654 | | | | |
| 3673 | 3688 | | | | |
| 3724 | 3739 | | | | |
| 3758 | 3773 | | | | |
| 3860 | 3875 | | | | |
| 3894 | 3909 | | | | |
| 3962 | 3977 | | | | |
| 4048 | 4063 | | | | |
| 4082 | 4097 | | | | |
| 4133 | 4148 | | | | |
| 4167 | 4182 | | | | |
| 4201 | 4216 | | | | |
| 4320 | 4335 | | | | |
| 4388 | 4403 | | | | |
| 4422 | 4437 | | | | |
| 4456 | 4471 | | | | |
| 2576 | 2591 | GGGACACCCACACTCC | 630 | 540168 | eekddddddddddkke |
| 2610 | 2625 | | | | |
| 2644 | 2659 | | | | |
| 2678 | 2693 | | | | |
| 2712 | 2727 | | | | |
| 2746 | 2761 | | | | |
| 2780 | 2795 | | | | |
| 2814 | 2829 | | | | |
| 2848 | 2863 | | | | |
| 2882 | 2897 | | | | |
| 2916 | 2931 | | | | |
| 2950 | 2965 | | | | |
| 3018 | 3033 | | | | |
| 3052 | 3067 | | | | |
| 3086 | 3101 | | | | |
| 3358 | 3373 | | | | |
| 3460 | 3475 | | | | |
| 3562 | 3577 | | | | |
| 3682 | 3697 | | | | |
| 3733 | 3748 | | | | |
| 3869 | 3884 | | | | |
| 3903 | 3918 | | | | |
| 3937 | 3952 | | | | |
| 4091 | 4106 | | | | |
| 4142 | 4157 | | | | |
| 4329 | 4344 | | | | |
| 4431 | 4446 | | | | |
| 4465 | 4480 | | | | |
| 2583 | 2598 | CGCCCCCGGGACACCC | 634 | 540172 | eekddddddddddkke |
| 2617 | 2632 | | | | |
| 2651 | 2666 | | | | |
| 2787 | 2802 | | | | |
| 2957 | 2972 | | | | |
| 2991 | 3006 | | | | |
| 3025 | 3040 | | | | |
| 3059 | 3074 | | | | |
| 3161 | 3176 | | | | |
| 3178 | 3193 | | | | |
| 3263 | 3278 | | | | |
| 3365 | 3380 | | | | |
| 3569 | 3584 | | | | |
| 3842 | 3857 | | | | |
| 3944 | 3959 | | | | |
| 4115 | 4130 | | | | |
| 4251 | 4266 | | | | |
| 4302 | 4317 | | | | |
| 4438 | 4453 | | | | |
| 4472 | 4487 | | | | |
| 2592 | 2607 | GGACACCCACGCCCCC | 637 | 540175 | eekddddddddddkke |
| 2626 | 2641 | | | | |
| 2660 | 2675 | | | | |
| 2796 | 2811 | | | | |
| 2966 | 2981 | | | | |
| 3000 | 3015 | | | | |
| 3034 | 3049 | | | | |
| 3068 | 3083 | | | | |
| 3153 | 3168 | | | | |
| 3170 | 3185 | | | | |
| 3272 | 3287 | | | | |
| 3374 | 3389 | | | | |
| 3578 | 3593 | | | | |
| 3851 | 3866 | | | | |
| 3953 | 3968 | | | | |
| 4124 | 4139 | | | | |
| 4260 | 4275 | | | | |
| 4311 | 4326 | | | | |
| 4447 | 4462 | | | | |
| 4532 | 4547 | | | | |
| 2593 | 2608 | GGGACACCCACGCCCC | 638 | 540176 | eekddddddddddkke |
| 2627 | 2642 | | | | |
| 2661 | 2676 | | | | |
| 2797 | 2812 | | | | |
| 2967 | 2982 | | | | |
| 3001 | 3016 | | | | |
| 3035 | 3050 | | | | |
| 3069 | 3084 | | | | |
| 3154 | 3169 | | | | |
| 3171 | 3186 | | | | |
| 3239 | 3254 | | | | |
| 3273 | 3288 | | | | |
| 3375 | 3390 | | | | |
| 3477 | 3492 | | | | |
| 3579 | 3594 | | | | |
| 3852 | 3867 | | | | |
| 3954 | 3969 | | | | |
| 4125 | 4140 | | | | |
| 4261 | 4276 | | | | |
| 4312 | 4327 | | | | |
| 4448 | 4463 | | | | |
| 4533 | 4548 | | | | |
| 2629 | 2644 | CCGGGACACCCACGCC | 640 | 540178 | eekddddddddddkke |
| 2663 | 2678 | | | | |
| 2799 | 2814 | | | | |
| 2969 | 2984 | | | | |
| 3003 | 3018 | | | | |
| 3037 | 3052 | | | | |
| 3071 | 3086 | | | | |
| 3156 | 3171 | | | | |
| 3173 | 3188 | | | | |
| 3241 | 3256 | | | | |
| 3275 | 3290 | | | | |
| 3377 | 3392 | | | | |
| 3479 | 3494 | | | | |
| 3581 | 3596 | | | | |
| 3854 | 3869 | | | | |
| 3956 | 3971 | | | | |
| 4127 | 4142 | | | | |
| 4263 | 4278 | | | | |
| 4314 | 4329 | | | | |
| 4450 | 4465 | | | | |
| 4535 | 4550 | | | | |
| 2630 | 2645 | CCCGGGACACCCACGC | 641 | 540179 | eekddddddddddkke |
| 2664 | 2679 | | | | |
| 2800 | 2815 | | | | |
| 2970 | 2985 | | | | |
| 2987 | 3002 | | | | |
| 3004 | 3019 | | | | |
| 3038 | 3053 | | | | |
| 3072 | 3087 | | | | |
| 3157 | 3172 | | | | |
| 3174 | 3189 | | | | |
| 3242 | 3257 | | | | |
| 3276 | 3291 | | | | |
| 3378 | 3393 | | | | |
| 3480 | 3495 | | | | |
| 3582 | 3597 | | | | |
| 3838 | 3853 | | | | |
| 3855 | 3870 | | | | |
| 3957 | 3972 | | | | |
| 4128 | 4143 | | | | |
| 4247 | 4262 | | | | |
| 4264 | 4279 | | | | |
| 4315 | 4330 | | | | |
| 4451 | 4466 | | | | |
| 4536 | 4551 | | | | |
| 2684 | 2699 | GCCTCCGGGACACCCA | 643 | 540181 | eekddddddddddkke |
| 2752 | 2767 | | | | |
| 2854 | 2869 | | | | |
| 2922 | 2937 | | | | |
| 3807 | 3822 | | | | |
| 3909 | 3924 | | | | |
| 2692 | 2707 | ACACCCTCGCCTCCGG | 644 | 540182 | eekddddddddddkke |
| 2760 | 2775 | | | | |
| 2862 | 2877 | | | | |
| 2930 | 2945 | | | | |
| 3117 | 3132 | | | | |
| 3338 | 3353 | | | | |
| 3440 | 3455 | | | | |
| 3508 | 3523 | | | | |
| 3542 | 3557 | | | | |
| 3628 | 3643 | | | | |
| 3662 | 3677 | | | | |
| 3781 | 3796 | | | | |
| 3815 | 3830 | | | | |
| 3917 | 3932 | | | | |
| 4190 | 4205 | | | | |
| 4224 | 4239 | | | | |
| 4377 | 4392 | | | | |
| 4411 | 4426 | | | | |
| 2695 | 2710 | GGGACACCCTCGCCTC | 645 | 540183 | eekddddddddddkke |
| 2763 | 2778 | | | | |
| 2865 | 2880 | | | | |
| 2933 | 2948 | | | | |
| 3120 | 3135 | | | | |
| 3341 | 3356 | | | | |
| 3443 | 3458 | | | | |
| 3511 | 3526 | | | | |
| 3545 | 3560 | | | | |
| 3631 | 3646 | | | | |
| 3665 | 3680 | | | | |
| 3784 | 3799 | | | | |
| 3818 | 3833 | | | | |
| 3920 | 3935 | | | | |
| 4074 | 4089 | | | | |
| 4193 | 4208 | | | | |
| 4227 | 4242 | | | | |
| 4380 | 4395 | | | | |
| 4414 | 4429 | | | | |
| 2697 | 2712 | CCGGGACACCCTCGCC | 646 | 540184 | eekddddddddddkke |
| 2765 | 2780 | | | | |
| 2867 | 2882 | | | | |
| 2935 | 2950 | | | | |
| 3122 | 3137 | | | | |
| 3343 | 3358 | | | | |
| 3445 | 3460 | | | | |
| 3513 | 3528 | | | | |
| 3547 | 3562 | | | | |
| 3633 | 3648 | | | | |
| 3667 | 3682 | | | | |
| 3820 | 3835 | | | | |
| 4076 | 4091 | | | | |
| 4195 | 4210 | | | | |
| 4229 | 4244 | | | | |
| 4382 | 4397 | | | | |
| 4416 | 4431 | | | | |
| 2701 | 2716 | ACTCCCGGGACACCCT | 648 | 540186 | eekddddddddddkke |
| 2769 | 2784 | | | | |
| 2871 | 2886 | | | | |
| 2939 | 2954 | | | | |
| 3126 | 3141 | | | | |
| 3313 | 3328 | | | | |
| 3347 | 3362 | | | | |
| 3415 | 3430 | | | | |
| 3449 | 3464 | | | | |
| 3517 | 3532 | | | | |
| 3551 | 3566 | | | | |
| 3637 | 3652 | | | | |
| 3671 | 3686 | | | | |
| 4080 | 4095 | | | | |
| 4199 | 4214 | | | | |
| 4386 | 4401 | | | | |
| 4420 | 4435 | | | | |
| 2974 | 2989 | CGCTCCCGGGACACCC | 649 | 540187 | eekddddddddddkke |
| 3825 | 3840 | | | | |
| 4234 | 4249 | | | | |
| 4268 | 4283 | | | | |
| 4575 | 4590 | | | | |
| 2988 | 3003 | CCCCGGGACACCCACG | 650 | 540188 | eekddddddddddkke |
| 3158 | 3173 | | | | |
| 3175 | 3190 | | | | |
| 3243 | 3258 | | | | |
| 3583 | 3598 | | | | |
| 3839 | 3854 | | | | |
| 4248 | 4263 | | | | |
| 4537 | 4552 | | | | |
| 3109 | 3124 | GCCTCCGGAACACCCA | 653 | 540191 | eekddddddddddkke |
| 3194 | 3209 | | | | |
| 3330 | 3345 | | | | |
| 3432 | 3447 | | | | |
| 3500 | 3515 | | | | |
| 3534 | 3549 | | | | |
| 3620 | 3635 | | | | |
| 3654 | 3669 | | | | |
| 3773 | 3788 | | | | |
| 4182 | 4197 | | | | |
| 4216 | 4231 | | | | |
| 4369 | 4384 | | | | |
| 4403 | 4418 | | | | |
| 3115 | 3130 | ACCCTCGCCTCCGGAA | 655 | 540193 | eekddddddddddkke |
| 3200 | 3215 | | | | |
| 3336 | 3351 | | | | |
| 3438 | 3453 | | | | |
| 3506 | 3521 | | | | |
| 3540 | 3555 | | | | |
| 3626 | 3641 | | | | |
| 3660 | 3675 | | | | |
| 3779 | 3794 | | | | |
| 4188 | 4203 | | | | |
| 4222 | 4237 | | | | |
| 4375 | 4390 | | | | |
| 4409 | 4424 | | | | |
| 3245 | 3260 | ACCCCCGGGACACCCA | 656 | 540194 | eekddddddddddkke |
| 3585 | 3600 | | | | |
| 3688 | 3703 | | | | |
| 3705 | 3720 | | | | |
| 4028 | 4043 | | | | |
| 4539 | 4554 | | | | |
| 4556 | 4571 | | | | |
| 6648 | 6663 | GAGTGTGAGGTGACCC | 547 | 544811 | eekddddddddddkke |
| 6770 | 6785 | | | | |
| 6892 | 6907 | | | | |
| 6958 | 6973 | | | | |
| 7076 | 7091 | | | | |
| 7194 | 7209 | | | | |
| 6646 | 6661 | GTGTGAGGTGACCCGT | 545 | 544812 | eekddddddddddkke |
| 6768 | 6783 | | | | |
| 6890 | 6905 | | | | |
| 6956 | 6971 | | | | |
| 7074 | 7089 | | | | |
| 7192 | 7207 | | | | |
| 7246 | 7261 | | | | |
| 6732 | 6747 | GAGTGTGAGGTGTCCT | 527 | 544813 | eekddddddddddkke |
| 6854 | 6869 | | | | |
| 7212 | 7227 | | | | |
| 7294 | 7309 | | | | |
| 7561 | 7576 | | | | |
| 7633 | 7648 | | | | |
| 8070 | 8085 | | | | |
| 8142 | 8157 | | | | |
| 6706 | 6721 | GGTGACCTGTGAGTGT | 557 | 544814 | eekddddddddddkke |
| 6828 | 6843 | | | | |
| 7016 | 7031 | | | | |
| 7134 | 7149 | | | | |
| 7391 | 7406 | | | | |
| 7457 | 7472 | | | | |
| 7607 | 7622 | | | | |
| 7643 | 7658 | | | | |
| 8116 | 8131 | | | | |
| 8152 | 8167 | | | | |
| 6647 | 6662 | AGTGTGAGGTGACCCG | 546 | 544815 | eekddddddddddkke |
| 6769 | 6784 | | | | |
| 6891 | 6906 | | | | |
| 6957 | 6972 | | | | |
| 7075 | 7090 | | | | |
| 7193 | 7208 | | | | |
| 6682 | 6697 | CCCTGTGTGAGGTGTC | 573 | 544816 | eekddddddddddkke |
| 6804 | 6819 | | | | |
| 6926 | 6941 | | | | |
| 7044 | 7059 | | | | |
| 7162 | 7177 | | | | |
| 7689 | 7704 | | | | |
| 8180 | 8195 | | | | |
| 6681 | 6696 | CCTGTGTGAGGTGTCC | 572 | 544817 | eekddddddddddkke |
| 6803 | 6818 | | | | |
| 6925 | 6940 | | | | |
| 7043 | 7058 | | | | |
| 7161 | 7176 | | | | |
| 7516 | 7531 | | | | |
| 7688 | 7703 | | | | |
| 8179 | 8194 | | | | |
| 6694 | 6709 | GTGTGAAGTGTGCCCT | 566 | 544818 | eekddddddddddkke |
| 6750 | 6765 | | | | |
| 6816 | 6831 | | | | |
| 6872 | 6887 | | | | |
| 6938 | 6953 | | | | |
| 7056 | 7071 | | | | |
| 7174 | 7189 | | | | |
| 7792 | 7807 | | | | |
| 8283 | 8298 | | | | |
| 6713 | 6728 | AGTGTGAGGTGACCTG | 510 | 544819 | eekddddddddddkke |
| 6835 | 6850 | | | | |
| 7398 | 7413 | | | | |
| 7863 | 7878 | | | | |
| 6730 | 6745 | GTGTGAGGTGTCCTGT | 525 | 544820 | eekddddddddddkke |
| 6852 | 6867 | | | | |
| 6974 | 6989 | | | | |
| 7040 | 7055 | | | | |
| 7092 | 7107 | | | | |
| 7158 | 7173 | | | | |
| 7210 | 7225 | | | | |
| 7292 | 7307 | | | | |
| 7351 | 7366 | | | | |
| 7513 | 7528 | | | | |
| 7559 | 7574 | | | | |
| 7631 | 7646 | | | | |
| 7685 | 7700 | | | | |
| 8068 | 8083 | | | | |
| 8140 | 8155 | | | | |
| 8176 | 8191 | | | | |
| 6695 | 6710 | AGTGTGAAGTGTGCCC | 567 | 544821 | eekddddddddddkke |
| 6751 | 6766 | | | | |
| 6817 | 6832 | | | | |
| 6873 | 6888 | | | | |
| 6939 | 6954 | | | | |
| 7005 | 7020 | | | | |
| 7057 | 7072 | | | | |
| 7123 | 7138 | | | | |
| 7175 | 7190 | | | | |
| 7380 | 7395 | | | | |
| 7446 | 7461 | | | | |
| 7793 | 7808 | | | | |
| 7943 | 7958 | | | | |
| 8284 | 8299 | | | | |
| 6760 | 6775 | TGACCCGTGAGTGTGA | 537 | 544826 | eekddddddddddkke |
| 6882 | 6897 | | | | |
| 6948 | 6963 | | | | |
| 7066 | 7081 | | | | |
| 7184 | 7199 | | | | |
| 7238 | 7253 | | | | |
| 7238 | 7253 | | | | |
| 6761 | 6776 | GTGACCCGTGAGTGTG | 538 | 544827 | eekddddddddddkke |
| 6883 | 6898 | | | | |
| 6949 | 6964 | | | | |
| 7067 | 7082 | | | | |
| 7185 | 7200 | | | | |
| 7239 | 7254 | | | | |
| 6762 | 6777 | GGTGACCCGTGAGTGT | 539 | 544828 | eekddddddddddkke |
| 6884 | 6899 | | | | |
| 6950 | 6965 | | | | |
| 7068 | 7083 | | | | |
| 7186 | 7201 | | | | |
| 7240 | 7255 | | | | |
| 6763 | 6778 | AGGTGACCCGTGAGTG | 540 | 544829 | eekddddddddddkke |
| 6885 | 6900 | | | | |
| 6951 | 6966 | | | | |
| 7069 | 7084 | | | | |
| 7187 | 7202 | | | | |
| 7241 | 7256 | | | | |
| 6764 | 6779 | GAGGTGACCCGTGAGT | 541 | 544830 | eekddddddddddkke |
| 6886 | 6901 | | | | |
| 6952 | 6967 | | | | |
| 7070 | 7085 | | | | |
| 7188 | 7203 | | | | |
| 7242 | 7257 | | | | |
| 6643 | 6658 | TGAGGTGACCCGTGAG | 542 | 545471 | eekddddddddddkke |
| 6765 | 6780 | | | | |
| 6887 | 6902 | | | | |
| 6953 | 6968 | | | | |
| 7071 | 7086 | | | | |
| 7189 | 7204 | | | | |
| 7243 | 7258 | | | | |
| 6644 | 6659 | GTGAGGTGACCCGTGA | 543 | 545472 | eekddddddddddkke |
| 6766 | 6781 | | | | |
| 6888 | 6903 | | | | |
| 6954 | 6969 | | | | |
| 7072 | 7087 | | | | |
| 7190 | 7205 | | | | |
| 7244 | 7259 | | | | |
| 6645 | 6660 | TGTGAGGTGACCCGTG | 544 | 545473 | eekddddddddddkke |
| 6767 | 6782 | | | | |
| 6889 | 6904 | | | | |
| 6955 | 6970 | | | | |
| 7073 | 7088 | | | | |
| 7191 | 7206 | | | | |
| 7245 | 7260 | | | | |
| 6707 | 6722 | AGGTGACCTGTGAGTG | 558 | 545474 | eekddddddddddkke |
| 6829 | 6844 | | | | |
| 7017 | 7032 | | | | |
| 7135 | 7150 | | | | |
| 7392 | 7407 | | | | |
| 7458 | 7473 | | | | |
| 7608 | 7623 | | | | |
| 7644 | 7659 | | | | |
| 8117 | 8132 | | | | |
| 8153 | 8168 | | | | |
| 6708 | 6723 | GAGGTGACCTGTGAGT | 559 | 545475 | eekddddddddddkke |
| 6830 | 6845 | | | | |
| 7018 | 7033 | | | | |
| 7136 | 7151 | | | | |
| 7393 | 7408 | | | | |
| 7459 | 7474 | | | | |
| 6709 | 6724 | TGAGGTGACCTGTGAG | 560 | 545476 | eekddddddddddkke |
| 6831 | 6846 | | | | |
| 7019 | 7034 | | | | |
| 7137 | 7152 | | | | |
| 7394 | 7409 | | | | |
| 7460 | 7475 | | | | |
| 7859 | 7874 | | | | |
| 6710 | 6725 | GTGAGGTGACCTGTGA | 561 | 545477 | eekddddddddddkke |
| 6832 | 6847 | | | | |
| 7020 | 7035 | | | | |
| 7138 | 7153 | | | | |
| 7395 | 7410 | | | | |
| 7461 | 7476 | | | | |
| 7860 | 7875 | | | | |
| 6711 | 6726 | TGTGAGGTGACCTGTG | 562 | 545478 | eekddddddddddkke |
| 6833 | 6848 | | | | |
| 7021 | 7036 | | | | |
| 7139 | 7154 | | | | |
| 7396 | 7411 | | | | |
| 7462 | 7477 | | | | |
| 7861 | 7876 | | | | |
| 6705 | 6720 | GTGACCTGTGAGTGTG | 556 | 545479 | eekddddddddddkke |
| 6827 | 6842 | | | | |
| 7015 | 7030 | | | | |
| 7133 | 7148 | | | | |
| 7390 | 7405 | | | | |
| 7456 | 7471 | | | | |
| 7606 | 7621 | | | | |
| 7642 | 7657 | | | | |
| 7803 | 7818 | | | | |
| 8115 | 8130 | | | | |
| 8151 | 8166 | | | | |
| 8294 | 8309 | | | | |
| 6718 | 6733 | CTGTGAGTGTGAGGTG | 514 | 537727 | kkkddddddddddeee |
| 6736 | 6751 | | | | |
| 6840 | 6855 | | | | |
| 6858 | 6873 | | | | |
| 6962 | 6977 | | | | |
| 7080 | 7095 | | | | |
| 7198 | 7213 | | | | |
| 7339 | 7354 | | | | |
| 7403 | 7418 | | | | |
| 7637 | 7652 | | | | |
| 7868 | 7883 | | | | |
| 8146 | 8161 | | | | |
| 8393 | 8408 | | | | |

### Treatment

Male BALB/c mice were injected subcutaneously with a single dose of 200 mg/kg of ISIS 422142, ISIS 457851, ISIS 473294, ISIS 473295, ISIS 473327, ISIS 484714, ISIS 515334, ISIS 515338, ISIS 515354, ISIS 515366, ISIS 515380, ISIS 515381, ISIS 515382, ISIS 515384, ISIS 515386, ISIS 515387, ISIS 515388, ISIS 515406, ISIS 515407, ISIS 515408, ISIS 515422, ISIS 515423, ISIS 515424, ISIS 515532, ISIS 515534, ISIS 515538, ISIS 515539, ISIS 515558, ISIS 515656, ISIS 515575, ISIS 515926, ISIS 515944, ISIS 515945, ISIS 515948, ISIS 515949, ISIS 515951, ISIS 515952, ISSI 516003, ISIS 516055, ISIS 516057, ISIS 516060, ISIS 516062, ISIS 529126, ISIS 529146, ISIS 529166, ISIS 529170, ISIS 529172, ISIS 529173, ISIS 529174, ISIS 529175, ISSI 529176, ISIS 529182, ISIS 529183, ISIS 529186, ISIS 529282, ISIS 529304, ISIS 529306, ISIS 529360, ISIS 529450, ISIS 529459, ISIS 529460, ISIS 529461, ISIS 529547, ISIS 529550, ISIS 529551, ISIS 529553, ISIS 529557, ISIS 529562, ISIS 529563, ISIS 529564, ISIS 529565, ISIS 529575, ISIS 529582, ISIS 529589, ISIS 529607, ISIS 529614, ISIS 529632, ISIS 529650, ISIS 529651, ISIS 529657, ISIS 529663, ISIS 529725, ISIS 529745, ISIS 529765, ISIS 529785, ISIS 529804, ISIS 529818, ISIS 529823, ISIS 529854, ISIS 534528, ISIS 534534, ISIS 534594, ISIS 534660, ISIS 534663, ISIS 534664, ISIS 534676, ISIS 534677, ISIS 537679, ISIS 537683, ISIS 534693, ISIS 534701, ISIS 534716, ISIS 534730, ISIS 534765, ISIS 534795, ISIS 534796, ISIS 534797, ISIS 534798, ISIS 534799, ISIS 534800, ISIS 534802, ISIS 534806, ISSI 534830, ISIS 534838, ISIS 534888, ISIS 534890, ISIS 534898, ISIS 534911, ISIS 534920, ISIS 534926, ISIS 534937, ISIS 534950, ISSI 534956, ISIS 534980, ISIS 534986, ISIS 535010, ISIS 535043, ISIS 535049, ISIS 535076, ISIS 535082, ISSI 535142, ISIS 537024, ISIS 537030, ISIS 537041, ISIS 537062, ISIS 537064, ISIS 537066, ISIS 537721, ISIS 537727, ISIS 537738, ISIS 537759, ISIS 537761, ISIS 537763, ISIS 537792, ISIS 537800, ISIS 537806, ISIS 537811, ISIS 537814, ISIS 537839, ISIS 537850, ISSI 537858, ISIS 537864, ISIS 537869, ISIS 537872, ISIS 537897, ISIS 538160, ISIS 538196, ISIS 538205, ISIS 538228, ISIS 538242, ISIS 538361, ISIS 538380, ISIS 540118, ISIS 540138, ISIS 540139, ISIS 540148, ISIS 540153, ISIS 540155, ISIS 540162, ISIS 540164, ISIS 540168, ISIS 540172, ISIS 540175, ISIS 540176, ISIS 540178, ISIS 540179, ISIS 540181, ISIS 540182, ISIS 540183, ISIS 540184, ISIS 540186, ISIS 540187, ISIS 540188, ISIS 540191, ISIS 540193, ISIS 540194, ISIS 544811, ISIS 544812, ISIS 544813, ISIS 544814, ISIS 544815, ISIS 544816, ISIS 544817, ISIS 544818, ISIS 544819, ISIS 544820, ISIS 544821, ISIS 544826, ISIS 544827, ISIS 544828, ISIS 544829, ISIS 544830, ISIS 545471, ISIS 545472, ISIS 545473, ISIS 545474, ISIS 545475, ISIS 545476, ISIS 545477, ISIS 545478, or ISIS 545479. One set of male BALB/c mice was injected with a single dose of PBS. Mice were euthanized 96 hours later, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, N.Y.).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 529166, ISIS 529170, ISIS 529175, ISIS 529176, ISIS 529186, ISIS 529282, ISIS 529360, ISIS 529450, ISIS 529459, ISIS 529460, ISIS 529547, ISIS 529549, ISIS 529551, ISIS 529553, ISIS 529557, ISIS 529562, ISIS 529575, ISIS 529582, ISIS 529607, ISIS 529589, ISIS 529632, ISIS 529657, ISIS 529725, ISIS 529745, ISIS 529785, ISIS 529799, ISIS 529804, ISIS 529818, ISIS 529823, ISIS 534950, ISIS 534980, ISIS 535010, ISIS 537030, ISIS 537041, ISIS 537062, ISIS 537064, ISIS 537066, ISIS 537759, ISIS 537792, ISIS 537800, ISIS 537839, ISIS 538228, ISIS 473294, ISIS 473295, ISIS 484714, ISIS 515338, ISIS 515366, ISIS 515380, ISIS 515381, ISIS 515387, ISIS 515408, ISIS 515423, ISIS 515424, ISIS 515532, ISIS 515534, ISIS 515538, ISIS 515539, ISIS 515558, ISIS 515575, ISIS 515926, ISIS 515944, ISIS 515945, ISIS 515951, ISIS 515952, ISIS 529126, ISIS 529765, ISIS 534528, ISIS 534534, ISIS 534594, ISIS 534663, ISIS 534676, ISIS 534677, ISIS 534679, ISIS 534683, ISIS 534693, ISIS 534701, ISIS 534716, ISIS 534730, ISIS 534806, ISIS 534830, ISIS 534838, ISIS 534890, ISIS 534898, ISIS 534911, ISIS 534937, ISIS 534956, ISIS 534986, ISIS 535043, ISIS 535049, ISIS 535076, ISIS 535082, ISIS 535142, ISIS 538160, ISIS 538242, ISIS 538361, ISIS 538380, ISIS 534795, ISIS 534796, ISIS 534797, ISIS 540162, ISIS 540164, ISIS 540168, ISIS 540172, ISIS 540175, ISIS 540176, ISIS 540178, ISIS 540179, ISIS 540181, ISIS 540182, ISIS 540183, ISIS 540184, ISIS 540186, ISIS 540187, ISIS 540188, ISIS 540191, ISIS 540193, ISIS 540194, ISIS 544813, ISIS 544814, ISIS 544816, ISIS 544826, ISIS 544827, ISIS 544828, ISIS 544829, ISIS 545473, and ISIS 545474 were considered very tolerable in terms of liver function. Based on these criteria, ISIS 529173, ISIS 529854, ISIS 529614, ISIS 515386, ISIS 515388, ISIS 515949, ISIS 544817, and ISIS 545479 were considered tolerable in terms of liver function.

### Example 24

### Tolerability of Modified Antisense Oligonucleotides Targeting Human Factor VII in Sprague-Dawley Rats

Sprague-Dawley rats are a multipurpose model used for safety and efficacy evaluations. The rats were treated with ISIS antisense oligonucleotides from the studies described in the Examples above and evaluated for changes in the levels of various plasma chemistry markers.

### Treatment

Six to eight week old male Sprague-Dawley rats were maintained on a 12-hour light/dark cycle and fed ad libitum with Teklad normal rat chow. Groups of four Sprague-Dawley rats each were injected subcutaneously twice a week for 6 weeks with 25 mg/kg of ISIS 473286, ISIS 473547, ISIS 473567, ISIS 473589, ISIS 473630, ISIS 484559, ISIS 515636, ISIS 515640, ISIS 515641, ISIS 515655, ISIS 515657, ISIS 516046, ISIS 516048, ISIS 516051, ISIS 516052, or ISIS 516062. A group of four Sprague-Dawley rats was injected subcutaneously twice a week for 6 weeks with PBS. Forty-eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, N.Y.). Plasma levels of ALT (alanine transaminase) and AST (aspartate transaminase) were measured. Plasma levels of Bilirubin and BUN were also measured using the same clinical chemistry analyzer.

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 473286, ISIS 473547, ISSI 473589, ISSI 473630, ISIS 484559, ISIS 515636, ISIS 515640, ISIS 515655, ISIS 516046, and ISIS 516051 were considered very tolerable in terms of liver function. Based on these criteria, ISIS 473567, ISIS 515641, ISIS 515657, ISIS 516048, and ISIS 516051 were considered tolerable in terms of liver function.

### Example 25

### Tolerability of Modified Antisense Oligonucleotides Comprising MOE Modifications Targeting Human Factor VII in Sprague-Dawley Rats

Sprague-Dawley rats were treated with ISIS antisense oligonucleotides from the studies described in the Examples above and evaluated for changes in the levels of various plasma chemistry markers.

### Treatment

Six-eight week old male Sprague-Dawley rats were maintained on a 12-hour light/dark cycle and fed ad libitum with Purina normal rat chow. Groups of four Sprague-Dawley rats each were injected subcutaneously twice a week for 6 weeks with 50 mg/kg of ISIS 407936, ISIS 416507, ISIS 416508, ISIS 490208, ISIS 490279, ISIS 490323, ISIS 490368, ISIS 490396, ISIS 490803, ISIS 491122, ISIS 513419, ISIS 513446, ISIS 513454, ISIS 513455, ISIS 513456, ISIS 513504, ISIS 513507, or ISIS 513508. A group of four Sprague-Dawley rats was injected subcutaneously twice a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, N.Y.). Plasma levels of Bilirubin and BUN were also measured using the same clinical chemistry analyzer.

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 416507, ISIS 490208, ISIS 490368, ISIS 490396, ISIS 490803, ISIS 491122, ISIS 513446, ISIS 513454, ISIS 513455, ISIS 513456, ISIS 513504, and ISIS 513508 were considered very tolerable in terms of liver function. Based on these criteria, ISIS 407936, ISIS 416508, ISIS 490279, and ISIS 513507 were considered tolerable in terms of liver function.

### Example 26

### Tolerability of Modified Oligonucleotides Comprising MOE Modifications Targeting Human Factor VII in CD-1 Mice

CD-1 mice are a multipurpose mice model, frequently utilized for safety and efficacy testing. The mice were treated with ISIS antisense oligonucleotides selected from studies described above and evaluated for changes in the levels of various plasma chemistry markers.

### Treatment

Groups of 3 male CD-1 mice each were injected subcutaneously twice a week for 6 weeks with 50 mg/kg of ISIS 473244, ISIS 473295, ISIS 484714, ISIS 515386, ISIS 515424, ISIS 515534, ISIS 515558, ISIS 515926, ISIS 515949, ISIS 515951, ISIS 515952, ISIS 529126, ISIS 529166, ISIS 529173, ISIS 529186, ISIS 529360, ISIS 529461, ISIS 529553, ISIS 529564, ISIS 529582, ISIS 529614, ISIS 529725, ISIS 529745, ISIS 529765, ISIS 529785, ISIS 529799, ISIS 529818, ISIS 529823, ISIS 534528, ISIS 534594, or ISIS 534664. One group of male CD-1 mice was injected subcutaneously twice a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, N.Y.).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 473295, ISIS 473714, ISIS 515558, ISIS 515926, 515951, ISIS 515952, ISIS 529126, ISIS 529166, 529564, ISIS 529582, ISIS 529614, ISIS 529725, ISIS 529765, ISIS 529799, ISIS 529823, and ISIS 534594 were considered very tolerable in terms of liver function. Based on these criteria, ISIS 515424, ISIS 515534, ISIS 515926, ISIS 529785, and ISIS 534664 were considered tolerable in terms of liver function.

### Example 27

### Tolerability of Modified Oligonucleotides Comprising MOE Modifications Targeting Human Factor VII in CD-1 Mice

CD-1 mice were treated with ISIS antisense oligonucleotides selected from studies described above and evaluated for changes in the levels of various plasma chemistry markers.

### Treatment

Groups of 3 male CD-1 mice each were injected subcutaneously twice a week for 6 weeks with 100 mg/kg of ISIS 490208, ISIS 490279, ISIS 490323, ISIS 490368, ISIS 490396, ISIS 490803, ISIS 491122, ISIS 513419, ISIS 513446, ISIS 513454, ISIS 513455, ISIS 513456, ISIS 513504, ISIS 513507, or ISIS 513508. Groups of 3 male CD-1 mice each were injected subcutaneously twice a week for 6 weeks with 100 mg/kg of ISIS 407936, ISIS 416507, or ISIS 416508, which are gapmers described in a previous publication. One group of male CD-1 mice was injected subcutaneously twice a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, and BUN were measured using an automated clinical chemistry analyzer (Hitachi Olympus AU400e, Melville, N.Y.).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 407936, ISIS 416507, ISIS 490279, ISIS 490368, ISIS 490396, ISIS 490803, ISIS 491122, ISIS 513446, ISIS 513454, ISIS 513456, and ISIS 513504 were considered very tolerable in terms of liver function. Based on these criteria, ISIS 490208, ISIS 513455, ISIS 513507, and ISIS 513508 were considered tolerable in terms of liver function.

### Example 28

### Efficacy of Modified Oligonucleotides Comprising MOE and cEt Modifications Targeting Human Factor VII in Transgenic Mice

Transgenic mice were treated with ISIS antisense oligonucleotides selected from studies described above and evaluated for efficacy.

### Treatment

Groups of 2-3 male and female transgenic mice were injected subcutaneously twice a week for 3 weeks with 2.5 mg/kg of ISIS 473244, ISIS 473295, ISIS 484714, ISIS 515926, ISIS 515951, ISIS 515952, ISIS 516062, ISIS 529126, ISIS 529553, ISIS 529745, ISIS 529799, ISIS 534664, ISIS 534826, ISIS 540168, ISIS 540175, ISIS 544826, ISIS 544827, ISIS 544828, or ISIS 544829. One group of mice was injected subcutaneously twice a week for 3 weeks with PBS. Mice were euthanized 48 hours after the last dose, and organs and plasma were harvested for further analysis.

### Protein Analysis

Plasma protein levels of Factor VII were estimated using a Zymutest FVII ELISA kit (Hyphen Bio-Med cat#ARK036A). Results are presented as percent inhibition of Factor VII, relative to control. As shown in Table 25, several antisense oligonucleotides achieved significant reduction of human Factor VII over the PBS control. ‘n.d.’ indicates that the value for that particular oligonucleotide was not measured.

TABLE 25
Percent inhibition of Factor VII plasma protein levels
in transgenic mice

| ISIS No | % inhibition |
|---|---|
| 473244 | 2 |
| 473295 | 13 |
| 484714 | 19 |
| 515926 | 11 |
| 515951 | 13 |
| 515952 | 0 |
| 516062 | 62 |
| 529126 | 0 |
| 529553 | 0 |
| 529745 | 22 |
| 529799 | 26 |
| 534664 | 32 |
| 534826 | n.d. |
| 540168 | 94 |
| 540175 | 98 |
| 544813 | 0 |
| 544826 | 23 |
| 544827 | 60 |
| 544828 | 33 |
| 544829 | 53 |

### Example 29

### Efficacy of Modified Oligonucleotides Comprising MOE and cEt Modifications Targeting Human Factor VII in Transgenic Mice

Transgenic mice were treated with ISIS antisense oligonucleotides selected from studies described above and evaluated for efficacy.

### Treatment

Groups of 2-3 male and female transgenic mice were injected subcutaneously twice a week for 3 weeks with 0.5 mg/kg of ISIS 407936, ISIS 490197, ISIS 490275, ISIS 490278, ISIS 490279, ISIS 490323, ISIS 490368, ISIS 490396, ISIS 490803, ISIS 491122, ISIS 513446, ISIS 513447, ISIS 513504, ISIS 516062, ISIS 529166, ISIS 529173, ISIS 529360, ISIS 529725, ISIS 534557, ISIS 534594, ISIS 534664, ISIS 534688, ISIS 534689, ISIS 534915, ISIS 534916, ISIS 534917, or ISIS 534980. One group of mice was injected subcutaneously twice a week for 3 weeks with PBS. Mice were euthanized 48 hours after the last dose, and organs and plasma were harvested for further analysis.

### Protein Analysis

Plasma protein levels of Factor VII were estimated using a Zymutest FVII ELISA kit (Hyphen Bio-Med cat#ARK036A). Results are presented as percent inhibition of Factor VII, relative to control. As shown in Table 26, several antisense oligonucleotides achieved significant reduction of human Factor VII over the PBS control.

TABLE 26
Percent inhibition of Factor VII plasm protein levels
in transgenic mice

| ISIS No | % inhibition |
|---|---|
| 407936 | 28 |
| 490197 | 50 |
| 490275 | 21 |
| 490278 | 20 |
| 490279 | 59 |
| 490323 | 54 |
| 490368 | 22 |
| 490396 | 31 |
| 490803 | 30 |
| 491122 | 51 |
| 513446 | 29 |
| 513447 | 44 |
| 513504 | 45 |
| 516062 | 75 |
| 529166 | 37 |
| 529173 | 64 |
| 529360 | 43 |
| 529725 | 53 |
| 534557 | 76 |
| 534594 | 40 |
| 534664 | 14 |
| 534687 | 12 |
| 534688 | 48 |
| 534689 | 25 |
| 534915 | 40 |
| 534916 | 45 |
| 534917 | 66 |
| 534980 | 62 |

### Example 30

### Tolerability of Antisense Oligonucleotides Targeting Human Factor VII in Sprague-Dawley Rats

Sprague-Dawley rats were treated with ISIS antisense oligonucleotides from the studies described in the Examples above and evaluated for changes in the levels of various plasma chemistry markers.

### Treatment

Six to eight week old male Sprague-Dawley rats were maintained on a 12-hour light/dark cycle and fed ad libitum with Teklad normal rat chow. Groups of four Sprague-Dawley rats each were injected subcutaneously twice a week for 4 weeks with ISIS 515380, ISIS 515381, ISIS 515387, ISIS 529175, ISIS 529176, ISIS 529575, ISIS 529804, or ISIS 537064. Doses 1, 5, 6, 7, and 8 were 25 mg/kg; dose 2 was 75 mg/kg; doses 3 and 4 were 50 mg/kg. One group of four Sprague-Dawley rats was injected subcutaneously twice a week for 4 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, N.Y.). Plasma levels of ALT (alanine transaminase) and AST (aspartate transaminase) were measured. Plasma levels of Bilirubin and BUN were also measured using the same clinical chemistry analyzer.

ISIS oligonucleotides that did not cause any increase in the levels of transaminases, or which caused increase in the levels within three times the upper limit of normal levels of transaminases were deemed very tolerable. ISIS oligonucleotides that caused increase in the levels of transaminases between three times and seven times the upper limit of normal levels were deemed tolerable. Based on these criteria, ISIS 515380, ISIS 515387, ISIS 529175, ISIS 529176, ISIS 529804, and ISIS 537064 were considered very tolerable in terms of liver function. Based on these criteria, ISIS 515381 was considered tolerable in terms of liver function.

### Example 31

### Efficacy of Modified Antisense Oligonucleotides Targeting Human Factor VII in Transgenic Mice

Transgenic mice were treated with ISIS antisense oligonucleotides selected from studies described above and evaluated for efficacy.

### Treatment

Two groups of 3 male and female transgenic mice were injected subcutaneously twice a week for 2 weeks with 0.25 mg/kg or 0.75 mg/kg of ISIS 407935 or ISIS 513455. Another group of mice was subcutaneously twice a week for 2 weeks with 0. mg/kg or 1.0 mg/kg of ISIS 473286. Another 16 groups of mice were subcutaneously twice a week for 2 weeks with 0.05 mg/kg or 0.15 mg/kg of ISIS 473589, ISIS 515380, ISIS 515423, ISIS 529804, ISIS 534676, ISIS 534796, ISIS 540162, ISIS 540164, ISIS 540175, ISIS 540179, ISIS 540181, ISIS 540182, ISIS 540186, ISIS 540191, ISIS 540193, ISIS 544827, or ISIS 545474. Another 3 groups of mice were injected subcutaneously twice a week for 2 weeks with 0.15 mg/kg of ISIS 516062, ISIS 534528 or ISIS 534693. One group of mice was injected subcutaneously twice a week for 2 weeks with PBS. Mice were euthanized 48 hours after the last dose, and organs and plasma were harvested for further analysis.

### Protein Analysis

Plasma protein levels of Factor VII were estimated using a Zymutest FVII ELISA kit (Hyphen Bio-Med cat#ARK036A). Results are presented as percent inhibition of Factor VII, relative to control. As shown in Table 27, several antisense oligonucleotides achieved significant reduction of human Factor VII over the PBS control.

TABLE 27
Percent inhibition of Factor VII plasma protein levels
in transgenic mice

| | Dose | % |
|---|---|---|
| ISIS No | (mg/kg/wk) | inhibition |
| 407935 | 1.5 | 65 |
| | 0.5 | 31 |
| 513455 | 1.5 | 64 |
| | 0.5 | 52 |
| 473286 | 2.0 | 67 |
| | 0.6 | 11 |
| 473589 | 0.3 | 42 |
| | 0.1 | 12 |
| 515380 | 0.3 | 64 |
| | 0.1 | 32 |
| 515423 | 0.3 | 72 |
| | 0.1 | 37 |
| 529804 | 0.3 | 36 |
| | 0.1 | 24 |
| 534676 | 0.3 | 31 |
| | 0.1 | 18 |
| 534796 | 0.3 | 54 |
| | 0.1 | 43 |
| 540162 | 0.3 | 84 |
| | 0.1 | 42 |
| 540164 | 0.3 | 25 |
| | 0.1 | 17 |
| 540175 | 0.3 | 90 |
| | 0.1 | 55 |
| 540179 | 0.3 | 29 |
| | 0.1 | 24 |
| 540181 | 0.3 | 53 |
| | 0.1 | 0 |
| 540182 | 0.3 | 78 |
| | 0.1 | 21 |
| 540186 | 0.3 | 72 |
| | 0.1 | 46 |
| 540191 | 0.3 | 62 |
| | 0.1 | 35 |
| 540193 | 0.3 | 74 |
| | 0.1 | 46 |
| 544827 | 0.3 | 28 |
| | 0.1 | 19 |
| 545474 | 0.3 | 59 |
| | 0.1 | 0 |
| 516062 | 0.3 | 33 |
| 534528 | 0.3 | 41 |
| 534693 | 0.3 | 34 |

### Example 32

### Tolerability of Antisense Oligonucleotides Targeting Human Factor VII in Sprague-Dawley Rats

Sprague-Dawley rats were treated with ISIS antisense oligonucleotides from the studies described in the Examples above and evaluated for changes in the levels of various plasma chemistry markers.

### Treatment

Five-six week old male Sprague-Dawley rats were maintained on a 12-hour light/dark cycle and fed ad libitum with Teklad normal rat chow. Groups of four Sprague-Dawley rats each were injected subcutaneously twice a week for 4 weeks with 50 mg/kg of ISIS 515423, ISIS 515424, ISIS 515640, ISIS 534676, ISIS 534796, ISIS 534797, ISIS 540162, ISIS 540164, ISIS 540172, ISIS 540175, ISIS 540179, ISIS 540181, ISIS 540182, ISIS 540183, ISIS 540186, ISIS 540191, or ISIS 545474. A group of four Sprague-Dawley rats was injected subcutaneously twice a week for 4 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, N.Y.). Plasma levels of ALT (alanine transaminase) and AST (aspartate transaminase) were measured. Plasma levels of Bilirubin and BUN were also measured using the same clinical chemistry analyzer.

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 540164, ISIS 540172, and ISIS 540175 were considered very tolerable in terms of liver function. Based on these criteria, ISIS 534676, ISIS 534796, ISIS 534797, ISIS 540162, and ISIS 540179 were considered tolerable in terms of liver function.

### Example 33

### Dose-Dependent Antisense Inhibition of Human Factor VII in Hep3B Cells

Antisense oligonucleotides selected from the studies described above were tested at various doses in Hep3B cells. Cells were plated at a density of 20,000 cells per well and transfected using electroporation with 0.05 μM, 0.15 μM, 0.44 μM, 1.33 μM, and 4.00 μM concentrations of antisense oligonucleotide, as specified in Table 28. After a treatment period of approximately 16 hours, RNA was isolated from the cells and Factor VII mRNA levels were measured by quantitative real-time PCR. Human Factor VII primer probe set RTS2927 was used to measure mRNA levels. Factor VII mRNA levels were adjusted according to total RNA content, as measured by RIBOGREEN®. Results are presented as percent inhibition of Factor VII expression, relative to untreated control cells.

The half maximal inhibitory concentration (IC₅₀) of each oligonucleotide is also presented in Table 28. As illustrated in Table 28, Factor VII mRNA levels were significantly reduced in a dose-dependent manner in several of the antisense oligonucleotide treated cells.

TABLE 28
Dose-dependent antisense inhibition (%) of human
Factor VII in Hep3B cells using electroporation

| | 0.05 | | | | | IC₅₀ |
|---|---|---|---|---|---|---|
| ISIS No | μM | 0.15 μM | 0.44 μM | 1.33 μM | 4.00 μM | (μM) |
| 473286 | 0 | 1 | 13 | 12 | 15 | >4.0 |
| 457851 | 23 | 32 | 57 | 80 | 93 | 0.3 |
| 473286 | 3 | 20 | 43 | 71 | 88 | 0.5 |
| 473286 | 15 | 26 | 24 | 28 | 36 | >4.0 |
| 473286 | 6 | 3 | 10 | 26 | 29 | >4.0 |
| 473327 | 14 | 28 | 35 | 67 | 90 | 0.5 |
| 473589 | 29 | 53 | 76 | 89 | 95 | 0.1 |
| 515380 | 44 | 72 | 85 | 93 | 95 | <0.05 |
| 515423 | 43 | 64 | 87 | 95 | 98 | <0.05 |
| 515424 | 38 | 55 | 85 | 92 | 97 | 0.1 |
| 515636 | 21 | 33 | 74 | 82 | 93 | 0.2 |
| 516046 | 29 | 23 | 29 | 48 | 78 | 0.9 |
| 516048 | 35 | 24 | 41 | 67 | 87 | 0.4 |
| 516052 | 18 | 6 | 48 | 63 | 80 | 0.6 |
| 516062 | 24 | 14 | 21 | 47 | 68 | 1.6 |
| 529166 | 16 | 47 | 75 | 87 | 94 | 0.2 |
| 529173 | 14 | 49 | 77 | 91 | 96 | 0.2 |
| 529175 | 30 | 69 | 88 | 93 | 96 | 0.1 |
| 529176 | 34 | 63 | 85 | 93 | 96 | 0.1 |
| 529360 | 35 | 53 | 74 | 91 | 93 | 0.1 |
| 529725 | 53 | 69 | 85 | 92 | 95 | <0.05 |
| 529804 | 37 | 41 | 71 | 90 | 94 | 0.1 |
| 534528 | 50 | 68 | 78 | 93 | 97 | <0.05 |
| 534557 | 48 | 78 | 90 | 94 | 95 | <0.05 |
| 534594 | 39 | 47 | 76 | 87 | 94 | 0.1 |
| 534676 | 29 | 20 | 40 | 64 | 87 | 0.5 |
| 534687 | 41 | 37 | 56 | 80 | 93 | 0.2 |
| 534688 | 16 | 56 | 88 | 94 | 96 | 0.1 |
| 534689 | 21 | 59 | 82 | 94 | 95 | 0.1 |
| 534693 | 18 | 58 | 81 | 93 | 95 | 0.1 |
| 534795 | 19 | 43 | 68 | 90 | 94 | 0.2 |
| 534796 | 25 | 59 | 80 | 93 | 96 | 0.1 |
| 534890 | 31 | 55 | 77 | 90 | 96 | 0.1 |
| 534898 | 22 | 61 | 80 | 94 | 97 | 0.1 |
| 534915 | 19 | 26 | 51 | 77 | 94 | 0.3 |
| 534916 | 20 | 36 | 66 | 86 | 93 | 0.2 |
| 534917 | 34 | 53 | 82 | 89 | 94 | 0.1 |
| 540162 | 40 | 64 | 84 | 90 | 92 | <0.05 |
| 540164 | 34 | 60 | 83 | 91 | 92 | 0.1 |
| 540168 | 51 | 79 | 90 | 92 | 94 | <0.05 |
| 540172 | 40 | 66 | 80 | 88 | 92 | <0.05 |
| 540175 | 30 | 61 | 80 | 88 | 91 | 0.1 |
| 540176 | 7 | 17 | 50 | 75 | 85 | 0.5 |
| 540179 | 11 | 22 | 25 | 16 | 19 | >4.0 |
| 540181 | 19 | 46 | 72 | 86 | 91 | 0.2 |
| 540182 | 16 | 66 | 83 | 86 | 92 | 0.1 |
| 540183 | 39 | 74 | 87 | 92 | 93 | <0.05 |
| 540186 | 31 | 69 | 85 | 91 | 94 | 0.1 |
| 540191 | 38 | 54 | 80 | 88 | 91 | 0.1 |
| 540193 | 57 | 67 | 84 | 94 | 97 | <0.05 |
| 540194 | 30 | 45 | 62 | 77 | 91 | 0.2 |
| 544827 | 37 | 42 | 67 | 82 | 96 | 0.1 |
| 544829 | 26 | 41 | 42 | 71 | 93 | 0.3 |
| 545473 | 28 | 27 | 49 | 80 | 97 | 0.3 |
| 545474 | 23 | 27 | 55 | 84 | 96 | 0.3 |

### Example 34

### Tolerability of Antisense Oligonucleotides Targeting Human Factor VII in CD-1 Mice

CD-1 mice were treated with ISIS antisense oligonucleotides selected from studies described above and evaluated for changes in the levels of various plasma chemistry markers.

### Treatment

Two groups of 4 male 6-8 week old CD-1 mice each were injected subcutaneously twice a week for 6 weeks with 50 mg/kg of ISIS 407935 or ISIS 490279. Another seven groups of 4 male 6-8 week old CD-1 mice each were injected subcutaneously twice a week for 6 weeks with 25 mg/kg of ISIS 473589, ISIS 529804, ISIS 534796, ISIS 540162, ISIS 540175, ISIS 540182, or ISIS 540191. One group of male CD-1 mice was injected subcutaneously twice a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, N.Y.). The results are presented in Table 29. ‘MOE’ indicates that the antisense oligonucleotide is a MOE gapmer. ‘DMC’ indicates that the antisense oligonucleotide comprises deoxy, cEt, and MOE modifications. Treatment with the newly designed antisense oligonucleotides are more tolerable compared to treatment with ISIS 407935 (disclosed in an earlier publication), which caused elevation of ALT levels greater than seven times the upper limit of normal (ULN).

TABLE 29
Effect of antisense oligonucleotide
treatment on liver function in CD-1 mice

| | | | | | BUN | |
|---|---|---|---|---|---|---|
| | | Dose | ALT | AST | (mg/ | Bilirubin |
| | Chemistry | (mg/kg/wk) | (IU/L) | (IU/L) | dL) | (mg/dL) |
| PBS | — | — | 37 | 47 | 28 | 0.2 |
| 407935 | MOE | 100 | 373 | 217 | 24 | 0.2 |
| 490279 | MOE | 100 | 96 | 82 | 24 | 0.2 |
| 473589 | DMC | 50 | 93 | 116 | 22 | 0.2 |
| 529804 | DMC | 50 | 54 | 74 | 27 | 0.2 |
| 534796 | DMC | 50 | 60 | 63 | 27 | 0.2 |
| 540162 | DMC | 50 | 43 | 55 | 29 | 0.2 |
| 540175 | DMC | 50 | 113 | 78 | 24 | 0.3 |
| 540182 | DMC | 50 | 147 | 95 | 26 | 0.1 |
| 540191 | DMC | 50 | 79 | 88 | 28 | 0.2 |

### Body and Organ Weights

Body weights, as well as liver, heart, lungs, spleen and kidney weights were measured at the end of the study, and are presented in Table 30. MOE′ indicates that the antisense oligonucleotide is a MOE gapmer. ‘DMC’ indicates that the antisense oligonucleotide comprises deoxy, cEt and MOE modifications. Several of the ISIS oligonucleotides did not cause any changes in organ weights outside the expected range and were therefore deemed tolerable in terms of organ weights.

TABLE 30
Body and organ weights (grams) of CD-1 mice

| | | Dose | Body | | | |
|---|---|---|---|---|---|---|
| | Chemistry | (mg/kg/wk) | weight | Liver | Spleen | Kidney |
| PBS | — | — | 42 | 2.2 | 0.12 | 0.64 |
| 407935 | MOE | 100 | 40 | 2.6 | 0.20 | 0.62 |
| 490279 | MOE | 100 | 42 | 2.8 | 0.17 | 0.61 |
| 473589 | DMC | 50 | 41 | 2.5 | 0.16 | 0.67 |
| 529804 | DMC | 50 | 40 | 2.3 | 0.14 | 0.62 |
| 534796 | DMC | 50 | 37 | 2.6 | 0.15 | 0.51 |
| 540162 | DMC | 50 | 42 | 2.4 | 0.15 | 0.60 |
| 540175 | DMC | 50 | 39 | 2.2 | 0.11 | 0.62 |
| 540182 | DMC | 50 | 41 | 2.6 | 0.16 | 0.61 |
| 540191 | DMC | 50 | 40 | 2.4 | 0.13 | 0.60 |

### Example 35

### Tolerability of Antisense Oligonucleotides Targeting Human Factor VII in Sprague-Dawley Rats

Sprague-Dawley rats were treated with ISIS antisense oligonucleotides selected from studies described above and evaluated for changes in the levels of various plasma chemistry markers.

### Treatment

Two groups of 4 male 7-8 week old Sprague-Dawley rats each were injected subcutaneously twice a week for 6 weeks with 50 mg/kg of ISIS 407935 or ISIS 490279. Another seven groups of 4 male 6-8 week old Sprague-Dawley rats each were injected subcutaneously twice a week for 6 weeks with 25 mg/kg of ISIS 473589, ISIS 529804, ISIS 534796, ISIS 540162, ISIS 540175, ISIS 540182, or ISIS 540191. One group of male Sprague-Dawley rats was injected subcutaneously twice a week for 6 weeks with PBS. The rats were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, N.Y.). The results are presented in Table 31. MOE′ indicates that the antisense oligonucleotide is a MOE gapmer. ‘DMC’ indicates that the antisense oligonucleotide comprises deoxy, cEt and MOE modifications. Treatment with the all antisense oligonucleotides was tolerable in terms of plasma chemistry markers in this model.

TABLE 31
Effect of antisense oligonucleotide treatment
on liver function in Sprague-Dawley rats

| | | | | | BUN | |
|---|---|---|---|---|---|---|
| | | Dose | ALT | AST | (mg/ | Bilirubin |
| | Chemistry | (mg/kg/wk) | (IU/L) | (IU/L) | dL) | (mg/dL) |
| PBS | — | — | 71 | 83 | 19 | 0.2 |
| 407935 | MOE | 100 | 74 | 96 | 22 | 0.2 |
| 490279 | MOE | 100 | 96 | 181 | 22 | 0.4 |
| 473589 | DMC | 50 | 57 | 73 | 21 | 0.2 |
| 529804 | DMC | 50 | 54 | 78 | 21 | 0.2 |
| 534796 | DMC | 50 | 68 | 98 | 22 | 0.2 |
| 540162 | DMC | 50 | 96 | 82 | 21 | 0.1 |
| 540175 | DMC | 50 | 55 | 73 | 18 | 0.2 |
| 540182 | DMC | 50 | 45 | 87 | 21 | 0.2 |
| 540191 | DMC | 50 | 77 | 104 | 21 | 0.2 |

### Body and Organ Weights

Body weights, as well as liver, heart, lungs, spleen and kidney weights were measured at the end of the study, and are presented in Table 32. MOE′ indicates that the antisense oligonucleotide is a MOE gapmer. ‘DMC’ indicates that the antisense oligonucleotide comprises deoxy, cEt and MOE modifications. Treatment with all the antisense oligonucleotides was tolerable in terms of body and organ weights in this model.

TABLE 32
Body and organ weights (grams) of Sprague-Dawley rats

| | | Dose | | | | |
|---|---|---|---|---|---|---|
| | | (mg/ | Body | | | |
| | Chemistry | kg/wk) | weight | Liver | Spleen | Kidney |
| PBS | | — | 443 | 16 | 0.8 | 3.5 |
| ISIS 407935 | MOE | 100 | 337 | 14 | 1.8 | 3.2 |
| ISIS 490279 | MOE | 100 | 365 | 18 | 2.2 | 2.9 |
| ISIS 473589 | DMC | 50 | 432 | 18 | 1.3 | 3.3 |
| ISIS 529804 | DMC | 50 | 429 | 18 | 2.2 | 3.4 |
| ISIS 534796 | DMC | 50 | 434 | 15 | 1.4 | 3.3 |
| ISIS 540162 | DMC | 50 | 446 | 18 | 1.1 | 3.3 |
| ISIS 540175 | DMC | 50 | 467 | 16 | 1.0 | 3.5 |
| ISIS 540182 | DMC | 50 | 447 | 22 | 2.5 | 4.5 |
| ISIS 540191 | DMC | 50 | 471 | 21 | 1.4 | 3.9 |

### Example 36

### Dose-Dependent Antisense Inhibition of Human Factor VII in Cynomolgos Monkey Primary Hepatocytes

Antisense oligonucleotides selected from the studies described above were tested at various doses in cynomolgous monkey primary hepatocytes. Cells were plated at a density of 35,000 cells per well and transfected using electroporation with 0.009 μM, 0.03 μM, 0.08 μM, 0.25 μM, 0.74 μM, 2.22 μM, 6.67 μM, and 20.00 μM concentrations of antisense oligonucleotide, as specified in Table 33. After a treatment period of approximately 16 hours, RNA was isolated from the cells and Factor VII mRNA levels were measured by quantitative real-time PCR. Factor VII primer probe set RTS2927 was used to measure mRNA levels. Factor VII mRNA levels were adjusted according to total RNA content, as measured by RIBOGREEN®. Results are presented as percent inhibition of Factor VII expression, relative to untreated control cells. As illustrated in Table 33, Factor VII mRNA levels were significantly reduced in a dose-dependent manner with some of the antisense oligonucleotides that are cross-reactive with the rhesus monkey genomic sequence (GENBANK Accession No. NW_{—}001104507.1 truncated from nucleotides 691000 to 706000; SEQ ID NO: 4). ‘n/a.’ indicates that the antisense oligonucleotide has more than 3 mismatches with SEQ ID NO: 4.

TABLE 33
Dose-dependent antisense inhibition (%) of Factor VII in
cynomolgous monkey primary hepatocytes using electroporation

| | Start | | | | | | | | |
|---|---|---|---|---|---|---|---|---|---|
| | Site on | | | | | | | | |
| | SEQ | | | | | | | | |
| | ID | 0.009 | 0.03 | 0.08 | 0.2 | 0.74 | 2.22 | 6.67 | 20.00 |
| ISIS No | NO: 4 | μM | μM | μM | μM | μM | μM | μM | μM |
| 490279 | 808 | 19 | 12 | 13 | 0 | 6 | 18 | 27 | 22 |
| 473589 | 12845 | 5 | 10 | 19 | 42 | 64 | 76 | 88 | 92 |
| 529804 | 12909 | 10 | 3 | 23 | 25 | 57 | 80 | 86 | 91 |
| 534796 | 12848 | 0 | 28 | 23 | 49 | 71 | 81 | 87 | 90 |
| 540162 | 2358 | 9 | 14 | 9 | 6 | 13 | 13 | 11 | 31 |
| 540175 | 2051 | 0 | 4 | 12 | 9 | 10 | 16 | 12 | 22 |
| | 2285 | | | | | | | | |
| 540182 | n/a | 0 | 7 | 0 | 6 | 36 | 12 | 10 | 0 |
| 540191 | n/a | 6 | 7 | 0 | 0 | 0 | 0 | 21 | 42 |
| 407935 | n/a | 10 | 18 | 15 | 29 | 56 | 73 | 82 | 88 |

### Example 37

### Dose-Dependent Antisense Inhibition of Human Factor VII in Hep3B Cells

Antisense oligonucleotides from the study described above were also tested at various doses in Hep3B cells. Cells were plated at a density of 20,000 cells per well and transfected using electroporation with 0.009 μM, 0.03 μM, 0.08 μM, 0.25 μM, 0.74 μM, 2.22 μM, 6.67 μM, and 20.00 μM concentrations of antisense oligonucleotide, as specified in Table 34. After a treatment period of approximately 16 hours, RNA was isolated from the cells and Factor VII mRNA levels were measured by quantitative real-time PCR. Factor VII primer probe set RTS2927 was used to measure mRNA levels. Factor VII mRNA levels were adjusted according to total RNA content, as measured by RIBOGREEN®. Results are presented as percent inhibition of Factor VII expression, relative to untreated control cells. As illustrated in Table 34, Factor VII mRNA levels were significantly reduced in a dose-dependent manner with several of the antisense oligonucleotides.

TABLE 34
Dose-dependent antisense inhibition (%)
of Factor VII in Hep3B cells using electroporation

| | 0.009 | 0.03 | 0.08 | 0.25 | 0.74 | 2.22 | 6.67 | 20.00 | IC₅₀ |
|---|---|---|---|---|---|---|---|---|---|
| ISIS No | μM | μM | μM | μM | μM | μM | μM | μM | (μM) |
| 407935 | 3 | 9 | 11 | 35 | 64 | 83 | 87 | 93 | 4.5 |
| 473244 | 20 | 33 | 50 | 69 | 77 | 89 | 7 | 14 | 0.9 |
| 473589 | 0 | 14 | 23 | 44 | 74 | 88 | 90 | 94 | 2.7 |
| 490279 | 0 | 5 | 7 | 15 | 25 | 61 | 76 | 78 | 11.6 |
| 515952 | 0 | 12 | 27 | 57 | 76 | 89 | 93 | 94 | 2.2 |
| 516066 | 6 | 0 | 12 | 26 | 52 | 70 | 81 | 86 | 6.0 |
| 529459 | 0 | 4 | 24 | 40 | 61 | 78 | 88 | 94 | 3.5 |
| 529553 | 9 | 7 | 17 | 40 | 58 | 74 | 87 | 93 | 4.6 |
| 529804 | 0 | 3 | 34 | 64 | 83 | 89 | 93 | 95 | 2.0 |
| 534796 | 8 | 18 | 43 | 67 | 82 | 89 | 95 | 96 | 1.4 |
| 537806 | 6 | 11 | 5 | 20 | 37 | 69 | 79 | 86 | 7.1 |
| 540162 | 18 | 33 | 63 | 75 | 87 | 91 | 91 | 92 | 0.7 |
| 540175 | 10 | 25 | 55 | 76 | 86 | 89 | 89 | 93 | 1.0 |
| 540182 | 13 | 36 | 61 | 75 | 84 | 88 | 90 | 93 | 0.7 |
| 540191 | 3 | 12 | 28 | 61 | 79 | 80 | 88 | 94 | 2.2 |

### Example 38

### Efficacy of Antisense Oligonucleotides Targeting Human Factor VII in Transgenic Mice

Transgenic mice were treated with ISIS antisense oligonucleotides selected from studies described above and evaluated for efficacy.

### Treatment

Eight groups of 3 transgenic mice each were injected subcutaneously twice a week for 3 weeks with 10 mg/kg, 5 mg/kg, 2.5 mg/kg, or 1.25 mg/kg of ISIS 407935 or ISIS 490279. Another 24 groups of 3 transgenic mice each were subcutaneously twice a week for 3 weeks with 2.5 mg/kg, 1.25 mg/kg, 0.625 mg/kg, or 0.313 mg/kg of ISIS 473589, ISIS 529804, ISIS 534796, ISIS 540162, ISIS 540175, or ISIS 540191. One group of mice was injected subcutaneously twice a week for 3 weeks with PBS. Mice were euthanized 48 hours after the last dose, and organs and plasma were harvested for further analysis.

### RNA Analysis

RNA was extracted from plasma for real-time PCR analysis of Factor VII, using primer probe set RTS2927. The mRNA levels were normalized using RIBOGREEN®. As shown in Table 35, several antisense oligonucleotides achieved significant reduction of human Factor VII over the PBS control. Results are presented as percent inhibition of Factor VII, relative to control. MOE′ indicates that the antisense oligonucleotide is a MOE gapmer. ‘DMC’ indicates that the antisense oligonucleotide comprises deoxy, cEt and MOE modifications. Treatment with newly designed MOE gapmer, ISIS 490279, caused greater reduction in human Factor VII mRNA levels than treatment with ISIS 407935, the MOE gapmer from the earlier publication. Treatment with several of the newly designed DMC oligonucleotides also caused greater reduction in human Factor VII mRNA levels than treatment with ISIS 407935.

TABLE 35
Percent inhibition of Factor VII mRNA in transgenic mice

| | | Dose | % |
|---|---|---|---|
| ISIS No | Chemistry | (mg/kg/wk) | inhibition |
| 407935 | MOE | 20.0 | 85 |
| | | 10.0 | 57 |
| | | 5.0 | 45 |
| | | 2.5 | 28 |
| 490279 | MOE | 20.0 | 88 |
| | | 10.0 | 70 |
| | | 5.0 | 51 |
| | | 2.5 | 33 |
| 473589 | DMC | 5.00 | 80 |
| | | 2.50 | 62 |
| | | 1.25 | 44 |
| | | 0.625 | 25 |
| 529804 | DMC | 5.00 | 55 |
| | | 2.50 | 41 |
| | | 1.25 | 0 |
| | | 0.625 | 1 |
| 534796 | DMC | 5.00 | 56 |
| | | 2.50 | 41 |
| | | 1.25 | 5 |
| | | 0.625 | 0 |
| 540162 | DMC | 5.00 | 97 |
| | | 2.50 | 92 |
| | | 1.25 | 69 |
| | | 0.625 | 78 |
| 540175 | DMC | 5.00 | 95 |
| | | 2.50 | 85 |
| | | 1.25 | 65 |
| | | 0.625 | 55 |
| 540182 | DMC | 5.00 | 97 |
| | | 2.50 | 83 |
| | | 1.25 | 54 |
| | | 0.625 | 10 |
| 540191 | DMC | 5.00 | 91 |
| | | 2.50 | 74 |
| | | 1.25 | 58 |
| | | 0.625 | 34 |

### Protein Analysis

Plasma protein levels of Factor VII were estimated using a Zymutest FVII ELISA kit (Hyphen Bio-Med cat#ARK036A). As shown in Table 36, several antisense oligonucleotides achieved significant reduction of human Factor VII over the PBS control. Results are presented as percent inhibition of Factor VII, relative to control. MOE′ indicates that the antisense oligonucleotide is a MOE gapmer. ‘DMC’ indicates that the antisense oligonucleotide comprises deoxy, cEt and MOE modifications. Treatment with newly designed MOE gapmer, ISIS 490279, caused greater reduction in human Factor VII protein levels than treatment with ISIS 407935, the MOE gapmer from the earlier publication. Treatment with several of the newly designed DMC oligonucleotides also caused greater reduction in human Factor VII protein levels than treatment with ISIS 407935.

TABLE 36
Percent inhibition of Factor VII plasm protein levels
in transgenic mice

| | | Dose | % |
|---|---|---|---|
| ISIS No | Chemistry | (mg/kg/wk) | inhibition |
| 407935 | MOE | 20 | 65 |
| | | 10 | 47 |
| | | 5 | 0 |
| | | 2.5 | 3 |
| 490279 | MOE | 20 | 91 |
| | | 10 | 75 |
| | | 5 | 31 |
| | | 2.5 | 23 |
| 473589 | DMC | 5 | 78 |
| | | 2.5 | 40 |
| | | 1.25 | 6 |
| | | 0.625 | 0 |
| 529804 | DMC | 5 | 50 |
| | | 2.5 | 36 |
| | | 1.25 | 0 |
| | | 0.625 | 8 |
| 534796 | DMC | 5 | 45 |
| | | 2.5 | 26 |
| | | 1.25 | 0 |
| | | 0.625 | 8 |
| 540162 | DMC | 5 | 98 |
| | | 2.5 | 96 |
| | | 1.25 | 78 |
| | | 0.625 | 74 |
| 540175 | DMC | 5 | 93 |
| | | 2.5 | 83 |
| | | 1.25 | 49 |
| | | 0.625 | 24 |
| 540182 | DMC | 5 | 97 |
| | | 2.5 | 71 |
| | | 1.25 | 50 |
| | | 0.625 | 0 |
| 540191 | DMC | 5 | 97 |
| | | 2.5 | 74 |
| | | 1.25 | 46 |
| | | 0.625 | 25 |

### Example 39

### Effect of ISIS Antisense Oligonucleotides Targeting Human Factor VII in Cynomolgus Monkeys

Cynomolgus monkeys were treated with ISIS antisense oligonucleotides selected from studies described above, including ISIS 407935, ISIS 490279, ISIS 473589, ISIS 529804, ISIS 534796, ISIS 540162, ISIS 540175, ISIS 540182, and ISIS 540191. Antisense oligonucleotide efficacy and tolerability were evaluated. ISIS 407935, from the earlier publication, was included in the study for comparison. The antisense oligonucleotides tested in the study are presented in Table 37. The ‘Sugar Chemistry’ column provides the sugar modifications throughout each oligonucleotide: ‘d’ indicates a 2′-deoxynucleoside, ‘k’ indicates a constrained ethyl (cEt) nucleoside, and ‘e’ indicates a 2′-O-methoxyethyl nucleoside. The ‘Sequence’ column provides the nucleobase sequence for each SEQ ID NO. Some of the human antisense oligonucleotides tested are also cross-reactive with the rhesus genomic sequence (GENBANK Accession No. NW_{—}001104507.1 truncated from nucleotides 691000 to 706000, designated herein as SEQ ID NO: 4). The greater the complementarity between the human oligonucleotide and the rhesus monkey sequence, the more likely the human oligonucleotide can cross-react with the rhesus monkey sequence. ‘Mismatches’ indicate the number of nucleotides between the human oligonucleotide and the rhesus monkey sequence that are mismatched. Mismatches of more than 3 have not been shown. “Start site” indicates the 5′-most nucleotide to which the oligonucleotide is targeted in the rhesus monkey gene sequence.

TABLE 37
Antisense oligonucleotides selected for the cynomolgous monkey study

| | | | SEQ | | |
|---|---|---|---|---|---|
| Start Site | Mismatches | Sequence | ID NO | ISIS No | Sugar Chemistry |
| 12908 | 0 | ATGCATGGTGATGCTTCTGA | 120 | 407935 | eeeeeddddddddddeeeee |
| 12845 | 0 | GCTAAACAACCGCCTT | 59 | 473589 | kdkdkdddddddddee |
| 808 | 0 | CCCTCCTGTGCCTGGATGCT | 93 | 490279 | eeeeeddddddddddeeeee |
| 12909 | 0 | CATGGTGATGCTTCTG | 259 | 529804 | kddddddddddkekee |
| 12848 | 0 | AGAGCTAAACAACCGC | 254 | 534796 | ekkddddddddddkke |
| 2041 | 2 | ACTCCCGGGACACCCA | 624 | 540162 | eekddddddddddkke |
| 2058 | 1 | | | | |
| 2075 | 3 | | | | |
| 2108 | 1 | | | | |
| 2125 | 3 | | | | |
| 2142 | 3 | | | | |
| 2159 | 3 | | | | |
| 2175 | 3 | | | | |
| 2191 | 3 | | | | |
| 2208 | 1 | | | | |
| 2225 | 3 | | | | |
| 2258 | 2 | | | | |
| 2292 | 1 | | | | |
| 2309 | 1 | | | | |
| 2324 | 1 | | | | |
| 2358 | 0 | | | | |
| 2358 | 0 | GGACACCCACGCCCCC | 637 | 540175 | eekddddddddddkke |
| 2017 | 2 | | | | |
| 2051 | 0 | | | | |
| 2068 | 3 | | | | |
| 2085 | 1 | | | | |
| 2101 | 3 | | | | |
| 2118 | 3 | | | | |
| 2135 | 1 | | | | |
| 2152 | 2 | | | | |
| 2168 | 1 | | | | |
| 2184 | 1 | | | | |
| 2201 | 1 | | | | |
| 2218 | 3 | | | | |
| 2234 | 3 | | | | |
| 2251 | 1 | | | | |
| 2268 | 2 | | | | |
| 2285 | 0 | | | | |
| 2302 | 1 | | | | |
| 2334 | 3 | | | | |
| 2351 | 1 | | | | |
| 2368 | 3 | | | | |
| 2049 | 2 | ACACCCTCGCCTCCGG | 644 | 540182 | eekddddddddddkke |
| 2133 | 3 | | | | |
| 2150 | 2 | | | | |
| 2166 | 3 | | | | |
| 2182 | 3 | | | | |
| 2199 | 3 | | | | |
| 2216 | 3 | | | | |
| 2266 | 3 | | | | |
| 2300 | 3 | | | | |
| 2041 | 2 | GCCTCCGGAACACCCA | 653 | 540191 | eekddddddddddkke |
| 2075 | 3 | | | | |
| 2125 | 3 | | | | |
| 2142 | 1 | | | | |
| 2191 | 3 | | | | |
| 2208 | 3 | | | | |
| 2258 | 2 | | | | |
| 2292 | 3 | | | | |
| 2309 | 3 | | | | |

### Treatment

Prior to the study, the monkeys were kept in quarantine for at least a 30-day period, during which the animals were observed daily for general health. Standard panels of serum chemistry and hematology, examination of fecal samples for ova and parasites, and a tuberculosis test were conducted immediately after the animals' arrival to the quarantine area. The monkeys were 2-4 years old at the start of treatment and weighed between 2 and 4 kg. Ten groups of four randomly assigned male cynomolgus monkeys each were injected subcutaneously with ISIS oligonucleotide or PBS using a stainless steel dosing needle and syringe of appropriate size into one of 4 sites on the back of the monkeys; each site used in clock-wise rotation per dose administered. Nine groups of monkeys were dosed four times a week for the first week (days 1, 3, 5, and 7) as loading doses, and subsequently once a week for weeks 2-12, with 35 mg/kg of ISIS 407935, ISIS 490279, ISIS 473589, ISIS 529804, ISIS 534796, ISIS 540162, ISIS 540175, ISIS 540182, or ISIS 540191. A control group of cynomolgus monkeys was injected with PBS subcutaneously thrice four times a week for the first week (days 1, 3, 5, and 7), and subsequently once a week for weeks 2-12.

During the study period, the monkeys were observed twice daily for signs of illness or distress. Any animal experiencing more than momentary or slight pain or distress due to the treatment, injury or illness was treated by the veterinary staff with approved analgesics or agents to relieve the pain after consultation with the Study Director. Any animal in poor health or in a possible moribund condition was identified for further monitoring and possible euthanasia. Terminal sacrifice was performed on day 86, approximately 48 hours after the final dosing on day 84. The protocols described in the Example were approved by the Institutional Animal Care and Use Committee (IACUC).

### Necroscopy

For terminal necroscopy on day 86, approximately 48 hours after the final dose, the animals were euthanized by exsanguination while under deep anesthesia. A full macroscopic examination was performed under the general supervision of a pathologist and all lesions were recorded. Of note, treatment with ISIS 407935 was observed to result in ascites in 2 out of 4 monkeys suggesting it is less well tolerated than the other compounds in the study. Specifically, compounds ISIS Nos: 490279, 473589, 540162, 534796, and 540175 did not show any of these findings.

### Hepatic Target Reduction

### RNA Analysis

On day 86, RNA was extracted from liver tissue for real-time PCR analysis of Factor VII using primer probe set RTS2927. Results are presented as percent inhibition of Factor VII mRNA, relative to PBS control, normalized to RIBOGREEN® or to the house keeping gene, GAPDH. As shown in Table 38, treatment with ISIS antisense oligonucleotides resulted in significant reduction of Factor VII mRNA in comparison to the PBS control.

TABLE 38
Percent Inhibition of cynomolgous monkey Factor VII mRNA
in the cynomolgus monkey liver relative to the PBS control

| ISIS No | RTS2927/Ribogreen | RTS2927/GAPDH |
|---|---|---|
| 407935 | 90 | 90 |
| 490279 | 72 | 66 |
| 473589 | 96 | 96 |
| 529804 | 90 | 87 |
| 534796 | 80 | 78 |
| 540162 | 66 | 58 |
| 540175 | 68 | 66 |
| 540182 | 0 | 0 |
| 540191 | 34 | 14 |

### Protein Levels and Activity Analysis

Plasma Factor VII levels were measured prior to dosing, and on day 3, day 5, day 7, day 16, day 30, day 44, day 65, and day 86 of treatment. Factor VII activity was measured using Factor VII deficient plasma. Approximately 1.5 mL of blood was collected from all available study animals into tubes containing 3.2% sodium citrate. The samples were placed on ice immediately after collection. Collected blood samples were processed to platelet poor plasma and the tubes were centrifuged at 3,000 rpm for 10 min at 4° C. to obtain plasma.

Protein levels of Factor VII were measured by a ZYMUTEST Factor VII elisa kit from Hyphen Bio-Med (cat#RK036A). The results are presented in Table 39. To measure Factor VII activity, 60 μL of sample plasma was diluted 1/20 in factor diluents buffer and then incubated with 60 μL of PT reagent (PT-Fibronogen HS, Instrumentation Laboratory Company, USA) and 60 μL of citrated human plasma deficient of Factor VII (George King Bio-Medical Inc., USA) at 37° C. for 5 min. Factor VII activity was then determined with ACL-9000 (Instrumentation Laboratory, Italy). The results, in seconds, for Factor VII activity was interpolated on a standard curve of serial dilutions from normal pooled monkey plasma. The results are presented in Table 40, expressed as a percentage reduction compared to the baseline values.

TABLE 39
Plasma Factor VII protein levels (% reduction compared
to the baseline) in the cynomolgus monkey plasma

| | Day | Day | Day | Day | Day | | | |
|---|---|---|---|---|---|---|---|---|
| ISIS No | 3 | 5 | 7 | 16 | 30 | Day 44 | Day 65 | Day 86 |
| 407935 | 21 | 62 | 69 | 82 | 84 | 85 | 84 | 90 |
| 490279 | 0 | 29 | 35 | 30 | 38 | 45 | 51 | 58 |
| 473589 | 12 | 67 | 85 | 97 | 98 | 98 | 98 | 98 |
| 529804 | 19 | 65 | 76 | 87 | 88 | 89 | 90 | 90 |
| 534796 | 1 | 46 | 54 | 64 | 64 | 67 | 66 | 70 |
| 540162 | 0 | 24 | 26 | 37 | 45 | 49 | 49 | 50 |
| 540175 | 0 | 28 | 36 | 38 | 47 | 52 | 55 | 55 |
| 540182 | 0 | 17 | 8 | 0 | 0 | 0 | 5 | 0 |
| 540191 | 0 | 12 | 4 | 0 | 0 | 4 | 9 | 10 |

TABLE 40
Plasma Factor VII activity levels (% reduction compared
to the baseline) in the cynomolgus monkey plasma

| | Day | Day | Day | Day | Day | | | |
|---|---|---|---|---|---|---|---|---|
| ISIS No | 3 | 5 | 7 | 16 | 30 | Day 44 | Day 65 | Day 86 |
| 407935 | 25 | 76 | 80 | 90 | 91 | 87 | 89 | 92 |
| 490279 | 0 | 8 | 4 | 31 | 40 | 57 | 56 | 66 |
| 473589 | 21 | 78 | 86 | 98 | 97 | 98 | 98 | 98 |
| 529804 | 25 | 69 | 81 | 93 | 87 | 92 | 93 | 93 |
| 534796 | 5 | 47 | 63 | 76 | 65 | 76 | 74 | 76 |
| 540162 | 0 | 0 | 7 | 30 | 26 | 50 | 49 | 51 |
| 540175 | 0 | 16 | 36 | 44 | 50 | 67 | 60 | 63 |
| 540182 | 0 | 0 | 12 | 5 | 24 | 15 | 0 | 4 |
| 540191 | 0 | 13 | 17 | 19 | 30 | 61 | 28 | 32 |

### Tolerability Studies

### Body and Organ Weight Measurement

To evaluate the effect of ISIS oligonucleotides on the overall health of the animals, body and organ weights were measured on different days. The data is presented in Table 41. The results indicate that effect of treatment with antisense oligonucleotides on body weights was within the normal range. However, treatment with ISIS 407935 resulted in a 2.2-fold increase in spleen weight, a 2.7-fold increase in liver weight, and a 1.3-fold increase in kidney weight compared to the control, indicating that ISIS 407935 had an effect on organ weights, which was not observed with the newly designed antisense oligonucleotides.

TABLE 41
Final body weights (grams) in the cynomolgus
monkey relative to pre-dose levels

| | Day | | | | | | |
|---|---|---|---|---|---|---|---|
| | 1 | Day 7 | Day 21 | Day 28 | Day 42 | Day 63 | Day 84 |
| PBS | 2651 | 2634 | 2672 | 2685 | 2694 | 2755 | 2767 |
| ISIS | 2567 | 2506 | 2536 | 2548 | 2545 | 2528 | 2537 |
| 407935 | | | | | | | |
| ISIS | 2597 | 2566 | 2597 | 2635 | 2713 | 2765 | 2850 |
| 490279 | | | | | | | |
| ISIS | 2606 | 2618 | 2656 | 2657 | 2692 | 2734 | 2777 |
| 473589 | | | | | | | |
| ISIS | 2597 | 2580 | 2590 | 2627 | 2651 | 2657 | 2734 |
| 529804 | | | | | | | |
| ISIS | 2569 | 2596 | 2628 | 2622 | 2666 | 2738 | 2789 |
| 534796 | | | | | | | |
| ISIS | 2715 | 2747 | 2743 | 2755 | 2799 | 2758 | 2934 |
| 540162 | | | | | | | |
| ISIS | 2644 | 2678 | 2675 | 2687 | 2720 | 2760 | 2812 |
| 540175 | | | | | | | |
| ISIS | 2517 | 2529 | 2528 | 2533 | 2674 | 2716 | 2790 |
| 540182 | | | | | | | |
| ISIS | 2590 | 2598 | 2661 | 2686 | 2750 | 2833 | 2938 |
| 540191 | | | | | | | |

### Serum Chemistry Markers

To evaluate the effect of ISIS oligonucleotides on serum chemistry markers, the monkeys were fasted overnight prior to blood collection. Approximately 1.5 mL of blood was collected into tubes without anticoagulant for serum separation. The tubes were kept at room temperature for a minimum of 90 min and then centrifuged at 3,000 rpm for 10 min at room temperature. Serum levels of various markers were measured on day 44 using a Toshiba 200FR NEO chemistry analyzer (Toshiba Co. Japan). Levels of ALT and AST were measured, and the results are presented in Table 42, expressed in IU/L. Serum creatinine, and BUN were similarly measured and also presented in Table 42, expressed in mg/dL. Serum C-reactive protein (CRP) was also similarly measured and is presented in Table 42, expressed as mg/L. Serum albumin was also similarly measured and is presented in Table 42, expressed in g/dL. In monkeys treated with ISIS 407935, there was an elevation in serum BUN, CRP, and creatinine levels, indicating the treatment with ISIS 407935 may have produced deleterious effects on kidney function and an acute stress response. Treatment with the newly designed oligonucleotides produced no changes within these parameters suggesting they have a more favorable safety profile than treatment with ISIS 407935.

TABLE 42
Effect of antisense oligonucleotide treatment on
liver function markers in cynomolgus monkey plasma

| | ALT | AST | Creatinine | BUN | Albumin | CRP |
|---|---|---|---|---|---|---|
| | (IU/L) | (IU/L) | (mg/dL) | (mg/dL) | (g/dL) | (mg/L) |
| PBS | 51 | 61 | 0.9 | 28 | 4.5 | 1.6 |
| 407935 | 52 | 64 | 1.5 | 52 | 4.0 | 5.2 |
| 490279 | 100 | 60 | 1.0 | 23 | 4.7 | 2.3 |
| 473589 | 55 | 52 | 1.0 | 24 | 4.8 | 2.7 |
| 529804 | 48 | 46 | 1.0 | 28 | 4.5 | 2.1 |
| 534796 | 40 | 57 | 1.0 | 30 | 4.5 | 1.6 |
| 540162 | 45 | 55 | 1.1 | 25 | 4.7 | 1.3 |
| 540175 | 46 | 44 | 0.9 | 21 | 4.7 | 1.1 |
| 540182 | 123 | 129 | 0.9 | 28 | 4.4 | 1.5 |
| 540191 | 36 | 41 | 1.0 | 24 | 4.7 | 1.6 |

### Urine Chemistry Markers

To evaluate the effect of ISIS oligonucleotides on kidney function, fresh urine from all animals was collected for urinalysis using a clean cage pan on ice. Food was removed overnight the day before urine collection but water was supplied. Levels of creatinine and total urine protein were measured on day 86 using a Toshiba 200FR NEO chemistry analyzer (Toshiba Co., Japan). The ratio of total urine protein to creatine was then calculated and the results are presented in Table 43.

The data indicate that most of the newly designed ISIS oligonucleotides did not have any effect on the kidney function outside the expected range. However, treatment with ISIS 407935 resulted in elevated urine protein to creatinine ratio in the monkeys, indicating treatment with ISIS 407935 perturbed kidney function. Hence, treatment with the newly designed oligonucleotides was more tolerable than treatment with ISIS 407935.

TABLE 43
Total urine protein to creatinine ratio in cynomolgus monkeys

| | Protein/creatinine |
|---|---|
| | ratio |
| PBS | 0.03 |
| ISIS 407935 | 0.64 |
| ISIS 490279 | 0.00 |
| ISIS 473589 | 0.01 |
| ISIS 529804 | 0.00 |
| ISIS 534796 | 0.00 |
| ISIS 540162 | 0.01 |
| ISIS 540175 | 0.00 |
| ISIS 540182 | 0.04 |
| ISIS 540191 | 0.26 |

### Complement C3 Analysis

To evaluate any effect of ISIS oligonucleotides on complement C3 levels, approximately 0.5 mL of blood was collected into tubes without anticoagulant for serum separation. The tubes were kept at room temperature for a minimum of 90 min and then centrifuged at 3,000 rpm for 10 min at room temperature to obtain serum. Complement C3 was measured at week 1, 24 hours after dosing, using a Toshiba 200FR NEO chemistry analyzer (Toshiba Co., Japan). The data is presented in Table 44, expressed in mg/dL. Treatment with ISIS 407935 resulted in reduced complement C3 levels, indicating treatment with ISIS 407935 may have resulted in repeated complement activation to a greater degree than with the newly designed oligonucleotides.

TABLE 44
Complement C3 levels in cynomolgus monkeys

| | mg/dL |
|---|---|
| PBS | 146 |
| ISIS 407935 | 92 |
| ISIS 490279 | 124 |
| ISIS 473589 | 140 |
| ISIS 529804 | 137 |
| ISIS 534796 | 137 |
| ISIS 540162 | 135 |
| ISIS 540175 | 121 |
| ISIS 540182 | 104 |
| ISIS 540191 | 141 |

### Hematology

To evaluate any effect of ISIS oligonucleotides in cynomolgus monkeys on hematologic parameters, blood samples of approximately 0.5 mL of blood was collected on day 44 from each of the available study animals in tubes containing K₂-EDTA. Samples were analyzed for red blood cell (RBC) count, as well as for platelet count, using an ADVIA120 hematology analyzer (Bayer, USA). The data is presented in Table 45.

TABLE 45
Complement C3 levels in cynomolgus monkeys

| | Platelet count | RBC count |
|---|---|---|
| | (×10³/μL) | (×10⁶/μL) |
| PBS | 378 | 6.0 |
| ISIS 407935 | 367 | 5.8 |
| ISIS 490279 | 457 | 6.0 |
| ISIS 473589 | 472 | 5.9 |
| ISIS 529804 | 343 | 5.7 |
| ISIS 534796 | 473 | 5.8 |
| ISIS 540162 | 379 | 5.9 |
| ISIS 540175 | 445 | 5.9 |
| ISIS 540182 | 481 | 5.7 |
| ISIS 540191 | 528 | 5.9 |

### Coagulation

To evaluate any effect of ISIS oligonucleotides on the coagulation cascade, blood samples of approximately 1.0 mL of blood was collected on day 44 from each of the available study animals in tubes containing 3.2% sodium citrate. Plasma samples were obtained after centrifugation at 3,000 rpm for 10 min at room temperature. PT and aPTT were measured using an ACL 9000 coagulation analyzer (Instrumentation Laboratory, Italy). The data is presented in Table 46.

Treatment with ISIS 407935, ISIS 473589 and ISIS 529804 caused an increase in PT, which is an expected outcome due to the reduction in Factor VII protein and activity as a result of antisense inhibition.

TABLE 46
PT and aPTT (seconds) in cynomolgus monkeys

| | PT | aPTT |
|---|---|---|
| PBS | 10.05 | 19.48 |
| ISIS 407935 | 13.05 | 49.73 |
| ISIS 490279 | 10.15 | 19.73 |
| ISIS 473589 | 21.33 | 18.38 |
| ISIS 529804 | 13.88 | 18.43 |
| ISIS 534796 | 11.10 | 18.23 |
| ISIS 540162 | 10.75 | 18.00 |
| ISIS 540175 | 10.50 | 19.05 |
| ISIS 540182 | 10.60 | 22.00 |
| ISIS 540191 | 10.93 | 19.30 |

### Example 40

### Dose-Dependent Antisense Inhibition of Human Factor VII in HepG2 Cells

Antisense oligonucleotides (from Example 37) were tested at various doses in HepG2 cells. Cells were plated at a density of 20,000 cells μM per well and transfected using electroporation with 0.003 μM, 0.016 μM, 0.800 μM, 4.000 μM, and 20.000 μM concentrations of antisense oligonucleotide, as specified in Table 47. After a treatment period of approximately 16 hours, RNA was isolated from the cells and Factor VII mRNA levels were measured by quantitative real-time PCR. Factor VII primer probe set RTS2927 was used to measure mRNA levels. Factor VII mRNA levels were adjusted according to total RNA content as measured by RIBOGREEN®. Results are presented as percent inhibition of Factor VII expression relative to untreated control cells. As illustrated in Table 47, Factor VII mRNA levels were significantly reduced in a dose-dependent manner with several of the antisense oligonucleotides.

TABLE 47
Dose-dependent antisense inhibition (%)
of Factor VII in HepG2 cells using electroporation

| | 0.003 | | | | | IC₅₀ |
|---|---|---|---|---|---|---|
| ISIS No | μM | 0.016 μM | 0.800 μM | 4.000 μM | 20.000 μM | (μM) |
| 407935 | 14 | 27 | 70 | 87 | 96 | 0.4 |
| 473589 | 15 | 39 | 72 | 89 | 88 | 0.3 |
| 490279 | 9 | 11 | 47 | 63 | 67 | 2.2 |
| 515533 | 0 | 13 | 53 | 78 | 85 | 1.1 |
| 515952 | 7 | 42 | 78 | 92 | 95 | 0.3 |
| 516066 | 5 | 26 | 45 | 73 | 84 | 1 |
| 529459 | 1 | 12 | 53 | 81 | 79 | 1.1 |
| 529553 | 11 | 13 | 57 | 79 | 91 | 0.8 |
| 529804 | 3 | 36 | 82 | 89 | 92 | 0.4 |
| 534796 | 17 | 46 | 76 | 90 | 87 | 0.3 |
| 537806 | 1 | 9 | 39 | 50 | 70 | 3.5 |
| 540162 | 27 | 59 | 76 | 86 | 93 | 0.1 |
| 540175 | 19 | 61 | 76 | 65 | 90 | 0.2 |
| 540182 | 40 | 66 | 81 | 85 | 89 | 0.04 |
| 540191 | 27 | 50 | 77 | 81 | 93 | 0.2 |

1. A compound comprising a modified oligonucleotide consisting of 12 to 30 linked nucleosides and comprising a nucleobase sequence comprising a portion of at least 8, at least 10, at least 12, at least 14, at least 16, at least 18, at least 19, or at least 20 contiguous nucleobases complementary to an equal length portion of nucleobases 1381 to 1406 of SEQ ID NO: 1, wherein the nucleobase sequence of the modified oligonucleotide is at least 90% complementary to SEQ ID NO: 1.

2. The compound of claim 1, wherein the modified oligonucleotide consists of 15 to 30, 18 to 24, 19 to 22, or 20 linked nucleosides.

3. A compound comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 15128 to 15150 of SEQ ID NO: 1, wherein the nucleobase sequence of the modified oligonucleotide is at least 90% complementary to SEQ ID NO: 1.

4. A compound comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2592 to 2607, 2626 to 2641, 2660 to 2675, 2796 to 2811, 2966 to 2981, 3000 to 3015, 3034 to 3049, 3068 to 3083, 3153 to 3168, 3170 to 3185, 3272 to 3287, 3374 to 3389, 3578 to 3593, 3851 to 3866, 3953 to 3968, 4124 to 4139, 4260 to 4275, 4311 to 4326, 4447 to 4462, and 4532 to 4547 of SEQ ID NO: 1, wherein the nucleobase sequence of the modified oligonucleotide is at least 90% complementary to SEQ ID NO: 1.

5. A compound comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2592 to 2607, 2626 to 2641, 2660 to 2675, 2796 to 2811, 2966 to 2981, 3000 to 3015, 3034 to 3049, 3068 to 3083, 3153 to 3168, 3170 to 3185, 3272 to 3287, 3374 to 3389, 3578 to 3593, 3851 to 3866, 3953 to 3968, 4124 to 4139, 4260 to 4275, 4311 to 4326, 4447 to 4462, or 4532 to 4547 of SEQ ID NO: 1, wherein the nucleobase sequence of the modified oligonucleotide is at least 90% complementary to SEQ ID NO: 1.

6. A compound comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 1387 to 1406, 15128 to 15143, 15192 to 15207, and 15131 to 15146 of SEQ ID NO: 1, wherein the nucleobase sequence of the modified oligonucleotide is at least 90% complementary to SEQ ID NO: 1.

7. A compound comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2692 to 2707, 2760 to 2775, 2862 to 2877, 2930 to 2945, 3117 to 3132, 3338 to 3353, 3440 to 3455, 3508 to 3523, 3542 to 3557, 3628 to 3643, 3662 to 3677, 3781 to 3796, 3815 to 3830, 3917 to 3932, 4190 to 4205, 4224 to 4239, 4377 to 4392, and/or 4411 to 4426 of SEQ ID NO: 1, wherein the nucleobase sequence of the modified oligonucleotide is at least 90% complementary to SEQ ID NO: 1.

8. A compound comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 3109 to 3124, 3194 to 3209, 3330 to 3345, 3432 to 3447, 3500 to 3515, 3534 to 3549, 3620 to 3635, 3654 to 3669, 3773 to 3788, 4182 to 4197, 4216 to 4231, 4369 to 4384, and/or 4403 to 4418 of SEQ ID NO: 1, wherein the nucleobase sequence of the modified oligonucleotide is at least 90% complementary to SEQ ID NO: 1.

9. A compound comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2565 to 2580, 2633 to 2648, 2667 to 2682, 2735 to 2750, 2803 to 2818, 2837 to 2852, 2905 to 2920, 3007 to 3022, 3041 to 3056, 3075 to 3090, 3092 to 3107, 3279 to 3294, 3381 to 3396, 3483 to 3498, 3603 to 3618, 3722 to 3737, 3756 to 3771, 3858 to 3873, 3892 to 3907, 3960 to 3975, 4046 to 4061, 4131 to 4146, 4165 to 4180, 4318 to 4333, and/or 4454 to 4469 of SEQ ID NO: 1, wherein the nucleobase sequence of the modified oligonucleotide is at least 90% complementary to SEQ ID NO: 1.

10. A compound comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 2558 to 4600 of SEQ ID NO: 1, wherein the nucleobase sequence of the modified oligonucleotide is at least 90% complementary to SEQ ID NO: 1.

11. A compound comprising a modified oligonucleotide consisting of 12 to 30 linked nucleosides and comprising a nucleobase sequence comprising a portion of at least 8, at least 10, at least 12, at least 14, at least 15, or at least 16 contiguous nucleobases complementary to an equal length portion of nucleobases 15128 to 15150, 15181 to 15224, 15128 to 15150, 2560 to 2609, 2684 to 2717, or 3103 to 3131 of SEQ ID NO: 1, wherein the nucleobase sequence of the modified oligonucleotide is at least 90% complementary to SEQ ID NO: 1.

12. The compound of any preceding claim, wherein the modified oligonucleotide consists of 13 to 25, 14 to 25, 15 to 25, or 16 linked nucleosides.

13. The compound of any preceding claim, wherein the nucleobase sequence of the modified oligonucleotide is at least 91%, at least 92%, at least 93%, at least 94%, at least 95%, at least 96%, at least 97%, at least 98%, at least 99%, or 100% complementary to SEQ ID NO: 1.

14. A compound comprising a modified oligonucleotide consisting of 12 to 30 linked nucleosides and having a nucleobase sequence comprising at least 12, at least 14, at least 15, or at least 16 contiguous nucleobases of the nucleobase sequence of SEQ ID NO: 59.

15. A compound comprising a modified oligonucleotide consisting of 12 to 30 linked nucleosides and having a nucleobase sequence comprising at least 12, at least 14, at least 16, at least 18, at least 19, or at least 20 contiguous nucleobases of the nucleobase sequence of SEQ ID NO: 93.

16. A compound comprising a modified oligonucleotide consisting of 12 to 30 linked nucleosides and having a nucleobase sequence comprising at least 12, at least 14, at least 15, or at least 16 contiguous nucleobases of the nucleobase sequence of SEQ ID NO: 637.

17. A compound comprising a modified oligonucleotide consisting of 12 to 30 linked nucleosides and having a nucleobase sequence comprising at least 12, at least 14, at least 15, or at least 16 contiguous nucleobases of any of the nucleobase sequences of SEQ ID NO: 59, 93, 259, 254, 624, 637, 644, or 653.

18. A compound comprising a modified oligonucleotide consisting of 12 to 30 linked nucleosides and having a nucleobase sequence comprising at least 12, at least 14, at least 15, or at least 16 contiguous nucleobases of any of the nucleobase sequences of SEQ ID NO: 21-559.

19. The compound of any preceding claim, consisting of a single-stranded modified oligonucleotide.

20. The compound of any preceding claim, wherein at least one internucleoside linkage is a modified internucleoside linkage.

21. The compound of claim 20, wherein each internucleoside linkage is a phosphorothioate internucleoside linkage.

22. The compound of any preceding claim, wherein at least one nucleoside comprises a modified nucleobase.

23. The compound of claim 22, wherein the modified nucleobase is a 5-methylcytosine.

24. The compound of any preceding claim, wherein the modified oligonucleotide comprises at least one modified sugar.

25. The compound of claim 24, wherein the modified sugar is any of a 2′-O-methoxyethyl, a constrained ethyl, or a 3′-fluoro-HNA.

26. The compound of any preceding claim, comprising at least one 2′-O-methoxyethyl nucleoside, a constrained ethyl nucleoside, or a 3′-fluoro-HNA nucleoside.

27. A compound comprising a modified oligonucleotide according to the following formula:
Gks mCds Tks Ads Aks Ads mCds Ads Ads mCds mCds Gds mCds mCds Tes Te;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

28. A compound consisting of a modified oligonucleotide according to the following formula:
Gks mCds Tks Ads Aks Ads mCds Ads Ads mCds mCds Gds mCds mCds Tes Te;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

29. A compound comprising of a modified oligonucleotide according to the following formula:
mCes mCes mCes Tes mCes mCds Tds Gds Tds Gds mCds mCds Tds Gds Gds Aes Tes Ges mCes Te;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

30. A compound consisting of a modified oligonucleotide according to the following formula:
mCes mCes mCes Tes mCes mCds Tds Gds Tds Gds mCds mCds Tds Gds Gds Aes Tes Ges mCes Te;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

31. A compound comprising of a modified oligonucleotide according to the following formula:
Ges Ges Aks mCds Ads mCds mCds mCds Ads mCds Gds mCds mCds mCks mCks mCe;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

32. A compound consisting of a modified oligonucleotide according to the following formula:
Ges Ges Aks mCds Ads mCds mCds mCds Ads mCds Gds mCds mCds mCks mCks mCe;
wherein,
each nucleobase is indicated according to the following:
A=adenine
T=thymine
G=guanine;
mC=5-methylcytosine; wherein
each sugar moiety is indicated according to the following:
k=cEt;
d=2′-deoxyribose;
e=2′-MOE; wherein
each internucleoside linkage is indicated according to the following:
s=phosphorothioate.

33. A composition comprising a compound according to any of claims 1-32 or a salt thereof and a pharmaceutically acceptable carrier or diluent.

34. A compound according to any of claims 1-32 or a composition according to claim 33, for use in therapy.

35. The compound or composition according to claim 34, for use in treating, preventing, or slowing progression of a thromboembolic complication.

36. The compound or composition according to claim 34, for use in treating, preventing, or slowing progression of a hyperproliferative disorder.

37. The compound or composition according to claim 34, for use in treating, preventing, or slowing progression of an inflammatory condition.
